SEQUENCE LISTING (216 sequences)

SEQ ID NO: 1 - DNA, 20 bp, synthetic construct
  source 1..20: mol_type = other DNA
cactcatgcgagcggctttt

SEQ ID NO: 2 - DNA, 20 bp, synthetic construct
  source 1..20: mol_type = other DNA
gcactcatgcgagcggcttt

SEQ ID NO: 3 - DNA, 20 bp, synthetic construct
  source 1..20: mol_type = other DNA
cgcactcatgcgagcggctt

SEQ ID NO: 4 - DNA, 20 bp, synthetic construct
  source 1..20: mol_type = other DNA
gcgcactcatgcgagcggct

SEQ ID NO: 5 - DNA, 20 bp, synthetic construct
  source 1..20: mol_type = other DNA
ggcgcactcatgcgagcggc

SEQ ID NO: 6 - DNA, 20 bp, synthetic construct
  source 1..20: mol_type = other DNA
gggcgcactcatgcgagcgg

SEQ ID NO: 7 - DNA, 20 bp, synthetic construct
  source 1..20: mol_type = other DNA
agggcgcactcatgcgagcg

SEQ ID NO: 8 - DNA, 20 bp, synthetic construct
  source 1..20: mol_type = other DNA
gagggcgcactcatgcgagc

SEQ ID NO: 9 - DNA, 20 bp, synthetic construct
  source 1..20: mol_type = other DNA
agagggcgcactcatgcgag

SEQ ID NO: 10 - DNA, 20 bp, synthetic construct
  source 1..20: mol_type = other DNA
tagagggcgcactcatgcga

SEQ ID NO: 11 - DNA, 20 bp, synthetic construct
  source 1..20: mol_type = other DNA
gggcagtctgctttgtacga

SEQ ID NO: 12 - DNA, 20 bp, synthetic construct
  source 1..20: mol_type = other DNA
cgatttgcgggcagtctact

SEQ ID NO: 13 - DNA, 20 bp, synthetic construct
  source 1..20: mol_type = other DNA
taccaccggtcgatttgcgg

SEQ ID NO: 14 - DNA, 20 bp, synthetic construct
  source 1..20: mol_type = other DNA
ttgcttcctgggtgctttac

SEQ ID NO: 15 - DNA, 20 bp, synthetic construct
  source 1..20: mol_type = other DNA
gaggtttcttcacccctcca

SEQ ID NO: 16 - DNA, 20 bp, synthetic construct
  source 1..20: mol_type = other DNA
agcaatttctcgcaccaaac

SEQ ID NO: 17 - DNA, 20 bp, synthetic construct
  source 1..20: mol_type = other DNA
ccacagtaccaggcctataa

SEQ ID NO: 18 - DNA, 20 bp, synthetic construct
  source 1..20: mol_type = other DNA
caaccagataggcctcactt

SEQ ID NO: 19 - DNA, 20 bp, synthetic construct
  source 1..20: mol_type = other DNA
gccaatacctgatttctgtc

SEQ ID NO: 20 - DNA, 20 bp, synthetic construct
  source 1..20: mol_type = other DNA
cactcgcaatcatatactta

SEQ ID NO: 21 - DNA, 20 bp, synthetic construct
  source 1..20: mol_type = other DNA
ggaacagcacagaagacaga

SEQ ID NO: 22 - DNA, 20 bp, synthetic construct
  source 1..20: mol_type = other DNA
cttttcctgccacgatcacc

SEQ ID NO: 23 - DNA, 20 bp, synthetic construct
  source 1..20: mol_type = other DNA
cctaccagcattgaaaaatc

SEQ ID NO: 24 - DNA, 20 bp, synthetic construct
  source 1..20: mol_type = other DNA
aattctgagcgacaaacggc

SEQ ID NO: 25 - DNA, 20 bp, synthetic construct
  source 1..20: mol_type = other DNA
tcatgcgagcggcttttgta

SEQ ID NO: 26 - DNA, 20 bp, synthetic construct
  source 1..20: mol_type = other DNA
ctctggaagcgcagatctgt

SEQ ID NO: 27 - DNA, 20 bp, synthetic construct
  source 1..20: mol_type = other DNA
ttacacgtttggcatggata

SEQ ID NO: 28 - DNA, 20 bp, synthetic construct
  source 1..20: mol_type = other DNA
tttcctgttttttaatacct

SEQ ID NO: 29 - DNA, 20 bp, synthetic construct
  source 1..20: mol_type = other DNA
ttcctgttttttaatacctg

SEQ ID NO: 30 - DNA, 20 bp, synthetic construct
  source 1..20: mol_type = other DNA
tcctgttttttaatacctgt

SEQ ID NO: 31 - DNA, 20 bp, synthetic construct
  source 1..20: mol_type = other DNA
cctgttttttaatacctgta

SEQ ID NO: 32 - DNA, 20 bp, synthetic construct
  source 1..20: mol_type = other DNA
ctgttttttaatacctgtaa

SEQ ID NO: 33 - DNA, 20 bp, synthetic construct
  source 1..20: mol_type = other DNA
tgttttttaatacctgtaac

SEQ ID NO: 34 - DNA, 20 bp, synthetic construct
  source 1..20: mol_type = other DNA
gttttttaatacctgtaacg

SEQ ID NO: 35 - DNA, 20 bp, synthetic construct
  source 1..20: mol_type = other DNA
ttttttaatacctgtaacga

SEQ ID NO: 36 - DNA, 20 bp, synthetic construct
  source 1..20: mol_type = other DNA
tttttaatacctgtaacgat

SEQ ID NO: 37 - DNA, 20 bp, synthetic construct
  source 1..20: mol_type = other DNA
ttttaatacctgtaacgatg

SEQ ID NO: 38 - DNA, 20 bp, synthetic construct
  source 1..20: mol_type = other DNA
tttaatacctgtaacgatga

SEQ ID NO: 39 - DNA, 20 bp, synthetic construct
  source 1..20: mol_type = other DNA
ttaatacctgtaacgatgag

SEQ ID NO: 40 - DNA, 20 bp, synthetic construct
  source 1..20: mol_type = other DNA
taatacctgtaacgatgagg

SEQ ID NO: 41 - DNA, 20 bp, synthetic construct
  source 1..20: mol_type = other DNA
aatacctgtaacgatgaggt

SEQ ID NO: 42 - DNA, 20 bp, synthetic construct
  source 1..20: mol_type = other DNA
atacctgtaacgatgaggtt

SEQ ID NO: 43 - DNA, 20 bp, synthetic construct
  source 1..20: mol_type = other DNA
tacctgtaacgatgaggttt

SEQ ID NO: 44 - DNA, 20 bp, synthetic construct
  source 1..20: mol_type = other DNA
acctgtaacgatgaggtttc

SEQ ID NO: 45 - DNA, 20 bp, synthetic construct
  source 1..20: mol_type = other DNA
tagagggcgcactcttgcga

SEQ ID NO: 46 - DNA, 20 bp, synthetic construct
  source 1..20: mol_type = other DNA
tccagtagagggcgcactct

SEQ ID NO: 47 - DNA, 20 bp, synthetic construct
  source 1..20: mol_type = other DNA
acccctccagtagagggcgc

SEQ ID NO: 48 - DNA, 20 bp, synthetic construct
  source 1..20: mol_type = other DNA
tcttcacccctccagtagag

SEQ ID NO: 49 - DNA, 20 bp, synthetic construct
  source 1..20: mol_type = other DNA
aggtttcttcacccctccag

SEQ ID NO: 50 - DNA, 20 bp, synthetic construct
  source 1..20: mol_type = other DNA
cgatgaggtttcttcacccc

SEQ ID NO: 51 - DNA, 20 bp, synthetic construct
  source 1..20: mol_type = other DNA
tgtaacgatgaggtttcttc

SEQ ID NO: 52 - DNA, 20 bp, synthetic construct
  source 1..20: mol_type = other DNA
ccagtagagggcgcactcat

SEQ ID NO: 53 - DNA, 20 bp, synthetic construct
  source 1..20: mol_type = other DNA
tccagtagagggcgcactca

SEQ ID NO: 54 - DNA, 20 bp, synthetic construct
  source 1..20: mol_type = other DNA
ctccagtagagggcgcactc

SEQ ID NO: 55 - DNA, 20 bp, synthetic construct
  source 1..20: mol_type = other DNA
cctccagtagagggcgcact

SEQ ID NO: 56 - DNA, 20 bp, synthetic construct
  source 1..20: mol_type = other DNA
ccctccagtagagggcgcac

SEQ ID NO: 57 - DNA, 20 bp, synthetic construct
  source 1..20: mol_type = other DNA
cccctccagtagagggcgca

SEQ ID NO: 58 - DNA, 20 bp, synthetic construct
  source 1..20: mol_type = other DNA
acccctccagtagagggcgc

SEQ ID NO: 59 - DNA, 20 bp, synthetic construct
  source 1..20: mol_type = other DNA
cagtagagggcgcactcatg

SEQ ID NO: 60 - DNA, 20 bp, synthetic construct
  source 1..20: mol_type = other DNA
agtagagggcgcactcatgc

SEQ ID NO: 61 - DNA, 20 bp, synthetic construct
  source 1..20: mol_type = other DNA
gtagagggcgcactcatgcg

SEQ ID NO: 62 - DNA, 20 bp, synthetic construct
  source 1..20: mol_type = other DNA
tagagggcgcactcatgcga

SEQ ID NO: 63 - DNA, 20 bp, synthetic construct
  source 1..20: mol_type = other DNA
agagggcgcactcatgcgag

SEQ ID NO: 64 - DNA, 20 bp, synthetic construct
  source 1..20: mol_type = other DNA
gagggcgcactcatgcgagc

SEQ ID NO: 65 - DNA, 20 bp, synthetic construct
  source 1..20: mol_type = other DNA
agggcgcactcatgcgagcg

SEQ ID NO: 66 - DNA, 20 bp, synthetic construct
  source 1..20: mol_type = other DNA
gggcgcactcatgcgagcgg

SEQ ID NO: 67 - DNA, 20 bp, synthetic construct
  source 1..20: mol_type = other DNA
ggcgcactcatgcgagcggc

SEQ ID NO: 68 - DNA, 20 bp, synthetic construct
  source 1..20: mol_type = other DNA
gcgcactcatgcgagcggct

SEQ ID NO: 69 - DNA, 20 bp, synthetic construct
  source 1..20: mol_type = other DNA
cgcactcatgcgagcggctt

SEQ ID NO: 70 - DNA, 20 bp, synthetic construct
  source 1..20: mol_type = other DNA
gcactcatgcgagcggcttt

SEQ ID NO: 71 - DNA, 20 bp, synthetic construct
  source 1..20: mol_type = other DNA
cactcatgcgagcggctttt

SEQ ID NO: 72 - DNA, 20 bp, synthetic construct
  source 1..20: mol_type = other DNA
actcatgcgagcggcttttg

SEQ ID NO: 73 - DNA, 20 bp, synthetic construct
  source 1..20: mol_type = other DNA
ctcatgcgagcggcttttgt

SEQ ID NO: 74 - DNA, 20 bp, synthetic construct
  source 1..20: mol_type = other DNA
tcatgcgagcggcttttgta

SEQ ID NO: 75 - DNA, 20 bp, synthetic construct
  source 1..20: mol_type = other DNA
catgcgagcggcttttgtag

SEQ ID NO: 76 - DNA, 20 bp, synthetic construct
  source 1..20: mol_type = other DNA
atgcgagcggcttttgtagc

SEQ ID NO: 77 - DNA, 20 bp, synthetic construct
  source 1..20: mol_type = other DNA
tgcgagcggcttttgtagcc

SEQ ID NO: 78 - DNA, 31 bp, synthetic construct
  source 1..31: mol_type = other DNA
ggatccggcggcgtgtgttgggggatagcct

SEQ ID NO: 79 - DNA, 33 bp, synthetic construct
  source 1..33: mol_type = other DNA
cttgaattctcactgcaaagcaccgatagctgc

SEQ ID NO: 80 - DNA, 20 bp, synthetic construct
  source 1..20: mol_type = other DNA
gtgctggtttttcgctcgtc

SEQ ID NO: 81 - DNA, 20 bp, synthetic construct
  source 1..20: mol_type = other DNA
ctttcgtggccagctgtttg

SEQ ID NO: 82 - DNA, 20 bp, synthetic construct
  source 1..20: mol_type = other DNA
tgaccagtcaacaggggaca

SEQ ID NO: 83 - DNA, 20 bp, synthetic construct
  source 1..20: mol_type = other DNA
tgcctgaccaaggaaagcaa

SEQ ID NO: 84 - DNA, 18 bp, synthetic construct
  source 1..18: mol_type = other DNA
aagcagactgcccgcaaa

SEQ ID NO: 85 - DNA, 18 bp, synthetic construct
  source 1..18: mol_type = other DNA
cgctctggaagcgcagat

SEQ ID NO: 86 - DNA, 128 bp, Homo sapiens
  source 1..128: mol_type = genomic DNA
atggctcgtacaaagcagactgcccgcaaatcgaccggtggtaaagcacccaggaagcaactggctacaaaagccgctcgcaagagtgcgccctctactggaggggtgaagaaacctcatcgttacag

SEQ ID NO: 87 - DNA, 128 bp, synthetic construct
  source 1..128: mol_type = other DNA
atggctcgtacaaagcagactgcccgcaaatcgaccggtggtaaagcacccaggaagcaactggctacaaaagccgctcgcatgagtgcgccctctactggaggggtgaagaaacctcatcgttacag

SEQ ID NO: 88 - DNA, 62 bp, synthetic construct
  source 1..62: mol_type = other DNA
actggctacaaaagccgctcgcatgagtgcgccctctactggaggggtgaagaaacctcatc

SEQ ID NO: 89 - DNA, 10189 bp, Homo sapiens
  source 1..10189: mol_type = genomic DNA
acacattgtttagaggccgacgcgacacagccatcttgcgtcgggggcccgggcccgggaggagagcgagccccgtcccctgatcccggaggccaacaccggcgaggggctgccctgcacgcaggcggccggcccaggagtcttagcggatcaagttgtctacggcggactcgggccggtcccgcaggacccggccgcacttggccgagcgcgcaggcgggaaggaggccgtcggtccgcccctcccgctgccgcagcggccaggtccgacagcgggcgcgggacgctggattcctatttgccagcttcctgctggggagcgttttccaatggggcgcggcgacccggccgaccgcgtcctcctcccccactgccgcccgggtctcctcgggggcgcgcgccggggcccgcgcgggggcagcccctccctcccctccccgccgcggcccgagacgcccgcaggccgggcgttcacccgccgcgcctccgcccgccttcccctccccccacttccctccgggcgccccaaacccgggcttcgggggttcgacgccgtgcgcacacggagcgggatgggagtgcagggccgggggcgggggctggccgggtgcgggcggcagtctcggcgggagccggcggcccctcagcgtgtcttttgtgtttgtacacacacggcgcgaggcggcctggaggagggagcgggcggcgcgcgggggaggggcgagcgcgcgccagcgaacgggcgcgcggggggaagggcgggggcggggcgcgagaggagctatgggggcggggccgagccttccctccattgtgtgtgattggctggcgcgcggcgcgggggcggggcggcgtgtgttgggggatagcctcggtgtcagccatctttcaattgtgttcgcagccgccgccgcgccgccgtcgctctccaacgccagcgccgcctctcgctcgccgagctccagccgaaggagaaggggggtaagtttccccgtctgcccgcttccccggaaccgagccccgcttgccgtcagccccgagccgggccctggcggtgccgggaggggacccatagcccctccggcttagcccacaaactttttggcccagaaatggaggtgaggagcgagtttccctgttccctctggcggcggcggctcccccgtctctcgccgcctcagcccaacagcagcaaccgccgcggcgccgagcctgctctccctcctcctccccgcgcccctggctcctctttcttcggtgaaatcccgcccgccgccccttccccggaccccaaaccttcaccatgacccgcgcgggcctcttaactaccgcccccgggccccagcccccagttttcgagcgggaaaggggtggaaatcgccgccgcttcgcaccctggggtaactcgctttttgctgcctccccacagttgttgctaagcttcaccatcttgtctctcttctctggtcaccgaccatattttttcccccgtttctctcttccccatttcaaaaaagcaagaatttttaaaagagggacgtttttttcccttttttggagaggcggaaacttgatgcatttgaaatgcaaaaaaaacttttgcattttggagggcggcacgtggcaggggataggggtattttcgagcgatttggtgttttgtctctttggaacggaatcggatacgttttgcgattgtttccattttgttctggtcggggagccgagttggttcctgctcgaggcgctgggagcgaggcgggcaggttgggggaccctgtgccgagggacccgaaggaggcaaaaccaaaagtttatgtggtgcttggggtggaccggtcggcggatgtcgtgcgttaattaaagcctgggtgcggggcactttttatttcactgcgaagcctgtgaggactggacgcgaggacggggctttgtagaatgctcgctggtggtagctgttgttcctctcttgttttttcgagacgcctttttgtcaacataactgtaaagatgcaaaaccaagaatattttcagatatttacagcaatccatcaacagtcagacgaagggggggcagccaaagtgggggggagtaagaccttttttttgctgtaatttgactcgaccttccagtggtttgtgccttttttcttttgacttgtttgtggatggaatgtttacagacatttctaattactgctttaattaaataaattggatcaaaggccgttcgaggtatttttgttttgccgtttgtcgctcagaattggcattttgagaggtgattgatactgctaacaattttctagtactctagtttgtttcaagaagagattttgggtagacgtaatcttcaccctttcaaattatataacaatacgaacattattttttatactgatcataatttccagatttggggagggggtgatcgtggcaggaaaagttgtatgtttggtagttgcatatggtgatttttgatttttcaatgctggtaggtaagtaaggaggtctctgtaccatggctcgtacaaagcagactgcccgcaaatcgaccggtggtaaagcacccaggaagcaactggctacaaaagccgctcgcaagagtgcgccctctactggaggggtgaagaaacctcatcgttacaggtattaaaaaacaggaaaaaaatgggacaaagtctctcttgtatgtatccacataatttaacaaaaagatggataacaggaaaactttttgctttagagaaacttttttttttcatttgaacacttaactactgcttaaataaatgtactgtatgatcatttatatataaagttaagtattaggttttattgaaaacgtttaactttgaagccataatcttacctggaggtctaaggagacctcgtatatcactgataatgttaatgggatatattgacattttagttaactattagtaattctttaaaaatagttaagtgttgctttcttgaatacacttttgaggttatctttcctagtttttggcaaaataataatataatcgagattgggtgttttataaagttcctaccccctcatttacttgattagcttatttccttctgtattagctcttttgattgtgaactacctgagccactgtgttgttaagggcatctgccattagagggcagaagttgcctagacttagcctccgcaaatattacgattaacctaaaataaaagctcatacagtggaagaaataagtcagaatgagaggtaaatttcttgctgacaattacttagtaaaaaaaacttttccacgtggaaggaacactgcacaatcgggtgtattcaaatttaatagttgatgaggggtgaactgtagtcagggtagaggtattctgggaaatagaacttgaatgccacaagcagatttttaaatgaagttcattgggttgtgttggtcatctttaagggctcttaagataatagagggtgtagggaaggagagtggtgctaaattgaggggagccaacttcttcattagcctgacagctaataccttcccaaggtttatatacatgaataactttttaaaatgaaaataaatagggctttgatgaatttcttgctaagaatgcattttaatttcatgctttttgctttaaaggtattgttactagatttgaatgttcacatcttaagttgatcagtgaaatttgatactgaagctgaagaattgttgggtggcttattttttgaaagattactgcatttctttgaagctgcccacttacctttttgtgctagttatgtttttggtaacagtttctttattaattttttaaaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggtaaaatggtgggtgggaagactcagagtttgtattcctgttgtgtaccaagaacagttccaaattgttgcatgtgcttatatcatttaatcacaagcctgtcaggtagttgatattgttacttcactgttgagacttcagaaaggttaaattgctcaaggtcatacacgtagaaaatggcaaaaccataatttgaacctatttgactccaaggcttagtgcacattccattataccatttaaaattttgaaacattgcactaaaaacaatatttaaagaaaatcctctggtttggtttatggatgctgcaggacattaagaagaacttgagactagaggtctatatttgtagtaacaatttcaaacaacagtgcctagaataaaagaaatgtctcttcaggtcctgaagaaacatataggtagagagagcttaatacctaggatgggaataggcagtattaaaaaattatgcattaagacgtaaaaggagcagtgagtggaggataggattggattggcaagattgggggaacggtgcttaaaggtgatactggacgtgtaaaggggccaggatttgccttgtggtttcctaaaggggattagggattgccacttacatgtggagcatattgaatttaatcttaatctctagcatgtcaggacttagagaaatactgttctagagatatttatgtatttgagatatttgtggtttaagttaagaccaaaacttgaaattcctacaagatacttgataaaggtatgtagacgtcattaacatcagtcacttaagtaacctattttatattgtgtggttggattctttagttgcaagtatcccagaatacagtaaagctaccacctccatcagaagcatgcagttgggggtcgttaaaaatgctgattgttaggcaccattgcagacctgaatcattctgggggtggggagggggcatccagaatctaagtacattccattggaggattttcaaggctgctgttgctgtgctgggcttttgctttatgtatgagaaagataggataggtggattccaccctgaagtgtaattggtcgagctgtttctatggttaatctaaacttcatttgattggtgaattccaggagtaggcatccattaaattctcagtgagttgctgaaatttcaactactaaaatcttccatgtttttacttaaaactttattttctgttttaatagccgattatcttcagttcttatttattactaaatacagaaatgttactaagattcgtaagcattggtacaaagctgctaatattttctagttagaactgtgcttaaagaacaatactgtacttgaaaagtgtagcgtttttagcttaaatgtcaataatgtagccttttaaattgaatttactaaacaatctgggtaaaagactcatctttctaaaattattcacatgtaaacttccattgttactattacatatttgtcattaagtgtggttgtattggttttaagcaaaatgttcacctcttctgtccacagttctcttagtgttagatagctctttattggaaagagtgatctttgttaagcttttgtgttttctgtttgcttttttatgcatatattttagttatcttaaaaatacagagagtgggccgggcgccgtgactcacatctgtaatcccaacactttgggaggctgaggtgggtagatcacctgaggtcaggagttccagaccagcgtggccaacatggtgtgaaaccccttctctactaaaaatacaaaaaattagcttggcgtggtggcaggcgcctataatcccagctactagggaggctgaggcaggagaattgcttaaacccggaaagcagaggttgcagtgagccgagatggcgccattgcactccagcctgggcaacaagagcaaaactcttgtctcaaaaaaaaaaaaaagataacagagggagaatttgttaatggaataggataatagaagatcgcaattgaagtagaaatccaaaggtcctttgtttttggccttagaattacacactttacaactaaaggatagcaggcagtccagtgagaatggtagaggagaaattagtctcagtttgccatagataatcaataattgacttacttaaaatatagtctttgcttttaatgtctgagaacttgaactcaaatattcttgtgttaagtggacagtgttgctttgaaatggtaaagaggcagacgttgaatatactcaaaatgtcctaaactaatactttcttgtagttcttttttttctttgccattaaatcaaaattaattcattaggtcataaattagtgtggcagtctttaattcccattacagccttaatgttttaagggactttgggttacttgtaataaaattggaaatgtatgggtcatggtcagatcctctcgtaggagttcagtctcctctcagagctgaccagacacaatccttataaactagtagccaggttctaagacagcagtgagagccccagctaccacatgccacatctgagtggttctaagtgataccactctgaaaggccaccagaaccatggtgcctgatggtttgcttgcatagtagcttgtaccacattttacaaaagcattctagctttctgtaatcccgaggtgccaggttaagcaaaattaaactgttttctttcctgatataatagctgatatgcgttgtgagcccctaaggaggctacaatatgccattttttccaggatttctacccacttctagaatatcttaagaacgaatctcggaatattaaggacttttgcttttaatacctgtggaataggtaagatgcgagggactacttgcatagaatagaaacattttttaaaactagcaaatattgaaaatctacttgaggacaaggcatttggagtattaaatgagatttctgttatcaagtaacttaagggcaccaaaaaatggctgataatgcaaggtcattgtgagtgccaaggaactaggagttcagggttgcctgcactggcaacaggagcaagagacaagtatgcgattttctccttcatggaatagtcttgagctgggccttaaagggtagataagtaagacttagttgttaataacaaatgctggagaaacacacgtgaaaatagttcaatttgaatgaaggaaacttgtagaagagtagtgatagggctaaaaaggatgaagctagaaatgtgaaagtaccttttttttttttttttgagacgagtcacactctgtcacctaggctggagtgcagtggtgcaatctcggcttactgcagcctccgcctcccgggttcaagcaattctcctgtctcagcctcccaagtagctgggactacaggcacatgccaccacgcccagctactttttgtatttttagtagagacaaggtttctccgtgttggtcagtctagtctcaagctcctgacctcgggtgatctgcctgcctcggcctcccaaagtgctgggattacaggcgtgagccaccacggcacctggtctggaattttttttattagaataaaaatgcaactataagactgttttctcccttaatatatcttcagcagagaaataacttttccttatagaaaaggagagagagccaactaacctatcatttcatgctccagatccaaaactgttggatttatgattattttttaaaatggtaatttctccatttcaaaatgagtaagcaggccgggcatggcggctcacacctgtaatcccagcactttgggaggctgaggtgggcagatcacctgaagttaggagttcaagaccagcctggccaacatggtgaaaccccatctctactaaaaatacaaaattagctggacgtgatggtgcatgcctgtaatcccagctactcgggaggctgaggcaggagaattgcctgagctcgggaggtggaggttgcagtgagccgaggttataccactgcactccagccagggctacagagcaagactcaaacctcaaaaaacaaaaacaaaaagagtaagcagatgttttggcttagactaaaagattcttcagcttttcagacagctataagtatactaagaatttgagttatgagttaattctaagtggaaacgcccctttttcctcttcacaagttaagtgtcaatgagtgattcatacactgtcatttttaagtggtagtaggaataagataacttgaaaggatcttacagtcaaatgggaaaaaccagagaaatcgatactagtactagagggcaacaaatgctgttacaaattggggtacgtagaggaaggtacttggtagaggacaggggcatgtttccggggcatagatcaaagtatataaataaggaactgccaggccagttgcggtggctcactcctgtaatcccagcactttgggaggccgaggcaggaggatcacgaggtcaggagatcgagaccatcctagctaacacaatgaagccccatctctactgaaaattagtcaggcgtggtggtgggcgcctgtagcccgagctactcgggaggctgaggcaggagaatgacatgaacctgggaggtggagcttgcagtgagctgagatcttgccactgcactccagcctgggcaacaaagagagactccgtctcaaaataaataaataaggaactgccgggcgcggtggctcacgcctataatcccagcactttgggaggccaaggtgggtggatcacgaggtcaggagttcgagaccagcctgaccaacatggtgaaaccccttctctactaaaaatacaaaaaagtagccaggcatggtggcgcatacctgtagtcccagctactcgggaggctgaggcaggagaatcgcttgaatccgggaggtggaggttgcagtgagccgagatcgcgctactgcactccagcctgggcgacagagtaagactccatctcagaaagaaagaaagaaatacggaactaacttgtgatatgttctggaatcaaaagtactcttatgataaaacaggtatgaaagggaacatagatgagaagcatgtgataaaaaccacttgttcaccatgttatactactggacaaggcagaggttcacatactgtgtgaatgggattcagagtgaggaggagactaggctgggatggggtatttggattggacatgattgcgtttataagaatgagagtgttaaattggatttcttgctttatttgtgacatttcagtttattagaaatcatgttaccattagaaaaattgaagtttcctagtaacaaagtaatttgatttgtgtaacttgataaaagatttactgacttaagcttttgttttttttcataagctgcttttgagctttgtcccacaggttgtaaaatgtaagcatttggtaaaattgtcagcatcttgcccagtcatttttttaaagggttcaaaaacctttttgttttaattcgtatagttgggtcttaactattggaaataacatcatcagtaattttttcttcattccttttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctcaaaaaaaaaaaaaaaaatttctcttcttcctgttattggtagttctgaacgttagatattttttttccatggggtcaaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttttttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaactatacctgcttttggtcttta

SEQ ID NO: 90 - DNA, 10189 bp, synthetic construct
  source 1..10189: mol_type = other DNA
acacattgtttagaggccgacgcgacacagccatcttgcgtcgggggcccgggcccgggaggagagcgagccccgtcccctgatcccggaggccaacaccggcgaggggctgccctgcacgcaggcggccggcccaggagtcttagcggatcaagttgtctacggcggactcgggccggtcccgcaggacccggccgcacttggccgagcgcgcaggcgggaaggaggccgtcggtccgcccctcccgctgccgcagcggccaggtccgacagcgggcgcgggacgctggattcctatttgccagcttcctgctggggagcgttttccaatggggcgcggcgacccggccgaccgcgtcctcctcccccactgccgcccgggtctcctcgggggcgcgcgccggggcccgcgcgggggcagcccctccctcccctccccgccgcggcccgagacgcccgcaggccgggcgttcacccgccgcgcctccgcccgccttcccctccccccacttccctccgggcgccccaaacccgggcttcgggggttcgacgccgtgcgcacacggagcgggatgggagtgcagggccgggggcgggggctggccgggtgcgggcggcagtctcggcgggagccggcggcccctcagcgtgtcttttgtgtttgtacacacacggcgcgaggcggcctggaggagggagcgggcggcgcgcgggggaggggcgagcgcgcgccagcgaacgggcgcgcggggggaagggcgggggcggggcgcgagaggagctatgggggcggggccgagccttccctccattgtgtgtgattggctggcgcgcggcgcgggggcggggcggcgtgtgttgggggatagcctcggtgtcagccatctttcaattgtgttcgcagccgccgccgcgccgccgtcgctctccaacgccagcgccgcctctcgctcgccgagctccagccgaaggagaaggggggtaagtttccccgtctgcccgcttccccggaaccgagccccgcttgccgtcagccccgagccgggccctggcggtgccgggaggggacccatagcccctccggcttagcccacaaactttttggcccagaaatggaggtgaggagcgagtttccctgttccctctggcggcggcggctcccccgtctctcgccgcctcagcccaacagcagcaaccgccgcggcgccgagcctgctctccctcctcctccccgcgcccctggctcctctttcttcggtgaaatcccgcccgccgccccttccccggaccccaaaccttcaccatgacccgcgcgggcctcttaactaccgcccccgggccccagcccccagttttcgagcgggaaaggggtggaaatcgccgccgcttcgcaccctggggtaactcgctttttgctgcctccccacagttgttgctaagcttcaccatcttgtctctcttctctggtcaccgaccatattttttcccccgtttctctcttccccatttcaaaaaagcaagaatttttaaaagagggacgtttttttcccttttttggagaggcggaaacttgatgcatttgaaatgcaaaaaaaacttttgcattttggagggcggcacgtggcaggggataggggtattttcgagcgatttggtgttttgtctctttggaacggaatcggatacgttttgcgattgtttccattttgttctggtcggggagccgagttggttcctgctcgaggcgctgggagcgaggcgggcaggttgggggaccctgtgccgagggacccgaaggaggcaaaaccaaaagtttatgtggtgcttggggtggaccggtcggcggatgtcgtgcgttaattaaagcctgggtgcggggcactttttatttcactgcgaagcctgtgaggactggacgcgaggacggggctttgtagaatgctcgctggtggtagctgttgttcctctcttgttttttcgagacgcctttttgtcaacataactgtaaagatgcaaaaccaagaatattttcagatatttacagcaatccatcaacagtcagacgaagggggggcagccaaagtgggggggagtaagaccttttttttgctgtaatttgactcgaccttccagtggtttgtgccttttttcttttgacttgtttgtggatggaatgtttacagacatttctaattactgctttaattaaataaattggatcaaaggccgttcgaggtatttttgttttgccgtttgtcgctcagaattggcattttgagaggtgattgatactgctaacaattttctagtactctagtttgtttcaagaagagattttgggtagacgtaatcttcaccctttcaaattatataacaatacgaacattattttttatactgatcataatttccagatttggggagggggtgatcgtggcaggaaaagttgtatgtttggtagttgcatatggtgatttttgatttttcaatgctggtaggtaagtaaggaggtctctgtaccatggctcgtacaaagcagactgcccgcaaatcgaccggtggtaaagcacccaggaagcaactggctacaaaagccgctcgcatgagtgcgccctctactggaggggtgaagaaacctcatcgttacaggtattaaaaaacaggaaaaaaatgggacaaagtctctcttgtatgtatccacataatttaacaaaaagatggataacaggaaaactttttgctttagagaaacttttttttttcatttgaacacttaactactgcttaaataaatgtactgtatgatcatttatatataaagttaagtattaggttttattgaaaacgtttaactttgaagccataatcttacctggaggtctaaggagacctcgtatatcactgataatgttaatgggatatattgacattttagttaactattagtaattctttaaaaatagttaagtgttgctttcttgaatacacttttgaggttatctttcctagtttttggcaaaataataatataatcgagattgggtgttttataaagttcctaccccctcatttacttgattagcttatttccttctgtattagctcttttgattgtgaactacctgagccactgtgttgttaagggcatctgccattagagggcagaagttgcctagacttagcctccgcaaatattacgattaacctaaaataaaagctcatacagtggaagaaataagtcagaatgagaggtaaatttcttgctgacaattacttagtaaaaaaaacttttccacgtggaaggaacactgcacaatcgggtgtattcaaatttaatagttgatgaggggtgaactgtagtcagggtagaggtattctgggaaatagaacttgaatgccacaagcagatttttaaatgaagttcattgggttgtgttggtcatctttaagggctcttaagataatagagggtgtagggaaggagagtggtgctaaattgaggggagccaacttcttcattagcctgacagctaataccttcccaaggtttatatacatgaataactttttaaaatgaaaataaatagggctttgatgaatttcttgctaagaatgcattttaatttcatgctttttgctttaaaggtattgttactagatttgaatgttcacatcttaagttgatcagtgaaatttgatactgaagctgaagaattgttgggtggcttattttttgaaagattactgcatttctttgaagctgcccacttacctttttgtgctagttatgtttttggtaacagtttctttattaattttttaaaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggtaaaatggtgggtgggaagactcagagtttgtattcctgttgtgtaccaagaacagttccaaattgttgcatgtgcttatatcatttaatcacaagcctgtcaggtagttgatattgttacttcactgttgagacttcagaaaggttaaattgctcaaggtcatacacgtagaaaatggcaaaaccataatttgaacctatttgactccaaggcttagtgcacattccattataccatttaaaattttgaaacattgcactaaaaacaatatttaaagaaaatcctctggtttggtttatggatgctgcaggacattaagaagaacttgagactagaggtctatatttgtagtaacaatttcaaacaacagtgcctagaataaaagaaatgtctcttcaggtcctgaagaaacatataggtagagagagcttaatacctaggatgggaataggcagtattaaaaaattatgcattaagacgtaaaaggagcagtgagtggaggataggattggattggcaagattgggggaacggtgcttaaaggtgatactggacgtgtaaaggggccaggatttgccttgtggtttcctaaaggggattagggattgccacttacatgtggagcatattgaatttaatcttaatctctagcatgtcaggacttagagaaatactgttctagagatatttatgtatttgagatatttgtggtttaagttaagaccaaaacttgaaattcctacaagatacttgataaaggtatgtagacgtcattaacatcagtcacttaagtaacctattttatattgtgtggttggattctttagttgcaagtatcccagaatacagtaaagctaccacctccatcagaagcatgcagttgggggtcgttaaaaatgctgattgttaggcaccattgcagacctgaatcattctgggggtggggagggggcatccagaatctaagtacattccattggaggattttcaaggctgctgttgctgtgctgggcttttgctttatgtatgagaaagataggataggtggattccaccctgaagtgtaattggtcgagctgtttctatggttaatctaaacttcatttgattggtgaattccaggagtaggcatccattaaattctcagtgagttgctgaaatttcaactactaaaatcttccatgtttttacttaaaactttattttctgttttaatagccgattatcttcagttcttatttattactaaatacagaaatgttactaagattcgtaagcattggtacaaagctgctaatattttctagttagaactgtgcttaaagaacaatactgtacttgaaaagtgtagcgtttttagcttaaatgtcaataatgtagccttttaaattgaatttactaaacaatctgggtaaaagactcatctttctaaaattattcacatgtaaacttccattgttactattacatatttgtcattaagtgtggttgtattggttttaagcaaaatgttcacctcttctgtccacagttctcttagtgttagatagctctttattggaaagagtgatctttgttaagcttttgtgttttctgtttgcttttttatgcatatattttagttatcttaaaaatacagagagtgggccgggcgccgtgactcacatctgtaatcccaacactttgggaggctgaggtgggtagatcacctgaggtcaggagttccagaccagcgtggccaacatggtgtgaaaccccttctctactaaaaatacaaaaaattagcttggcgtggtggcaggcgcctataatcccagctactagggaggctgaggcaggagaattgcttaaacccggaaagcagaggttgcagtgagccgagatggcgccattgcactccagcctgggcaacaagagcaaaactcttgtctcaaaaaaaaaaaaaagataacagagggagaatttgttaatggaataggataatagaagatcgcaattgaagtagaaatccaaaggtcctttgtttttggccttagaattacacactttacaactaaaggatagcaggcagtccagtgagaatggtagaggagaaattagtctcagtttgccatagataatcaataattgacttacttaaaatatagtctttgcttttaatgtctgagaacttgaactcaaatattcttgtgttaagtggacagtgttgctttgaaatggtaaagaggcagacgttgaatatactcaaaatgtcctaaactaatactttcttgtagttcttttttttctttgccattaaatcaaaattaattcattaggtcataaattagtgtggcagtctttaattcccattacagccttaatgttttaagggactttgggttacttgtaataaaattggaaatgtatgggtcatggtcagatcctctcgtaggagttcagtctcctctcagagctgaccagacacaatccttataaactagtagccaggttctaagacagcagtgagagccccagctaccacatgccacatctgagtggttctaagtgataccactctgaaaggccaccagaaccatggtgcctgatggtttgcttgcatagtagcttgtaccacattttacaaaagcattctagctttctgtaatcccgaggtgccaggttaagcaaaattaaactgttttctttcctgatataatagctgatatgcgttgtgagcccctaaggaggctacaatatgccattttttccaggatttctacccacttctagaatatcttaagaacgaatctcggaatattaaggacttttgcttttaatacctgtggaataggtaagatgcgagggactacttgcatagaatagaaacattttttaaaactagcaaatattgaaaatctacttgaggacaaggcatttggagtattaaatgagatttctgttatcaagtaacttaagggcaccaaaaaatggctgataatgcaaggtcattgtgagtgccaaggaactaggagttcagggttgcctgcactggcaacaggagcaagagacaagtatgcgattttctccttcatggaatagtcttgagctgggccttaaagggtagataagtaagacttagttgttaataacaaatgctggagaaacacacgtgaaaatagttcaatttgaatgaaggaaacttgtagaagagtagtgatagggctaaaaaggatgaagctagaaatgtgaaagtaccttttttttttttttttgagacgagtcacactctgtcacctaggctggagtgcagtggtgcaatctcggcttactgcagcctccgcctcccgggttcaagcaattctcctgtctcagcctcccaagtagctgggactacaggcacatgccaccacgcccagctactttttgtatttttagtagagacaaggtttctccgtgttggtcagtctagtctcaagctcctgacctcgggtgatctgcctgcctcggcctcccaaagtgctgggattacaggcgtgagccaccacggcacctggtctggaattttttttattagaataaaaatgcaactataagactgttttctcccttaatatatcttcagcagagaaataacttttccttatagaaaaggagagagagccaactaacctatcatttcatgctccagatccaaaactgttggatttatgattattttttaaaatggtaatttctccatttcaaaatgagtaagcaggccgggcatggcggctcacacctgtaatcccagcactttgggaggctgaggtgggcagatcacctgaagttaggagttcaagaccagcctggccaacatggtgaaaccccatctctactaaaaatacaaaattagctggacgtgatggtgcatgcctgtaatcccagctactcgggaggctgaggcaggagaattgcctgagctcgggaggtggaggttgcagtgagccgaggttataccactgcactccagccagggctacagagcaagactcaaacctcaaaaaacaaaaacaaaaagagtaagcagatgttttggcttagactaaaagattcttcagcttttcagacagctataagtatactaagaatttgagttatgagttaattctaagtggaaacgcccctttttcctcttcacaagttaagtgtcaatgagtgattcatacactgtcatttttaagtggtagtaggaataagataacttgaaaggatcttacagtcaaatgggaaaaaccagagaaatcgatactagtactagagggcaacaaatgctgttacaaattggggtacgtagaggaaggtacttggtagaggacaggggcatgtttccggggcatagatcaaagtatataaataaggaactgccaggccagttgcggtggctcactcctgtaatcccagcactttgggaggccgaggcaggaggatcacgaggtcaggagatcgagaccatcctagctaacacaatgaagccccatctctactgaaaattagtcaggcgtggtggtgggcgcctgtagcccgagctactcgggaggctgaggcaggagaatgacatgaacctgggaggtggagcttgcagtgagctgagatcttgccactgcactccagcctgggcaacaaagagagactccgtctcaaaataaataaataaggaactgccgggcgcggtggctcacgcctataatcccagcactttgggaggccaaggtgggtggatcacgaggtcaggagttcgagaccagcctgaccaacatggtgaaaccccttctctactaaaaatacaaaaaagtagccaggcatggtggcgcatacctgtagtcccagctactcgggaggctgaggcaggagaatcgcttgaatccgggaggtggaggttgcagtgagccgagatcgcgctactgcactccagcctgggcgacagagtaagactccatctcagaaagaaagaaagaaatacggaactaacttgtgatatgttctggaatcaaaagtactcttatgataaaacaggtatgaaagggaacatagatgagaagcatgtgataaaaaccacttgttcaccatgttatactactggacaaggcagaggttcacatactgtgtgaatgggattcagagtgaggaggagactaggctgggatggggtatttggattggacatgattgcgtttataagaatgagagtgttaaattggatttcttgctttatttgtgacatttcagtttattagaaatcatgttaccattagaaaaattgaagtttcctagtaacaaagtaatttgatttgtgtaacttgataaaagatttactgacttaagcttttgttttttttcataagctgcttttgagctttgtcccacaggttgtaaaatgtaagcatttggtaaaattgtcagcatcttgcccagtcatttttttaaagggttcaaaaacctttttgttttaattcgtatagttgggtcttaactattggaaataacatcatcagtaattttttcttcattccttttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctcaaaaaaaaaaaaaaaaatttctcttcttcctgttattggtagttctgaacgttagatattttttttccatggggtcaaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttttttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaactatacctgcttttggtcttta

SEQ ID NO: 91 - protein, 135 aa, synthetic construct
ARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 92 - protein, 135 aa, synthetic construct
ARTKQTARKSTGGKAPRKQLATKAARMSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 93 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ccttccctgaaggttcctcc

SEQ ID NO: 94 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cactcatgcgagcggctttt

SEQ ID NO: 95 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gcactcatgcgagcggcttt

SEQ ID NO: 96 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cgcactcatgcgagcggctt

SEQ ID NO: 97 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gcgcactcatgcgagcggct

SEQ ID NO: 98 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ggcgcactcatgcgagcggc

SEQ ID NO: 99 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gggcgcactcatgcgagcgg

SEQ ID NO: 100 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
agggcgcactcatgcgagcg

SEQ ID NO: 101 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gagggcgcactcatgcgagc

SEQ ID NO: 102 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
agagggcgcactcatgcgag

SEQ ID NO: 103 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
tagagggcgcactcatgcga

SEQ ID NO: 104 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gggcagtctgctttgtacga

SEQ ID NO: 105 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cgatttgcgggcagtctact

SEQ ID NO: 106 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
taccaccggtcgatttgcgg

SEQ ID NO: 107 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ttgcttcctgggtgctttac

SEQ ID NO: 108 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gaggtttcttcacccctcca

SEQ ID NO: 109 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
agcaatttctcgcaccaaac

SEQ ID NO: 110 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ccacagtaccaggcctataa

SEQ ID NO: 111 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
caaccagataggcctcactt

SEQ ID NO: 112 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
gccaatacctgatttctgtc

SEQ ID NO: 113 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cactcgcaatcatatactta

SEQ ID NO: 114 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ggaacagcacagaagacaga

SEQ ID NO: 115 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cttttcctgccacgatcacc

SEQ ID NO: 116 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
cctaccagcattgaaaaatc

SEQ ID NO: 117 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = phosphorothioate
aattctgagcgacaaacggc

SEQ ID NO: 118 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
tcatgcgagcggcttttgta

SEQ ID NO: 119 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ctctggaagcgcagatctgt

SEQ ID NO: 120 - DNA, 20 bp, synthetic construct
  source 1..20: mol_type = other DNA
  modified_base 1..5: mod_base = OTHER; note = Modified by 2prime-O-methoxyethyl
  modified_base 16..20: mod_base = OTHER; note = Modified by 2prime-O-methoxyethyl
  modified_base 1..20: mod_base = OTHER; note = Modified by phosphorothioate
ttacacgtttggcatggata

SEQ ID NO: 121 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tttcctgttttttaatacct

SEQ ID NO: 122 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ttcctgttttttaatacctg

SEQ ID NO: 123 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tcctgttttttaatacctgt

SEQ ID NO: 124 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cctgttttttaatacctgta

SEQ ID NO: 125 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
ctgttttttaatacctgtaa

SEQ ID NO: 126 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tgttttttaatacctgtaac

SEQ ID NO: 127 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
gttttttaatacctgtaacg

SEQ ID NO: 128 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ttttttaatacctgtaacga

SEQ ID NO: 129 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tttttaatacctgtaacgat

SEQ ID NO: 130 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ttttaatacctgtaacgatg

SEQ ID NO: 131 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tttaatacctgtaacgatga

SEQ ID NO: 132 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ttaatacctgtaacgatgag

SEQ ID NO: 133 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
taatacctgtaacgatgagg

SEQ ID NO: 134 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
aatacctgtaacgatgaggt

SEQ ID NO: 135 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
atacctgtaacgatgaggtt

SEQ ID NO: 136 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tacctgtaacgatgaggttt

SEQ ID NO: 137 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
acctgtaacgatgaggtttc

SEQ ID NO: 138 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tagagggcgcactcttgcga

SEQ ID NO: 139 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tccagtagagggcgcactct

SEQ ID NO: 140 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
acccctccagtagagggcgc

SEQ ID NO: 141 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tcttcacccctccagtagag

SEQ ID NO: 142 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
aggtttcttcacccctccag

SEQ ID NO: 143 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
cgatgaggtttcttcacccc

SEQ ID NO: 144 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tgtaacgatgaggtttcttc

SEQ ID NO: 145 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
ccagtagagggcgcactcat

SEQ ID NO: 146 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tccagtagagggcgcactca

SEQ ID NO: 147 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
ctccagtagagggcgcactc

SEQ ID NO: 148 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cctccagtagagggcgcact

SEQ ID NO: 149 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
ccctccagtagagggcgcac

SEQ ID NO: 150 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cccctccagtagagggcgca

SEQ ID NO: 151 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
acccctccagtagagggcgc

SEQ ID NO: 152 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cagtagagggcgcactcatg

SEQ ID NO: 153 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
agtagagggcgcactcatgc

SEQ ID NO: 154 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
gtagagggcgcactcatgcg

SEQ ID NO: 155 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tagagggcgcactcatgcga

SEQ ID NO: 156 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
agagggcgcactcatgcgag

SEQ ID NO: 157 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
gagggcgcactcatgcgagc

SEQ ID NO: 158 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
agggcgcactcatgcgagcg

SEQ ID NO: 159 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
gggcgcactcatgcgagcgg

SEQ ID NO: 160 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ggcgcactcatgcgagcggc

SEQ ID NO: 161 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
gcgcactcatgcgagcggct

SEQ ID NO: 162 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cgcactcatgcgagcggctt

SEQ ID NO: 163 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
gcactcatgcgagcggcttt

SEQ ID NO: 164 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
cactcatgcgagcggctttt

SEQ ID NO: 165 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
actcatgcgagcggcttttg

SEQ ID NO: 166 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
ctcatgcgagcggcttttgt

SEQ ID NO: 167 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
tcatgcgagcggcttttgta

SEQ ID NO: 168 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
catgcgagcggcttttgtag

SEQ ID NO: 169 - DNA, 20 bp, synthetic construct
  source 1..20: mol_type = other DNA
  modified_base 1..20: mod_base = OTHER; note = Modified by phosphorothioate
  modified_base 1..20: mod_base = OTHER; note = Modified by 2prime-O-methoxyethyl
atgcgagcggcttttgtagc

SEQ ID NO: 170 - DNA, 20 bp, synthetic construct
  source 1..20: mol_type = other DNA
  modified_base 1..20: mod_base = OTHER; note = Modified by 2prime-O-methoxyethyl
  modified_base 1..20: mod_base = OTHER; note = Modified by phosphorothioate
tgcgagcggcttttgtagcc

SEQ ID NO: 171 - DNA, 24 bp, synthetic construct
  source 1..24: mol_type = other DNA
ggactttaaaacagatctgcgctt

SEQ ID NO: 172 - DNA, 25 bp, synthetic construct
  source 1..25: mol_type = other DNA
gtcttttggcataattgttacacgt

SEQ ID NO: 173 - DNA, 20 bp, synthetic construct
  source 1..20: mol_type = other DNA
gctacaaaagccgctctcaa

SEQ ID NO: 174 - DNA, 20 bp, synthetic construct
  source 1..20: mol_type = other DNA
gctacaaaagccgctcgaat

SEQ ID NO: 175 - DNA, 20 bp, synthetic construct
  source 1..20: mol_type = other DNA
ccagacgctggaagggaagt

SEQ ID NO: 176 - DNA, 128 bp, synthetic construct; identical to SEQ ID NO: 86
  source 1..128: mol_type = other DNA

SEQ ID NO: 177 - DNA, 60 bp, synthetic construct
  source 1..60: mol_type = other DNA
ttggccactaaggcagcccgcaaaagcgctccggccaccggcggcgtgaaaaagccccac

SEQ ID NO: 178 - DNA, 60 bp, synthetic construct
  source 1..60: mol_type = other DNA
ctggctaccaaggctgctcgcaagagcgcgccggctaccggcggcgtgaaaaagcctcac

SEQ ID NO: 179 - DNA, 60 bp, synthetic construct
  source 1..60: mol_type = other DNA
cttgctactaaagcagcccgtaagagcgctccggccaccggtggcgtgaagaaacctcat

SEQ ID NO: 180 - DNA, 60 bp, synthetic construct
  source 1..60: mol_type = other DNA
ctggccaccaaggctgctcgaaagagcgctccagccaccggcggcgtgaagaagccccac

SEQ ID NO: 181 - DNA, 60 bp, synthetic construct
  source 1..60: mol_type = other DNA
ctggccactaaggcagctcgcaagagcgctccggccacgggcggcgtgaagaagccccat

SEQ ID NO: 182 - DNA, 60 bp, synthetic construct
  source 1..60: mol_type = other DNA
ctggccactaaggcggctcgcaaaagcgcgccagccaccggtggcgtgaagaagccccac

SEQ ID NO: 183 - DNA, 60 bp, synthetic construct
  source 1..60: mol_type = other DNA
ctggccactaaggcggctcggaaaagcgcgccggccaccggcggcgtgaagaaacctcat

SEQ ID NO: 184 - DNA, 60 bp, synthetic construct
  source 1..60: mol_type = other DNA
ctggccaccaaggcggctcggaagagcgctccggccaccggcggtgtcaagaagccccat

SEQ ID NO: 185 - DNA, 60 bp, synthetic construct
  source 1..60: mol_type = other DNA
ctggccaccaaggcggctcgcaagagcgctccggccaccggtggcgtcaagaagccccac

SEQ ID NO: 186 - DNA, 60 bp, synthetic construct
  source 1..60: mol_type = other DNA
ctggccaccaaggcagcgcgcaaaagcgctccagcgactggcggtgtgaagaagccccac

SEQ ID NO: 187 - DNA, 60 bp, synthetic construct
  source 1..60: mol_type = other DNA
ctggccaccaaggcggcccgcaagagcgcgccggccacgggcggggtgaagaagccgcac

SEQ ID NO: 188 - DNA, 60 bp, synthetic construct; identical to SEQ ID NO: 187
  source 1..60: mol_type = other DNA

SEQ ID NO: 189 - DNA, 60 bp, synthetic construct
  source 1..60: mol_type = other DNA
ctggctaccaaagcggcccgcaagagcgcgccggccacgggcggggtgaagaagccgcac

SEQ ID NO: 190 - DNA, 60 bp, synthetic construct
  source 1..60: mol_type = other DNA
ctggctacaaaagccgctcgcaagagtgcgccctctactggaggggtgaagaaacctcat

SEQ ID NO: 191 - DNA, 60 bp, synthetic construct
  source 1..60: mol_type = other DNA
ctggctacaaaagccgctcgcatgagtgcgccctctactggaggggtgaagaaacctcat

SEQ ID NO: 192 - DNA, 60 bp, synthetic construct
  source 1..60: mol_type = other DNA
ctggccacgaaagccgccaggaaaagcgctccctctaccggcggggtgaagaagcctcat

SEQ ID NO: 193 - RNA, 89 nt, synthetic construct
  source 1..89: mol_type = other RNA
cagctggccaccaaggcggctcggaaaagcgcgccctctaccggcggggtgaagaagcctcaccgctacaggtaggcagagggctggga

SEQ ID NO: 194 - RNA, 89 nt, synthetic construct
  source 1..89: mol_type = other RNA
caactggctacaaaagccgctcgcaagagtgcgccctctactggaggggtgaagaaacctcatcgttacaggtattaaaaaacaggaaa

SEQ ID NO: 195 - DNA, 498 bp, synthetic construct
  source 1..498: mol_type = other DNA
nncgctgctgcgttaacttaagcttggtaccgagctcggatccggcggcgtgtgttgggggatagcctcggtgtcagccatctttcaattgtgttcgcagccgccgccgcgccgccgtcgctctccaacgccagcgccgcctctcgctcgccgagctccagccgaaggagaaggggggtaagtaaggaggtctctgtaccatggctcgtacaaagcaaactgcccggaaatcgaccggtggtaaagcaccctggaagcaactggctacaaaagccgctcgcgagagtgcagccctctactggagggcgtgaagaaacctcatcgtgccttgctgggtactgtcgcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcatggaagaattcaaaa

SEQ ID NO: 196 - DNA, 344 bp, synthetic construct
  source 1..344: mol_type = other DNA
cccgctgctgcgttaacttaagcttggtaccgagctcggatccggcggcgtgtgttgggggatagcctcggtgtcagccatctttcaattgtgttcgcagccgccgccgcgccgccgtcgctctccaacgccagcgccgcctctcgctcgccgagctccagccgaaggagaaggggggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcagtggagaattcaaa

SEQ ID NO: 197 - DNA, 146 bp, synthetic construct
  source 1..146: mol_type = other DNA
atggctcgtacaaagcagactgcccgcaaatcgaccggtggtaaagcacccaggaagcaactggctacaaaagccgctcgcaagagtgcgccctctactggaggggtgaagaaacctcatcgttacaggtattaaaaaacaggaaa

SEQ ID NO: 198 - protein, 136 aa, synthetic construct
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 199 - protein, 136 aa, synthetic construct
MARTKQTARKSTGGKAPRKQLATKAARMSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 200 - RNA, 128 nt, synthetic construct
  source 1..128: mol_type = other RNA
atggctcgtacaaagcagactgcccgcaaatcgaccggtggtaaagcacccaggaagcaactggctacaaaagccgctcgcaagagtgcgccctctactggaggggtgaagaaacctcatcgttacag

SEQ ID NO: 201 - DNA, 60 bp, synthetic construct; identical to SEQ ID NO: 177
  source 1..60: mol_type = other DNA

SEQ ID NO: 202 - DNA, 60 bp, synthetic construct; identical to SEQ ID NO: 178
  source 1..60: mol_type = other DNA

SEQ ID NO: 203 - DNA, 60 bp, synthetic construct; identical to SEQ ID NO: 179
  source 1..60: mol_type = other DNA

SEQ ID NO: 204 - DNA, 60 bp, synthetic construct; identical to SEQ ID NO: 180
  source 1..60: mol_type = other DNA

SEQ ID NO: 205 - DNA, 60 bp, synthetic construct; identical to SEQ ID NO: 181
  source 1..60: mol_type = other DNA

SEQ ID NO: 206 - DNA, 60 bp, synthetic construct; identical to SEQ ID NO: 182
  source 1..60: mol_type = other DNA

SEQ ID NO: 207 - DNA, 60 bp, synthetic construct; identical to SEQ ID NO: 183
  source 1..60: mol_type = other DNA

SEQ ID NO: 208 - DNA, 60 bp, synthetic construct; identical to SEQ ID NO: 184
  source 1..60: mol_type = other DNA

SEQ ID NO: 209 - DNA, 60 bp, synthetic construct; identical to SEQ ID NO: 185
  source 1..60: mol_type = other DNA

SEQ ID NO: 210 - DNA, 60 bp, synthetic construct; identical to SEQ ID NO: 186
  source 1..60: mol_type = other DNA

SEQ ID NO: 211 - DNA, 60 bp, synthetic construct; identical to SEQ ID NO: 187
  source 1..60: mol_type = other DNA

SEQ ID NO: 212 - DNA, 60 bp, synthetic construct; identical to SEQ ID NO: 187
  source 1..60: mol_type = other DNA

SEQ ID NO: 213 - DNA, 60 bp, synthetic construct; identical to SEQ ID NO: 189
  source 1..60: mol_type = other DNA

SEQ ID NO: 214 - DNA, 60 bp, synthetic construct; identical to SEQ ID NO: 190
  source 1..60: mol_type = other DNA

SEQ ID NO: 215 - DNA, 60 bp, synthetic construct; identical to SEQ ID NO: 191
  source 1..60: mol_type = other DNA

SEQ ID NO: 216 - DNA, 60 bp, synthetic construct; identical to SEQ ID NO: 192
  source 1..60: mol_type = other DNA

(19) United States
(12) Patent Application Publication
(10) Pub. No.: US 2024/0360450 A1
(43) Pub. Date: Oct. 31, 2024

(54) ANTISENSE OLIGONUCLEOTIDE THERAPY FOR H3.3 K27M DIFFUSE MIDLINE GLIOMAS

(71) Applicant: COLD SPRING HARBOR HARBOR LABORATORY, Cold Spring Harbor, NY (US)

(72) Inventors: Adrian Krainer, Huntington Station, NY (US); Qian Zhang, Methuen, MA (US)

(73) Assignee: COLD SPRING HARBOR HARBOR LABORATORY, Cold Spring Harbor, NY (US)

(21) Appl. No.: 18/685,880

(22) PCT Filed: Aug. 23, 2022
(86) PCT No.: PCT/US2022/041273

§ 371 Date: Feb. 22, 2024

Related U.S. Application Data
(60) Provisional application No. 63/236,219, filed on Aug. 23, 2021.

(51) Int. Cl. C12N 15/113

(52) U.S. Cl. CPC C12N 15/113; C12N 2310/11; C12N 2310/315; C12N 2310/322; C12N 2310/341; C12N 2320/33

(57) ABSTRACT

Disclosed herein are compositions and methods for modulating, such as decreasing, the expression of a mutant histone 3.3 (H3.3) allele or a mutant H3.3 gene. In some embodiments, the compositions are antisense oligonucleotides (ASOs), such as gapmer ASOs and splice-modulating ASOs, that target a region of a mutant H3.3 allele or a mutant H3.3 gene, or products thereof, to reduce expression of the mutant H3.3 allele or a mutant H3.3 gene. The compositions and methods are useful in the treatment of diseases and disorders associated with a mutant H3.3 allele or a mutant H3.3 gene, such as cancer.

### CROSS-REFERENCE TO RELATED APPLICATIONS

This application claims the benefit of the filing date under 35 U.S.C. § 119(e) of U.S. Provisional Application Ser. No. 63/236,219, filed Aug. 23, 2021. The entire teachings of the referenced U.S. Provisional Application are incorporated herein by reference.

### STATEMENT REGARDING FEDERALLY SPONSORED RESEARCH

This invention was made with government support under CA045508 awarded by the National Institutes of Health. The government has certain rights in the invention.

### BACKGROUND

Pediatric high-grade gliomas (pHGGs) represent 10-15% of brain tumors in children, and have exceedingly poor outcomes (1,2). About half of pHGGs, known as diffuse midline gliomas (DMG), exhibit a diffuse pattern in the midline, including thalamus, midbrain, and mostly the pons—the latter representing a subgroup termed diffuse intrinsic pontine glioma (DIPG). Approximately 300 children in the U.S. are diagnosed annually. These tumors occur in boys and girls equally, and do not usually appear in adults. Most patients die within 2 years, with a mean survival of 9 months. The brain-stem location limits the clinical management of DIPG: surgical resection is not possible, and localized chemotherapy is ineffective and has severe side effects. Thus, new effective therapies are urgently needed.

### SUMMARY

Provided herein are antisense oligonucleotides (ASOs), such as gapmer ASOs and splice-modulating ASOs that target a histone 3.3 (H3.3) gene found in certain cancers, such as gliomas (e.g., pediatric high-grade gliomas), and uses thereof. In some embodiments, an ASO disclosed herein specifically targets a dominant mutation in an H3F3A allele, such as a dominant mutation which replaces lysine 27 with methionine (K27M). H3.3 K27M is a toxic gain-of-function mutation that inhibits the EZH2 methyltransferase subunit of the Polycomb repressive complex (PRC2), leading to global reduction of tri-methylation on K27 of histone H3 proteins. The epigenetic alteration may be a driving event in tumorigenesis.

In some embodiments, the mutant H3F3A gene or allele, or a product thereof, is targeted using gapmer ASOs or splice-modulating ASOs. Such targeted use of ASOs can result, for example, in one or more positive outcomes, such as reduction of tumor growth, promotion of neural-stem-cell differentiation, and increased survival in an individual with brain cancer, such as diffuse intrinsic pontine glioma (DIPG). In some embodiments, splice-modulating ASOs unexpectedly preferentially downregulate expression of a mutant H3F3A allele relative to expression of a corresponding wild-type H3F3A allele, relative to expression of a H3.3 histone B (H3F3B) allele or relative to expression of both a corresponding wild-type H3F3A allele and an H3F3B allele. This may be because the mutation in a mutant H3F3A allele or a mutant H3F3A gene, or a product thereof, creates a binding site for an RNA-binding protein, which contributes to the aberrant splicing the splice-modulating ASOs elicit to downregulate expression, even though the splice-modulating ASOs bind to a region that does not include the mutation.

According to some aspects, an antisense oligonucleotide (ASO) of about 15 to about 30 nucleosides is provided herein. In some embodiments, the nucleic acid sequence of the ASO is identical to the sequence of any one of SEQ ID NOs.: 1-15, and comprises one or more nucleoside chemical modifications, or an ASO of about 15 nucleosides to about 30 nucleosides, wherein the nucleic acid sequence of the ASO is at least 70% identical to the nucleic acid sequence of any one of SEQ ID NOs.: 1-15, and comprises one or more nucleoside chemical modifications In some embodiments, the ASO comprises a nucleic acid sequence of about 15 nucleosides to about 30 nucleosides is at least 80%, at least 95%, or at least 99% identical to the nucleic acid sequence of any one of SEQ ID NOs.: 1-15.

In some embodiments, the ASO comprises a nucleic acid sequence of about 15 nucleosides to about 30 nucleosides is 100% identical to the nucleic acid sequence of any one of SEQ ID NOs.: 1-15.

According to some aspects, the ASO comprises a nucleic acid sequence of about 15 to about 30 nucleosides that is at least 80% complementary to a region of the nucleic acid sequence ATGGCTCGTACAAAGCAGACTGCCCGCAAATCGACCGGTGGTAAAGCACCCAGG AAGCAACTGGCTACAAAAGCCGCTCGCATGAGTGCGCCCTCTACTGGAGGGGTG AAGAAACCTCATCGTTACAG (SEQ ID NO: 87), wherein the ASO comprises one or more nucleoside chemical modifications.

In some embodiments, the ASO comprises a nucleic acid sequence of about 15 nucleosides to about 30 nucleosides is at least 95%, or at least 99% complementary to a region of the nucleic acid sequence

(SEQ ID NO: 87)
ATGGCTCGTACAAAGCAGACTGCCCGCAAATCGACCGGTGGTAAAGCACC
CAGGAAGCAACTGGCTACAAAAGCCGCTCGCATGAGTGCGCCCTCTACTG
GAGGGGTGAAGAAACCTCATCGTTACAG.

In some embodiments, the ASO comprises a nucleic acid sequence of about 15 nucleosides to about 30 nucleosides 100% complementary to a region of the nucleic acid sequence

(SEQ ID NO: 87)
ATGGCTCGTACAAAGCAGACTGCCCGCAAATCGACCGGTGGTAAAGCACC
CAGGAAGCAACTGGCTACAAAAGCCGCTCGCATGAGTGCGCCCTCTACTG
GAGGGGTGAAGAAACCTCATCGTTACAG.

According to some aspects, the ASO comprises a nucleic acid sequence of about 15 nucleosides to about 30 nucleosides that is at least 80% complementary to a region of the nucleic acid sequence ACTGGCTACAAAAGCCGCTCGCATGAGTGCGCCCTCTACTGGAGGGGTGAAGAA ACCTCATC (SEQ ID NO: 88), wherein the ASO comprises one or more nucleoside chemical modifications.

In some embodiments, the ASO comprises a nucleic acid sequence from about 15 to about 30 nucleosides is at least 95%, or at least 99% complementary to the nucleic acid sequence

(SEQ ID NO: 88)
ACTGGCTACAAAAGCCGCTCGCATGAGTGCGCCCTCTACTGGAGGGGTGA
AGAAACCTCATC.

In some embodiments, the ASO comprises a nucleic acid sequence from about 15 to about 30 nucleosides is 100% complementary to the nucleic acid sequence

(SEQ ID NO: 88)
ACTGGCTACAAAAGCCGCTCGCATGAGTGCGCCCTCTACTGGAGGGGTGA
AGAAACCTCATC.

According to some aspects, the ASO comprises about 15 nucleosides to about 30 nucleosides. In some embodiments, the nucleic acid sequence of the ASO of about 15 nucleosides to about 30 nucleosides is complementary to a region of a mutant H3.3 histone A (H3F3A) allele that comprises a mutation in exon 2 and the ASO of about 15 nucleosides to about 30 nucleosides comprises one or more nucleoside chemical modifications.

According to some aspects, the ASO comprises about 15 nucleosides to about 30 nucleosides. In some embodiments, the nucleic acid sequence of the ASO of about 15 nucleosides to about 30 nucleosides is complementary to a region of a mutant H3.3 histone A (H3F3A) allele that comprises a mutation in exon 2, wherein the ASO of about 15 nucleosides to about 30 nucleosides hybridizes to the mutant H3F3A allele, and does not hybridize to a H3.3 histone B (H3F3B) allele.

According to some aspects, the ASO comprises about 15 nucleosides to about 30 nucleosides. In some embodiments, the nucleic acid sequence of the ASO of about 15 nucleosides to about 30 nucleosides is complementary to a region of a mutant H3.3 histone A (H3F3A) allele that comprises a mutation in exon 2, wherein the ASO of about 15 nucleosides to about 30 nucleosides hybridizes to the mutant H3F3A allele more than it hybridizes to a corresponding wild-type H3F3A allele, or to a H3.3 histone B (H3F3B) allele.

According to some aspects, the ASO comprises about 15 nucleosides to about 30 nucleosides. In some embodiments, the nucleic acid sequence of the ASO of about 15 nucleosides to about 30 nucleosides is complementary to a region of a mutant H3.3 histone A (H3F3A) gene that comprises a mutation in exon 2, wherein the ASO of about 15 nucleosides to about 30 nucleosides hybridizes to the mutant H3F3A gene more than it hybridizes to a H3.3 histone B (H3F3B) gene.

In some embodiments, the mutant H3F3A allele encodes a mutant histone 3.3 (H3.3) protein comprising a lysine (K) to methionine (M) mutation.

In some embodiments, the ASO of about 15 nucleosides to about 30 nucleosides comprises one or more nucleoside chemical modifications.

In some embodiments, the ASO of about 15 nucleosides to about 30 nucleosides is from about 18 nucleosides to about 22 nucleosides.

In some embodiments, the ASO of about 15 nucleosides to about 30 nucleosides is a gapmer ASO of about 15 nucleosides to about 30 nucleosides comprising a 3′-wing, a gap segment and a 5′-wing, wherein the gap segment comprises DNA and one or more chemical modifications in one more internucleoside linkages of the nucleic acid sequence.

In some embodiments, the ASO of about 15 nucleosides to about 30 nucleosides is a splice-modulating ASO.

In some embodiments, the one or more nucleoside chemical modifications are a 2′-O-methoxyethyl (MOE) modification, a locked nucleic acid (LNA) modification, a S-constrained ethyl (cET) modification, a phosphorodiamidate (PDA) morpholino oligomer (PMO) modification, or a 5′-methylcytosine modification.

In some embodiments, the ASO of about 15 nucleosides to about 30 nucleosides comprises one or more chemical modifications in one or more internucleoside linkages of the nucleic acid sequence.

In some embodiments, the one or more chemical modifications in one or more internucleoside linkages comprise a phosphorothioate (PS) modification.

In some embodiments, all of the internucleoside linkages comprise PS modifications.

In some embodiments, the ASO of about 15 nucleosides to about 30 nucleosides is from about 15 nucleosides to about 25 nucleosides.

In some embodiments, the ASO of about 15 nucleosides to about 30 nucleosides is a gapmer ASO of about 15 nucleosides to about 30 nucleosides comprising a 3′-wing, a gap segment and a 5′-wing, and the one or more nucleoside chemical modifications is on one or more nucleosides of the 3′-wing; on one or more nucleosides of the 5′-wing; or on one or more nucleosides of the 3′-wing and one or more nucleosides of the 5′-wing.

In some embodiments, the 3′-wing is from about 5 nucleosides to about 10 nucleosides.

In some embodiments, the 5′-wing is from about 5 nucleosides to about 10 nucleosides.

In some embodiments, the gap segment is from about 5 nucleosides to about 20 nucleosides.

In some embodiments, the ASO of about 15 nucleosides to about 30 nucleosides is from about 19 nucleosides to about 21 nucleosides.

In some embodiments, the ASO of about 15 nucleosides to about 30 nucleosides is about 20 nucleosides.

In some embodiments, the ASO of about 15 nucleosides to about 30 nucleosides is a splice-modulating ASO.

In some embodiments, the mutation in a mutant H3F3A allele is at position 2604 of the nucleic acid sequence of SEQ ID NO: 89.

In some embodiments, the nucleoside chemical modification is a 2′-MOE modification.

In some embodiments, the K to M mutation is at position 27 of the amino acid sequence of SEQ ID NO: 91.

In some embodiments, the region is within the nucleic acid sequence

(SEQ ID NO: 88)
ACTGGCTACAAAAGCCGCTCGCATGAGTGCGCCCTCTACTGGAGGGGTGA
AGAAACCTCATC.

In some embodiments, the ASO comprises the nucleic acid sequence

(SEQ ID NO: 1)
CACTCATGCGAGCGGCTTTT,
(SEQ ID NO: 4)
GCGCACTCATGCGAGCGGCT,
(SEQ ID NO: 5)
GGCGCACTCATGCGAGCGGC,
(SEQ ID NO: 6)
GGGCGCACTCATGCGAGCGG,
(SEQ ID NO: 58)
ACCCCTCCAGTAGAGGGCGC,
(SEQ ID NO: 59)
CAGTAGAGGGCGCACTCATG,
or
(SEQ ID NO: 60)
AGTAGAGGGCGCACTCATGC.

In some embodiments, the ASO consists of the nucleic acid sequence

(SEQ ID NO: 1)
CACTCATGCGAGCGGCTTTT,
(SEQ ID NO: 4)
GCGCACTCATGCGAGCGGCT,
(SEQ ID NO: 5)
GGCGCACTCATGCGAGCGGC,
(SEQ ID NO: 6)
GGGCGCACTCATGCGAGCGG,
(SEQ ID NO: 58)
ACCCCTCCAGTAGAGGGCGC,
(SEQ ID NO: 59)
CAGTAGAGGGCGCACTCATG,
or
(SEQ ID NO: 60)
AGTAGAGGGCGCACTCATGC.

In some embodiments, the ASO comprises the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

In some embodiments, the ASO consists of the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

In some embodiments, the ASO reduces expression of the mutant H3F3A allele and does not reduce expression of the H3F3B allele.

In some embodiments, the ASO is a single-stranded ASO.

In some embodiments, the mutant H3F3A allele is a dominant mutation that encodes a point mutation in non-canonical H3.3 protein found in/characteristic of pediatric diffuse midline gliomas.

In some embodiments, the ASO hybridizes under physiological conditions.

### BRIEF DESCRIPTION OF THE DRAWINGS

Two sets of drawings, one in color and one in grayscale, are being filed with this application.

FIGS. 1A-1J. CRISPR-Cas9 depletion of H3.3-K27M rescued H3-K27 trimethylation and delayed growth of patient-derived neurospheres and orthotopic xenografts. (FIGS. 1A-1B) Immunoblots assessing H3.3-K27M knockout efficiency and epigenetic changes in DIPG patient cells; (FIG. 1C) Representative IF image showing restoration of H3K27me3 in DIPG cells after knockout of H3.3-K27M, and slower proliferation measured by EdU staining; (FIG. 1D) Quantification of EdU-positive cells; (FIG. 1E) Cell-viability assays at each time point were performed in triplicate experiments, with 3 wells per condition; (FIGS. 1F-1G) Effect of H3.3-K27M knockout in patient cells on soft-agar colony formation; (FIG. 1H) K27M-mutant DIPG cells (SU-DIPG-XIII) and cells with H3F3A knocked out were implanted in the brain of immunocompromised mice. Tumor growth was monitored by in vivo luciferase-activity imaging. Equal numbers of cells for each condition were implanted at P2, and representative images at P38 are shown; (FIG. 1I) Kaplan-Meier estimates of overall survival showed a significant delay in tumor growth upon knockout of H3.3-K27M (P=0.039); (FIG. 1J) Representative IHC images for H3K27me3, NEUN, GFAP and KI67; For immunoblot and IF, the measurements for each experimental group/treatment were analyzed by ANOVA, followed by pairwise comparisons using two-sample t-tests. For cell viability, a linear mixed-effects model was used for the comparison.

FIGS. 2A-2F. Schematic representation of tested chemically modified “gapmer” ASOs targeting H3F3A exon 2. (FIG. 2A) Diagram of a single-stranded “gapmer” ASO with a central DNA region (black), 2′-O-methoxyethyl (MOE) wings (white) and phosphorothioate (PS) backbones; (FIG. 2B) Mechanism of RNA knockdown by gapmer ASOs, depicting RNase-H-mediated cleavage of the RNA in DNA-RNA hybrids; (FIG. 2C) Modified ASO chemistry; (FIG. 2D) The 151-nucleotide exon 2 was targeted by overlapping 20-mer ASOs at various intervals, and each underlined nucleotide in the mRNA marks the start of the sequence targeted by an ASO (SEQ ID NO: 176); (FIG. 2E) Sequence alignment around the mutation region, showing the H3F3A mutant allele with T (underlined) and the wt allele with A (boxed) (SEQ ID NOs: 177-192); divergent nucleotides relative to the H3F3A mutant allele are show in bold; (FIG. 2F) Schematic representation of the wt and mut minigene constructs, comprising exons 1 to 3 and the natural introns.

FIGS. 3A-3E. ASO-mediated H3.3-K27M depletion restored global H3K27me3. (FIG. 3A) ASO screen using H3F3A wt and mut minigenes. HeLa cells were co-transfected with the minigenes, along with individual 20-mer PO-MOE-ASOs, using Lipofectamine 2000; two days later, the extent of knock down was quantified via radioactive RT-PCR, with allele-specific primers; (FIG. 3B) ASO screen in patient-derived (SU_DIPG_XIII) neurosphere cultures by free uptake, using RT-qPCR of total RNA extracted after 5 days; (FIG. 3C) Dose-response experiment in patient cells by lipofectamine transfection; (FIG. 3D) Decrease of H3F3A mRNA measured by RT-qPCR in three patient-derived cell lines, detected with allele-specific primers to distinguish mutant and wild-type alleles; (FIG. 3E) Immunoblot of acid-extracted histones from each patient cell line, showing that the level of H3.3-K27M protein loss correlates with a reciprocal H3K27me3 gain. For immunoblots and RT-qPCR experiments, the measurements for each experimental group/treatment were analyzed by ANOVA, followed by pairwise comparisons using two-sample t-tests.

FIGS. 4A-4G. ASO-mediated H3.3 K27M depletion delayed neurosphere growth and changed cell morphology. (FIG. 4A) Cell-viability assays at each time point for each cell line were performed in triplicate experiments, with 3 wells per condition; solid line for 8atrigel-coated monolayer culture, and dashed line for neurosphere culture; for all pairwise comparisons between treatment and control, P<0.001 (adjusted p values by single-step method); (FIG. 4B) Cell-viability assays at each time point for each ASO at low or high dose were performed in triplicate (n=3 wells per condition) (FIGS. 4C-4E) Cell-viability assays at each time point for each cell line were performed in triplicate (n=3 wells per condition); (FIG. 4F) Representative images of SU-DIPG-XIII, SU-DIPG-50, and SU-DIPG-35 patient cells treated with ASO2 or control Scramble ASO by free uptake for 5 days. Black arrows indicate neurite-like processes. Scale bars, 1000 μm; (FIG. 4G) Quantification of average sphere size in pixels (μm) from the images in (FIG. 4F).

FIG. 5A-5C. RCAS-TVA mouse model to study the effect of H3.3 K27M mutation during a brain-development window. (FIG. 5A) Diagram of RCAS plasmids; 10⁵ RCAS-Pdgfb, RCAS-Cre and RCAS-H3F3A-mutant cDNA expressing producer cells (DF1) were injected into the brainstem of Nestin-TVA; p53^{fl/fl} mice at postnatal day 3; tumorigenesis started from week 3 post-infection, as confirmed by IHC staining; (FIG. 5B) Representative H&E (40×) stained murine tumors driven by PDGFB signaling and loss of p53 in combination with H3.3K27M, and IHC staining (40×) of these tumors with FLAG-tag antibody (n=8); (FIG. 5C) H&E (20×) and IHC staining with anti-H3K27me3 (20×) in representative tumor lesions (n=8).

FIGS. 6A-6E. ICV administration of ASO at the time of tumor onset. (FIG. 6A) Stereotactic ICV injection of a single dose (500 μg) of lead ASO or CTRL ASO in saline was given at the time of tumor onset (˜day 21); RNA, protein and histology samples were collected at the end points when the mice were symptomatic, including an enlarged head, ataxia, or >25% weight loss; (FIG. 6B) Quantification of mRNA levels of H3F3A mutant allele, flag, endogenous murine H3F3A and H3f3b (n=5 independent ICV injections for each ASO); (FIG. 6C) Immunoblot of acid-extracted histones from each treated mouse; (FIG. 6D-FIG. 6E) Representative H&E (10×) stained and high-magnification (40×) murine tumors, and IHC staining with GFAP and NeuN antibodies. For immunoblots and RT-qPCR experiments, the measurements for each experimental group/treatment were analyzed by ANOVA, followed by pairwise comparisons using two-sample t-tests.

FIGS. 7A-7D. ASO-mediated H3.3-K27M depletion induced inflammation, promoted A2-specific reactive astrocyte differentiation, and decreased tumor proliferation. (FIG. 7A) Heat map of A1/2-specific astrocyte markers in uninfected saline-treated mice (n=5), and ASO CTRL or ASO 5 treated H3.3 K27M-tumor mice (n=5 each); (FIG. 7B) Quantification of microglia-activation marker Aif1 mRNA level (n=3); (FIG. 7C) Quantification of IF staining in (FIG. 7D) of GFAP- and Ki67-positive cells, for each condition; cells were counted in 5 randomly picked fields at 40× magnification. The cell counts were analyzed by ANOVA, followed by t-tests for the pairwise comparisons; (FIG. 7D) Representative IF images showing strikingly elevated GFAP-positive cells (green) and slower proliferation by Ki67 staining (red); DAPI staining shows nuclei (blue) (left); higher-magnification images (right).

FIGS. 8A-8G. ASO-mediated H3.3-K27M depletion induced neuron and oligodendrocyte differentiation, and decreased tumor proliferation. (FIGS. 8A-8C) Representative IF images showing strikingly elevated NeuN-positive cells and slower proliferation by Ki67 staining; DAPI staining shows nuclei (left); higher-magnification images (right). (FIGS. 8D-8F) Representative IF images showing strikingly elevated MBP-positive cells and slower proliferation by Ki67 staining; DAPI staining shows nuclei (left); higher-magnification images (right, scale bar, 20 μm). (FIG. 8G) Quantification of IF staining in (FIGS. 8A-8C) and Ki67-positive cells, for each condition; cells were counted in 5 randomly picked fields at 40× magnification. The cell counts were analyzed by ANOVA, followed by t-tests for the pairwise comparisons.

FIGS. 9A-9C. ICV injection of ASO 5 decreased the NESTIN⁺ cell population and extended the latency of tumor growth in the mouse model. (FIG. 9A) Representative IF images showing a striking decrease in NESTIN-positive cells and elevated GFAP-positive in ASO-treated tumor lesions; DAPI staining shows nuclei; higher-magnification images with scale bar 20 μm; (FIG. 9B) Kaplan-Meier survival analysis of cohorts with induced PDGFR signaling and p53 depletion, combined with CTRL ASO treatment (n=22) or ASO 5 treatment (n=21); (FIG. 9C) Schematic model of ASO treatment in RCAS-TVA mouse model; CTRL ASO treated mice retained a large NESTIN⁺ population with a highly proliferative state; ASO-5-mediated H3.3 K37M depletion decreased proliferation, and promoted differentiation into NeuN⁺, GFAP⁺ and MBP⁺ cells.

FIG. 10. Schematic representation of 1-nt microwalk of the splice modulating ASOs targeting H3F3A 5′ splice site downstream of the exon 2. 20-mer ASOs uniformly modified by 2′-O-methoxyethyl (MOE) and phosphorothioate (PS). Sequence alignment around the 5′ splice site of H3F3A and H3F3B, respectively, showing the H3F3A mutant allele with U (italics) and the wt allele with A (boxed); divergent nucleotides relative to the H3F3A 5′ splice site are underlined (SEQ ID NOs: 193-194).

FIGS. 11A-11C. (FIG. 11A) one of lead ASOs targeting 5′ splice site of H3F3A exon 2, ASO 58 promoted 100% mutant exon skipping using H3F3A wt and mut minigenes. HeLa cells were co-transfected with the minigenes, along with ASO 58, using Lipofectamine 2000; two days later, the splicing changes was detected and quantified via radioactive RT-PCR, (FIG. 11B) schema of a vector specific primer pair across T7 promoter region and exon3; (FIG. 11C) sanger sequencing confirmed the mutant exon 2 skipping (SEQ ID NOs: 195-196).

FIGS. 12A-12B. ASO screen in patient-derived (SU_DIPG_XIII) neurosphere cultures by free uptake, using radioactive RT-PCR of total RNA extracted after 5 days. (FIG. 12A) both full length and skipped products were detected using a primer pair across exon 1 and 3 on 5% native PAGE gel, and quantification; (FIG. 12B) with allele-specific primers, and quantification. n=3 biological replicates.

FIG. 13. Schematic representation of 1-nt microwalk of the splice modulating ASOs targeting H3F3A exon 2. 20-mer ASOs uniformly modified by 2′-O-methoxyethyl (MOE) and phosphorothioate (PS). Sequence showing the H3F3A mutant allele with T (italics) and the wt allele with A (boxed); and 5′ spice site underlined (SEQ ID NO: 197).

FIG. 14. ASOs screen using H3F3A wt and mut minigenes. HeLa cells were co-transfected with the minigenes, along with individual 20-mer ASOs, using Lipofectamine 2000; two days later, the splicing changes was detected via radioactive RT-PCR, with a primer set across exon 1 and 3, ASO 58 targeting 5′splice site as a positive control.

FIGS. 15A-15D. Splice-switching ASO in patient neurospheres. (FIG. 15A) ASO58 MOE promoted skipping of the exon comprising K27, resulting in downregulation of the full-length mRNA, detected by radioactive RT-PCR; (FIG. 15B) immunoblot of acid-extracted histones shows the reduction in H3.3-K27M protein and restoration of H3K27me3, as well as total H3 histones; (FIG. 15C) The treatment slows down tumor-cell growth; cell-viability assays at each time point were performed in triplicate experiments, with 3 wells per condition (*** P<0.001); (FIG. 15D) Representative images of SU-DIPG-XIII cells treated with ASO58 MOE by free uptake for 5 days. Black arrowheads indicate neurite-like processes.

FIGS. 16A-16F. Schematic representation of tested chemically modified “gapmer” ASOs targeting H3F3A exon 2. (FIG. 16A) Diagram of a single-stranded “gapmer” ASO with a central DNA region, 2′-O-methoxyethyl (MOE) wings and phosphorothioate (PS) backbones; (FIG. 16B) Mechanism of RNA knockdown by gapmer ASOs, depicting RNase-H-mediated cleavage of the RNA in DNA-RNA hybrids, (diagram created with BioRender.com); (FIG. 16C) Modified ASO chemistry; (FIG. 16D) The 151-nucleotide exon 2 (SEQ ID NO: 200) was targeted by overlapping 20-mer ASOs at various intervals, and each underlined nucleotide in the mRNA marks the start of the sequence targeted by an ASO; (FIG. 16E) Sequence alignment around the mutation region (SEQ ID NOs: 201-216), showing the H3F3A mutant allele with T and the WT allele with A; divergent nucleotides relative to the H3F3A mutant allele are also shown; (FIG. 16F) Schematic representation of the WT and MUT minigene constructs, comprising exons 1 to 3 and the natural introns.

FIGS. 17A-17D. ASO-mediated H3.3-K27M depletion restored global H3K27me3. (FIG. 17A) ASO screen using H3F3A WT and MUT minigenes. HeLa cells were co-transfected with the minigenes, along with individual 20-mer PO-MOE-ASOs, using Lipofectamine 2000; two days later, the extent of knockdown was quantified by radioactive RT-PCR with allele-specific primers, band intensities were quantified; (FIG. 17B) ASO screen by free uptake in patient-derived (SU-DIPG-XIII) neurosphere cultures, using RT-qPCR of total RNA extracted after 5 days; (FIG. 17C) Dose response experiment with co-transfected minigenes in HeLa cells; (FIG. 17D) Immunoblot of acid-extracted histones from each patient cell line, showing that the level of H3.3-K27M protein loss correlates with a reciprocal H3K27me3 gain, band intensities were quantified. For immunoblots and RT-qPCR experiments, the measurements for each experimental group/treatment were analyzed by ANOVA, followed by pairwise comparisons using two-sample t-tests.

FIGS. 18A-18F. ASO-mediated H3.3 K27M depletion delayed neurosphere growth and changed cell morphology. (FIGS. 18A-18C) Cell-viability assays at each time point for each H3.3 K27M cell line were performed in triplicate (n=3 wells per condition); (FIG. 18D) Cell-viability assays at each time point for each H3.3 WT cell line were performed in triplicate (n=3 wells per condition); (FIG. 18E) Representative images of SU-DIPG-XIII, SU-DIPG-50, and SU-DIPG-35 patient cells treated with ASO1, ASO5, or control Scramble ASO by free uptake for 5 days. Black arrows indicate neurite-like processes. Scale bars, 1000 m; (FIG. 18F) Quantification of average neurospheres size in (m) from the images in FIG. 18E. For viability assays, P-values were adjusted for multiple comparisons by controlling familywise error rate using the single-step method. Significance codes: 0.001 ‘***’; 0.01 ‘**’; >0.05 ‘n.s.’. For neurosphere size, the measurements for each experimental group/treatment were analyzed by ANOVA, followed by pairwise comparisons using twosample t-tests.

FIGS. 19A-19D. ICV administration of ASO at the time of tumor onset in RCAS-TVA mouse model. (FIG. 19A) Diagram (created with BioRender) of RCAS plasmids; 105 RCAS-Pdgfb, RCAS-Cre, and RCAS-H3F3A-mutant cDNA-expressing producer cells (DF1) were injected into the brainstem of Nestin-Tva; p53fl/fl mice at postnatal day 3; a single dose (500 μg) of lead ASO or CTRL ASO in saline was stereotaxically injected ICV on day 21; RNA, protein, and histology samples were collected at the end points when the mice were symptomatic, including an enlarged head, ataxia, or >25% weight loss, created with BioRender.com; (FIG. 19B) Quantification of mRNA levels of H3F3A mutant allele, flag tag, endogenous murine H3f3a and H3f3b (n=5 independent ICV injections for each ASO); (FIG. 19C) Immunoblot of acid-extracted histones from each treated mouse, band intensities were quantified; (FIG. 19D) Representative H&E stained tumors confirming their location in the midline region (left); control-ASO-treated cohorts developed high-grade tumors; ASO5-treated cohorts developed lower grade tumors with elongated morphology. For RT-qPCR experiments, the measurements for each experimental group/treatment were analyzed by Welch's two-sample t-test to compare the H3F3A MUT expression normalized to the GAPDH loading control between CTRL ASO and ASO5.

FIGS. 20A-20G. ASO-mediated H3.3-K27M depletion induced astrocyte, neuron, and oligodendrocyte differentiation, decreased tumor proliferation and the NESTIN+ cell population, and extended the latency of tumor growth in the Nestin-Tva mouse model. (FIG. 20A) Representative IF images showing normal differentiation in H3.3 WT tumors; (FIG. 20B) Representative IF images showing strikingly elevated GFAP+, NeuN+, and MBP+ cells and slower proliferation by Ki67 staining; DAPI staining shows nuclei (left: scale bar 100 m); higher-magnification images (right: 20 m); (FIGS. 20C-20D) Quantification of IF staining in (FIG. 20C) GFAP+ and Ki67+ cells and in (FIG. 20D) NeuN+ and Ki67+ cells; (FIG. 20E) Immunoblot of differentiation markers (GFAP, NeuN, and MBP) in tissue samples prepared from normal adjacent and tumor lesions band intensities were quantified; (FIG. 20F) Representative IF images showing a striking decrease in NESTIN+ cells and elevated GFAP+ cells in ASO-treated tumor lesions; DAPI staining shows nuclei; higher-magnification images with scale bar 20 m; (FIG. 20G) Kaplan-Meier survival analysis of cohorts with induced PDGFR signaling and p53 depletion, combined with CTRL ASO treatment (n=22) or ASO5 treatment (n=21). For IF quantification, cells were counted in 5 randomly picked fields at 40× magnification. The cell counts were analyzed by ANOVA, followed by t-tests for the pairwise comparisons.

FIGS. 21A-21E. ICV administration of ASO at the time of tumor onset induced human specific astrocyte, neuron, and oligodendrocyte differentiation, decreased tumor proliferation, and extended the latency of tumor growth in a patient-cell-derived xenograft mouse model. (FIG. 21A) A single dose (200 μg) of lead ASO or CTRL ASO in saline was stereotaxically injected ICV at the time of tumor onset (˜day 21) (diagram created with BioRender); (FIG. 21B) Kaplan-Meier survival analysis of SU-DIPG-XIII-Luc xenograft cohorts after CTRL ASO (n=5) or ASO5 treatment (n=5); (FIG. 21C) Representative IF images showing strikingly elevated GFAP+, NeuN+ and MBP+ cells and slower proliferation by Ki67 staining; DAPI staining shows nuclei (left: scale bar 100 m); higher-magnification images (right: 20 m); (FIG. 21D) SU-DIPG-XIII-Luc xenograft sections were co-stained with human-specific SMN plus GFAP, NeuN, or MBP antibodies; (FIG. 21E) Schematic model (created with BioRender) of ASO treatment in H3.3 K27M mouse models; CTRL-ASO-treated mice retained a highly proliferative state; ASO-5-mediated H3.3 K37M depletion decreased proliferation, and promoted differentiation into NeuN+, GFAP+, and MBP+ cells.

FIGS. 22A-22J. CRISPR-Cas9 depletion of H3.3-K27M rescued H3-K27 trimethylation and delayed growth of patient-derived neurospheres and orthotopic xenografts. (FIGS. 22A-22B) Immunoblots assessing H3.3-K27M knockout efficiency and epigenetic changes in DIPG patient cells; (FIG. 22C) Representative IF image showing restoration of H3K27me3 in DIPG cells after knockout of H3.3-K27M, and slower proliferation measured by EdU staining; (FIG. 22D) Quantification of EdU-positive cells; (FIG. 22E) Cell-viability assays at each time point were performed in triplicate experiments, with 3 wells per condition; (FIGS. 22F-22G) Effect of H3.3-K27M knockout in patient cells on softagar colony formation; (FIG. 22H) K27M-mutant DIPG cells (SU-DIPG-XIII) and cells with H3F3A knocked out were implanted in the brain of immunocompromised mice. Tumor growth was monitored by in vivo luciferase-activity imaging. Equal numbers of cells for each condition were implanted at P2, and representative images at P38 are shown; (FIG. 22I) Kaplan-Meier estimates of overall survival showed a significant delay in tumor growth upon knockout of H3.3-K27M (P=0.039); (FIG. 22J) Representative IHC images for H3K27me3, NeuN, GFAP and Ki67; For immunoblotting and IF, the measurements for each experimental group/treatment were analyzed by ANOVA, followed by pairwise comparisons using two-sample t-tests. For cell viability, a linear mixed-effects model was used for the comparison.

FIGS. 23A-23C. (FIG. 23A) ASO5 had no effect on H3F3A or H3F3B mRNA levels in H3.3 WT glioma cells, quantified by real-time RT-PCR with gene-specific and allele-specific primers; (FIG. 23B) likewise at the H3.3 protein level, quantified by western blotting using H3.3 WT and total H3 antibodies. (FIG. 23C) Representative images of H3.3 WT glioma neurosphere cultures after ASO5 treatment by free uptake for 5 days at 1, 5, and 10 μM concentrations.

FIGS. 24A-24C. (FIG. 24A) Representative H&E-stained sections (40×) of murine normal-adjacent and tumor tissues, and IHC staining of Flag-tagged H3K27M (scale bar 200 m); (FIG. 24B) Representative H&E-stained sections (20×) of murine tumors, and IHC staining with Flag and H3K27me3 antibodies (scale bar: 500 m); (FIG. 24C) Representative H&E-stained sections (20× (left, middle) and 40× (right)) of murine tumors, and IHC staining with Olig2 antibody (scale bar: 200 m).

FIGS. 25A-25B. ASO-mediated H3.3-K27M depletion induced inflammation, and promoted A2-specific reactive-astrocyte differentiation. (FIG. 25A) Quantification of microglia-activation marker Aif1 mRNA level (n=3). (FIG. 25B) Heat map of A1/2-specific astrocyte markers in uninfected saline-treated mice (n=5), and ASO CTRL or ASO5-treated H3.3 K27M tumor mice (n=5 each).

### DETAILED DESCRIPTION

A specific heterozygous point mutation in the non-canonical histone H3.3 is found in 70-80% of diffuse midline glioma (DMG) tumors. This dominant mutation in H3F3A one of two genes encoding identical H3.3 proteins-replaces lysine 27 with methionine (K27M). H3.3 K27M is a toxic gain-of-function mutation that inhibits the EZH2 methyltransferase subunit of the Polycomb repressive complex (PRC2), leading to global reduction of di- and tri-methylation on histone proteins. This is thought to be a driving event in tumorigenesis (3,4). It was hypothesized that DMG tumors remain dependent on H3.3 K27M, such that reducing H3.3 K27M expression will have anti-tumor effects. Importantly, K27M tumors can be diagnosed by MRI and stereotactic biopsy (5,6).

To achieve targeted gene-specific or allele-specific knockdown, the potential of antisense oligonucleotides (ASOs) in DIPG patient cells was evaluated, using two distinct ASO modalities. First, “gapmer” ASOs were designed with a DNA-like central region that directs cleavage of a complementary mRNA (or pre-mRNA) target by endogenous RNase H, and chemically modified “wings” that promote tighter RNA binding, enhanced stability, and improved cellular uptake. Second, splice-modulating ASOs were designed that promote skipping of the exon with the mutation in H3F3A. These uniformly modified ASOs do not elicit RNase H cleavage. Uniformly modified ASOs for DMG are designed to sterically block a 5′ or 3′ splice site, or a splicing-enhancer element, reducing the expression of correctly spliced H3F3A mRNA, and therefore the expression of the mutant histone protein. Crucially, wild-type H3.3 protein is still expressed from the H3F3B gene. Promising ASOs, representing both approaches, were identified. Lead ASOs were evaluated using mouse models of DMG.

### Antisense Oligonucleotides (ASOs)

One embodiment of the present disclosure is a composition comprising nucleic acids and/or nucleic acid analogs, such as polynucleotides, that inhibit expression of mutant genes that are associated with brain cancer. The nucleic acids or polynucleotides are, for example, antisense oligonucleotides (ASOs) that bind to a specific region of an mRNA transcript (e.g., a mutation) modulate pre-mRNA splicing, direct cleavage of a complementary mRNA (or pre-mRNA) target by endogenous RNase H, or promote skipping of the exon comprising the mutation of interest. In all embodiments herein, referring to “an mRNA,” or “the mRNA” means one or more (at least one) mRNA molecules. The terms “antisense oligonucleotide,” “ASO” and “antisense oligomer” are used interchangeably and refer to a polynucleotide, comprising nucleotides, that hybridizes to/with a target nucleic acid (e.g., mRNA) sequence by Watson-Crick base pairing or wobble base pairing (G-U). The terms gapmers (gapmer ASO) and splice modulating ASOs are both encompassed by the term ASO. An ASO may have exact sequence complementarity to a target sequence or near complementarity (e.g., sufficient complementarity to bind the target sequence and inhibit splicing or direct mRNA degradation). An ASO is designed so that it binds (hybridizes) to/with a target nucleic acid (e.g., a mRNA transcript) and remains hybridized under physiological conditions. Design of an ASO can take into consideration the occurrence of the target nucleic acid sequence or a sufficiently similar nucleic acid sequence in other locations in the genome or cellular mRNA/transcriptome, such that the likelihood the ASO will bind other sites and cause “off-target” effects is limited. In some embodiments, ASOs are chemically modified, such as with chemical modifications described herein. ASOs are single stranded oligonucleotides. The term ASO does not include a small hairpin RNA (shRNA) or a CRISPR guide RNA.

In some embodiments, an ASO “specifically hybridizes” to/with or is “specific” to a target nucleic acid. Such hybridization occurs with a Tm substantially greater than 37° C., preferably at least 50° C., and typically 60° C.-80° C. or higher. Such hybridization preferably corresponds to stringent hybridization conditions. At a given ionic strength and pH, the Tm is the temperature at which 50% of a target sequence hybridizes to/with a complementary oligonucleotide. As used herein, the term “specifically binding”, “specific binding”, “specifically hybridize” or “specific” in the context of an ASO refers to an ASO that has a higher binding affinity for one gene allele over another gene allele (e.g. a higher binding affinity for the mRNA encoded by the H3F3A K27M allele than the mRNA encoded by the H3F3A wildtype allele under the same conditions or when assessed under the same conditions, such as under physiological conditions). In some embodiments, an ASO that is specific for a mutant allele has a higher binding affinity for the mutant allele than for the corresponding wildtype (WT) allele. In some embodiments, a specific ASO preferentially decreases the expression of one gene allele over one or more other gene allele(s) (e.g., preferentially decreases the expression of the mRNA encoded by the H3F3A K27M allele over the mRNA encoded by the H3F3A wildtype allele under the same conditions or when assessed under the same conditions, such as under physiological conditions).

Polynucleotides (e.g., oligonucleotides, ASOs, mRNA, etc.) are “complementary” to one another when hybridization occurs in an antiparallel configuration between two single-stranded polynucleotides. A double-stranded polynucleotide can be “complementary” to another polynucleotide when hybridization can occur between one of the strands of the first polynucleotide and one strand of the second polynucleotide. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an oligomeric compound, e.g., an ASO, need not be 100% complementary to that of its target nucleic acid to hybridize. In certain embodiments, ASOs can comprise at least about 70%, at least about 80%, at least about 90%, at least about 95%, or at least about 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining noncomplementary nucleobases may be clustered together or interspersed with complementary nucleobases and need not be contiguous to each other or to complementary nucleobases. An ASO which is 18 nucleobases in length having 4 (four) noncomplementary nucleobases which are flanked by two regions of complete complementarity with the target nucleic acid would have 77.8% overall complementarity with the target nucleic acid and would thus fall within this scope. Percent complementarity of an ASO with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

An ASO need not hybridize to all nucleobases in a target sequence and the nucleobases to which it hybridizes may be contiguous or noncontiguous. ASOs may hybridize over one or more segments of a target nucleic acid, such that intervening or adjacent segments are not involved in the hybridization event (e.g., a loop structure or hairpin structure). In certain embodiments, an ASO hybridizes to/with noncontiguous nucleobases in a target nucleic acid. For example, an ASO can hybridize to/with nucleobases in a target nucleic acid that are separated by one or more nucleobase(s) to which the ASO does not hybridize to/with.

The ASO may be comprised of naturally-occurring nucleotides, nucleotide analogs, modified nucleotides, or any combination of two or three of the preceding (naturally-occurring nucleotides and nucleotide analogs; naturally-occurring nucleotides and modified nucleotides; nucleotide analogs and modified nucleotides; naturally-occurring nucleotides, nucleotide analogs and modified nucleotides). The term “naturally occurring nucleotides” includes deoxyribonucleotides and ribonucleotides. The term “modified nucleotides” includes nucleotides with modified or substituted sugar groups and/or having modified oligonucleotide linkages. The term “oligonucleotide linkages” includes (but is not limited to) oligonucleotides linkages such as phosphorothioate, phosphorodithioate, phosphoroselerloate, phosphorodiselenoate, phosphoroanilothioate, phoshoraniladate, phosphoramidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990).

In some embodiments, the ASO is comprised of 2′-O-(2-methoxyethyl) (MOE) phosphorothioate-modified nucleotides. An ASO comprised of such nucleotides is especially well-suited to the present methods; oligonucleotides having such modifications have been shown to have significantly enhanced resistance to nuclease degradation and increased bioavailability, making them suitable, for example, for oral delivery. See e.g., Geary et al., J Pharmacol Exp Ther. 2001; 296(3):890-7; Geary et al., J Pharmacol Exp Ther. 2001; 296(3):898-904.

In some embodiments, the ASO comprises naturally occurring nucleotides and nucleotide linkages. In some embodiments, the ASO comprises deoxynucleotides. In some embodiments, the ASO comprise modified nucleotides. In some embodiments, the ASOs modified deoxynucleotides. In some embodiments, ASO comprise 2′-H modified nucleotides. In some embodiments, the central block of the ASO comprise phosphorothioate (PS) backbone, a morpholino backbone or a peptide nucleic acid (PNA) backbone. In some embodiments, the ASO comprises 2′-O-methyl modified ribose (2′OMe), 2′-O-methoxyethyl modified ribose (2′-MOE), or 2′-fluoro modified ribose (2-′F) modified nucleotides. In some embodiments, the ASO comprises a locked nucleic acid (LNA), a constrained ethyl ribose (cEt) nucleic acid, a Tricycol-DNA (tc-DNA) nucleic acid, a 5′methylcytosine (m⁵C) nucleic acid, or a N-acetylgalactosamine (GalNAc) nucleic acid.

In some embodiments, the ASOs bind to or is specific to (e.g., bind in a gene or allele-specific manner) to a region of a nucleic acid (e.g., DNA or RNA), or a product thereof, that comprises a mutation. In some embodiments, the ASO is complementary to a product of the mutant H3.3 histone A (H3F3A) allele or gene that comprises a mutation in exon 2 and encodes a mutant histone 3.3 (H3.3) protein comprising a lysine (K) to methionine (M) mutation. In some embodiments, a product thereof, such as the product of a gene or allele disclosed herein, refers to a nucleic acid, such as pre-splicing mRNA (pre-mRNA), messenger RNA (mRNA), non-coding RNA (e.g., transfer RNA (tRNA), ribosomal RNA (rRNA), or small nuclear RNA (snRNA). In some embodiments, the ASOs bind to or is specific to (e.g., bind in a gene-specific manner to) a region of a nucleic acid (e.g., mRNA) that comprises a mutation or the ASO modulates pre-mRNA splicing. Unless specified otherwise, the left-hand end of single-stranded nucleic acid (e.g., mRNA, oligonucleotide, ASO etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., a mutation) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

The ASOs may be of any length suitable for specific binding to an mRNA comprising a mutation or modulating pre-mRNA splicing. For example, the ASO may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides or nucleosides in length. In some embodiments, the ASO is between about 10 and about 30 nucleotides or nucleosides in length, about 10 and about 20 nucleotides or nucleosides in length, or about 15 nucleotides or nucleosides in length. In some embodiments, the ASO is 18-22 nucleotides or nucleosides in length.

In some embodiments, the ASO is designed to specifically bind an mRNA encoding a given gene comprising a mutation over a mRNA encoding a wildtype copy of the given gene. In some embodiments, the ASO is designed to specifically bind a pre-mRNA and block pre-mRNA splicing. In some embodiments, two or more ASOs are designed and used to specifically target an mRNA comprising a mutation or to block splicing of a pre-mRNA. In some embodiments, the given gene is H3F3A or a mutated H3F3A. In some embodiments, the ASO comprises a nucleic acid sequence that is at least 70% identical to any one of SEQ ID NOs: 1-77 (e.g., at least 70% complementary, at least 80% complementary, at least 95% complementary, or at least 99% complementary to any one of SEQ ID NOs: 1-77). In some embodiments, the ASO comprises a nucleic acid sequence that is at least 70% identical to any one of SEQ ID NOs: 1-77 (e.g., at least 70% identical, at least 80% identical, at least 95% identical, or at least 99% identical to any one of SEQ ID NOs: 1-77). In some embodiments, the ASO comprises any one of SEQ ID NOs: 1-77. In some embodiments, the ASO comprises any one of SEQ ID NOs: 1-77. In some embodiments, the ASO comprises any one of SEQ ID NOs: 94-170. In some embodiments, the ASO comprises a nucleic acid sequence that is at least 70% identical to any one of SEQ ID NOs: 94-170 (e.g., at least 70% identical, at least 80% identical, at least 95% identical, or at least 99% identical to any one of SEQ ID NOs: 94-170).

### Gapmers

In some embodiments, the ASOs described herein are gapmers. Gapmers are antisense oligonucleotides that comprise a central block and modified nucleotides at both the 5′ and 3′ ends of the central block. The modified nucleotides at the 5′ and 3′ ends of the gapmer can be referred to as the 3′ wing and 5′ wing. The wings enhance gapmer binding affinity for RNA and increase gapmer nuclease resistance. The central block of the gapmer supports RNase-H1 cleavage of the target mRNA.

In some embodiments, the central block of the gapmer comprises naturally occurring nucleotides and nucleotide linkages. In some embodiments, the central block comprises deoxynucleotides. In some embodiments, the central block comprises modified nucleotides. In some embodiments, the central block comprises modified deoxynucleotides. In some embodiments, the central block comprises 2′-H modified nucleotides. In some embodiments, the central block of the gapmer comprises phosphorothioate (PS) backbone, a morpholino backbone or a peptide nucleic acid (PNA) backbone. In some embodiments, the central block of the gapmer comprises 2′-O-methyl modified ribose (2′OMe), 2′-O-methoxyethyl modified ribose (2′-MOE), or 2′-fluoro modified ribose (2-′F) modified nucleotides. In some embodiments, the central block of the gapmer comprises a locked nucleic acid (LNA), a constrained ethyl ribose (cEt) nucleic acid, a Tricycol-DNA (tc-DNA) nucleic acid, a 5′methylcytosine (m⁵C) nucleic acid, or a N-acetylgalactosamine (GalNAc) nucleic acid. gapmer modifications are further described in Adachi, Hironori, et al. “From Antisense RNA to RNA Modification: Therapeutic Potential of RNA-Based Technologies.” Biomedicines 9.5 (2021): 550, which is incorporated by reference in its entirety.

In some embodiments, the wings of the gapmer comprise naturally occurring nucleotides and nucleotide linkages. In some embodiments, the wings of the gapmer comprise modified ribonucleotides. In some embodiments, the wings of the gapmer comprise 2′-H modified nucleotides. In some embodiments, the wings of the gapmer comprise phosphorothioate (PS) backbone, a morpholino backbone, or a peptide nucleic acid (PNA) backbone. In some embodiments, the wings of the gapmer comprise 2′-O-methyl modified ribose (2′OMe), 2′-O-methoxyethyl modified ribose (2′-MOE), and/or 2′-fluoro modified ribose (2-′F) modifications. In some embodiments, the 2′ modified oligonucleotides comprise 2′-O-methoxyethyl (2′-MOE) modifications. In some embodiments, the wings of the gapmer comprise a locked nucleic acid (LNA), a constrained ethyl ribose (cEt) nucleic acid, a Tricycol-DNA (tc-DNA) nucleic acid, a 5′methylcytosine (m⁵C) nucleic acid, and/or a N-acetylgalactosamine (GalNAc) nucleic acid.

In some embodiments, the central block comprises 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the central block of the gapmer comprises 5-10, 7-12, 10-15, 13-18, 15-20, 18-23, 20-25, or 25-30 nucleotides. In some embodiments, the central block comprises 8-12 nucleotides. In some embodiments, the 5′ wing comprises 1, 2, 3, 4, 5, 6, 7, 8, 9, or 10 nucleotides. In some embodiments, the 3′ wing comprises 1, 2, 3, 4, 5, 6, 7, 8, 9, or 10 nucleotides. In some embodiments, the gapmer comprises a 5 ribonucleotide 5′ wing, a 10 deoxynucleotide central block, and a 5 ribonucleotide 3′ wing, which is also referred to as a 5-10-5 gapmer. In some embodiments, wings of the 5-10-5 gapmer comprise 2′-MOE modifications. In some embodiments, the 5-10-5 gapmer comprises a phosphorothioate backbone. In some embodiments, the gapmer binds to a pre-mRNA at a location such that splicing of the pre-mRNA is decreased. In some embodiments, the gapmer binds to a mutation in an mRNA and directs degradation of the mRNA by RNase H.

In some embodiments, the gapmer is at least 70% complementary to the target mRNA (e.g., at least 70% complementary, at least 80% complementary, at least 95% complementary, or at least 99% complementary). In some embodiments, the gapmer is 100% complementary to the target mRNA. In some embodiments, the gapmer comprises a nucleic acid sequence that is at least 70% identical to any one of SEQ ID NOs: 1-27 (e.g., at least 70% complementary, at least 80% complementary, at least 95% complementary, or at least 99% complementary to any one of SEQ ID NOs: 1-27). In some embodiments, the gapmer comprises a nucleic acid sequence that is at least 70% identical to any one of SEQ ID NOs: 1-27 (e.g., at least 70% identical, at least 80% identical, at least 95% identical, or at least 99% identical to any one of SEQ ID NOs: 1-27). In some embodiments, the gapmer comprises any one of SEQ ID NOs: 1-27. In some embodiments, the gapmer comprises any one of SEQ ID NOs: 1-27 and is a 5-10-5 gapmer. In some embodiments, the gapmer comprises any one of SEQ ID NOs: 1-27, nucleotide positions 1-5 and 16-20 are ribonucleic acids and positions 6-15 are deoxyribonucleic acids. In some embodiments, the gapmer comprises any one of SEQ ID NOs: 1-27 and further comprises a phosphorothioate (PS) backbone and 2′-MOE modification on positions 1-5 and 16-20. In some embodiments, the gapmer consists of the nucleic acid sequence of any one of SEQ ID NOs: 1-27 having a phosphorothioate (PS) backbone and 2′-MOE modification on positions 1-5 and 16-20.

In some embodiments, the gapmer is complementary to a region comprising a mutation in exon 2 of H3F3A. In some embodiments, the gapmer comprises any one of SEQ ID NOs: 1-10. In some embodiments, the gapmer comprises any one of SEQ ID NOs: 1-10, nucleotide positions 1-5 and 16-20 are ribonucleic acids and nucleotide positions 6-15 are deoxyribonucleic acids. In some embodiments, the gapmer comprises any one of SEQ ID NOs: 1-10 and further comprises a phosphorothioate (PS) backbone and 2′-MOE modification on positions 1-5 and 16-20. In some embodiments, the gapmer consists of the nucleic acid sequence of any one of SEQ ID NOs: 1-10 having a phosphorothioate (PS) backbone and 2′-MOE modification on positions 1-5 and 16-20. In some embodiments, the gapmer comprises any one of SEQ ID NOs: 94-103.

In some embodiments, the gapmer is complementary to a region of H3F3A exon 2 that does not comprise a mutation. In some embodiments, the gapmer comprises any one of SEQ ID NOs: 11-15 and 25. In some embodiments, the gapmer comprises any one of SEQ ID NOs: 11-15 and 25, nucleotide positions 1-5 and 16-20 are ribonucleic acids and nucleotide positions 6-15 and 25 are deoxyribonucleic acids. In some embodiments, the gapmer comprises any one of SEQ ID NOs: 11-15 and 25 and further comprises a phosphorothioate (PS) backbone and 2′-MOE modification on positions 1-5 and 16-20. In some embodiments, the gapmer consists of the nucleic acid sequence of any one of SEQ ID NOs: 11-15 and 25 having a phosphorothioate (PS) backbone and 2′-MOE modification on positions 1-5 and 16-20. In some embodiments, the gapmer comprises any one of SEQ ID NOs: 104-108 and 118.

In some embodiments, the gapmer is complementary to a region of H3F3A exon 3 that does not comprise a mutation. In some embodiments, the gapmer comprises any one of SEQ ID NOs: 16-18. In some embodiments, the gapmer comprises any one of SEQ ID NOs: 16-18, nucleotide positions 1-5 and 16-20 are ribonucleic acids and nucleotide positions 6-15 are deoxyribonucleic acids. In some embodiments, the gapmer comprises any one of SEQ ID NOs: 16-18 and further comprises a phosphorothioate (PS) backbone and 2′-MOE modification on positions 1-5 and 16-20. In some embodiments, the gapmer consists of the nucleic acid sequence of any one of SEQ ID NOs: 16-18 having a phosphorothioate (PS) backbone and 2′-MOE modification on positions 1-5 and 16-20. In some embodiments, the gapmer comprises any one of SEQ ID NOs: 109-110.

In some embodiments, the gapmer is complementary the 5′ untranslated region (UTR) of H3F3A. In some embodiments, the gapmer comprises any one of SEQ ID NOs: 19-21. In some embodiments, the gapmer comprises any one of SEQ ID NOs: 19-21, nucleotide positions 1-5 and 16-20 are ribonucleic acids and nucleotide positions 6-15 are deoxyribonucleic acids. In some embodiments, the gapmer comprises any one of SEQ ID NOs: 19-21 and further comprises a phosphorothioate (PS) backbone and 2′-MOE modification on positions 1-5 and 16-20. In some embodiments, the gapmer consists of the nucleic acid sequence of any one of SEQ ID NOs: 19-21 having a phosphorothioate (PS) backbone and 2′-MOE modification on positions 1-5 and 16-20. In some embodiments, the gapmer comprises any one of SEQ ID NOs: 112-114.

In some embodiments, the gapmer is complementary to the 3′ untranslated region (UTR) of H3F3A. In some embodiments, the gapmer comprises any one of SEQ ID NOs: 22-24. In some embodiments, the gapmer comprises any one of SEQ ID NOs: 22-24, nucleotide positions 1-5 and 16-20 are ribonucleic acids and nucleotide positions 6-15 are deoxyribonucleic acids. In some embodiments, the gapmer comprises any one of SEQ ID NOs: 22-24 and further comprises a phosphorothioate (PS) backbone and 2′-MOE modification on positions 1-5 and 16-20. In some embodiments, the gapmer consists of the nucleic acid sequence of any one of SEQ ID NOs: 22-24 having a phosphorothioate (PS) backbone and 2′-MOE modification on positions 1-5 and 16-20. In some embodiments, the gapmer comprises any one of SEQ ID NOs: 115-117.

In some embodiments, the gapmer is complementary to the exon 3 of H3F3A. In some embodiments, the gapmer comprises SEQ ID NO: 26. In some embodiments, the gapmer comprises SEQ ID NO: 26, nucleotide positions 1-5 and 16-20 are ribonucleic acids and nucleotide positions 6-15 are deoxyribonucleic acids. In some embodiments, the gapmer comprises SEQ ID NO: 26 and further comprises a phosphorothioate (PS) backbone and 2′-MOE modification on positions 1-5 and 16-20. In some embodiments, the gapmer consists of the nucleic acid sequence of SEQ ID NO: 26 having a phosphorothioate (PS) backbone and 2′-MOE modification on positions 1-5 and 16-20. In some embodiments, the gapmer comprises SEQ ID NO: 119.

In some embodiments, the gapmer is complementary to the exon 4 of H3F3A. In some embodiments, the gapmer comprises SEQ ID NO: 27. In some embodiments, the gapmer comprises SEQ ID NO: 27, nucleotide positions 1-5 and 16-20 are ribonucleic acids and nucleotide positions 6-15 are deoxyribonucleic acids. In some embodiments, the gapmer comprises SEQ ID NO: 27 and further comprises a phosphorothioate (PS) backbone and 2′-MOE modification on positions 1-5 and 16-20. In some embodiments, the gapmer consists of the nucleic acid sequence of SEQ ID NO: 27 having a phosphorothioate (PS) backbone and 2′-MOE modification on positions 1-5 and 16-20. In some embodiments, the gapmer comprises SEQ ID NO: 120.

In some embodiments, the ASO comprises or consists of the nucleic acid sequence CACTCATGCGAGCGGCTTTT (SEQ ID NO: 1). In some embodiments, the ASO comprises or consists of the nucleic acid sequence GCGCACTCATGCGAGCGGCT (SEQ ID NO: 4). In some embodiments, the ASO comprises or consists of the nucleic acid sequence GGCGCACTCATGCGAGCGGC (SEQ ID NO: 5). In some embodiments, the ASO comprises or consists of the nucleic acid sequence GGGCGCACTCATGCGAGCGG (SEQ ID NO: 6).

In some embodiments, the ASO comprises or consists of the nucleic acid sequence CACTCATGCGAGCGGCTTTT with the first 5 nucleosides and last 5 nucleosides each comprising a 2′-MOE modification and all internucleoside linkages each comprising a PS modification (SEQ ID NO: 94). In some embodiments, the ASO comprises or consists of the nucleic acid sequence GCGCACTCATGCGAGCGGCT with the first 5 nucleosides and last 5 nucleosides each comprising a 2′-MOE modification and all internucleoside linkages each comprising a PS modification (SEQ ID NO: 97). In some embodiments, the ASO comprises or consists of the nucleic acid sequence GGCGCACTCATGCGAGCGGC with the first 5 nucleosides and last 5 nucleosides each comprising a 2′-MOE modification and all internucleoside linkages each comprising a PS modification (SEQ ID NO: 98). In some embodiments, the ASO comprises or consists of the nucleic acid sequence GGGCGCACTCATGCGAGCGG with the first 5 nucleosides and last 5 nucleosides each comprising a 2′-MOE modification and all internucleoside linkages each comprising a PS modification (SEQ ID NO: 99).

### Splice Modulating ASOs

In some embodiments, the ASO modifies the splicing of an mRNA encoding a mutation associated with a disease. In some embodiments, the ASO modifies splicing by promoting splice-skipping of the exon comprising the mutation. As used herein, splice-skipping refers modifying the splicing processes of a pre-mRNA such that one or more exons are not included in the spliced mRNA. Splice modulating ASOs are designed to sterically block a 5′ or 3′ splice site or a splicing-enhancer element, which in turn promotes exclusion of one or more exons from an mRNA. For example, splice skipping can be used to exclude an H3F3A Exon 2 that contains the K27M mutant from the H3F3A mRNA.

In some embodiments, the splice modulating ASO comprises naturally occurring nucleotides and nucleotide linkages. In some embodiments, splice modulating ASO comprises deoxynucleotides. In some embodiments, the splice modulating ASO comprises modified nucleotides. In some embodiments, splice modulating ASO comprises modified deoxynucleotides. In some embodiments, splice modulating ASO comprises 2′-H modified nucleotides. In some embodiments, the splice modulating ASO comprises phosphorothioate (PS) backbone, a morpholino backbone or a peptide nucleic acid (PNA) backbone. In some embodiments, splice modulating ASO comprises 2′-O-methyl modified ribose (2′OMe), 2′-O-methoxyethyl modified ribose (2′-MOE), or 2′-fluoro modified ribose (2-′F) modified nucleotides. In some embodiments, splice modulating ASO comprises a locked nucleic acid (LNA), a constrained ethyl ribose (cEt) nucleic acid, a Tricycol-DNA (tc-DNA) nucleic acid, a 5′methylcytosine (m⁵C) nucleic acid, or a N-acetylgalactosamine (GalNAc) nucleic acid. In some embodiments, the splice modulating ASO comprises 2′-MOE modification and phosphorothioate backbone modifications. In some embodiments, each nucleotide in the splice modulating ASO comprises a 2′-MOE modification and each backbone linkage is a phosphorothioate linkage.

The splice modulating ASO may be of any length suitable for specific binding to a modulating splicing (e.g., pre-mRNA splicing). For example, the splice modulating ASO may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides or nucleosides in length. In some embodiments, the splice modulating ASO is between about 10 to about 30 nucleotides or nucleosides in length, about 10 to about 20 nucleotides or nucleosides in length, or about 15 nucleotides or nucleosides in length. In some embodiments, the splice modulating ASO is 18-22 nucleotides or nucleosides in length.

In some embodiments, the splice modulating ASO is at least 70% complementary to the target pre-mRNA (e.g., at least 70% complementary, at least 80% complementary, at least 95% complementary, or at least 99% complementary). In some embodiments, the splice modulating ASO is 100% complementary to the target pre-mRNA. In some embodiments, the splice modulating ASO comprises a nucleic acid sequence that is at least 70% identical to any one of SEQ ID NOs: 28-77 (e.g., at least 70% complementary, at least 80% complementary, at least 95% complementary, or at least 99% complementary to any one of SEQ ID NOs: 28-77). In some embodiments, the splice modulating ASO comprises a nucleic acid sequence that is at least 70% identical to any one of SEQ ID NOs: 28-77 (e.g., at least 70% identical, at least 80% identical, at least 95% identical, or at least 99% identical to any one of SEQ ID NOs: 28-77). In some embodiments, the splice modulating ASO comprises any one of SEQ ID NOs: 28-77. In some embodiments, the splice modulating ASO comprises any one of SEQ ID NOs: 28-77 and the splice modulating ASO comprises 2′-MOE modification and phosphorothioate backbone modifications. In some embodiments, the splice modulating ASO comprises any one of SEQ ID NOs: 28-44, each nucleotide in the splice modulating ASO comprises a 2′-MOE modification and each backbone linkage is a phosphorothioate linkage. In some embodiments, the splice modulating ASO comprises any one of SEQ ID NOs: 121-170.

In some embodiments, the splice modulating ASO comprises a nucleic acid sequence that is complementary to the 5′ splice site of H3F3A. In some embodiments, the splice modulating ASO comprises a nucleic acid sequence that is at least 70% identical to any one of SEQ ID NOs: 28-44 (e.g., at least 70% complementary, at least 80% complementary, at least 95% complementary, or at least 99% complementary to any one of SEQ ID NOs: 28-44). In some embodiments, the splice modulating ASO comprises a nucleic acid sequence that is at least 70% identical to any one of SEQ ID NOs: 28-44 (e.g., at least 70% identical, at least 80% identical, at least 95% identical, or at least 99% identical to any one of SEQ ID NOs: 28-44). In some embodiments, the splice modulating ASO comprises any one of SEQ ID NOs: 28-44. In some embodiments, the splice modulating ASO comprises any one of SEQ ID NOs: 28-44 and the splice modulating ASO comprises 2′-MOE modification and phosphorothioate backbone modifications. In some embodiments, the splice modulating ASO comprises any one of SEQ ID NOs: 28-44, each nucleotide in the splice modulating ASO comprises a 2′-MOE modification and each backbone linkage is a phosphorothioate linkage. In some embodiments, the splice modulating ASO comprises any one of SEQ ID NOs: 121-137.

In some embodiments, the splice modulating ASO comprises a nucleic acid sequence that is complementary to exon 2 of H3F3A. In some embodiments, the splice modulating ASO comprises a nucleic acid sequence that is complementary to a mutated region of exon 2 of H3F3A. In some embodiments, the splice modulating ASO comprises a nucleic acid sequence that is complementary to a mutated region of exon 2 of H3F3A that comprises the K27M mutation. In some embodiments, the splice modulating ASO comprises a nucleic acid sequence that is at least 70% identical to any one of SEQ ID NOs: 45-77 (e.g., at least 70% complementary, at least 80% complementary, at least 95% complementary, or at least 99% complementary to any one of SEQ ID NOs: 45-77). In some embodiments, the splice modulating ASO comprises a nucleic acid sequence that is at least 70% identical to any one of SEQ ID NOs: 45-77 (e.g., at least 70% identical, at least 80% identical, at least 95% identical, or at least 99% identical to any one of SEQ ID NOs: 45-77). In some embodiments, the splice modulating ASO comprises any one of SEQ ID NOs: 45-77. In some embodiments, the splice modulating ASO comprises any one of SEQ ID NOs: 45-77 and the splice modulating ASO comprises 2′-MOE modification and phosphorothioate backbone modifications. In some embodiments, the splice modulating ASO comprises any one of SEQ ID NOs: 45-77, each nucleotide in the splice modulating ASO comprises a 2′-MOE modification and each backbone linkage is a phosphorothioate linkage. In some embodiments, the splice modulating ASO comprises any one of SEQ ID NOs: 138-170.

In some embodiments, the ASO comprises or consists of the nucleic acid sequence ACCCCTCCAGTAGAGGGCGC (SEQ ID NO: 58). In some embodiments, the ASO comprises or consists of the nucleic acid sequence CAGTAGAGGGCGCACTCATG (SEQ ID NO: 59). In some embodiments, the ASO comprises or consists of the nucleic acid sequence AGTAGAGGGCGCACTCATGC (SEQ ID NO: 60).

In some embodiments, the ASO comprises or consists of the nucleic acid sequence ACCCCTCCAGTAGAGGGCGC with each nucleoside comprising a 2′-MOE modification and all internucleoside linkages each comprising a PS modification (SEQ ID NO: 151). In some embodiments, the ASO comprises or consists of the nucleic acid sequence CAGTAGAGGGCGCACTCATG with each nucleoside comprising a 2′-MOE modification and all internucleoside linkages each comprising a PS modification (SEQ ID NO: 152). In some embodiments, the ASO comprises or consists of the nucleic acid sequence AGTAGAGGGCGCACTCATGC with each nucleoside comprising a 2′-MOE modification and all internucleoside linkages each comprising a PS modification (SEQ ID NO: 153).

### ASO Targets

In some embodiments, the nucleic acid to be targeted by an ASO is an mRNA transcript expressed in a cell, such as a eukaryotic cell. In some embodiments, the eukaryotic cell is a human cell. In some embodiments, the cell is a brain cell. In some embodiments, the cell is a neuron cell. In some embodiments, the cell is a glial cell. In some embodiments, the cell is a macroglia cell. In some embodiments, the cell is a microglia cell. In some embodiments, the cell is an astrocyte, oligodendrocyte, or an ependymal cell. In some embodiments, the cell is an oligodendrocyte precursor-like cell. In some embodiments, the cell is positive for a neural stem cell marker. In some embodiments, the cell is positive for Nestin (Nestin+). In some embodiments, the cell is positive for Vimentin (Vimentin+). In some embodiments, the cell is positive for Sox2 (Sox2+). In some embodiments, the cell is positive for Nestin (Nestin+), Vimentin (Vimentin+), and Sox2 (Sox2+).

In some embodiments, the mRNA transcript comprises a mutation. In some embodiments, the mutation is a disease associated mutation. In some embodiments, the mutation is associated with cancer. In some embodiments, the mutation is associated with brain cancer. In some embodiments, the mutation is associated with high-grade glioma (pHGG). In some embodiments, the mutation is associated diffuse midline glioma (DMG). In some embodiments, the mutation is associated diffuse intrinsic pontine glioma (DIPG). In some embodiments the mutation that is associated with diffuse intrinsic pontine glioma (DIPG) is an H3F3A mutation. In some embodiments, the H3F3A mutation results in a K27M mutation in the protein encoding H3F3A (histone H3.3 A). In some embodiments, the H3F3A mutant allele is dominant negative.

In some embodiments, the ASO is complementary to an mRNA encoding an H3F3A mutant allele. In some embodiments, the ASO is complementary to a region comprising a mutation in exon 2 of a pre-mRNA encoding an H3F3A mutant allele. In some embodiments, the ASO is complementary to a region of an H3F3A K27M mutant allele that comprises a mutation. In some embodiments, the ASO is complementary to a region of an H3F3A K27M mutant allele that comprises a mutation at position 83 of H3F3A Exon 2 (SEQ ID NO: 86). In some embodiments, the ASO is complementary to a region of an H3F3A K27M mutant allele that comprises an A to T mutation at position 83 of H3F3A Exon 2 (SEQ ID NO: 86). In some embodiments, the ASO is complementary to an mRNA encoding an H3F3A K27M mutant allele that is at least 70% identical to any one of SEQ ID NOs: 86-88. (e.g., at least 70% complementary, at least 80% complementary, at least 95% complementary, or at least 99% complementary to any one of SEQ ID NOs: 86-88). In some embodiments, the ASO is complementary to an mRNA encoding an H3F3A K27M mutant that is at least 70% identical to any one of SEQ ID NOs: 86-88. (e.g., at least 70% identical, at least 80% identical, at least 95% identical, or at least 99% identical to any one of SEQ ID NOs: 86-88).

In some embodiments, the ASO is complementary to a non-mutated region of H3F3A. In some embodiments, the ASO is completely to non-mutated region of H3F3A in exon 2. In some embodiments, the ASO is complementary to the 5′ splice site of H3F3A exon 2. In some embodiments, the ASO is completely exon 3 of H3F3A. In some embodiments, the ASO is completely exon 4 of H3F3A. In some embodiments, the ASO is complementary to the 5′ untranslated region (UTR) of H3F3A. In some embodiments, the ASO is complementary to the 3′ untranslated region (UTR) of H3F3A.

In some embodiments, the expression of the gene or allele (e.g., mutant H3F3A) is reduced by at least 10% (e.g., at least 10%, at least 20%, at least 30%, at least 40%, at least 50%, at least 60%, at least 70%, at least 80%, at least 90%, at least 95%, or at least 99%). In some embodiments, the ASO directs RNAse H-mediated degradation of the target pre-mRNA, mRNA or both pre-mRNA and mRNA (e.g., mutant H3F3A). In some embodiments, the quantity of the target pre-mRNA, mRNA or both pre-mRNA and mRNA is reduced by at least 10% (e.g., at least 10%, at least 20%, at least 30%, at least 40%, at least 50%, at least 60%, at least 70%, at least 80%, at least 90%, at least 95%, or at least 99%).

In some embodiments, administration of the ASO specifically decreases the quantity of pre-mRNA, mRNA or pre-mRNA encoding a mutant allele compared to the quantity of pre-mRNA, mRNA or pre-mRNA encoding a corresponding wildtype allele. For example, an ASO may specifically decrease the quantity of pre-mRNA, mRNA or pre-mRNA encoding an H3F3A K27M allele compared to the quantity of a corresponding wildtype H3F3A allele. In some embodiments, administration of the ASO specifically decreases the quantity of pre-mRNA, mRNA or both pre-mRNA and mRNA encoded by a mutant allele compared to the quantity of pre-mRNA, mRNA or both pre-mRNA and mRNA encoded by a corresponding wildtype allele. For example, an ASO may specifically decrease the quantity of pre-mRNA, mRNA or both pre-mRNA and mRNA encoded by an H3F3A K27M allele compared to the quantity of a corresponding wildtype H3F3A allele.

In some embodiments, an ASO may specifically decrease the quantity of mRNA encoding an H3F3A K27M by 10%, 20%, 30%, 40%, 50%, 60%, 70%, 80%, 90%, 95% or 99% more than the ASO decreases the quantity of the wildtype H3F3A allele. In some embodiments, an ASO may specifically decrease the quantity of pre-mRNA, mRNA or both pre-mRNA and mRNA encoded by an H3F3A K27M by 10%, 20%, 30%, 40%, 50%, 60%, 70%, 80%, 90%, 95% or 99% more than the ASO decreases the quantity of pre-mRNA, mRNA or both pre-mRNA and mRNA encoded by the wildtype H3F3A allele. In some embodiments, administration of the ASO specifically decreases the concentration of pre-mRNA, mRNA or both pre-mRNA and mRNA encoding a mutant allele compared to a reference. In some embodiments, administration of the ASO specifically decreases the concentration of pre-mRNA, mRNA or both pre-mRNA and mRNA encoded by a mutant allele compared to a reference. In some embodiments, the reference is the quantity of the pre-mRNA, mRNA or both pre-mRNA and mRNA encoded by the mutant allele prior administration of the ASO. In some embodiments, the reference is the concentration of the pre-mRNA, mRNA or both pre-mRNA and mRNA encoded by the mutant allele prior administration of the ASO.

In some embodiments, the reference is the quantity of the protein encoded by the mutant allele prior administration of the ASO. In some embodiments, the reference is the concentration of the protein encoded by the mutant allele prior administration of the ASO. In some embodiments, the reference is the quantity of the protein encoded by the mutant allele compared to administration of an alternative treatment. In some embodiments, the reference is the concentration of the protein encoded by the mutant allele compared to administration of an alternative treatment. In some embodiments, the reference is the quantity of the pre-mRNA, mRNA or protein encoded by the mutant allele after administration of a control (non-targeting) ASO. In some embodiments, the reference is the concentration of the pre-mRNA, mRNA or protein encoded by the mutant allele after administration of a control (non-targeting) ASO. In some embodiments, the quantity or concentration or the pre-mRNA, mRNA or protein is determined.

### Exon 2 of Wildtype H3F3A:

(SEQ ID NO: 86)
ATGGCTCGTACAAAGCAGACTGCCCGCAAATCGACCGGTGGTAAAGCACC
CAGGAAGCAACTGGCTACAAAAGCCGCTCGCAAGAGTGCGCCCTCTACTG
GAGGGGTGAAGAAACCTCATCGTTACAG

### Exon 2 of K27M H3F3A:

(SEQ ID NO: 87)
ATGGCTCGTACAAAGCAGACTGCCCGCAAATCGACCGGTGGTAAAGCACC
CAGGAAGCAACTGGCTACAAAAGCCGCTCGCATGAGTGCGCCCTCTACTG
GAGGGGTGAAGAAACCTCATCGTTACAG

### Exon 2 of K27M H3F3A Positions 60-121:

(SEQ ID NO: 88)
ACTGGCTACAAAAGCCGCTCGCATGAGTGCGCCCTCTACTGGAGGGGTGA
AGAAACCTCATC

Wildtype H3F3A Gene Sequence (NCBI NC_000001.11:226061831-226072019 Homo sapiens Chromosome 1, GRCh38.p13 Primary Assembly):

(SEQ ID NO: 89)
ACACATTGTTTAGAGGCCGACGCGACACAGCCATCTTGCGTCGGGGGCCCGGGC
CCGGGAGGAGAGCGAGCCCCGTCCCCTGATCCCGGAGGCCAACACCGGCGAGGG
GCTGCCCTGCACGCAGGCGGCCGGCCCAGGAGTCTTAGCGGATCAAGTTGTCTA
CGGCGGACTCGGGCCGGTCCCGCAGGACCCGGCCGCACTTGGCCGAGCGCGCAG
GCGGGAAGGAGGCCGTCGGTCCGCCCCTCCCGCTGCCGCAGCGGCCAGGTCCGA
CAGCGGGCGCGGGACGCTGGATTCCTATTTGCCAGCTTCCTGCTGGGGAGCGTTT
TCCAATGGGGCGCGGCGACCCGGCCGACCGCGTCCTCCTCCCCCACTGCCGCCCG
GGTCTCCTCGGGGGCGCGCGCCGGGGCCCGCGCGGGGGCAGCCCCTCCCTCCCC
TCCCCGCCGCGGCCCGAGACGCCCGCAGGCCGGGCGTTCACCCGCCGCGCCTCC
GCCCGCCTTCCCCTCCCCCCACTTCCCTCCGGGCGCCCCAAACCCGGGCTTCGGG
GGTTCGACGCCGTGCGCACACGGAGCGGGATGGGAGTGCAGGGCCGGGGGCGG
GGGCTGGCCGGGTGCGGGCGGCAGTCTCGGCGGGAGCCGGCGGCCCCTCAGCGT
GTCTTTTGTGTTTGTACACACACGGCGCGAGGCGGCCTGGAGGAGGGAGCGGGC
GGCGCGCGGGGGAGGGGCGAGCGCGCGCCAGCGAACGGGCGCGCGGGGGGAAG
GGCGGGGGCGGGGCGCGAGAGGAGCTATGGGGGCGGGGCCGAGCCTTCCCTCC
ATTGTGTGTGATTGGCTGGCGCGCGGCGCGGGGGGGGGGGGCGTGTGTTGGGG
GATAGCCTCGGTGTCAGCCATCTTTCAATTGTGTTCGCAGCCGCCGCCGCGCCGC
CGTCGCTCTCCAACGCCAGCGCCGCCTCTCGCTCGCCGAGCTCCAGCCGAAGGAG
AAGGGGGGTAAGTTTCCCCGTCTGCCCGCTTCCCCGGAACCGAGCCCCGCTTGCC
GTCAGCCCCGAGCCGGGCCCTGGCGGTGCCGGGAGGGGACCCATAGCCCCTCCG
GCTTAGCCCACAAACTTTTTGGCCCAGAAATGGAGGTGAGGAGCGAGTTTCCCTG
TTCCCTCTGGCGGCGGCGGCTCCCCCGTCTCTCGCCGCCTCAGCCCAACAGCAGC
AACCGCCGCGGCGCCGAGCCTGCTCTCCCTCCTCCTCCCCGCGCCCCTGGCTCCT
CTTTCTTCGGTGAAATCCCGCCCGCCGCCCCTTCCCCGGACCCCAAACCTTCACC
ATGACCCGCGCGGGCCTCTTAACTACCGCCCCCGGGCCCCAGCCCCCAGTTTTCG
AGCGGGAAAGGGGTGGAAATCGCCGCCGCTTCGCACCCTGGGGTAACTCGCTTT
TTGCTGCCTCCCCACAGTTGTTGCTAAGCTTCACCATCTTGTCTCTCTTCTCTGGT
CACCGACCATATTTTTTCCCCCGTTTCTCTCTTCCCCATTTCAAAAAAGCAAGAAT
TTTTAAAAGAGGGACGTTTTTTTCCCTTTTTTGGAGAGGCGGAAACTTGATGCATT
TGAAATGCAAAAAAAACTTTTGCATTTTGGAGGGCGGCACGTGGCAGGGGATAG
GGGTATTTTCGAGCGATTTGGTGTTTTGTCTCTTTGGAACGGAATCGGATACGTTT
TGCGATTGTTTCCATTTTGTTCTGGTCGGGGAGCCGAGTTGGTTCCTGCTCGAGGC
GCTGGGAGCGAGGCGGGCAGGTTGGGGGACCCTGTGCCGAGGGACCCGAAGGA
GGCAAAACCAAAAGTTTATGTGGTGCTTGGGGTGGACCGGTCGGCGGATGTCGT
GCGTTAATTAAAGCCTGGGTGCGGGGCACTTTTTATTTCACTGCGAAGCCTGTGA
GGACTGGACGCGAGGACGGGGCTTTGTAGAATGCTCGCTGGTGGTAGCTGTTGTT
CCTCTCTTGTTTTTTCGAGACGCCTTTTTGTCAACATAACTGTAAAGATGCAAAAC
CAAGAATATTTTCAGATATTTACAGCAATCCATCAACAGTCAGACGAAGGGGGG
GCAGCCAAAGTGGGGGGGAGTAAGACCTTTTTTTTGCTGTAATTTGACTCGACCT
TCCAGTGGTTTGTGCCTTTTTTCTTTTGACTTGTTTGTGGATGGAATGTTTACAGA
CATTTCTAATTACTGCTTTAATTAAATAAATTGGATCAAAGGCCGTTCGAGGTAT
TTTTGTTTTGCCGTTTGTCGCTCAGAATTGGCATTTTGAGAGGTGATTGATACTGC
TAACAATTTTCTAGTACTCTAGTTTGTTTCAAGAAGAGATTTTGGGTAGACGTAA
TCTTCACCCTTTCAAATTATATAACAATACGAACATTATTTTTTATACTGATCATA
ATTTCCAGATTTGGGGAGGGGGTGATCGTGGCAGGAAAAGTTGTATGTTTGGTAG
TTGCATATGGTGATTTTTGATTTTTCAATGCTGGTAGGTAAGTAAGGAGGTCTCTG
TACCATGGCTCGTACAAAGCAGACTGCCCGCAAATCGACCGGTGGTAAAGCACC
CAGGAAGCAACTGGCTACAAAAGCCGCTCGCAAGAGTGCGCCCTCTACTGGAGG
GGTGAAGAAACCTCATCGTTACAGGTATTAAAAAACAGGAAAAAAATGGGACA
AAGTCTCTCTTGTATGTATCCACATAATTTAACAAAAAGATGGATAACAGGAAAA
CTTTTTGCTTTAGAGAAACTTTTTTTTTTCATTTGAACACTTAACTACTGCTTAAAT
AAATGTACTGTATGATCATTTATATATAAAGTTAAGTATTAGGTTTTATTGAAAA
CGTTTAACTTTGAAGCCATAATCTTACCTGGAGGTCTAAGGAGACCTCGTATATC
ACTGATAATGTTAATGGGATATATTGACATTTTAGTTAACTATTAGTAATTCTTTA
AAAATAGTTAAGTGTTGCTTTCTTGAATACACTTTTGAGGTTATCTTTCCTAGTTT
TTGGCAAAATAATAATATAATCGAGATTGGGTGTTTTATAAAGTTCCTACCCCCT
CATTTACTTGATTAGCTTATTTCCTTCTGTATTAGCTCTTTTGATTGTGAACTACCT
GAGCCACTGTGTTGTTAAGGGCATCTGCCATTAGAGGGCAGAAGTTGCCTAGACT
TAGCCTCCGCAAATATTACGATTAACCTAAAATAAAAGCTCATACAGTGGAAGA
AATAAGTCAGAATGAGAGGTAAATTTCTTGCTGACAATTACTTAGTAAAAAAAA
CTTTTCCACGTGGAAGGAACACTGCACAATCGGGTGTATTCAAATTTAATAGTTG
ATGAGGGGTGAACTGTAGTCAGGGTAGAGGTATTCTGGGAAATAGAACTTGAAT
GCCACAAGCAGATTTTTAAATGAAGTTCATTGGGTTGTGTTGGTCATCTTTAAGG
GCTCTTAAGATAATAGAGGGTGTAGGGAAGGAGAGTGGTGCTAAATTGAGGGGA
GCCAACTTCTTCATTAGCCTGACAGCTAATACCTTCCCAAGGTTTATATACATGA
ATAACTTTTTAAAATGAAAATAAATAGGGCTTTGATGAATTTCTTGCTAAGAATG
CATTTTAATTTCATGCTTTTTGCTTTAAAGGTATTGTTACTAGATTTGAATGTTCA
CATCTTAAGTTGATCAGTGAAATTTGATACTGAAGCTGAAGAATTGTTGGGTGGC
TTATTTTTTGAAAGATTACTGCATTTCTTTGAAGCTGCCCACTTACCTTTTTGTGCT
AGTTATGTTTTTGGTAACAGTTTCTTTATTAATTTTTTAAAGGCCTGGTACTGTGG
CGCTCCGTGAAATTAGACGTTATCAGAAGTCCACTGAACTTCTGATTCGCAAACT
TCCCTTCCAGCGTCTGGTGCGAGAAATTGCTCAGGACTTTAAAACAGATCTGCGC
TTCCAGAGCGCAGCTATCGGTGCTTTGCAGGTAAAATGGTGGGTGGGAAGACTC
AGAGTTTGTATTCCTGTTGTGTACCAAGAACAGTTCCAAATTGTTGCATGTGCTTA
TATCATTTAATCACAAGCCTGTCAGGTAGTTGATATTGTTACTTCACTGTTGAGAC
TTCAGAAAGGTTAAATTGCTCAAGGTCATACACGTAGAAAATGGCAAAACCATA
ATTTGAACCTATTTGACTCCAAGGCTTAGTGCACATTCCATTATACCATTTAAAAT
TTTGAAACATTGCACTAAAAACAATATTTAAAGAAAATCCTCTGGTTTGGTTTAT
GGATGCTGCAGGACATTAAGAAGAACTTGAGACTAGAGGTCTATATTTGTAGTA
ACAATTTCAAACAACAGTGCCTAGAATAAAAGAAATGTCTCTTCAGGTCCTGAA
GAAACATATAGGTAGAGAGAGCTTAATACCTAGGATGGGAATAGGCAGTATTAA
AAAATTATGCATTAAGACGTAAAAGGAGCAGTGAGTGGAGGATAGGATTGGATT
GGCAAGATTGGGGGAACGGTGCTTAAAGGTGATACTGGACGTGTAAAGGGGCCA
GGATTTGCCTTGTGGTTTCCTAAAGGGGATTAGGGATTGCCACTTACATGTGGAG
CATATTGAATTTAATCTTAATCTCTAGCATGTCAGGACTTAGAGAAATACTGTTCT
AGAGATATTTATGTATTTGAGATATTTGTGGTTTAAGTTAAGACCAAAACTTGAA
ATTCCTACAAGATACTTGATAAAGGTATGTAGACGTCATTAACATCAGTCACTTA
AGTAACCTATTTTATATTGTGTGGTTGGATTCTTTAGTTGCAAGTATCCCAGAATA
CAGTAAAGCTACCACCTCCATCAGAAGCATGCAGTTGGGGGTCGTTAAAAATGC
TGATTGTTAGGCACCATTGCAGACCTGAATCATTCTGGGGGGGGGAGGGGGCA
TCCAGAATCTAAGTACATTCCATTGGAGGATTTTCAAGGCTGCTGTTGCTGTGCT
GGGCTTTTGCTTTATGTATGAGAAAGATAGGATAGGTGGATTCCACCCTGAAGTG
TAATTGGTCGAGCTGTTTCTATGGTTAATCTAAACTTCATTTGATTGGTGAATTCC
AGGAGTAGGCATCCATTAAATTCTCAGTGAGTTGCTGAAATTTCAACTACTAAAA
TCTTCCATGTTTTTACTTAAAACTTTATTTTCTGTTTTAATAGCCGATTATCTTCAG
TTCTTATTTATTACTAAATACAGAAATGTTACTAAGATTCGTAAGCATTGGTACA
AAGCTGCTAATATTTTCTAGTTAGAACTGTGCTTAAAGAACAATACTGTACTTGA
AAAGTGTAGCGTTTTTAGCTTAAATGTCAATAATGTAGCCTTTTAAATTGAATTTA
CTAAACAATCTGGGTAAAAGACTCATCTTTCTAAAATTATTCACATGTAAACTTC
CATTGTTACTATTACATATTTGTCATTAAGTGTGGTTGTATTGGTTTTAAGCAAAA
TGTTCACCTCTTCTGTCCACAGTTCTCTTAGTGTTAGATAGCTCTTTATTGGAAAG
AGTGATCTTTGTTAAGCTTTTGTGTTTTCTGTTTGCTTTTTTATGCATATATTTTAG
TTATCTTAAAAATACAGAGAGTGGGCCGGGCGCCGTGACTCACATCTGTAATCCC
AACACTTTGGGAGGCTGAGGTGGGTAGATCACCTGAGGTCAGGAGTTCCAGACC
AGCGTGGCCAACATGGTGTGAAACCCCTTCTCTACTAAAAATACAAAAAATTAG
CTTGGCGTGGTGGCAGGCGCCTATAATCCCAGCTACTAGGGAGGCTGAGGCAGG
AGAATTGCTTAAACCCGGAAAGCAGAGGTTGCAGTGAGCCGAGATGGCGCCATT
GCACTCCAGCCTGGGCAACAAGAGCAAAACTCTTGTCTCAAAAAAAAAAAAAAG
ATAACAGAGGGAGAATTTGTTAATGGAATAGGATAATAGAAGATCGCAATTGAA
GTAGAAATCCAAAGGTCCTTTGTTTTTGGCCTTAGAATTACACACTTTACAACTA
AAGGATAGCAGGCAGTCCAGTGAGAATGGTAGAGGAGAAATTAGTCTCAGTTTG
CCATAGATAATCAATAATTGACTTACTTAAAATATAGTCTTTGCTTTTAATGTCTG
AGAACTTGAACTCAAATATTCTTGTGTTAAGTGGACAGTGTTGCTTTGAAATGGT
AAAGAGGCAGACGTTGAATATACTCAAAATGTCCTAAACTAATACTTTCTTGTAG
TTCTTTTTTTTCTTTGCCATTAAATCAAAATTAATTCATTAGGTCATAAATTAGTG
TGGCAGTCTTTAATTCCCATTACAGCCTTAATGTTTTAAGGGACTTTGGGTTACTT
GTAATAAAATTGGAAATGTATGGGTCATGGTCAGATCCTCTCGTAGGAGTTCAGT
CTCCTCTCAGAGCTGACCAGACACAATCCTTATAAACTAGTAGCCAGGTTCTAAG
ACAGCAGTGAGAGCCCCAGCTACCACATGCCACATCTGAGTGGTTCTAAGTGAT
ACCACTCTGAAAGGCCACCAGAACCATGGTGCCTGATGGTTTGCTTGCATAGTAG
CTTGTACCACATTTTACAAAAGCATTCTAGCTTTCTGTAATCCCGAGGTGCCAGG
TTAAGCAAAATTAAACTGTTTTCTTTCCTGATATAATAGCTGATATGCGTTGTGAG
CCCCTAAGGAGGCTACAATATGCCATTTTTTCCAGGATTTCTACCCACTTCTAGA
ATATCTTAAGAACGAATCTCGGAATATTAAGGACTTTTGCTTTTAATACCTGTGG
AATAGGTAAGATGCGAGGGACTACTTGCATAGAATAGAAACATTTTTTAAAACT
AGCAAATATTGAAAATCTACTTGAGGACAAGGCATTTGGAGTATTAAATGAGAT
TTCTGTTATCAAGTAACTTAAGGGCACCAAAAAATGGCTGATAATGCAAGGTCAT
TGTGAGTGCCAAGGAACTAGGAGTTCAGGGTTGCCTGCACTGGCAACAGGAGCA
AGAGACAAGTATGCGATTTTCTCCTTCATGGAATAGTCTTGAGCTGGGCCTTAAA
GGGTAGATAAGTAAGACTTAGTTGTTAATAACAAATGCTGGAGAAACACACGTG
AAAATAGTTCAATTTGAATGAAGGAAACTTGTAGAAGAGTAGTGATAGGGCTAA
AAAGGATGAAGCTAGAAATGTGAAAGTACCTTTTTTTTTTTTTTTTGAGACGAGT
CACACTCTGTCACCTAGGCTGGAGTGCAGTGGTGCAATCTCGGCTTACTGCAGCC
TCCGCCTCCCGGGTTCAAGCAATTCTCCTGTCTCAGCCTCCCAAGTAGCTGGGAC
TACAGGCACATGCCACCACGCCCAGCTACTTTTTGTATTTTTAGTAGAGACAAGG
TTTCTCCGTGTTGGTCAGTCTAGTCTCAAGCTCCTGACCTCGGGTGATCTGCCTGC
CTCGGCCTCCCAAAGTGCTGGGATTACAGGCGTGAGCCACCACGGCACCTGGTCT
GGAATTTTTTTTATTAGAATAAAAATGCAACTATAAGACTGTTTTCTCCCTTAATA
TATCTTCAGCAGAGAAATAACTTTTCCTTATAGAAAAGGAGAGAGAGCCAACTA
ACCTATCATTTCATGCTCCAGATCCAAAACTGTTGGATTTATGATTATTTTTTAAA
ATGGTAATTTCTCCATTTCAAAATGAGTAAGCAGGCCGGGCATGGCGGCTCACAC
CTGTAATCCCAGCACTTTGGGAGGCTGAGGTGGGCAGATCACCTGAAGTTAGGA
GTTCAAGACCAGCCTGGCCAACATGGTGAAACCCCATCTCTACTAAAAATACAA
AATTAGCTGGACGTGATGGTGCATGCCTGTAATCCCAGCTACTCGGGAGGCTGA
GGCAGGAGAATTGCCTGAGCTCGGGAGGTGGAGGTTGCAGTGAGCCGAGGTTAT
ACCACTGCACTCCAGCCAGGGCTACAGAGCAAGACTCAAACCTCAAAAAACAAA
AACAAAAAGAGTAAGCAGATGTTTTGGCTTAGACTAAAAGATTCTTCAGCTTTTC
AGACAGCTATAAGTATACTAAGAATTTGAGTTATGAGTTAATTCTAAGTGGAAAC
GCCCCTTTTTCCTCTTCACAAGTTAAGTGTCAATGAGTGATTCATACACTGTCATT
TTTAAGTGGTAGTAGGAATAAGATAACTTGAAAGGATCTTACAGTCAAATGGGA
AAAACCAGAGAAATCGATACTAGTACTAGAGGGCAACAAATGCTGTTACAAATT
GGGGTACGTAGAGGAAGGTACTTGGTAGAGGACAGGGGCATGTTTCCGGGGCAT
AGATCAAAGTATATAAATAAGGAACTGCCAGGCCAGTTGCGGTGGCTCACTCCT
GTAATCCCAGCACTTTGGGAGGCCGAGGCAGGAGGATCACGAGGTCAGGAGATC
GAGACCATCCTAGCTAACACAATGAAGCCCCATCTCTACTGAAAATTAGTCAGG
CGTGGTGGTGGGCGCCTGTAGCCCGAGCTACTCGGGAGGCTGAGGCAGGAGAAT
GACATGAACCTGGGAGGTGGAGCTTGCAGTGAGCTGAGATCTTGCCACTGCACT
CCAGCCTGGGCAACAAAGAGAGACTCCGTCTCAAAATAAATAAATAAGGAACTG
CCGGGCGCGGTGGCTCACGCCTATAATCCCAGCACTTTGGGAGGCCAAGGTGGG
TGGATCACGAGGTCAGGAGTTCGAGACCAGCCTGACCAACATGGTGAAACCCCT
TCTCTACTAAAAATACAAAAAAGTAGCCAGGCATGGTGGCGCATACCTGTAGTC
CCAGCTACTCGGGAGGCTGAGGCAGGAGAATCGCTTGAATCCGGGAGGTGGAGG
TTGCAGTGAGCCGAGATCGCGCTACTGCACTCCAGCCTGGGCGACAGAGTAAGA
CTCCATCTCAGAAAGAAAGAAAGAAATACGGAACTAACTTGTGATATGTTCTGG
AATCAAAAGTACTCTTATGATAAAACAGGTATGAAAGGGAACATAGATGAGAAG
CATGTGATAAAAACCACTTGTTCACCATGTTATACTACTGGACAAGGCAGAGGTT
CACATACTGTGTGAATGGGATTCAGAGTGAGGAGGAGACTAGGCTGGGATGGGG
TATTTGGATTGGACATGATTGCGTTTATAAGAATGAGAGTGTTAAATTGGATTTC
TTGCTTTATTTGTGACATTTCAGTTTATTAGAAATCATGTTACCATTAGAAAAATT
GAAGTTTCCTAGTAACAAAGTAATTTGATTTGTGTAACTTGATAAAAGATTTACT
GACTTAAGCTTTTGTTTTTTTTCATAAGCTGCTTTTGAGCTTTGTCCCACAGGTTGT
AAAATGTAAGCATTTGGTAAAATTGTCAGCATCTTGCCCAGTCATTTTTTTAAAG
GGTTCAAAAACCTTTTTGTTTTAATTCGTATAGTTGGGTCTTAACTATTGGAAATA
ACATCATCAGTAATTTTTTCTTCATTCCTTTTGCAGGAGGCAAGTGAGGCCTATCT
GGTTGGCCTTTTTGAAGACACCAACCTGTGTGCTATCCATGCCAAACGTGTAACA
ATTATGCCAAAAGACATCCAGCTAGCACGCCGCATACGTGGAGAACGTGCTTAA
GAATCCACTATGATGGGAAACATTTCATTCTCAAAAAAAAAAAAAAAAATTTCT
CTTCTTCCTGTTATTGGTAGTTCTGAACGTTAGATATTTTTTTTCCATGGGGTCAA
AAGGTACCTAAGTATATGATTGCGAGTGGAAAAATAGGGGACAGAAATCAGGTA
TTGGCAGTTTTTCCATTTTCATTTGTGTGTGAATTTTTAATATAAATGCGGAGACG
TAAAGCATTAATGCAAGTTAAAATGTTTCAGTGAACAAGTTTCAGCGGTTCAACT
TTATAATAATTATAAATAAACCTGTTAAATTTTTCTGGACAATGCCAGCATTTGG
ATTTTTTTAAAACAAGTAAATTTCTTATTGATGGCAACTAAATGGTGTTTGTAGCA
TTTTTATCATACAGTAGATTCCATCCATTCACTATACTTTTCTAACTGAGTTGTCC
TACATGCAAGTACATGTTTTTAATGTTGTCTGTCTTCTGTGCTGTTCCTGTAAGTT
TGCTATTAAAATACATTAAACTATACCTGCTTTTGGTCTTTA.

### H3F3A K27M Gene Sequence:

(SEQ ID NO: 90)
ACACATTGTTTAGAGGCCGACGCGACACAGCCATCTTGCGTCGGGGGCCCGGGC
CCGGGAGGAGAGCGAGCCCCGTCCCCTGATCCCGGAGGCCAACACCGGCGAGGG
GCTGCCCTGCACGCAGGCGGCCGGCCCAGGAGTCTTAGCGGATCAAGTTGTCTA
CGGCGGACTCGGGCCGGTCCCGCAGGACCCGGCCGCACTTGGCCGAGCGCGCAG
GCGGGAAGGAGGCCGTCGGTCCGCCCCTCCCGCTGCCGCAGCGGCCAGGTCCGA
CAGCGGGCGCGGGACGCTGGATTCCTATTTGCCAGCTTCCTGCTGGGGAGCGTTT
TCCAATGGGGCGCGGCGACCCGGCCGACCGCGTCCTCCTCCCCCACTGCCGCCCG
GGTCTCCTCGGGGGCGCGCGCCGGGGCCCGCGCGGGGGCAGCCCCTCCCTCCCC
TCCCCGCCGCGGCCCGAGACGCCCGCAGGCCGGGCGTTCACCCGCCGCGCCTCC
GCCCGCCTTCCCCTCCCCCCACTTCCCTCCGGGCGCCCCAAACCCGGGCTTCGGG
GGTTCGACGCCGTGCGCACACGGAGCGGGATGGGAGTGCAGGGCCGGGGGCGG
GGGCTGGCCGGGTGCGGGCGGCAGTCTCGGCGGGAGCCGGCGGCCCCTCAGCGT
GTCTTTTGTGTTTGTACACACACGGCGCGAGGCGGCCTGGAGGAGGGAGCGGGC
GGCGCGCGGGGGAGGGGCGAGCGCGCGCCAGCGAACGGGCGCGCGGGGGGAAG
GGCGGGGGCGGGGCGCGAGAGGAGCTATGGGGGCGGGGCCGAGCCTTCCCTCC
ATTGTGTGTGATTGGCTGGCGCGCGGCGCGGGGGCGGGGCGGCGTGTGTTGGGG
GATAGCCTCGGTGTCAGCCATCTTTCAATTGTGTTCGCAGCCGCCGCCGCGCCGC
CGTCGCTCTCCAACGCCAGCGCCGCCTCTCGCTCGCCGAGCTCCAGCCGAAGGAG
AAGGGGGGTAAGTTTCCCCGTCTGCCCGCTTCCCCGGAACCGAGCCCCGCTTGCC
GTCAGCCCCGAGCCGGGCCCTGGCGGTGCCGGGAGGGGACCCATAGCCCCTCCG
GCTTAGCCCACAAACTTTTTGGCCCAGAAATGGAGGTGAGGAGCGAGTTTCCCTG
TTCCCTCTGGCGGCGGCGGCTCCCCCGTCTCTCGCCGCCTCAGCCCAACAGCAGC
AACCGCCGCGGCGCCGAGCCTGCTCTCCCTCCTCCTCCCCGCGCCCCTGGCTCCT
CTTTCTTCGGTGAAATCCCGCCCGCCGCCCCTTCCCCGGACCCCAAACCTTCACC
ATGACCCGCGCGGGCCTCTTAACTACCGCCCCCGGGCCCCAGCCCCCAGTTTTCG
AGCGGGAAAGGGGTGGAAATCGCCGCCGCTTCGCACCCTGGGGTAACTCGCTTT
TTGCTGCCTCCCCACAGTTGTTGCTAAGCTTCACCATCTTGTCTCTCTTCTCTGGT
CACCGACCATATTTTTTCCCCCGTTTCTCTCTTCCCCATTTCAAAAAAGCAAGAAT
TTTTAAAAGAGGGACGTTTTTTTCCCTTTTTTGGAGAGGCGGAAACTTGATGCATT
TGAAATGCAAAAAAAACTTTTGCATTTTGGAGGGCGGCACGTGGCAGGGGATAG
GGGTATTTTCGAGCGATTTGGTGTTTTGTCTCTTTGGAACGGAATCGGATACGTTT
TGCGATTGTTTCCATTTTGTTCTGGTCGGGGAGCCGAGTTGGTTCCTGCTCGAGGC
GCTGGGAGCGAGGCGGGCAGGTTGGGGGACCCTGTGCCGAGGGACCCGAAGGA
GGCAAAACCAAAAGTTTATGTGGTGCTTGGGGTGGACCGGTCGGCGGATGTCGT
GCGTTAATTAAAGCCTGGGTGCGGGGCACTTTTTATTTCACTGCGAAGCCTGTGA
GGACTGGACGCGAGGACGGGGCTTTGTAGAATGCTCGCTGGTGGTAGCTGTTGTT
CCTCTCTTGTTTTTTCGAGACGCCTTTTTGTCAACATAACTGTAAAGATGCAAAAC
CAAGAATATTTTCAGATATTTACAGCAATCCATCAACAGTCAGACGAAGGGGGG
GCAGCCAAAGTGGGGGGGAGTAAGACCTTTTTTTTGCTGTAATTTGACTCGACCT
TCCAGTGGTTTGTGCCTTTTTTCTTTTGACTTGTTTGTGGATGGAATGTTTACAGA
CATTTCTAATTACTGCTTTAATTAAATAAATTGGATCAAAGGCCGTTCGAGGTAT
TTTTGTTTTGCCGTTTGTCGCTCAGAATTGGCATTTTGAGAGGTGATTGATACTGC
TAACAATTTTCTAGTACTCTAGTTTGTTTCAAGAAGAGATTTTGGGTAGACGTAA
TCTTCACCCTTTCAAATTATATAACAATACGAACATTATTTTTTATACTGATCATA
ATTTCCAGATTTGGGGAGGGGGTGATCGTGGCAGGAAAAGTTGTATGTTTGGTAG
TTGCATATGGTGATTTTTGATTTTTCAATGCTGGTAGGTAAGTAAGGAGGTCTCTG
TACCATGGCTCGTACAAAGCAGACTGCCCGCAAATCGACCGGTGGTAAAGCACC
CAGGAAGCAACTGGCTACAAAAGCCGCTCGCATGAGTGCGCCCTCTACTGGAGG
GGTGAAGAAACCTCATCGTTACAGGTATTAAAAAACAGGAAAAAAATGGGACA
AAGTCTCTCTTGTATGTATCCACATAATTTAACAAAAAGATGGATAACAGGAAAA
CTTTTTGCTTTAGAGAAACTTTTTTTTTTCATTTGAACACTTAACTACTGCTTAAAT
AAATGTACTGTATGATCATTTATATATAAAGTTAAGTATTAGGTTTTATTGAAAA
CGTTTAACTTTGAAGCCATAATCTTACCTGGAGGTCTAAGGAGACCTCGTATATC
ACTGATAATGTTAATGGGATATATTGACATTTTAGTTAACTATTAGTAATTCTTTA
AAAATAGTTAAGTGTTGCTTTCTTGAATACACTTTTGAGGTTATCTTTCCTAGTTT
TTGGCAAAATAATAATATAATCGAGATTGGGTGTTTTATAAAGTTCCTACCCCCT
CATTTACTTGATTAGCTTATTTCCTTCTGTATTAGCTCTTTTGATTGTGAACTACCT
GAGCCACTGTGTTGTTAAGGGCATCTGCCATTAGAGGGCAGAAGTTGCCTAGACT
TAGCCTCCGCAAATATTACGATTAACCTAAAATAAAAGCTCATACAGTGGAAGA
AATAAGTCAGAATGAGAGGTAAATTTCTTGCTGACAATTACTTAGTAAAAAAAA
CTTTTCCACGTGGAAGGAACACTGCACAATCGGGTGTATTCAAATTTAATAGTTG
ATGAGGGGTGAACTGTAGTCAGGGTAGAGGTATTCTGGGAAATAGAACTTGAAT
GCCACAAGCAGATTTTTAAATGAAGTTCATTGGGTTGTGTTGGTCATCTTTAAGG
GCTCTTAAGATAATAGAGGGTGTAGGGAAGGAGAGTGGTGCTAAATTGAGGGGA
GCCAACTTCTTCATTAGCCTGACAGCTAATACCTTCCCAAGGTTTATATACATGA
ATAACTTTTTAAAATGAAAATAAATAGGGCTTTGATGAATTTCTTGCTAAGAATG
CATTTTAATTTCATGCTTTTTGCTTTAAAGGTATTGTTACTAGATTTGAATGTTCA
CATCTTAAGTTGATCAGTGAAATTTGATACTGAAGCTGAAGAATTGTTGGGTGGC
TTATTTTTTGAAAGATTACTGCATTTCTTTGAAGCTGCCCACTTACCTTTTTGTGCT
AGTTATGTTTTTGGTAACAGTTTCTTTATTAATTTTTTAAAGGCCTGGTACTGTGG
CGCTCCGTGAAATTAGACGTTATCAGAAGTCCACTGAACTTCTGATTCGCAAACT
TCCCTTCCAGCGTCTGGTGCGAGAAATTGCTCAGGACTTTAAAACAGATCTGCGC
TTCCAGAGCGCAGCTATCGGTGCTTTGCAGGTAAAATGGTGGGTGGGAAGACTC
AGAGTTTGTATTCCTGTTGTGTACCAAGAACAGTTCCAAATTGTTGCATGTGCTTA
TATCATTTAATCACAAGCCTGTCAGGTAGTTGATATTGTTACTTCACTGTTGAGAC
TTCAGAAAGGTTAAATTGCTCAAGGTCATACACGTAGAAAATGGCAAAACCATA
ATTTGAACCTATTTGACTCCAAGGCTTAGTGCACATTCCATTATACCATTTAAAAT
TTTGAAACATTGCACTAAAAACAATATTTAAAGAAAATCCTCTGGTTTGGTTTAT
GGATGCTGCAGGACATTAAGAAGAACTTGAGACTAGAGGTCTATATTTGTAGTA
ACAATTTCAAACAACAGTGCCTAGAATAAAAGAAATGTCTCTTCAGGTCCTGAA
GAAACATATAGGTAGAGAGAGCTTAATACCTAGGATGGGAATAGGCAGTATTAA
AAAATTATGCATTAAGACGTAAAAGGAGCAGTGAGTGGAGGATAGGATTGGATT
GGCAAGATTGGGGGAACGGTGCTTAAAGGTGATACTGGACGTGTAAAGGGGCCA
GGATTTGCCTTGTGGTTTCCTAAAGGGGATTAGGGATTGCCACTTACATGTGGAG
CATATTGAATTTAATCTTAATCTCTAGCATGTCAGGACTTAGAGAAATACTGTTCT
AGAGATATTTATGTATTTGAGATATTTGTGGTTTAAGTTAAGACCAAAACTTGAA
ATTCCTACAAGATACTTGATAAAGGTATGTAGACGTCATTAACATCAGTCACTTA
AGTAACCTATTTTATATTGTGTGGTTGGATTCTTTAGTTGCAAGTATCCCAGAATA
CAGTAAAGCTACCACCTCCATCAGAAGCATGCAGTTGGGGGTCGTTAAAAATGC
TGATTGTTAGGCACCATTGCAGACCTGAATCATTCTGGGGGTGGGGAGGGGGCA
TCCAGAATCTAAGTACATTCCATTGGAGGATTTTCAAGGCTGCTGTTGCTGTGCT
GGGCTTTTGCTTTATGTATGAGAAAGATAGGATAGGTGGATTCCACCCTGAAGTG
TAATTGGTCGAGCTGTTTCTATGGTTAATCTAAACTTCATTTGATTGGTGAATTCC
AGGAGTAGGCATCCATTAAATTCTCAGTGAGTTGCTGAAATTTCAACTACTAAAA
TCTTCCATGTTTTTACTTAAAACTTTATTTTCTGTTTTAATAGCCGATTATCTTCAG
TTCTTATTTATTACTAAATACAGAAATGTTACTAAGATTCGTAAGCATTGGTACA
AAGCTGCTAATATTTTCTAGTTAGAACTGTGCTTAAAGAACAATACTGTACTTGA
AAAGTGTAGCGTTTTTAGCTTAAATGTCAATAATGTAGCCTTTTAAATTGAATTTA
CTAAACAATCTGGGTAAAAGACTCATCTTTCTAAAATTATTCACATGTAAACTTC
CATTGTTACTATTACATATTTGTCATTAAGTGTGGTTGTATTGGTTTTAAGCAAAA
TGTTCACCTCTTCTGTCCACAGTTCTCTTAGTGTTAGATAGCTCTTTATTGGAAAG
AGTGATCTTTGTTAAGCTTTTGTGTTTTCTGTTTGCTTTTTTATGCATATATTTTAG
TTATCTTAAAAATACAGAGAGTGGGCCGGGCGCCGTGACTCACATCTGTAATCCC
AACACTTTGGGAGGCTGAGGTGGGTAGATCACCTGAGGTCAGGAGTTCCAGACC
AGCGTGGCCAACATGGTGTGAAACCCCTTCTCTACTAAAAATACAAAAAATTAG
CTTGGCGTGGTGGCAGGCGCCTATAATCCCAGCTACTAGGGAGGCTGAGGCAGG
AGAATTGCTTAAACCCGGAAAGCAGAGGTTGCAGTGAGCCGAGATGGCGCCATT
GCACTCCAGCCTGGGCAACAAGAGCAAAACTCTTGTCTCAAAAAAAAAAAAAAG
ATAACAGAGGGAGAATTTGTTAATGGAATAGGATAATAGAAGATCGCAATTGAA
GTAGAAATCCAAAGGTCCTTTGTTTTTGGCCTTAGAATTACACACTTTACAACTA
AAGGATAGCAGGCAGTCCAGTGAGAATGGTAGAGGAGAAATTAGTCTCAGTTTG
CCATAGATAATCAATAATTGACTTACTTAAAATATAGTCTTTGCTTTTAATGTCTG
AGAACTTGAACTCAAATATTCTTGTGTTAAGTGGACAGTGTTGCTTTGAAATGGT
AAAGAGGCAGACGTTGAATATACTCAAAATGTCCTAAACTAATACTTTCTTGTAG
TTCTTTTTTTTCTTTGCCATTAAATCAAAATTAATTCATTAGGTCATAAATTAGTG
TGGCAGTCTTTAATTCCCATTACAGCCTTAATGTTTTAAGGGACTTTGGGTTACTT
GTAATAAAATTGGAAATGTATGGGTCATGGTCAGATCCTCTCGTAGGAGTTCAGT
CTCCTCTCAGAGCTGACCAGACACAATCCTTATAAACTAGTAGCCAGGTTCTAAG
ACAGCAGTGAGAGCCCCAGCTACCACATGCCACATCTGAGTGGTTCTAAGTGAT
ACCACTCTGAAAGGCCACCAGAACCATGGTGCCTGATGGTTTGCTTGCATAGTAG
CTTGTACCACATTTTACAAAAGCATTCTAGCTTTCTGTAATCCCGAGGTGCCAGG
TTAAGCAAAATTAAACTGTTTTCTTTCCTGATATAATAGCTGATATGCGTTGTGAG
CCCCTAAGGAGGCTACAATATGCCATTTTTTCCAGGATTTCTACCCACTTCTAGA
ATATCTTAAGAACGAATCTCGGAATATTAAGGACTTTTGCTTTTAATACCTGTGG
AATAGGTAAGATGCGAGGGACTACTTGCATAGAATAGAAACATTTTTTAAAACT
AGCAAATATTGAAAATCTACTTGAGGACAAGGCATTTGGAGTATTAAATGAGAT
TTCTGTTATCAAGTAACTTAAGGGCACCAAAAAATGGCTGATAATGCAAGGTCAT
TGTGAGTGCCAAGGAACTAGGAGTTCAGGGTTGCCTGCACTGGCAACAGGAGCA
AGAGACAAGTATGCGATTTTCTCCTTCATGGAATAGTCTTGAGCTGGGCCTTAAA
GGGTAGATAAGTAAGACTTAGTTGTTAATAACAAATGCTGGAGAAACACACGTG
AAAATAGTTCAATTTGAATGAAGGAAACTTGTAGAAGAGTAGTGATAGGGCTAA
AAAGGATGAAGCTAGAAATGTGAAAGTACCTTTTTTTTTTTTTTTTGAGACGAGT
CACACTCTGTCACCTAGGCTGGAGTGCAGTGGTGCAATCTCGGCTTACTGCAGCC
TCCGCCTCCCGGGTTCAAGCAATTCTCCTGTCTCAGCCTCCCAAGTAGCTGGGAC
TACAGGCACATGCCACCACGCCCAGCTACTTTTTGTATTTTTAGTAGAGACAAGG
TTTCTCCGTGTTGGTCAGTCTAGTCTCAAGCTCCTGACCTCGGGTGATCTGCCTGC
CTCGGCCTCCCAAAGTGCTGGGATTACAGGCGTGAGCCACCACGGCACCTGGTCT
GGAATTTTTTTTATTAGAATAAAAATGCAACTATAAGACTGTTTTCTCCCTTAATA
TATCTTCAGCAGAGAAATAACTTTTCCTTATAGAAAAGGAGAGAGAGCCAACTA
ACCTATCATTTCATGCTCCAGATCCAAAACTGTTGGATTTATGATTATTTTTTAAA
ATGGTAATTTCTCCATTTCAAAATGAGTAAGCAGGCCGGGCATGGCGGCTCACAC
CTGTAATCCCAGCACTTTGGGAGGCTGAGGTGGGCAGATCACCTGAAGTTAGGA
GTTCAAGACCAGCCTGGCCAACATGGTGAAACCCCATCTCTACTAAAAATACAA
AATTAGCTGGACGTGATGGTGCATGCCTGTAATCCCAGCTACTCGGGAGGCTGA
GGCAGGAGAATTGCCTGAGCTCGGGAGGTGGAGGTTGCAGTGAGCCGAGGTTAT
ACCACTGCACTCCAGCCAGGGCTACAGAGCAAGACTCAAACCTCAAAAAACAAA
AACAAAAAGAGTAAGCAGATGTTTTGGCTTAGACTAAAAGATTCTTCAGCTTTTC
AGACAGCTATAAGTATACTAAGAATTTGAGTTATGAGTTAATTCTAAGTGGAAAC
GCCCCTTTTTCCTCTTCACAAGTTAAGTGTCAATGAGTGATTCATACACTGTCATT
TTTAAGTGGTAGTAGGAATAAGATAACTTGAAAGGATCTTACAGTCAAATGGGA
AAAACCAGAGAAATCGATACTAGTACTAGAGGGCAACAAATGCTGTTACAAATT
GGGGTACGTAGAGGAAGGTACTTGGTAGAGGACAGGGGCATGTTTCCGGGGCAT
AGATCAAAGTATATAAATAAGGAACTGCCAGGCCAGTTGCGGTGGCTCACTCCT
GTAATCCCAGCACTTTGGGAGGCCGAGGCAGGAGGATCACGAGGTCAGGAGATC
GAGACCATCCTAGCTAACACAATGAAGCCCCATCTCTACTGAAAATTAGTCAGG
CGTGGTGGTGGGCGCCTGTAGCCCGAGCTACTCGGGAGGCTGAGGCAGGAGAAT
GACATGAACCTGGGAGGTGGAGCTTGCAGTGAGCTGAGATCTTGCCACTGCACT
CCAGCCTGGGCAACAAAGAGAGACTCCGTCTCAAAATAAATAAATAAGGAACTG
CCGGGCGCGGTGGCTCACGCCTATAATCCCAGCACTTTGGGAGGCCAAGGTGGG
TGGATCACGAGGTCAGGAGTTCGAGACCAGCCTGACCAACATGGTGAAACCCCT
TCTCTACTAAAAATACAAAAAAGTAGCCAGGCATGGTGGCGCATACCTGTAGTC
CCAGCTACTCGGGAGGCTGAGGCAGGAGAATCGCTTGAATCCGGGAGGTGGAGG
TTGCAGTGAGCCGAGATCGCGCTACTGCACTCCAGCCTGGGCGACAGAGTAAGA
CTCCATCTCAGAAAGAAAGAAAGAAATACGGAACTAACTTGTGATATGTTCTGG
AATCAAAAGTACTCTTATGATAAAACAGGTATGAAAGGGAACATAGATGAGAAG
CATGTGATAAAAACCACTTGTTCACCATGTTATACTACTGGACAAGGCAGAGGTT
CACATACTGTGTGAATGGGATTCAGAGTGAGGAGGAGACTAGGCTGGGATGGGG
TATTTGGATTGGACATGATTGCGTTTATAAGAATGAGAGTGTTAAATTGGATTTC
TTGCTTTATTTGTGACATTTCAGTTTATTAGAAATCATGTTACCATTAGAAAAATT
GAAGTTTCCTAGTAACAAAGTAATTTGATTTGTGTAACTTGATAAAAGATTTACT
GACTTAAGCTTTTGTTTTTTTTCATAAGCTGCTTTTGAGCTTTGTCCCACAGGTTGT
AAAATGTAAGCATTTGGTAAAATTGTCAGCATCTTGCCCAGTCATTTTTTTAAAG
GGTTCAAAAACCTTTTTGTTTTAATTCGTATAGTTGGGTCTTAACTATTGGAAATA
ACATCATCAGTAATTTTTTCTTCATTCCTTTTGCAGGAGGCAAGTGAGGCCTATCT
GGTTGGCCTTTTTGAAGACACCAACCTGTGTGCTATCCATGCCAAACGTGTAACA
ATTATGCCAAAAGACATCCAGCTAGCACGCCGCATACGTGGAGAACGTGCTTAA
GAATCCACTATGATGGGAAACATTTCATTCTCAAAAAAAAAAAAAAAAATTTCT
CTTCTTCCTGTTATTGGTAGTTCTGAACGTTAGATATTTTTTTTCCATGGGGTCAA
AAGGTACCTAAGTATATGATTGCGAGTGGAAAAATAGGGGACAGAAATCAGGTA
TTGGCAGTTTTTCCATTTTCATTTGTGTGTGAATTTTTAATATAAATGCGGAGACG
TAAAGCATTAATGCAAGTTAAAATGTTTCAGTGAACAAGTTTCAGCGGTTCAACT
TTATAATAATTATAAATAAACCTGTTAAATTTTTCTGGACAATGCCAGCATTTGG
ATTTTTTTAAAACAAGTAAATTTCTTATTGATGGCAACTAAATGGTGTTTGTAGCA
TTTTTATCATACAGTAGATTCCATCCATTCACTATACTTTTCTAACTGAGTTGTCC
TACATGCAAGTACATGTTTTTAATGTTGTCTGTCTTCTGTGCTGTTCCTGTAAGTT
TGCTATTAAAATACATTAAACTATACCTGCTTTTGGTCTTTA.

### H3.3 (H3F3A) Protein Sequence (Mature):

(SEQ ID NO: 91)
ARTKQTARKSTGGKAPRKQLATKAARKSAPSTGGVKKPHRYRPGTVALRE
IRRYQKSTELLIRKLPFQRLVREIAQDFKTDLRFQSAAIGALQEASEAYL
VGLFEDTNLCAIHAKRVTIMPKDIQLARRIRGERA

### H3.3 (H3F3A) K27M Protein Sequence (Mature):

(SEQ ID NO: 92)
ARTKQTARKSTGGKAPRKQLATKAARMSAPSTGGVKKPHRYRPGTVALRE
IRRYQKSTELLIRKLPFQRLVREIAQDFKTDLRFQSAAIGALQEASEAYL
VGLFEDTNLCAIHAKRVTIMPKDIQLARRIRGERA

### H3.3 (H3F3A) Protein Sequence:

(SEQ ID NO: 198)
MARTKQTARKSTGGKAPRKQLATKAARKSAPSTGGVKKPHRYRPGTVAL
REIRRYQKSTELLIRKLPFQRLVREIAQDFKTDLRFQSAAIGALQEASE
AYLVGLFEDTNLCAIHAKRVTIMPKDIQLARRIRGERA

### H3.3 (H3F3A) K27M Protein Sequence:

(SEQ ID NO: 199)
MARTKQTARKSTGGKAPRKQLATKAARMSAPSTGGVKKPHRYRPGTVA
LREIRRYQKSTELLIRKLPFQRLVREIAQDFKTDLRFQSAAIGALQEA
SEAYLVGLFEDTNLCAIHAKRVTIMPKDIQLARRIRGERA

It is understood that when the amino acid sequence of H3.3 protein or H3.3 K27M protein is translated, there is a methionine (M) at position 1 of the amino acid sequence of H3.3 protein (SEQ ID NO: 198) or H3.3 K27M protein (SEQ ID NO: 199). The M at position 1 in the amino acid sequence of H3.3 protein or H3.3 K27M protein is removed post-translationally resulting in a mature H3.3 protein having the amino acid sequence of SEQ ID NO: 91 or in a mature H3.3 K27M protein having the amino acid sequence of SEQ ID NO: 92. A person of ordinary skill in the art understands a H3.3 K27M protein to include a lysine (K) to M mutation at position 27 of the mature H3.3 K27M protein (SEQ ID NO: 92).

### Pharmaceutical Compositions

In some embodiments, pharmaceutical compositions comprising one or more ASO(s) are provided. In some aspects, the ASO(s) are referred to as agents or active ingredients of the pharmaceutical compositions provided herein. The compositions comprising ASO(s) can be mixed with a pharmaceutically acceptable carrier, either taken alone or in combination with the one or more additional therapeutic agents described above, to form pharmaceutical compositions.

A pharmaceutically acceptable carrier is compatible with the active ingredient(s) of the composition (and preferably, capable of stabilizing it). In some embodiments, the pharmaceutical composition comprises a pharmaceutically acceptable salt of an ASO. Such compositions are delivered or administered in effective amounts to treat an individual, such as a human having a disease or disorder resulting from mutation, for example those described herein.

To “treat” a disease, means to reduce or eliminate a sign or symptom of the disease, to stabilize the disease, and/or to reduce or slow further progression of the disease. In some embodiments, “treat”, “treatment” or “treating” is intended to include prophylaxis, amelioration, prevention or cure from the disease. For example, treatment of brain cancer according to use of the compositions and methods provided herein may result in e.g., decreasing tumor size, slowing or eliminating metastasis, or prolonging patient survival.

Actual dosage levels of active ingredients in the pharmaceutical compositions of the invention can be varied to obtain an amount of the active ASO and optionally other agent(s) that is effective to achieve the desired therapeutic response for a particular patient, combination, and mode of administration. The selected dosage level depends upon the activity of the particular ASO and other agent(s), the route of administration, the severity of the condition being treated, the condition, and prior medical history of the patient being treated. However, it is within the skill of one in the art to start doses of the compositions described herein at levels lower than required to achieve the desired therapeutic effort and to gradually increase the dosage until the desired effect is achieved. An “effective amount” refers to an amount of one or more ASOs that results in improvement (complete or partial) of a disease or disorder caused by a mutation, such as a disease or disorder caused by mutant H3F3A allele or mutant H3F3A gene, which replaces lysine 27 with methionine (K27M). The resulting H3.3K27M is a toxic gain-of-function mutation found, for example, in DMG tumors. An effective amount of one or more ASOs, such as one or more gapmer ASOs; one or more splice modulating ASOs; or a combination of one or more gapmer ASOs and one or more splice modulating ASOs, is an amount that reduces (totally or partially) the effects of pHGGs, such as the effects of DMG tumors, including DIPG. An effective amount can, for example, delay or prevent the onset, severity or progression of a disease or disorder caused by a mutant H3F3A allele or H3F3A gene, such as by delaying or preventing the onset, severity or progression of brain tumors in children (e.g., DMG tumors, including DIPG). The effective amount used will vary, for example, with the stage of or the size of the brain tumor, the severity of the effects of the brain tumor, the age and physical condition of the individual to whom the one or more ASO(s) are administered, etc. An effective amount can also be referred to as a therapeutically effective amount. Such ASOs can be administered alone or in combination with another (different) therapeutic agent.

In some embodiments, an effective amount of an ASO is administered to an individual. In some embodiments, the effective amount of an ASO is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, about 1000 mg, about 1500 mg, about 2000 mg, about 2500 mg, about 3000 mg, about 3500 mg, about 4000 mg, about 4500 mg, or about 5000 mg.

In some embodiments, the effective amount of an ASO is about 1 mg to about 5 mg, about 5 mg to about 10 mg, about 10 mg to about 20 mg, about 20 mg to about 40 mg, about 40 mg to about 80 mg, 50 mg to about 100 mg, about 100 mg to about 200 mg, about 200 mg to about 400 mg, about 400 mg to about 800 mg, about 500 mg to about 1000 mg, about 1000 mg to about 1500 mg, about 1500 mg to about 2000 mg, about 2000 mg to about 3000 mg, about 3000 mg to about 4000 mg, or about 4000 mg to about 5000 mg. In some embodiments, the effective amount of an ASO is about 5 mg.

In some embodiments, the effective amount of an ASO is about 5 mg. In some embodiments, the effective amount of an ASO is about 10 mg. In some embodiments, the effective amount of an ASO is about 15 mg. In some embodiments, the effective amount of an ASO is about 20 mg. In some embodiments, the effective amount of an ASO is about 25 mg. In some embodiments, the effective amount of an ASO is about 30 mg. In some embodiments, the effective amount of an ASO is about 35 mg. In some embodiments, the effective amount of an ASO is about 40 mg. In some embodiments, the effective amount of an ASO is about 45 mg. In some embodiments, the effective amount of an ASO is about 50 mg.

In the combination therapies, an effective amount can refer to each individual agent or to the combination as a whole, wherein the amounts of all agents administered are together effective, but wherein the component agent of the combination may not be present individually in an effective amount.

The pharmaceutical compositions described herein (e.g., those comprising ASOs can be administered to a subject by any suitable route). For example, compositions can be administered orally, including sublingually, rectally, parenterally, intracisternally, intravaginally, intraperitoneally, topically and transdermally (as by powders, ointments, or drops), bucally, or nasally. The term “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. In some embodiments, a composition (e.g., comprising an ASO) is administered via intrathecal administration or via reservoir (e.g., an Ommaya reservoir).

Surgical implantation also is contemplated, including, for example, embedding a composition of the disclosure in the body such as, for example, in the brain, in the abdominal cavity, under the splenic capsule, brain, or in the cornea.

The pharmaceutical compositions described herein can also be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to an agent of the present disclosure, stabilizers, preservatives, excipients, and the like. Lipids used can be, for example, phospholipids and phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of the pharmaceutical compositions described herein include powders, sprays, ointments, and inhalants as described herein. The active agent(s) is mixed under sterile conditions with a pharmaceutically acceptable carrier and any needed preservatives, buffers, or propellants which may be required. Ophthalmic formulations, eye ointments, powders, and solutions also are contemplated as being within the scope of this disclosure.

Pharmaceutical compositions (e.g., those comprising ASOs) for parenteral injection comprise pharmaceutically acceptable sterile aqueous or non-aqueous solutions, dispersions, suspensions, or emulsions, as well as sterile powders for reconstitution into sterile injectable solutions or dispersions just prior to use. Examples of suitable aqueous and non-aqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

Compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the pharmaceutical compositions described herein (e.g., those comprising ASOs), it is desirable to slow the absorption of the drug from subcutaneous or intramuscular injection. This result can be accomplished by the use of a liquid suspension of crystalline or amorphous materials with poor water solubility. The rate of absorption of the active agent(s) then depends upon its rate of dissolution which, in turn, may depend upon crystal size and crystalline form. Alternatively, delayed absorption of a parenterally administered active agent(s) is accomplished by dissolving or suspending the agent(s) in an oil vehicle.

Injectable depot forms are made by forming microencapsule matrices of the agent(s) (e.g., ASOs, anti-cancer drugs) in biodegradable polymers such a polylactide-polyglycolide. Depending upon the ratio of agent(s) to polymer and the nature of the particular polymer employed, the rate of agent(s) release can be controlled. Examples of other biodegradable polymers include poly(orthoesters) and poly(anhydrides). Depot injectable formulations also are prepared by entrapping the agent(s) in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

Also described here are methods for oral administration of the pharmaceutical compositions described herein. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes the agent(s) (e.g., ASOs and optionally readthrough drugs) and inert ingredients which protect against degradation in the stomach and which permit release of the biologically active material in the intestine.

In such solid dosage forms, the agent(s) is mixed with, or chemically modified to include, a least one inert, pharmaceutically acceptable excipient or carrier. The excipient or carrier preferably permits (a) inhibition of proteolysis and/or nucleic acid degradation, and (b) uptake into the blood stream from the stomach or intestine. In a most preferred embodiment, the excipient or carrier increases uptake of the agent(s), overall stability of the agent(s) and/or circulation time of the agent(s) in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropyhnethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents. Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The agent(s) also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the agents(s) (e.g., ASOs and optionally anti-cancer drugs), the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage. Oral composition can also be administered by oral gavage.

Suspensions, in addition to the active ingredient(s), can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Pulmonary delivery of the ASOs is also possible. The agents are delivered to the lungs of a mammal, such as a human, while the mammal is inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl.5): s.143-146 (1989)(endothelin-1); Hubbard et al., Annals of Internal Medicine 3:206-212 (1989)(al-antitrypsin); Smith et al., J. Clin. Invest. 84:1145-1146 (1989) (al-proteinase); Oswein et al., “Aerosolization of Proteins,” Proceedings of Symposium on Respiratory Drug Delivery II, Keystone, Colorado, March, 1990 (recombinant human growth hormone); Debs et al., The Journal of Immunology 140:3482-3488 (1988) (interferon gamma and tumor necrosis factor α) and Platz et al., U.S. Pat. No. 5,284,656 (granulocyte colony stimulating factor). The composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

In addition, the use of liposomes, microcapsules or microspheres, inclusion complexes, or other types of carriers is contemplated.

In order to facilitate delivery of agent(s) across cell and/or nuclear membranes, compositions of relatively high hydrophobicity are preferred. Agent(s) can be modified in a manner which increases hydrophobicity, or the agents can be encapsulated in hydrophobic carriers or solutions which result in increased hydrophobicity.

In one aspect, the invention provides kits comprising a pharmaceutical composition comprising an effective amount of one or more ASO and an effective amount of one or anti-cancer drugs and instructions for administration of the pharmaceutical composition. In some aspects of the invention, the kit can include a pharmaceutical preparation vial, a pharmaceutical preparation diluent vial, and the ASO(s) and additional agent(s). The diluent vial contains a diluent such as physiological saline for diluting what could be a concentrated solution or lyophilized powder of the agent of the invention. In some embodiments, the instructions include instructions for mixing a particular amount of the diluent with a particular amount of the concentrated pharmaceutical preparation, whereby a final formulation for injection or infusion is prepared. In some embodiments, the instructions include instructions for use in a syringe or other administration device. In some embodiments, the instructions include instructions for treating a patient with an effective amount of the ASO(s) and optional additional agent(s). It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

### Methods

In some aspects, this disclosure provides methods for decreasing the expression of or modulating the sequence of a mutated gene associated with a disease. In some embodiments, the method comprises administering to an individual an ASO that is complementary to the mutated gene associated with a disease. In some embodiments, the ASO specifically binds to transcript of the mutated gene to a greater extent than the extent to which it binds the transcript of the wildtype gene. In some embodiments, the ASO decreases the expression of the wildtype gene by less than 20% (e.g., less than 10%, less than 20%, less than 30%, less than 40%, less than 50%, less than 75%, or less than 90%). In some embodiments, the ASO inhibits splicing of an exon comprising the disease associated mutation into an mRNA. In some embodiments, the concentration of the mRNA comprising the exon comprising the disease causing mutation is decreased by at least 10% (e.g., at least 10%, at least 20%, at least 30%, at least 40%, at least 50%, at least 60%, at least 70%, at least 80%, at least 90%, at least 95% or at least 99%).

In some embodiment, this disclosure provides methods of treating disease in an individual by administering ASOs. In some embodiments, the disease is cancer. In some embodiments, the disease is brain cancer. In some embodiments, the disease is high-grade glioma (pHGG). In some embodiments, the disease is diffuse midline glioma (DMG). In some embodiments, the disease is diffuse intrinsic pontine glioma (DIPG). In some embodiments, the disease is diffuse intrinsic pontine glioma (DIPG) associated with an H3F3A mutation. In some embodiments, the H3F3A mutation results in a K27M mutation that inhibit methylation of Histone 3.3a at position K27M. In some embodiments, the disease is associated with an H3F3A mutation. In some embodiments, the disease is associated with an H3F3A mutation at a position corresponding to K27M of Histone 3.3a.

In some embodiments, the individual is a human. In some embodiments, the individual is a child. In some embodiments, the individual is less than 18 years old (e.g., less than 18 years old, less than 15 years old, less than 12 years old, less than 8 years old, less than 6 years old, less than 5 years old, less than 4 years old, less than 3 years old, less than 2 years old, less than 1 year old, less than 6 months old, or less than 1 month old). In some embodiments, the subject is 0-3, 2-4, 3-6, 4-8, 5-10 or 8-16 years old.

In some embodiments, the individual is heterozygous for a mutant H3F3A allele. In some embodiments, the mutant H3F3A allele comprises a mutation at position 84 of SEQ ID NO: 86. In some embodiments, the mutation is an A to T mutation.

### Examples

### Example 1: ASOs that Mediate RNase H Cleavage of Mutant H3F3A mRNA—or Both Mutant and Wild Type-In DIPG-Patient-Derived Cells

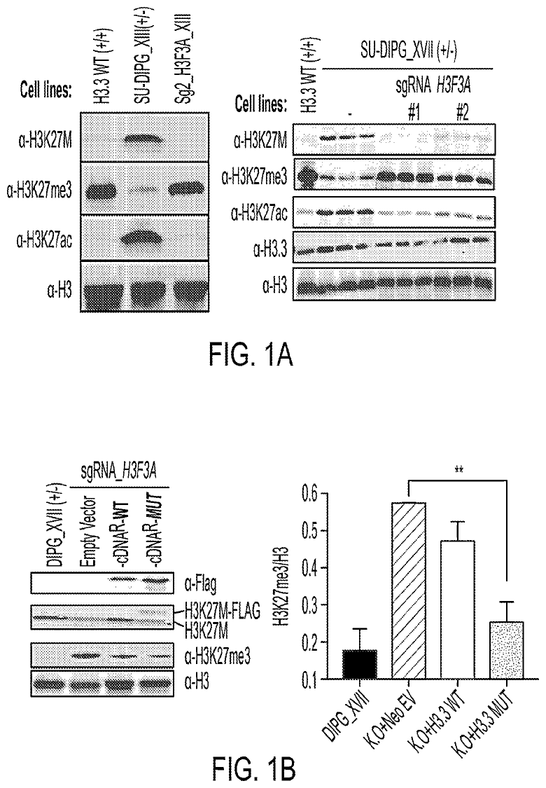

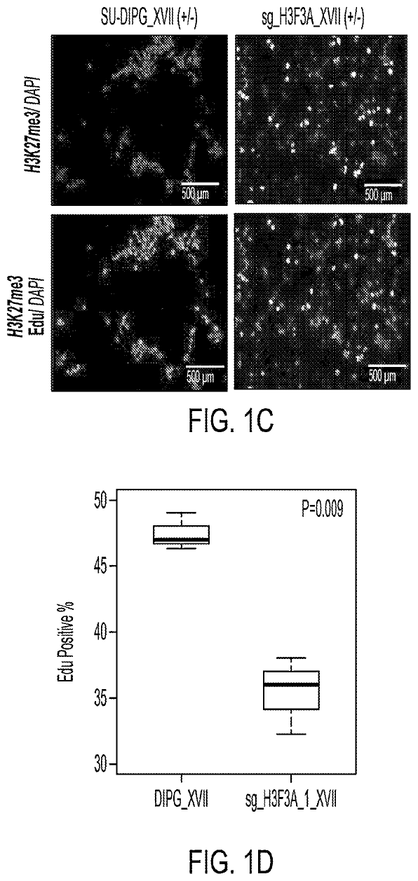

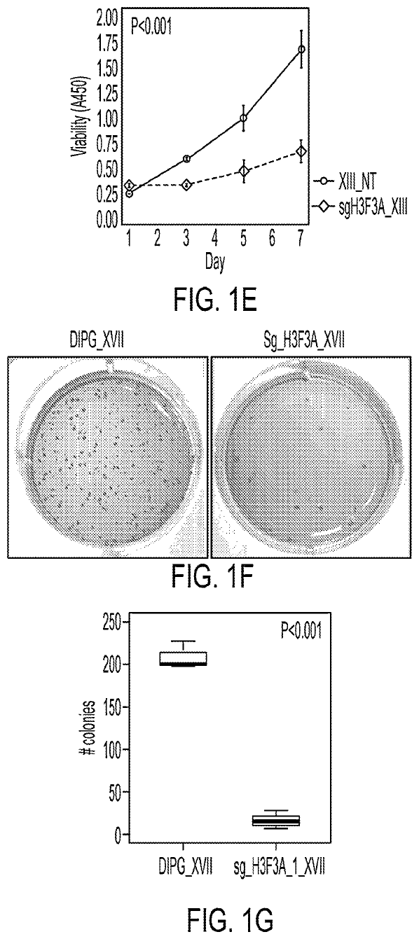

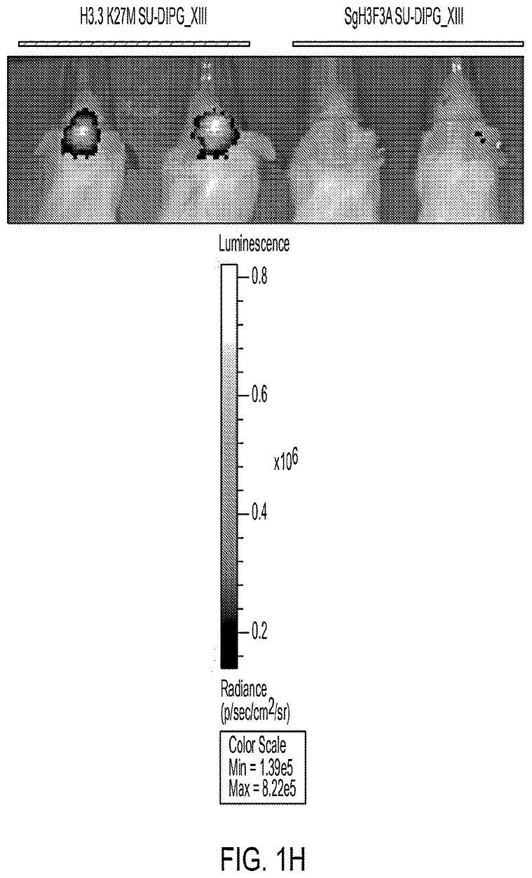

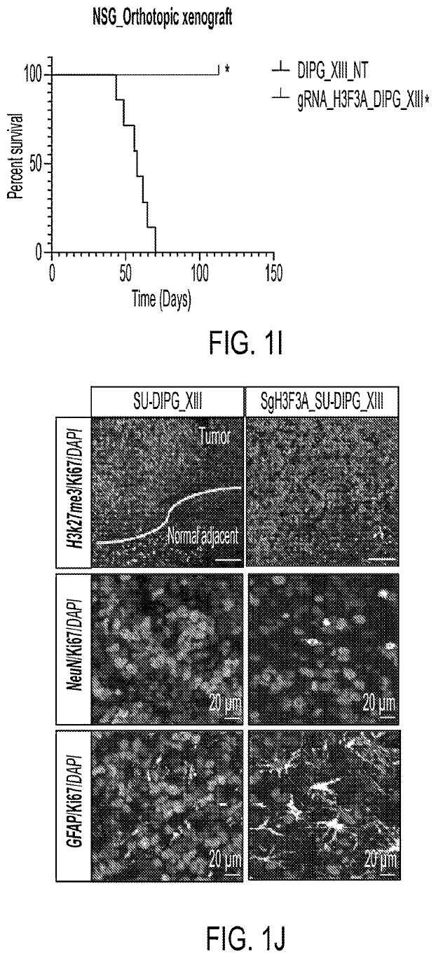

As a control to determine the biological consequence of full genetic knockout of H3F3A, CRISPR-Cas9 with sgRNA targeting both mutant and wild-type alleles was used to knock out H3F3A, but leave H3F3B (which encodes the identical protein) unaffected in DIPG-patient cells (FIGS. 1A-1J). H3.3-K27M depletion restored the repressive H3K27me3 trimethylation mark and reduced the permissive H3K27ac acetylation mark, assayed by immunoblotting and immunofluorescence (IF) (FIGS. 1A-1C). This in turn reduced proliferation and soft-agar colony formation (FIGS. 1D-1G), and significantly extended survival in a DIPG orthotopic-xenograft mouse model (FIGS. 1H-1J). These results identified mutant H3.3-K27M as a promising therapeutic target.

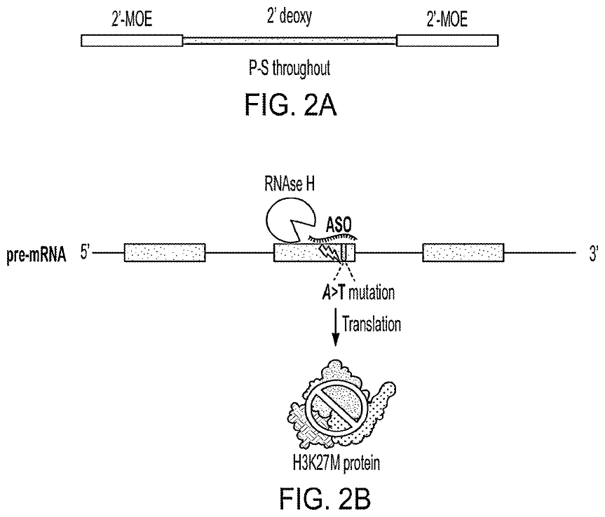

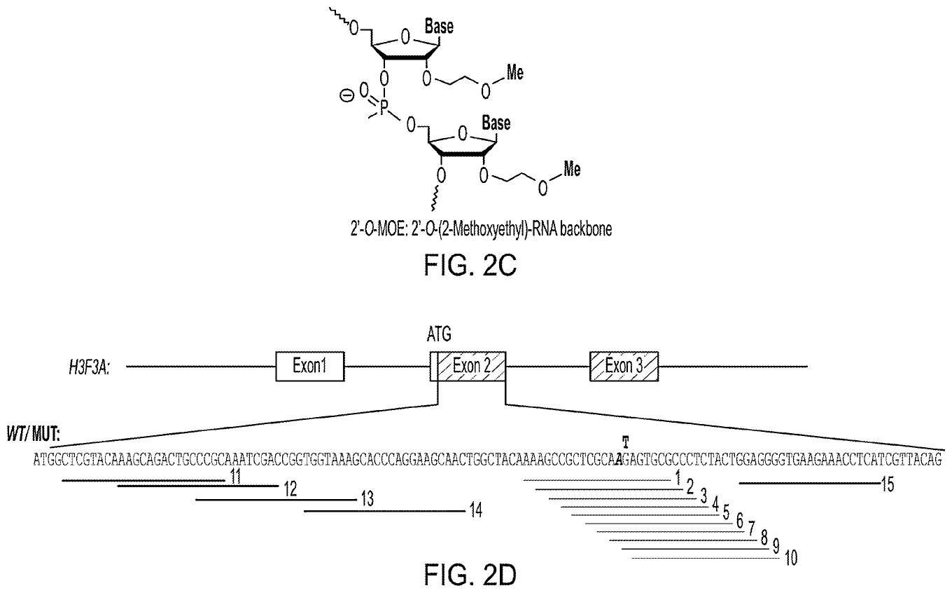

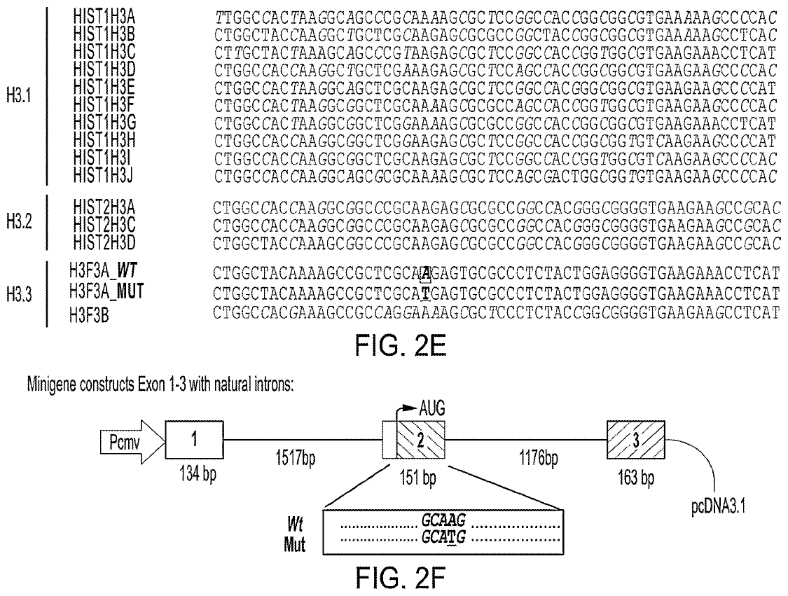

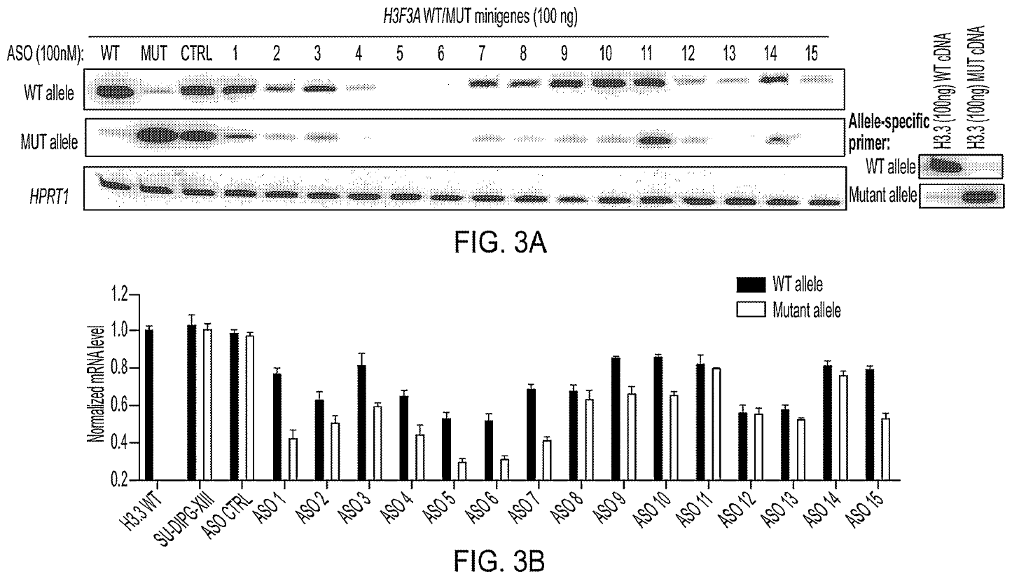

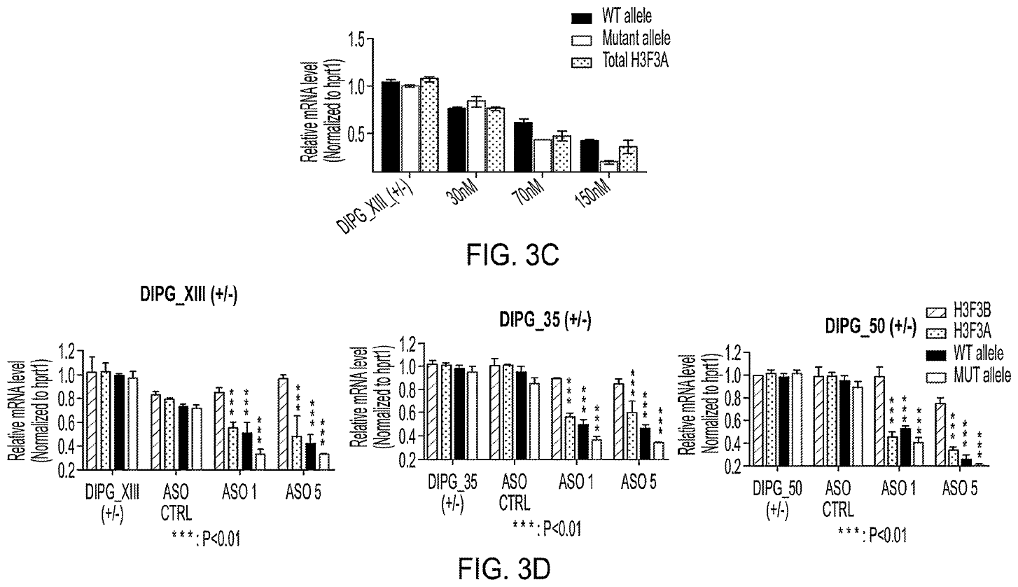

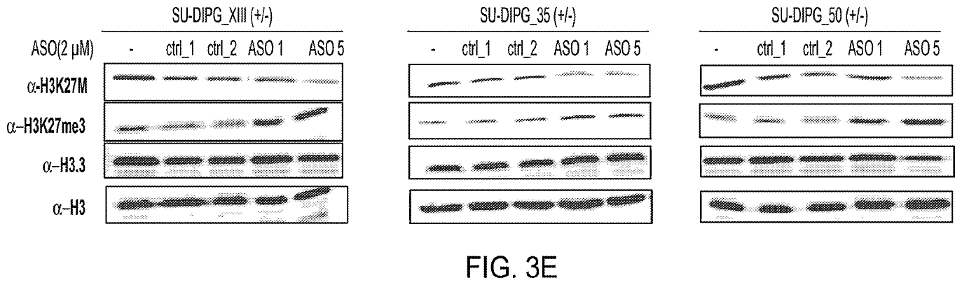

To pharmacologically target H3F3A mRNA, gapmer PS ASOs with MOE wings targeting H3F3A mRNA were designed (FIGS. 2A-2F). An initial screen identified ASOs with the desired properties (FIGS. 3A-3E). Total RNA and protein was isolated at set times after incubation with ASOs. RNA analysis and quantitation of H3F3A transcripts by radioactive RT-PCR and RT-qPCR was performed (FIG. 3A-3B). Consistent with the phenotypes of the knockout, the lead ASOs, ASO 1 and ASO 5, knocked down ˜60-70% of the mutant mRNA and H3.3-K27M protein, resulting in 2-to-3-fold H3K27me3 restoration. ASO 1 and ASO 5 were also shown to be effective at decreasing in three different patient derived cell lines (FIGS. 3D-3E). The level of histone H3.3 protein was measured by immunoblotting with infrared detection and antibody specific for H3.3 with the K27M mutation in the presence of ASO 1 and ASO 5 (FIG. 3E). Downstream epigenetic changes were measured using antibodies to K27 tri-methylated or acetylated histones (FIG. 3E).

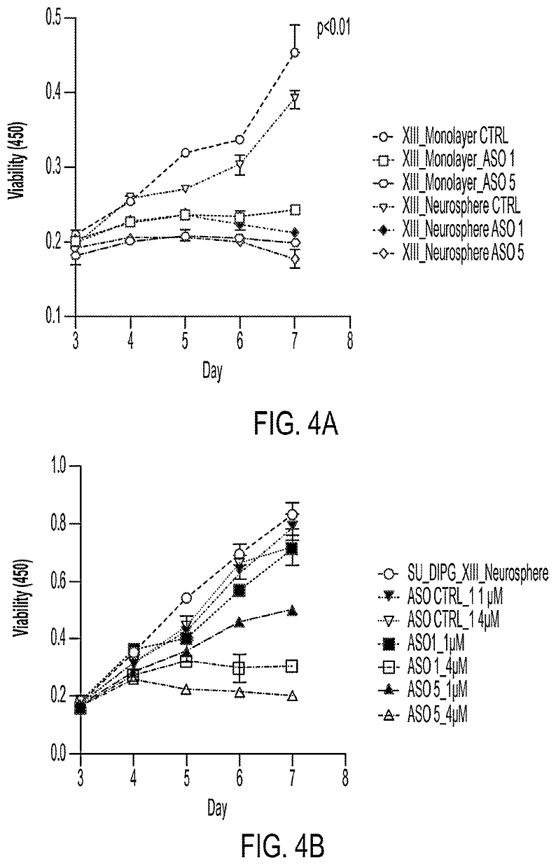

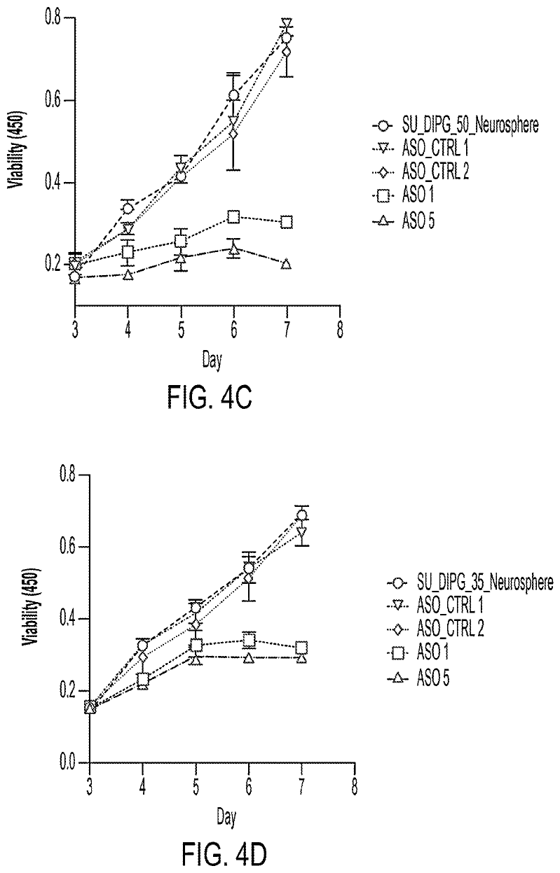

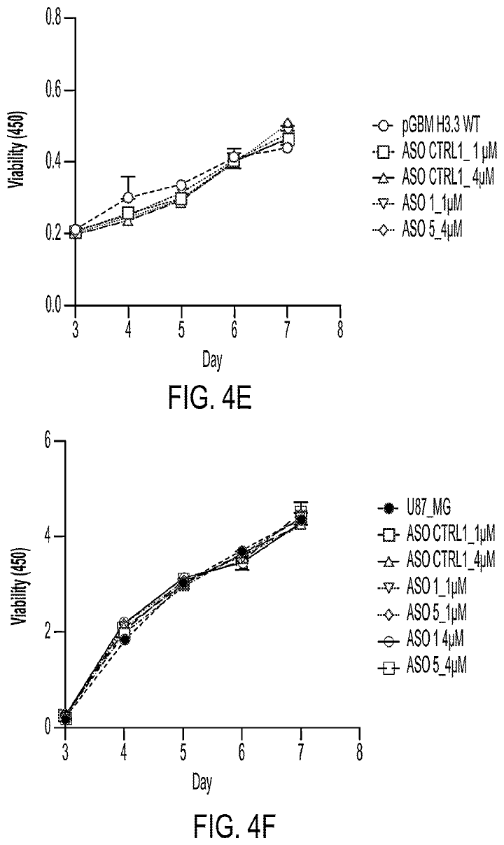

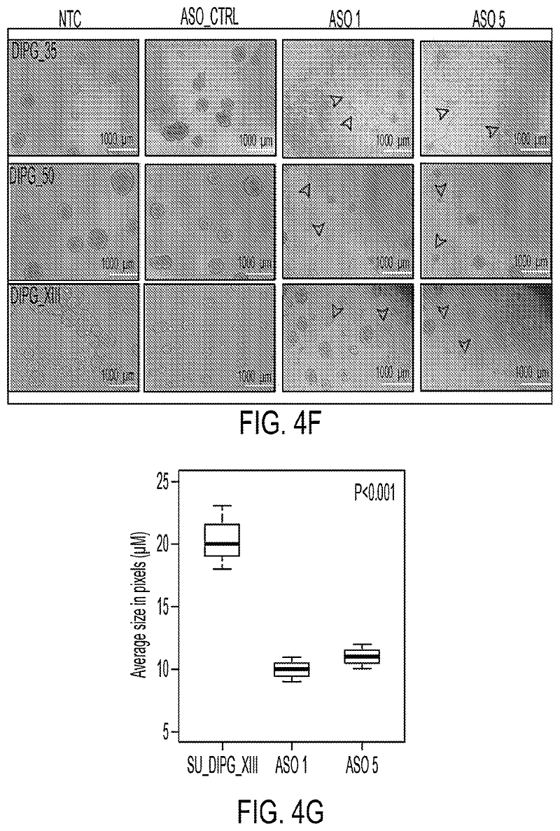

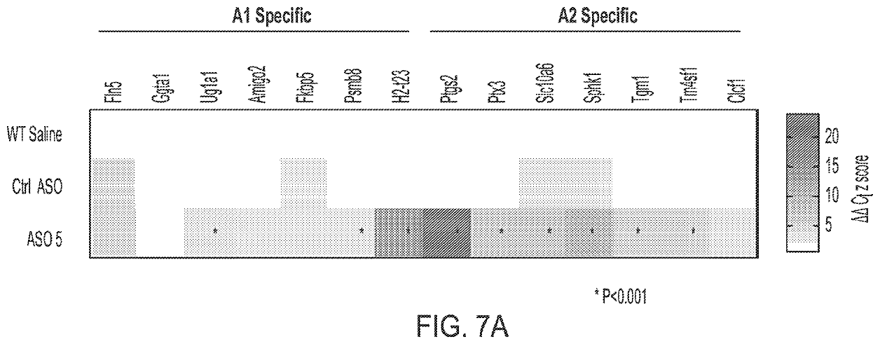

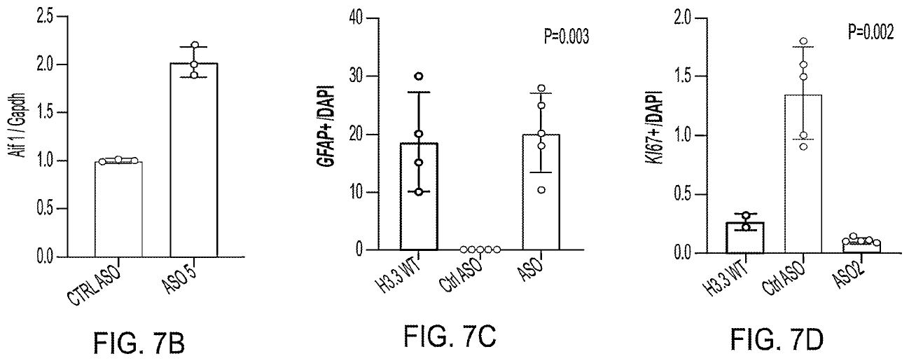

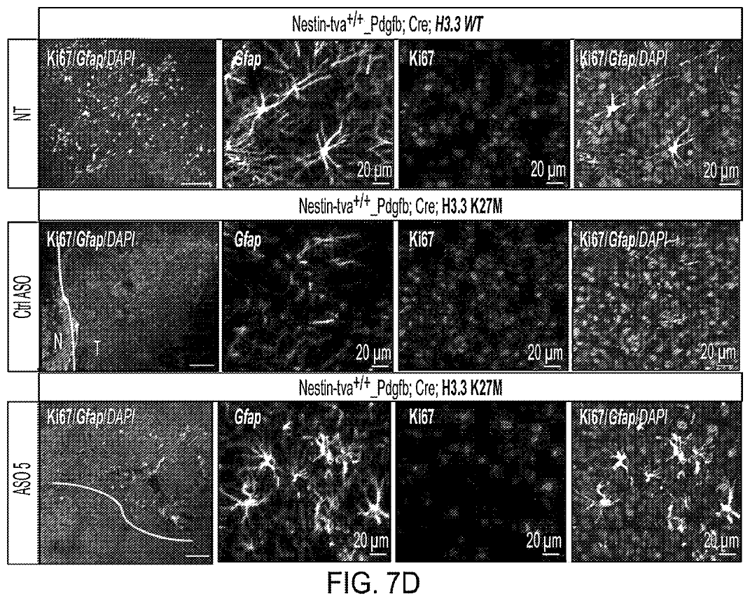

ASO 1 and ASO 5 were introduced into patient-cell neurospheres by free uptake (gymnotic delivery). This method required high ASO concentrations, due to scavenger-receptor downregulation in cell culture, but it was a better surrogate for in vivo delivery than transfection or electroporation. Characterization of phenotypic changes elicited by treatments with ASO 1 and ASO 5 ASOs was performed. Viable cells were counted using standard proliferation and cytotoxicity assays (FIGS. 4A-4E) and cellular morphology was examined under light microscopy (FIGS. 4F-4G). Results showed that ASO 1 and ASO 5 specifically delayed H3.3-K27M tumor-cell growth in several DIPG-patient-derived cells grown as neurospheres (FIGS. 4A-4E), induced neurite-like processes (FIG. 7F), and decreased the average size of the tumor cells (FIG. 7G).

### Example 2: Gapmer ASOs in an RCAS-Tva Mouse Model, and Characterization of Cellular Phenotypes

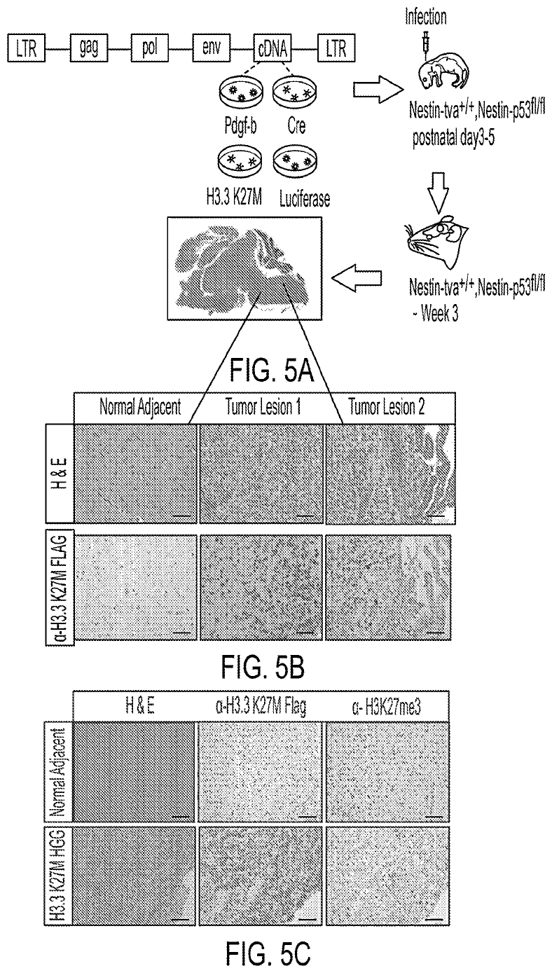

For in vivo experiments, a genetic mouse model was generated, adapting the RCAS-Tva system (9,10), but introducing a human H3F3A-K27M cDNA, whose transcripts can be targeted by human-specific ASOs (FIGS. 5A-5C). RCAS stands for replication-competent avian sarcoma-leukosis virus long terminal repeat (LTR) with splice acceptor (11). This viral vector only infects cells expressing the avian Tva receptor. Chicken DF1 cells producing viruses encoding Cre recombinase, H3.3-K27M, and PDGFB, were delivered into the brainstem of neonate mice with Tva driven by the nestin promoter, and a p53-floxed allele.

The original model (with a different murine mutant histone) developed high-grade DMG in 4-6 weeks, showed global loss of H3K27me3 and histologically resembled human tumors (10). This mouse model developed high-grade DMG in 3-6 weeks, confirmed by histology. ˜90% of H3.3-K27M tumors showed global H3K27me3 reduction, compared to normal adjacent tissue. Tumors were graded in blinded fashion by a pathologist. High-grade and low-grade tumors were classified by the presence or absence of vascular proliferation and/or pseudopalisading necrosis, respectively.

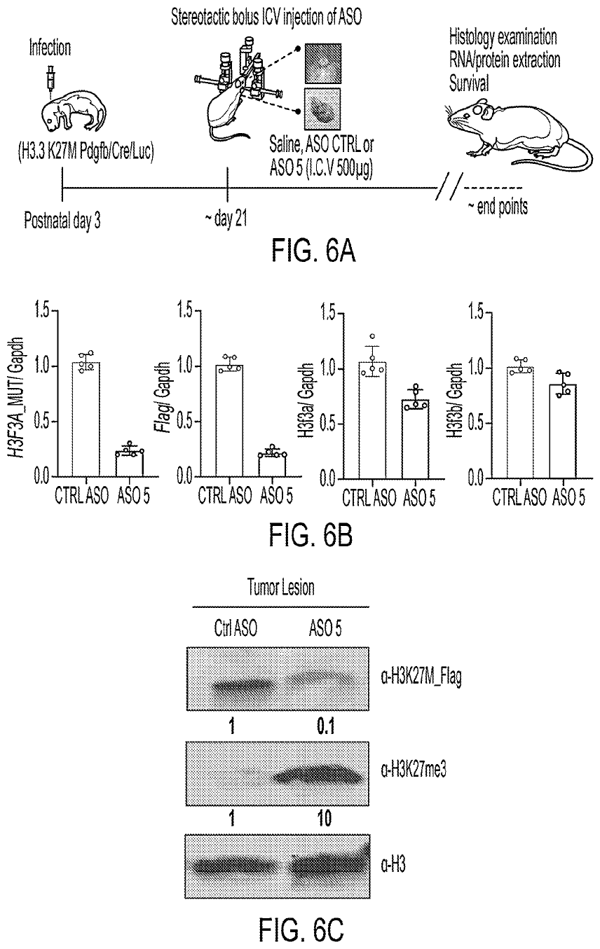

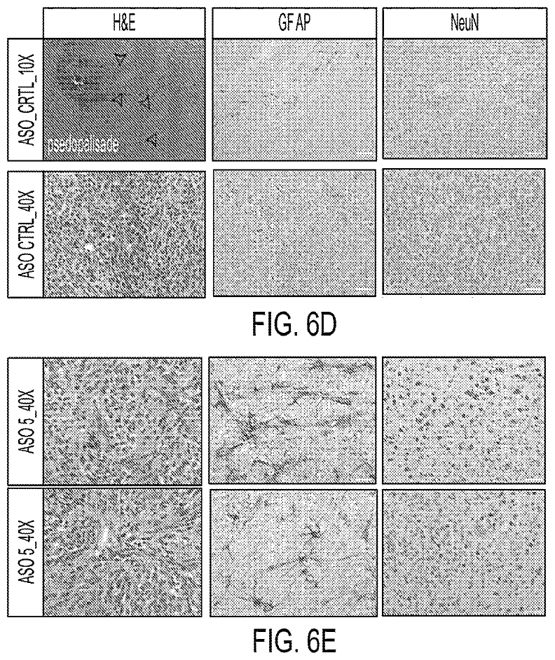

Next the efficacy of the lead ASO, ASO 5, was determined using the in vivo mouse model described in FIGS. 5A-5C. Saline-treated and ASO-treated cohorts were used as controls, and the tumorigenesis was monitored over time using luciferase-bioluminescence imaging. ASO 5 was administered by intracerebroventricular (ICV) injection, together with viral producer cells expressing H3F3A mutant cDNA, Pdgf and Cre, into postnatal-day-3 (P3) nestin; p53^{fl} transgenic mice (FIG. 6A). The mice developed tumors starting at 3 weeks post-injection, and the ASO-treated animals developed tumors later than the controls. The control and ASO-treated cohorts were euthanized on day 36. RNA and protein were extracted from tumors and normal adjacent tissue to confirm that the lead ASO knocked down flag-tagged H3.3-K27M in vivo and elicited downstream epigenetic changes. Additionally, microscopy experiments showed that GFAP and NeuN expression were increased (FIGS. 6C-6E, 7C-7D and 8A-8G), which is indicative of A2-specific reactive astrocyte differentiation.

RNA and protein were extracted from tumors with and without ASO treatment to measure knockdown efficiency, and downstream expression and epigenetic changes. Results showed that ASO 5 substantially decreased the relative expression of the H3F3A mutant gene, and minimally decreased the expression of wildtype H3F3A and H3f3b (FIG. 6B). ASO 5 also increased alpha-H3K27me3 (FIG. 6C), which is consistent with knockdown of the mutant allele. An increase in A2-specific reactive astrocyte differentiation markers was also observed (FIGS. 7A-7B).

Cellular phenotypic changes in the mouse model, related to the in vitro phenotypes were also characterized. H&E staining under light microscopy and immunofluorescence demonstrated that that GFAP and NeuN expression were increased (FIGS. 6C-6E, 7C-7D and 8A-8G), which was also indicative of A2-specific reactive astrocyte differentiation.

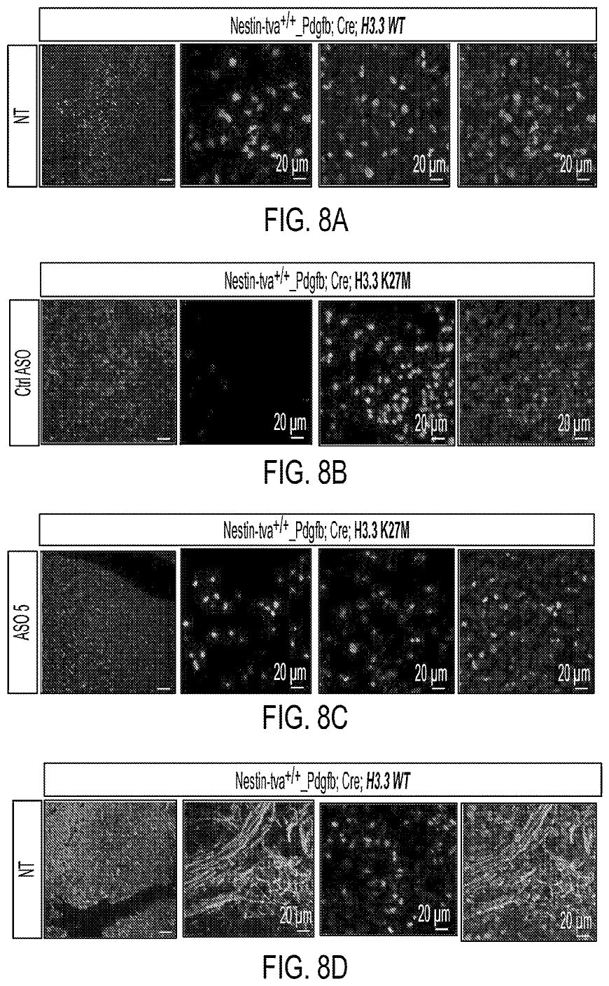

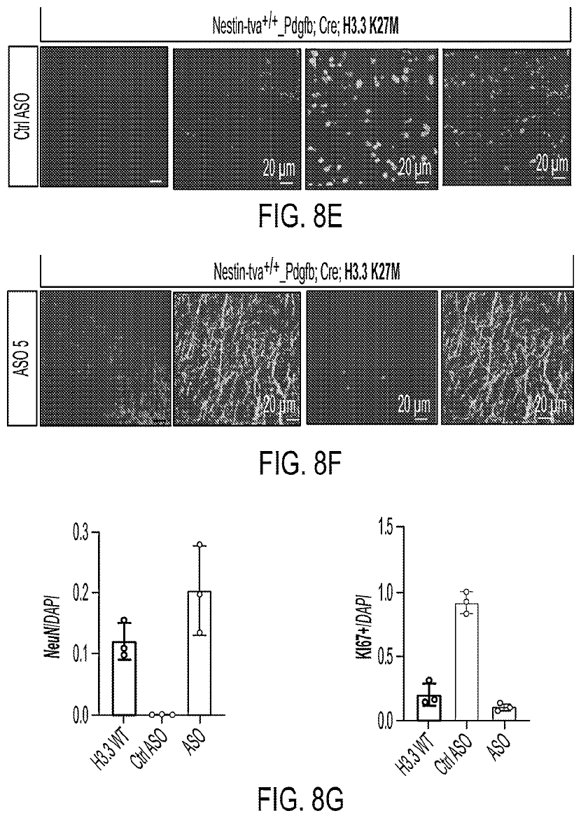

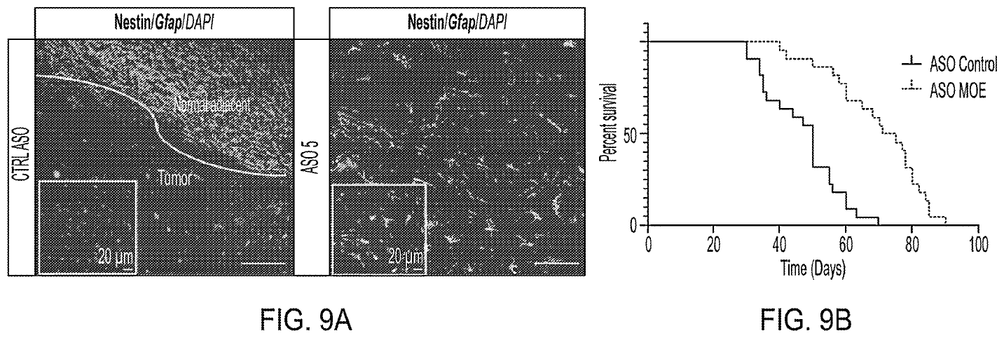

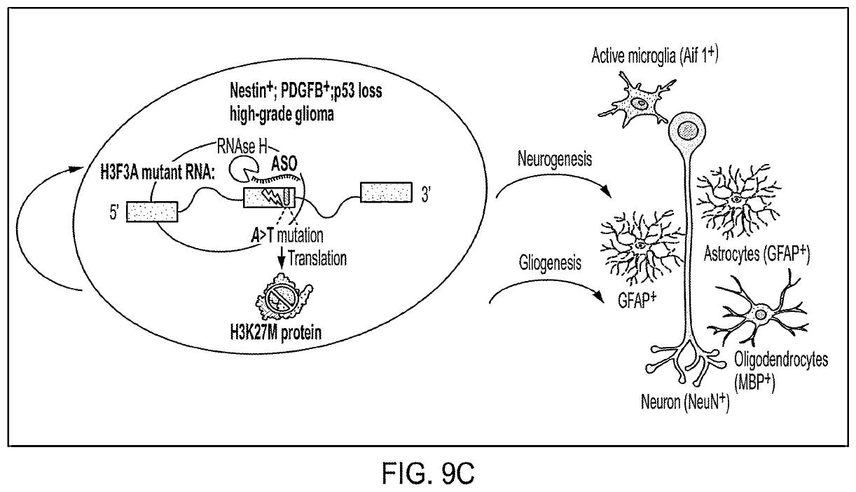

Proliferating cells in the tumors were also counted using antibody to Ki67. Results showed that ASO 5 treatment decreases cancer cell proliferation (FIGS. 7D and 8A-8F). ASO 5 treatment also decreased the NESTIN⁺ cell population (FIG. 9A) and increased survival (FIG. 9B).

Without being bound by theory, the potential mechanism of ASO therapeutic efficacy for treating DIPG is outlined in FIG. 9C. The ASO binds to the mRNA encoding the mutant H3K27M, which results in RNAse H degradation of the mRNA. This decreases the concentration of H3K27M protein, which in term increases H3K27 methylation. The increase in H3K27 methylation results in increased neurogenesis and Gliogenesis and decreased tumor growth.

To further understand ASO 5 therapeutic efficacy, confocal laser-scanning microscopy is used to characterize tumor lesions and compare the differences between untreated tumor/normal adjacent tissue and treated tumor lesions. Tumor size and grade is also measured by histology to assess effects on survival. Dose-response experiments are performed, and the time of initial dosing and the interval between maintenance doses is optimized. Lead ASOs, like ASO 5, are using in combination with approaches that target other cancer mutations found in DIPG tumors. For example, DMG patients' symptoms transiently improve after radiotherapy, and deletion of Atm radiosensitizes p53-deficient brainstem gliomas in an RCAS-Tva mouse model (13). Therefore, ASO treatments are tested in combination with radiotherapy, looking for enhanced effectiveness and increased survival in mouse models.

### Example 3: ASOs that Promote Skipping of the H3F3A Mutant Exon in DIPG Patient-Derived Cells and in Orthotopic Xenografts

Whereas the canonical histone genes are intronless, the histone-variant genes have introns—a property that can be exploited for therapy. This was accomplished by disrupting splicing of H3F3A (but not H3F3B) pre-mRNA, thus reducing the expression of full-length mRNA. Standard splice-switching ASOs are uniformly modified MOE with a PS backbone and 5-methyl cytosines. These ASOs do not elicit RNase-H cleavage of H3F3A transcripts, but are instead designed to sterically block a 5′ or 3′ splice site or a splicing-enhancer element in the pre-mRNA, reducing the expression of correctly spliced mRNA.

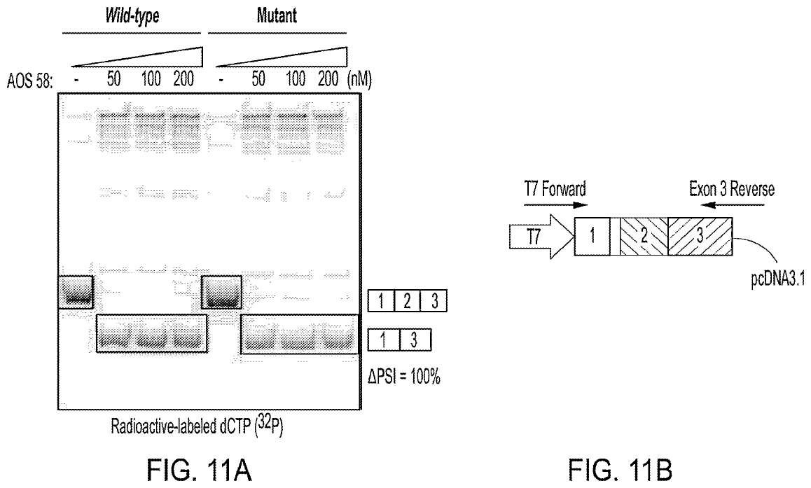

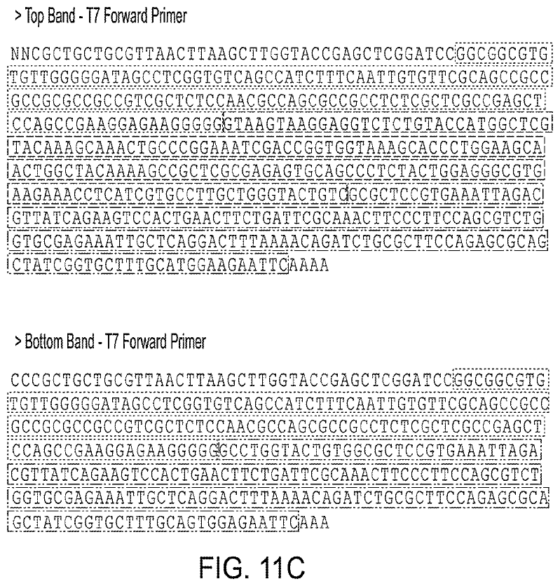

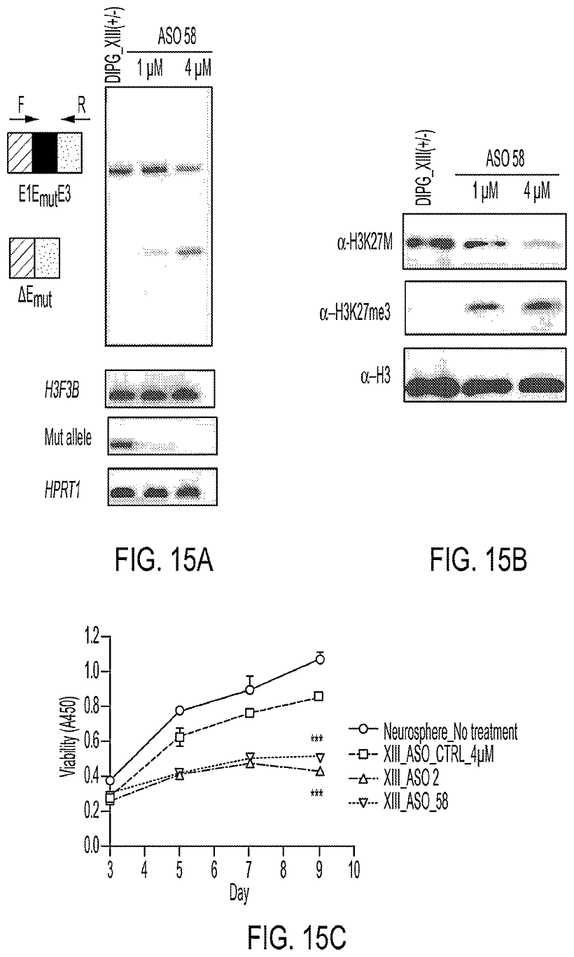

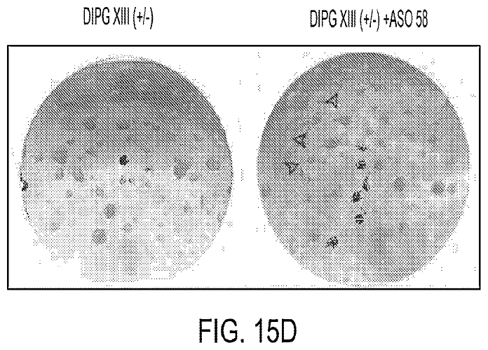

ASOs were designed to target the 5′ splice site of the exon harboring the K27M mutation. A lead ASO (ASO 58) promoted 100% mutant exon skipping using H3F3A wt and mut minigenes (FIG. 11A-11C). The exon-skipped mRNA lacks an AUG codon, resulting in downregulation of H3.3-K27M mutant protein. This in turn resulted in H3K27me3 restoration and delayed tumorigenesis in xenografts, similar to the phenotypes obtained with gapmer-ASO-treated patient cells (FIGS. 15A-15B).

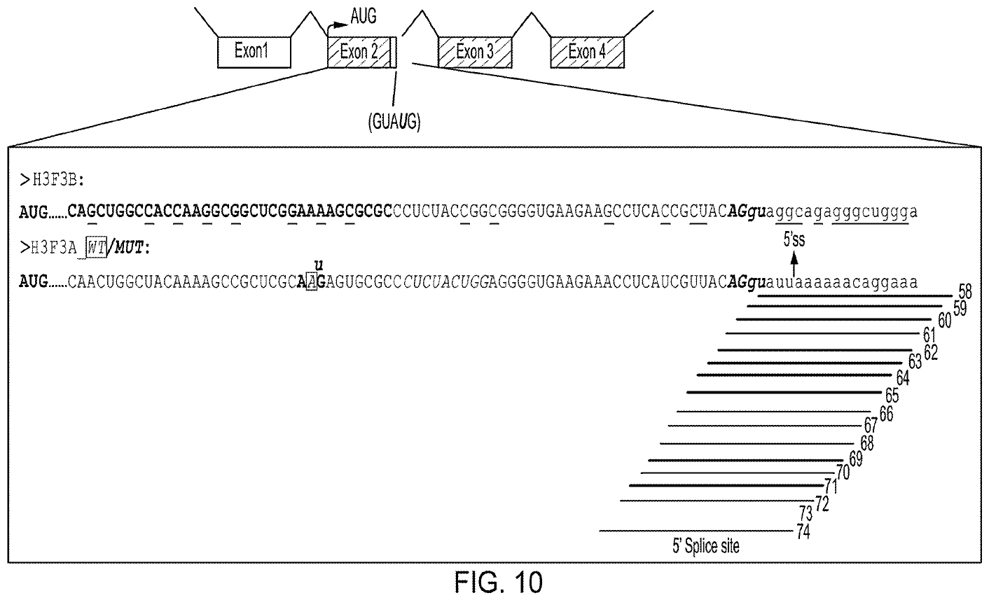

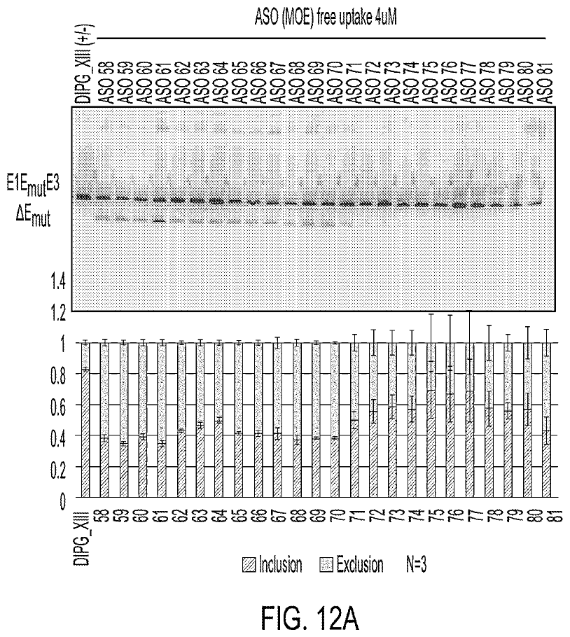

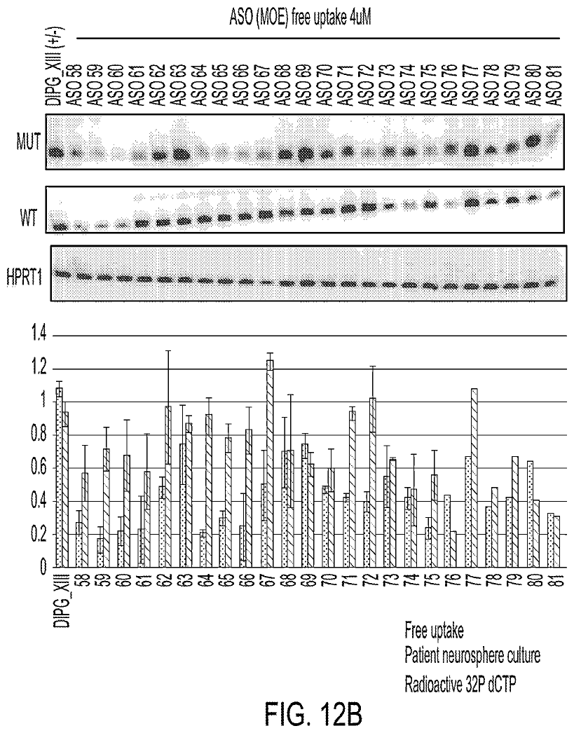

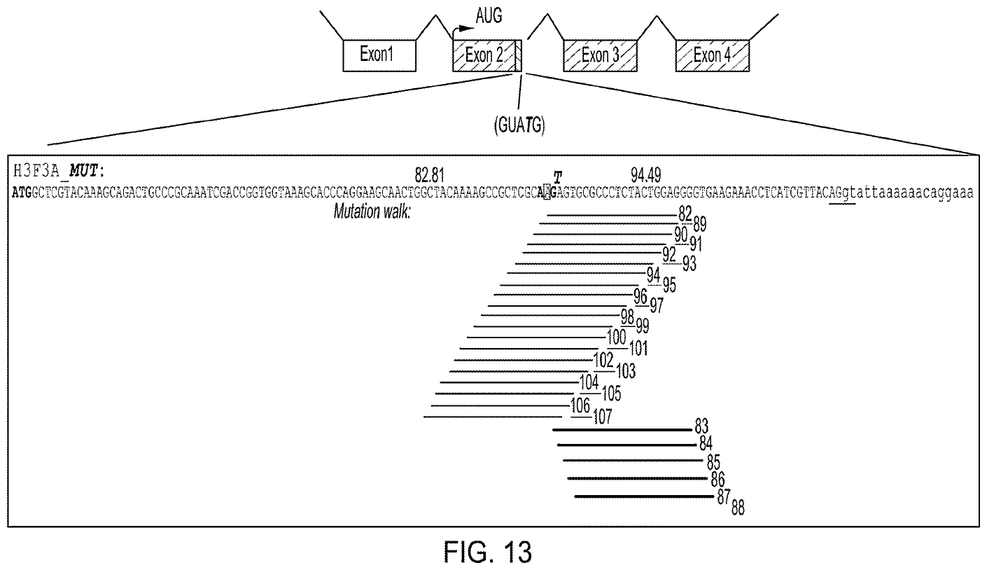

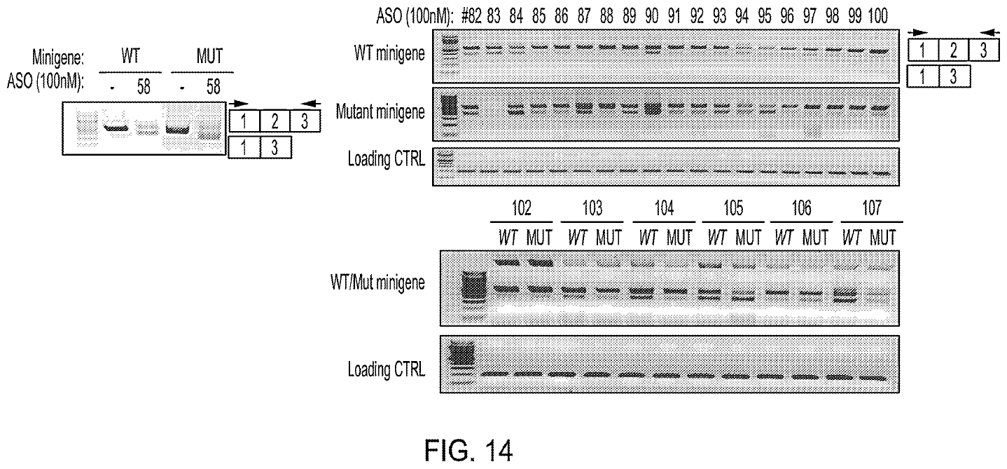

A large number of ASOs with this exon-skipping design targeting 5′ splice sight of exon 2 were further designed using a single-nucleotide microwalk (FIG. 10). These splice modulating ASO were screened in patient-derived (SU_DIPG_XIII) neurosphere cultures by free uptake. Results showed that most splice modulating ASO increased the number of mRNA transcripts that do not include exon 2 (FIG. 12A-12B). Additional splice-modulating ASOs were designed to target exon 2 again using a single-nucleotide ASO walk. Results showed that some ASOs greatly reduced the production of mRNA encoding exon 2 (e.g., ASO 58) (FIG. 14).

The most promising ASO(s) are further optimized, e.g., by changing the length and reducing the PS content (mixed PS/PO), and then testing the lead splice-modulating ASO(s) in vivo. Because this approach targets H3F3A pre-mRNA splicing, the retrovirus-based mouse model described in Example 2 cannot be used to test ASO efficacy. Instead, as mentioned above, an orthotopic xenograft mouse model was produced using a published procedure (14). This mouse model involves NOD-SCID-IL2 γ-chain-deficient mice (NSG) and the SU-DIPG-XIII H3F3A-K27M patient-derived cell line. Single-cell suspensions from SU-DIPG-XIII-luc neurospheres are prepared, follow by stereotaxically injection of 100,000 cells in 2 μL into the mouse midbrain at P2 through a 31G burr hole. Within 6 weeks tumors are detected by bioluminescence imaging and histology, as in Example 2.

Control-ASO- and test-ASO-treated cohorts are established once tumors are detected, and then the efficacy of the lead splice-modulating ASOs are assessed. ICV injection and infusion via Alzet microosmotic pumps are compared (12, 15); subcutaneous injection is also tested, as the tumors may disrupt the blood brain barrier (BBB), making it permeable to ASOs. Tumorigenesis following ASO treatment is monitored using bioluminescence as above. Mice are euthanized when symptoms are evident (enlarged head, ataxia, weight loss up to 25%) or at a preset endpoint. Then RNA and protein from tumors and normal adjacent tissue are extracted to measure knockdown efficiency, and downstream expression and epigenetic changes. Tumor size is measured, compared and graded by histology, to assess the ASOs' efficacy. ASO dosing will be optimized by performing dose-response experiments, varying the time of initial dosing, and/or the interval between maintenance doses.

For MOA experiments, cis-elements and cognate splicing factors whose binding is blocked by the lead ASOs are characterized. Potential off-target effects in patient cells and safety/tolerability in mice is assessed as in previous work with other ASOs (16-17). RNA-seq analysis of gene expression and splicing changes will follow previously published methods (18-20).

Statistical analysis in the above-mentioned examples may be performed as follows. For in-vivo experiments, time-to-event endpoints are summarized using the Kaplan-Meier method. Endpoint differences among groups are identified using a log-rank test. P-value adjustments use Benjamini-Hochberg's procedure, and a two-sided P<0.05 is considered significant. For quantifying RT-qPCR, immunoblots, and IHC/IF, differences across experimental groups are examined using ANOVA followed by simultaneous tests for general linear hypotheses (GLH) of contrasts of interest. P-values are adjusted for multiple comparisons by Tukey's method for pairwise comparisons or Benjamini-Hochberg's procedure. For longitudinal cell viability, a linear mixed-effects model with experimental group, time and group-by-time interaction as fixed effects and cell-line-specific random intercept is used to fit the A₄₅₀ measurements; differences are examined using simultaneous tests for GLH of contrasts of interest. For data analyzed using parametric approaches, data transformation for each endpoint is considered, to ensure that the underlying model assumptions are satisfied.

### Example 4: Methods

Antisense oligonucleotides. PS-MOE-ASOs were ordered from IDT (Coralville, Iowa). ASOs synthesized in large scale for animal work were purified by HPLC. ASOs were dissolved in water and diluted in saline before use. A list of oligonucleotide sequences is provided below. The ASOs tested in mice were: ASO 5 (MOE/PS-DNA/PS-MOE/PS:5-10-5) GGCGCACTCATGCGAGCGGC (SEQ ID NO: 5); Control ASO (MOE/PS-DNA/PS-MOE/PS) CCTTCCCTGAAGGTTCCTCC (SEQ ID NO: 93).

TABLE 1
Unmodified/Base ASO Nucleic Acid Sequences

| SEQ | | |
|---|---|---|
| ID NO: | Sequence | Target Region |
| 1 | CACTCATGCGAGCGGCTTTT | H3F3A exon 2 mutation |
| | | surrounding region |
| 2 | GCACTCATGCGAGCGGCTTT | H3F3A exon 2 mutation |
| | | surrounding region |
| 3 | CGCACTCATGCGAGCGGCTT | H3F3A exon 2 mutation |
| | | surrounding region |
| 4 | GCGCACTCATGCGAGCGGCT | H3F3A exon 2 mutation |
| | | surrounding region |
| 5 | GGCGCACTCATGCGAGCGGC | H3F3A exon 2 mutation |
| | | surrounding region |
| 6 | GGGCGCACTCATGCGAGCGG | H3F3A exon 2 mutation |
| | | surrounding region |
| 7 | AGGGCGCACTCATGCGAGCG | H3F3A exon 2 mutation |
| | | surrounding region |
| 8 | GAGGGCGCACTCATGCGAGC | H3F3A exon 2 mutation |
| | | surrounding region |
| 9 | AGAGGGCGCACTCATGCGAG | H3F3A exon 2 mutation |
| | | surrounding region |
| 10 | TAGAGGGCGCACTCATGCGA | H3F3A exon 2 mutation |
| | | surrounding region |
| 11 | GGGCAGTCTGCTTTGTACGA | H3F3A exon 2, non-mutation |
| | | region |
| 12 | CGATTTGCGGGCAGTCTACT | H3F3A exon 2, non-mutation |
| | | region |
| 13 | TACCACCGGTCGATTTGCGG | H3F3A exon 2, non-mutation |
| | | region |
| 14 | TTGCTTCCTGGGTGCTTTAC | H3F3A exon 2, non-mutation |
| | | region |
| 15 | GAGGTTTCTTCACCCCTCCA | H3F3A exon 2, non-mutation |
| | | region |
| 16 | AGCAATTTCTCGCACCAAAC | H3F3A exon3 |
| 17 | CCACAGTACCAGGCCTATAA | H3F3A exon3 |
| 18 | CAACCAGATAGGCCTCACTT | H3F3A exon4 |
| 19 | GCCAATACCTGATTTCTGTC | H3F3A 5utr |
| 20 | CACTCGCAATCATATACTTA | H3F3A 5utr |
| 21 | GGAACAGCACAGAAGACAGA | H3F3A 5utr |
| 22 | CTTTTCCTGCCACGATCACC | H3F3A3 utr |
| 23 | CCTACCAGCATTGAAAAATC | H3F3A 3utr |
| 24 | AATTCTGAGCGACAAACGGC | H3F3A 3utr |
| 25 | TCATGCGAGCGGCTTTTGTA | H3F3A exon 2 |
| 26 | CTCTGGAAGCGCAGATCTGT | H3F3A exon 3 |
| 27 | TTACACGTTTGGCATGGATA | H3F3A exon 4 |
| 28 | TTTCCTGTTTTTTAATACCT | 5′ splice site of H3F3A exon 2 |
| 29 | TTCCTGTTTTTTAATACCTG | 5′ splice site of H3F3A exon 2 |
| 30 | TCCTGTTTTTTAATACCTGT | 5′ splice site of H3F3A exon 2 |
| 31 | cctgttttttaatacCTGTA | 5′ splice site of H3F3A exon 2 |
| 32 | ctgttttttaatacCTGTAA | 5′ splice site of H3F3A exon 2 |
| 33 | tgttttttaatacCTGTAAC | 5′ splice site of H3F3A exon 2 |
| 34 | gttttttaatacCTGTAACG | 5′ splice site of H3F3A exon 2 |
| 35 | ttttttaatacCTGTAACGA | 5′ splice site of H3F3A exon 2 |
| 36 | tttttaatacCTGTAACGAT | 5′ splice site of H3F3A exon 2 |
| 37 | ttttaatacCTGTAACGATG | 5′ splice site of H3F3A exon 2 |
| 38 | tttaatacCTGTAACGATGA | 5′ splice site of H3F3A exon 2 |
| 39 | ttaatacCTGTAACGATGAG | 5′ splice site of H3F3A exon 2 |
| 40 | taatacCTGTAACGATGAGG | 5′ splice site of H3F3A exon 2 |
| 41 | aatacCTGTAACGATGAGGT | 5′ splice site of H3F3A exon 2 |
| 42 | atacCTGTAACGATGAGGTT | 5′ splice site of H3F3A exon 2 |
| 43 | tacCTGTAACGATGAGGTTT | 5′ splice site of H3F3A exon 2 |
| 44 | acCTGTAACGATGAGGTTTC | 5′ splice site of H3F3A exon 2 |
| 45 | TAGAGGGCGCACTCTTGCGA | H3F3A exon 2 |
| 46 | TCCAGTAGAGGGCGCACTCT | H3F3A exon 2 |
| 47 | ACCCCTCCAGTAGAGGGCGC | H3F3A exon 2 |
| 48 | TCTTCACCCCTCCAGTAGAG | H3F3A exon 2 |
| 49 | AGGTTTCTTCACCCCTCCAG | H3F3A exon 2 |
| 50 | CGATGAGGTTTCTTCACCCC | H3F3A exon 2 |
| 51 | TGTAACGATGAGGTTTCTTC | H3F3A exon 2 |
| 52 | CCAGTAGAGGGCGCACTCAT | H3F3A exon 2 |
| 53 | TCCAGTAGAGGGCGCACTCA | H3F3A exon 2 |
| 54 | CTCCAGTAGAGGGCGCACTC | H3F3A exon 2 |
| 55 | CCTCCAGTAGAGGGCGCACT | H3F3A exon 2 |
| 56 | CCCTCCAGTAGAGGGCGCAC | H3F3A exon 2 |
| 57 | CCCCTCCAGTAGAGGGCGCA | H3F3A exon 2 |
| 58 | ACCCCTCCAGTAGAGGGCGC | H3F3A exon 2 |
| 59 | CAGTAGAGGGCGCACTCATG | H3F3A exon 2 |
| 60 | AGTAGAGGGCGCACTCATGC | H3F3A exon 2 |
| 61 | GTAGAGGGCGCACTCATGCG | H3F3A exon 2 |
| 62 | TAGAGGGCGCACTCATGCGA | H3F3A exon 2 |
| 63 | AGAGGGCGCACTCATGCGAG | H3F3A exon 2 |
| 64 | GAGGGCGCACTCATGCGAGC | H3F3A exon 2 |
| 65 | AGGGCGCACTCATGCGAGCG | H3F3A exon 2 |
| 66 | GGGCGCACTCATGCGAGCGG | H3F3A exon 2 |
| 67 | GGCGCACTCATGCGAGCGGC | H3F3A exon 2 |
| 68 | GCGCACTCATGCGAGCGGCT | H3F3A exon 2 |
| 69 | CGCACTCATGCGAGCGGCTT | H3F3A exon 2 |
| 70 | GCACTCATGCGAGCGGCTTT | H3F3A exon 2 |
| 71 | CACTCATGCGAGCGGCTTTT | H3F3A exon 2 |
| 72 | ACTCATGCGAGCGGCTTTTG | H3F3A exon 2 |
| 73 | CTCATGCGAGCGGCTTTTGT | H3F3A exon 2 |
| 74 | TCATGCGAGCGGCTTTTGTA | H3F3A exon 2 |
| 75 | CATGCGAGCGGCTTTTGTAG | H3F3A exon 2 |
| 76 | ATGCGAGCGGCTTTTGTAGC | H3F3A exon 2 |
| 77 | TGCGAGCGGCTTTTGTAGCC | H3F3A exon 2 |

*The following SEQ ID NO pairs are associated with the same sequence (1 and 71; 2 and 70; 3 and 69; 4 and 68; 5 and 67; 6 and 66; 7 and 65; 8 and 64; 9 and 63; 10 and 62; 25 and 74; 47 and 58).

TABLE 2
Modified ASO Nucleic Acid Sequences

| SEQ | | | | | |
|---|---|---|---|---|---|
| ID | | | | | Target |
| NO: | Name | Sequence | Type | Modification | Region |
| 94 | ASO1 | CACTCATGCGAGCGGCTTTT | gapmer | 2′MOE- | H3F3A |
| | | | | DNA-2′MOE; | exon 2 |
| | | | | PS | mutation |
| | | | | throughout | surrounding |
| | | | | | region |
| 95 | ASO2 | GCACTCATGCGAGCGGCTTT | gapmer | 2′MOE- | H3F3A |
| | | | | DNA-2′MOE; | exon 2 |
| | | | | PS | mutation |
| | | | | throughout | surrounding |
| | | | | | region |
| 96 | ASO3 | CGCACTCATGCGAGCGGCTT | gapmer | 2′MOE- | H3F3A |
| | | | | DNA-2′MOE; | exon 2 |
| | | | | PS | mutation |
| | | | | throughout | surrounding |
| | | | | | region |
| 97 | ASO4 | GCGCACTCATGCGAGCGGCT | gapmer | 2′MOE- | H3F3A |
| | | | | DNA-2′MOE; | exon 2 |
| | | | | PS | mutation |
| | | | | throughout | surrounding |
| | | | | | region |
| 98 | ASO5 | GGCGCACTCATGCGAGCGGC | gapmer | 2′MOE- | H3F3A |
| | | | | DNA-2′MOE; | exon 2 |
| | | | | PS | mutation |
| | | | | throughout | surrounding |
| | | | | | region |
| 99 | ASO6 | GGGCGCACTCATGCGAGCGG | gapmer | 2′MOE- | H3F3A |
| | | | | DNA-2′MOE; | exon 2 |
| | | | | PS | mutation |
| | | | | throughout | surrounding |
| | | | | | region |
| 100 | ASO7 | AGGGCGCACTCATGCGAGCG | gapmer | 2′MOE- | H3F3A |
| | | | | DNA-2′MOE; | exon 2 |
| | | | | PS | mutation |
| | | | | throughout | surrounding |
| | | | | | region |
| 101 | ASO8 | GAGGGCGCACTCATGCGAGC | gapmer | 2′MOE- | H3F3A |
| | | | | DNA-2′MOE; | exon 2 |
| | | | | PS | mutation |
| | | | | throughout | surrounding |
| | | | | | region |
| 102 | ASO9 | AGAGGGCGCACTCATGCGAG | gapmer | 2′MOE- | H3F3A |
| | | | | DNA-2′MOE; | exon 2 |
| | | | | PS | mutation |
| | | | | throughout | surrounding |
| | | | | | region |
| 103 | ASO | TAGAGGGCGCACTCATGCGA | gapmer | 2′MOE- | H3F3A |
| | 10 | | | DNA-2′MOE; | exon 2 |
| | | | | PS | mutation |
| | | | | throughout | surrounding |
| | | | | | region |
| 104 | ASO | GGGCAGTCTGCTTTGTACGA | gapmer | 2′MOE- | H3F3A |
| | 11 | | | DNA-2′MOE; | exon 2, |
| | | | | PS | non- |
| | | | | throughout | mutation |
| | | | | | region |
| 105 | ASO | CGATTTGCGGGCAGTCTACT | gapmer | 2′MOE- | H3F3A |
| | 12 | | | DNA-2′MOE; | exon 2, |
| | | | | PS | non- |
| | | | | throughout | mutation |
| | | | | | region |
| 106 | ASO | TACCACCGGTCGATTTGCGG | gapmer | 2′MOE- | H3F3A |
| | 13 | | | DNA-2′MOE; | exon 2, |
| | | | | PS | non- |
| | | | | throughout | mutation |
| | | | | | region |
| 107 | ASO | TTGCTTCCTGGGTGCTTTAC | gapmer | 2′MOE- | H3F3A |
| | 14 | | | DNA-2′MOE; | exon 2, |
| | | | | PS | non- |
| | | | | throughout | mutation |
| | | | | | region |
| 108 | ASO | GAGGTTTCTTCACCCCTCCA | gapmer | 2′MOE- | H3F3A |
| | 15 | | | DNA-2′MOE; | exon 2, |
| | | | | PS | non- |
| | | | | throughout | mutation |
| | | | | | region |
| 109 | ASO | AGCAATTTCTCGCACCAAAC | gapmer | 2′MOE- | H3F3A |
| | 16 | | | DNA-2′MOE; | exon3 |
| | | | | PS | |
| | | | | throughout | |
| 110 | ASO | CCACAGTACCAGGCCTATAA | gapmer | 2′MOE- | H3F3A |
| | 20 | | | DNA-2′MOE; | exon3 |
| | | | | PS | |
| | | | | throughout | |
| 111 | ASO21 | CAACCAGATAGGCCTCACTT | gapmer | 2′MOE- | H3F3A |
| | | | | DNA-2′MOE; | exon4 |
| | | | | PS | |
| | | | | throughout | |
| 112 | ASO22 | GCCAATACCTGATTTCTGTC | gapmer | 2′MOE- | H3F3A 5utr |
| | | | | DNA-2′MOE; | |
| | | | | PS | |
| | | | | throughout | |
| 113 | ASO | CACTCGCAATCATATACTTA | gapmer | 2′MOE- | H3F3A 5utr |
| | 23 | | | DNA-2′MOE; | |
| | | | | PS | |
| | | | | throughout | |
| 114 | ASO | GGAACAGCACAGAAGACAGA | gapmer | 2′MOE- | H3F3A 5utr |
| | 26 | | | DNA-2′MOE; | |
| | | | | PS | |
| | | | | throughout | |
| 115 | ASO | CTTTTCCTGCCACGATCACC | gapmer | 2′MOE- | H3F3A3 utr |
| | 27 | | | DNA-2′MOE; | |
| | | | | PS | |
| | | | | throughout | |
| 116 | ASO | CCTACCAGCATTGAAAAATC | gapmer | 2′MOE- | H3F3A 3utr |
| | 28 | | | DNA-2′MOE; | |
| | | | | PS | |
| | | | | throughout | |
| 117 | ASO | AATTCTGAGCGACAAACGGC | gapmer | 2′MOE- | H3F3A 3utr |
| | 30 | | | DNA-2′MOE; | |
| | | | | PS | |
| | | | | throughout | |
| 118 | ASO | TCATGCGAGCGGCTTTTGTA | gapmer | 2′MOE- | H3F3A |
| | 32 | | | DNA-2′MOE; | exon 2 |
| | | | | PS | |
| | | | | throughout | |
| 119 | ASO | CTCTGGAAGCGCAGATCTGT | gapmer | 2′MOE- | H3F3A |
| | 35 | | | DNA-2′MOE; | exon 3 |
| | | | | PS | |
| | | | | throughout | |
| 120 | ASO | TTACACGTTTGGCATGGATA | gapmer | 2′MOE- | H3F3A |
| | 36 | | | DNA-2′MOE; | exon 4 |
| | | | | PS | |
| | | | | throughout | |
| 121 | ASO | TTTCCTGTTTTTTAATACCT | splice | uniform | 5′ splice |
| | 58 | | modulating | 2′MOE and | site of |
| | | | | PS | H3F3A |
| | | | | throughout | exon 2 |
| 122 | ASO | TTCCTGTTTTTTAATACCTG | splice | uniform | 5′ splice |
| | 59 | | modulating | 2′MOE and | site of |
| | | | | PS | H3F3A |
| | | | | throughout | exon 2 |
| 123 | ASO | TCCTGTTTTTTAATACCTGT | splice | uniform | 5′ splice |
| | 60 | | modulating | 2′MOE and | site of |
| | | | | PS | H3F3A |
| | | | | throughout | exon 2 |
| 124 | ASO | cctgttttttaatacCTGTA | splice | uniform | 5′ splice |
| | 61 | | modulating | 2′MOE and | site of |
| | | | | PS | H3F3A |
| | | | | throughout | exon 2 |
| 125 | ASO | ctgttttttaatacCTGTAA | splice | uniform | 5′ splice |
| | 62 | | modulating | 2′MOE and | site of |
| | | | | PS | H3F3A |
| | | | | throughout | exon 2 |
| 126 | ASO | tgttttttaatacCTGTAAC | splice | uniform | 5′ splice |
| | 63 | | modulating | 2′MOE and | site of |
| | | | | PS | H3F3A |
| | | | | throughout | exon 2 |
| 127 | ASO | gttttttaatacCTGTAACG | splice | uniform | 5′ splice |
| | 64 | | modulating | 2′MOE and | site of |
| | | | | PS | H3F3A |
| | | | | throughout | exon 2 |
| 128 | ASO | ttttttaatacCTGTAACGA | splice | uniform | 5′ splice |
| | 65 | | modulating | 2′MOE and | site of |
| | | | | PS | H3F3A |
| | | | | throughout | exon 2 |
| 129 | ASO | tttttaatacCTGTAACGAT | splice | uniform | 5′ splice |
| | 66 | | modulating | 2′MOE and | site of |
| | | | | PS | H3F3A |
| | | | | throughout | exon 2 |
| 130 | ASO | ttttaatacCTGTAACGATG | splice | uniform | 5′ splice |
| | 67 | | modulating | 2′MOE and | site of |
| | | | | PS | H3F3A |
| | | | | throughout | exon 2 |
| 131 | ASO | tttaatacCTGTAACGATGA | splice | uniform | 5′ splice |
| | 68 | | modulating | 2′MOE and | site of |
| | | | | PS | H3F3A |
| | | | | throughout | exon 2 |
| 132 | ASO | ttaatacCTGTAACGATGAG | splice | uniform | 5′ splice |
| | 69 | | modulating | 2′MOE and | site of |
| | | | | PS | H3F3A |
| | | | | throughout | exon 2 |
| 133 | ASO | taatacCTGTAACGATGAGG | splice | uniform | 5′ splice |
| | 70 | | modulating | 2′MOE and | site of |
| | | | | PS | H3F3A |
| | | | | throughout | exon 2 |
| 134 | ASO | aatacCTGTAACGATGAGGT | splice | uniform | 5′ splice |
| | 71 | | modulating | 2′MOE and | site of |
| | | | | PS | H3F3A |
| | | | | throughout | exon 2 |
| 135 | ASO | atacCTGTAACGATGAGGTT | splice | uniform | 5′ splice |
| | 72 | | modulating | 2′MOE and | site of |
| | | | | PS | H3F3A |
| | | | | throughout | exon 2 |
| 136 | ASO | tacCTGTAACGATGAGGTTT | splice | uniform | 5′ splice |
| | 73 | | modulating | 2′MOE and | site of |
| | | | | PS | H3F3A |
| | | | | throughout | exon 2 |
| 137 | ASO | acCTGTAACGATGAGGTTTC | splice | uniform | 5′ splice |
| | 74 | | modulating | 2′MOE and | site of |
| | | | | PS | H3F3A |
| | | | | throughout | exon 2 |
| 138 | ASO | TAGAGGGCGCACTCTTGCGA | splice | uniform | H3F3A |
| | 75 | | modulating | 2′MOE and | exon 2 |
| | | | | PS | |
| | | | | throughout | |
| 139 | ASO | TCCAGTAGAGGGCGCACTCT | splice | uniform | H3F3A |
| | 76 | | modulating | 2′MOE and | exon 2 |
| | | | | PS | |
| | | | | throughout | |
| 140 | ASO | ACCCCTCCAGTAGAGGGCGC | splice | uniform | H3F3A |
| | 77 | | modulating | 2′MOE and | exon 2 |
| | | | | PS | |
| | | | | throughout | |
| 141 | ASO | TCTTCACCCCTCCAGTAGAG | splice | uniform | H3F3A |
| | 78 | | modulating | 2′MOE and | exon 2 |
| | | | | PS | |
| | | | | throughout | |
| 142 | ASO | AGGTTTCTTCACCCCTCCAG | splice | uniform | H3F3A |
| | 79 | | modulating | 2′MOE and | exon 2 |
| | | | | PS | |
| | | | | throughout | |
| 143 | ASO | CGATGAGGTTTCTTCACCCC | splice | uniform | H3F3A |
| | 80 | | modulating | 2′MOE and | exon 2 |
| | | | | PS | |
| | | | | throughout | |
| 144 | ASO | TGTAACGATGAGGTTTCTTC | splice | uniform | H3F3A |
| | 81 | | modulating | 2′MOE and | exon 2 |
| | | | | PS | |
| | | | | throughout | |
| 145 | ASO | CCAGTAGAGGGCGCACTCAT | splice | uniform | H3F3A |
| | 82 | | modulating | 2′MOE and | exon 2 |
| | | | | PS | |
| | | | | throughout | |
| 146 | ASO | TCCAGTAGAGGGCGCACTCA | splice | uniform | H3F3A |
| | 83 | | modulating | 2′MOE and | exon 2 |
| | | | | PS | |
| | | | | throughout | |
| 147 | ASO | CTCCAGTAGAGGGCGCACTC | splice | uniform | H3F3A |
| | 84 | | modulating | 2′MOE and | exon 2 |
| | | | | PS | |
| | | | | throughout | |
| 148 | ASO | CCTCCAGTAGAGGGCGCACT | splice | uniform | H3F3A |
| | 85 | | modulating | 2′MOE and | exon 2 |
| | | | | PS | |
| | | | | throughout | |
| 149 | ASO | CCCTCCAGTAGAGGGCGCAC | splice | uniform | H3F3A |
| | 86 | | modulating | 2′MOE and | exon 2 |
| | | | | PS | |
| | | | | throughout | |
| 150 | ASO | CCCCTCCAGTAGAGGGCGCA | splice | uniform | H3F3A |
| | 87 | | modulating | 2′MOE and | exon 2 |
| | | | | PS | |
| | | | | throughout | |
| 151 | ASO | ACCCCTCCAGTAGAGGGCGC | splice | uniform | H3F3A |
| | 88 | | modulating | 2′MOE and | exon 2 |
| | | | | PS | |
| | | | | throughout | |
| 152 | ASO | CAGTAGAGGGCGCACTCATG | splice | uniform | H3F3A |
| | 89 | | modulating | 2′MOE and | exon 2 |
| | | | | PS | |
| | | | | throughout | |
| 153 | ASO | AGTAGAGGGCGCACTCATGC | splice | uniform | H3F3A |
| | 90 | | modulating | 2′MOE and | exon 2 |
| | | | | PS | |
| | | | | throughout | |
| 154 | ASO | GTAGAGGGCGCACTCATGCG | splice | uniform | H3F3A |
| | 91 | | modulating | 2′MOE and | exon 2 |
| | | | | PS | |
| | | | | throughout | |
| 155 | ASO | TAGAGGGCGCACTCATGCGA | splice | uniform | H3F3A |
| | 92 | | modulating | 2′MOE and | exon 2 |
| | | | | PS | |
| | | | | throughout | |
| 156 | ASO | AGAGGGCGCACTCATGCGAG | splice | uniform | H3F3A |
| | 93 | | modulating | 2′MOE and | exon 2 |
| | | | | PS | |
| | | | | throughout | |
| 157 | ASO | GAGGGCGCACTCATGCGAGC | splice | uniform | H3F3A |
| | 94 | | modulating | 2′MOE and | exon 2 |
| | | | | PS | |
| | | | | throughout | |
| 158 | ASO | AGGGCGCACTCATGCGAGCG | splice | uniform | H3F3A |
| | 95 | | modulating | 2′MOE and | exon 2 |
| | | | | PS | |
| | | | | throughout | |
| 159 | ASO | GGGCGCACTCATGCGAGCGG | splice | uniform | H3F3A |
| | 96 | | modulating | 2′MOE and | exon 2 |
| | | | | PS | |
| | | | | throughout | |
| 160 | ASO | GGCGCACTCATGCGAGCGGC | splice | uniform | H3F3A |
| | 97 | | modulating | 2′MOE and | exon 2 |
| | | | | PS | |
| | | | | throughout | |
| 161 | ASO | GCGCACTCATGCGAGCGGCT | splice | uniform | H3F3A |
| | 98 | | modulating | 2′MOE and | exon 2 |
| | | | | PS | |
| | | | | throughout | |
| 162 | ASO | CGCACTCATGCGAGCGGCTT | splice | uniform | H3F3A |
| | 99 | | modulating | 2′MOE and | exon 2 |
| | | | | PS | |
| | | | | throughout | |
| 163 | ASO | GCACTCATGCGAGCGGCTTT | splice | uniform | H3F3A |
| | 100 | | modulating | 2′MOE and | exon 2 |
| | | | | PS | |
| | | | | throughout | |
| 164 | ASO | CACTCATGCGAGCGGCTTTT | splice | uniform | H3F3A |
| | 101 | | modulating | 2′MOE and | exon 2 |
| | | | | PS | |
| | | | | throughout | |
| 165 | ASO | ACTCATGCGAGCGGCTTTTG | splice | uniform | H3F3A |
| | 102 | | modulating | 2′MOE and | exon 2 |
| | | | | PS | |
| | | | | throughout | |
| 166 | ASO | CTCATGCGAGCGGCTTTTGT | splice | uniform | H3F3A |
| | 103 | | modulating | 2′MOE and | exon 2 |
| | | | | PS | |
| | | | | throughout | |
| 167 | ASO | TCATGCGAGCGGCTTTTGTA | splice | uniform | H3F3A |
| | 104 | | modulating | 2′MOE and | exon 2 |
| | | | | PS | |
| | | | | throughout | |
| 168 | ASO | CATGCGAGCGGCTTTTGTAG | splice | uniform | H3F3A |
| | 105 | | modulating | 2′MOE and | exon 2 |
| | | | | PS | |
| | | | | throughout | |
| 169 | ASO | ATGCGAGCGGCTTTTGTAGC | splice | uniform | H3F3A |
| | 106 | | modulating | 2′MOE and | exon 2 |
| | | | | PS | |
| | | | | throughout | |
| 170 | ASO | TGCGAGCGGCTTTTGTAGCC | splice | uniform | H3F3A |
| | 107 | | modulating | 2′MOE and | exon 2 |
| | | | | PS | |
| | | | | throughout | |

*Gapmer sequences are 5-10-5 (5 nt on each wing, and 10 nt in the center) 2′-MOE: 2′-O-methoxyethyl; PS: phosphorothioate. When 2′-MOE is used, those nucleotides are neither DNA nor RNA. They are closer to RNA than DNA, since they have an O at the 2′ position of the ribose. But the O is not OH, it is methoxyethyl.

### Primary Pediatric Human Glioma Cell Lines.

SU-DIPG-XIII, SU-DIPG-35, and SU-DIPG-50 patient cells heterozygous for the H3F3A mutation (A>T) and derived from autopsy tissue. The cells were grown as tumor neurospheres in tumor stem media (TSM) consisting of DMEM/F12 (Invitrogen), Neurobasal (-A) (Invitrogen), B27 (-A) (Invitrogen), human-bFGF (20 ng/mL; Protech), human-EGF (20 ng/mL; Peprotech), human PDGF-AB (20 ng/mL; Peprotech), and heparin (10 ng/mL; Stemcell). The point mutation in H3F3A was confirmed by Sanger sequencing using primers listed below.

Sanger Sequencing Primers for gDNA H3F3A Minigene:

Fwd
(SEQ ID NO: 78)
5′-GGATCCGGCGGCGTGTGTTGGGGGATAGCCT
Rev
(SEQ ID NO: 79)
5′-CTTGAATTCTCACTGCAAAGCACCGATAGCTGC

### Sanger Sequencing Primers for H3F3B:

Fwd
(SEQ ID NO: 80)
5′-GTGCTGGTTTTTCGCTCGTC
Rev
(SEQ ID NO: 81)
5′-CTTTCGTGGCCAGCTGTTTG

### Sanger Sequencing Primers for HPRT1:

Fwd
(SEQ ID NO: 82)
5′-TGACCAGTCAACAGGGGACA
Rev
(SEQ ID NO: 83)
5′-TGCCTGACCAAGGAAAGCAA

### Sanger Sequencing Primers for H3F3A Mutation:

Fwd
(SEQ ID NO: 84)
5′-AAGCAGACTGCCCGCAAA
Rev
(SEQ ID NO: 85)
5′-CGCTCTGGAAGCGCAGAT

### Lentivirus Preparation and Infection.

Two 20-nucleotide gRNA pairs against human H3F3A were annealed and cloned in the pSpCas9 (BB) vector (pX459; Addgene plasmid #62988) expressing Cas9 lentiviral constructs; Cas9-resistance gRNA pairs were annealed and cloned in the lentiV-neo vector (Addgene plasmid #108101). Lentiviral particles were generated by co-transfection of lentiviral-expressing constructs with packaging plasmids (pspAX2, VSV-G) into HEK-293T cells, and then concentrated by polyethylene glycol (PEG-it) precipitation (SBI). For lentiviral infection, dissociated DIPG cells were seeded on a 1% matrigel-coated plate (Corning), and incubated with gRNA-expressing lentivirus for 12 hours before replacing with fresh medium. Puromycin (0.5 μg/ml) was added at 48 hours post-infection to select infected cells. After 7 days, puromycin was removed and the cells were allowed to recover in regular growth medium. Bulk cells were used by immunostaining, western blotting, and functional assays.

### Transfection and Free Uptake of ASOs.

Patient neurospheres were grown to 70-80% confluence in 12-well plates, and transfected for 3 days with 2 μL of Lipofectamine 2000 transfection reagent (Invitrogen) and different amounts of ASOs, ranging from 30 nM to 150 nM, following the manufacturer's recommendations. For free uptake, patient cells were dissociated into single cells using TrypLE Express (Invitrogen), and 15,000/well cells were seeded in a 96-well plate and incubated at 37° C. for 1 h; 4-10 μM ASO was then added for 3 to 5 days, cell medium was replaced with fresh medium, and a second ASO dose was added on day 3.

### Cell Viability and Proliferation Assay.

Primary patient cells were starved in TSM base with B27 for 3 days. Then, 3,000 cells/well were plated in a 96-well plate in TSM base with normal growth medium with EGF, FGF, and PDGF-AB, as described (Nagaraja et al., 2019). Cell viability and growth were measured using a cell-counting kit (CCK-8; Sigma #96992) at set time points.

### RCAS-TVA Mouse Model.

All animal procedures were performed with approval from Cold Spring Harbor Laboratory's Institutional Animal Care and Use Committee (IACUC). DF1 cells (AATC Catalog #CRL-12203) were cultured in DMEM supplemented with 10% FBS, 2 mM L-glutamine, 100 units/mL penicillin and 100 μg/mL streptomycin, and incubated at 39° C. and 5% CO₂. 5 μg of each RCAS plasmid was transfected into DF1 cells using X-TremeGENE 9 (Roche) following the manufacturer's instructions. The Nestin-tva; p53^{fl/fl} mouse strain was a generous gift from Dr. Oren Becher at Northwestern University (Evanston, IL). For generation of midline gliomas, transfected cells were passaged at least three times prior to injection; 10⁵ virus-producing DF1 cells were injected intracranially into a depth of 1-3 mm below the lambda suture of neonatal N-tva; p53^{fl/fl} pups (postnatal days 3-5) in 1 μL, using a Hamilton syringe (7659-01) and a 30-gauge needle. Four viruses, RCAS-Pdgfb, RCAS-Cre, RCAS-H3F3A mutant, and Rcas-Luciferase, were injected together in equal amounts. Mice were monitored daily and euthanized with C02 when they became symptomatic (including an enlarged head, ataxia, or weight loss up to 25%) or at 6 months post-injection if they remained asymptomatic.

### DIPG Orthotopic Xenograft Mouse Model.

All animal procedures were approved by the IACUC. This procedure was as described (Grasso C S et al., 2015). Briefly, a single-cell suspension of SU-DIPG-XIII-luc neurospheres was prepared (pLenti PGK V5-LUC Neo (w623-2); addgene, plasmid #21471), and 10⁵ cells (50,000 cells/μL) were injected into the fourth ventricle/pons of NOD-SCID-gamma (NSG) immunocompromised (strain 005557; The Jackson Laboratory), cold-anesthetized, postnatal day 3 mouse pups by injection through a 30-gauge burr hole (stereotactic coordinates: 3 mm posterior to the lambda suture and 3 mm deep).

### Bioluminescence Imaging.

D-Luciferin was reconstituted as per the manufacturer's protocol (Goldbio, LUCK-100) and administered intraperitoneally (10 μg/g in PBS) into isoflurane-anesthetized animals, 12 minutes prior to imaging. Animals were excluded if no tumors were present, and the remaining animals were randomized into control and treatment groups with equivalent distribution of initial tumor sizes.

### Intracerebroventricular Injection of ASOs.

The presence of tumors was confirmed through luminescence imaging, as described above. Mice bearing tumors were then randomized and treated with a single ICV injection of CTRL ASO or ASO 5 (500 μg for the RCAS-TVA mouse model; 200 μg for the orthotopic xenograft model) using a Hamilton syringe with a 28-gauge burr hole needle in isoflurane-anesthetized animals (stereotactic coordinates: 1.0 mm posterior to the bregma, 0.2 mm lateral, and 3 mm in depth).

### Immunofluorescence and Immunohistochemistry.

Tumor tissue was fixed in 4% paraformaldehyde, cut into 5-μm sections, and embedded in paraffin. IHC was performed using heat-induced antigen retrieval with sodium citrate buffer, followed by primary antibodies to GFAP (1:1000; Millipore Sigma, rabbit polyclonal, AB5804), NeuN (1:100; Sigma, rabbit, monoclonal, 13E6), MBP (1:5000; Abcam, rabbit monoclonal, EPR21188), Ki67 (1:50; BD biosciences, mouse monoclonal, B56), Nestin (1:100, R&D, mouse monoclonal, 307501), or H3K27me3 (1:1000; CST rabbit polyclonal, C36B11). For IHC, the signal was visualized with HRP-labeled anti-rabbit polyclonal (1:200, Agilent, P0448) and DAB (Agilent, K346711). Slides were counterstained with hematoxylin (Sigma) and captured on a Zeiss Observer microscope. For IF, the signal was visualized with fluoro-conjugated secondary antibody (Thermo Fisher) and captured on a Zeiss LSM780 confocal laser-scanning microscope.

### EdU Staining Assays.

Primary human glioma cells (SU-DIPG-XIII, 5×10³ cells/well) were seeded onto 1% matrigel-coated 8-well chamber slides (Falcon) and treated with 4 μM ASO by free uptake for five days. On day 5, 10 μM EdU was added to the cells, and incubated at 37° C. for 2 h. EdU incorporation was measured using a Click-it Plus EdU Alexa Fluor 594 Imaging Kit (Invitrogen) in accordance with the manufacturer's instructions. Images were captured on a Zeiss Observer microscope. All images within the same figure panel were taken with the same exposure setting, and identically processed using Image J software.

### Soft-Agar Assay.

Primary human glioma cells (SU-DIPG-XIII, 10³ cells/well) were incubated in an upper layer of 0.3% agar (ThermoFisher Scientific) in TSM. The bottom layer consists of the same medium with supplements, but 0.6% solidified basal agar, in a 12-well plate. Plates were incubated at 37° C./5% CO₂ for at least 3 weeks, before staining with crystal violet. Visible colonies were then counted.

### RNA and Protein Extraction.

Cells or tissues were harvested at the end points and snap-frozen in liquid nitrogen. For RNA extraction, 1 mL of Trizol (Invitrogen, 15596-018) was added to homogenized brain tissue or cells, following the standard Trizol protocol with chloroform extraction, isopropanol precipitation, and 70% EtOH RNA-pellet wash. RNA was resuspended in 20-40 L of nuclease-free water. For protein extraction, cells or tissues were harvested and lysed on ice using Triton Extraction Buffer (TEB: PBS containing 0.5% Triton X 100 (v/v), protease inhibitor cocktail (Roche)), followed by centrifugation at 6,500×g for 10 minutes at 4° C. to spin down the nuclei; the supernatant was removed and discarded; the pellet was resuspended in 0.2 N HCl to perform acid extraction overnight; the supernatant was collected after centrifugation at 6,500×g for 10 minutes at 4° C.; and the protein concentration was measured by Bradford assay (Bio-Rad).

### Radioactive RT-PCR and RT-qPCR.

Total RNA was extracted from cells or tissues as described above, and reverse-transcribed with ImProm-II reverse transcriptase (Promega) using oligo-dT primers. Total H3F3A cDNA was amplified with AmpliTaq DNA polymerase (Thermo Fisher) using Fwd 5′-GGACTTTAAAACAGATCTGCGCTT (SEQ ID NO: 171) and Rev 5′-GTCTTTTGGCATAATTGTTACACGT (SEQ ID NO: 172) primers that sit on exon 3 and exon 4 (downstream of the H3F3A mutation site in exon 2), respectively. The H3F3A WT allele was amplified using Fwd 5′-GCTACAAAAGCCGCTCTCAA (SEQ ID NO: 173); the H3F3A mutant allele was amplified using Fwd 5′-GCTACAAAAGCCGCTCGAAT (SEQ ID NO: 174); and the same Rev 5′-CCAGACGCTGGAAGGGAAGT (SEQ ID NO: 175) primer was used for both mutant and WT allele amplification. cDNA from minigenes was amplified using vector-specific (pcDNA3.1) primers, listed as SEQ ID NOs: 78-85. For radioactive PCR, 0.16 μL of fresh [α-³²P]-dCTP was added to a 20-μL PCR reaction. Amplicons were separated by 5% native PAGE (Bio-Rad), followed by phosphorimage analysis on a Typhoon 9410 phosphoimager (GE Healthcare). Band intensities were quantified using Image J, and the values normalized for the G+C content according to the DNA sequence. For RT-qPCR, 2× Syber green master mix (Applied Biosystems) was used, and the cDNA was analyzed on a QuantStudio 6 Flex Real-Time PCR system (ThermoFisher Scientific). Fold changes were calculated using the ΔΔCq method.

### Western Blotting.

One microgram of acid-extracted protein was resolved by 8-20% precast protein gel (Bio-Rad), transferred onto a nitrocellulose membrane, and probed with rabbit polyclonal anti-H3K27M (1:1000; ABE419, Millipore), rabbit monoclonal anti-H3K27me3 (1:1000; C36B11, CST) or rabbit polyclonal H3 (1:1000, ab1791, Abcam) antibodies. The membranes were incubated with infrared-dye conjugated secondary antibodies (1:10000; LI-COR Biosciences), and protein bands were visualized by quantitative fluorescence using Odyssey software (LI-COR Biosciences). Molecular weight markers confirmed the sizes of the bands. Band intensities were quantified using Image J and normalized to total H3 protein.

### REFERENCES

1. Phillips R E, Soshnev A A, Allis C D (2020) Epigenomic Reprogramming as a Driver of Malignant Glioma. Cancer Cell 38: 647-660.
2. www.danafarberbostonchildrens.org/conditions/brain-tumor/diffuse-pontine-glioma.aspx
3. Lewis P W, Müller M M, Koletsky M S, Cordero F, Lin S, Banaszynski L A, Garcia B A, Muir T W, Becher O J, Allis C D (2013) Inhibition of PRC2 activity by a gain-of-function H3 mutation found in pediatric glioblastoma. Science 340: 857-861.
4. Herz H M, Morgan M, Gao X, Jackson J, Rickels R, Swanson S K, Florens L, Washburn M P, Eissenberg J C, Shilatifard A (2014) Histone H3 lysine-to-methionine mutants as a paradigm to study chromatin signaling. Science 345: 1065-1070.
5. Fontebasso A M, Papillon-Cavanagh S, Schwartzentruber J, Nikbakht H, Gerges N, Fiset P O, Bechet D, Faury D, De Jay N, Ramkissoon L A, Corcoran A, Jones D T, Sturm D, Johann P, Tomita T, Goldman S, Nagib M, Bendel A, Goumnerova L, Bowers D C, Leonard J R, Rubin J B, Alden T, Browd S, Geyer J R, Leary S, Jallo G, Cohen K, Gupta N, Prados M D, Carret A S, 6. Ellezam B, Crevier L, Klekner A, Bognar L, Hauser P, Garami M, Myseros J, Dong Z, Siegel P M, Malkin H, Ligon A H, Albrecht S, Pfister S M, Ligon K L, Majewski J, Jabado N, Kieran M W (2014) Recurrent somatic mutations in ACVR1 in pediatric midline high-grade astrocytoma. Nat Genet 46: 462-466.
6. dipgregistry.org/patients-families/biopsy/
7. Liddelow S A, Guttenplan K A, Clarke L E, Bennett F C, Bohlen C J, Schirmer L, Bennett M L, Münch A E, Chung W S, Peterson T C, Wilton D K, Frouin A, Napier B A, Panicker N, Kumar M, Buckwalter M S, Rowitch D H, Dawson V L, Dawson T M, Stevens B, Barres B A (2017) Neurotoxic reactive astrocytes are induced by activated microglia. Nature 541: 481-487.
8. Qian H, Kang X, Hu J, Zhang D, Liang Z, Meng F, Zhang X, Xue Y, Maimon R, Dowdy S F, Devaraj N K, Zhou Z, Mobley W C, Cleveland D W, Fu X D (2020) Reversing a model of Parkinson's disease with in situ converted nigral neurons. Nature 582: 550-556.
9. Hoeman C M, Cordero F J, Hu G, Misuraca K, Romero M M, Cardona H J, Nazarian J, Hashizume R, McLendon R, Yu P, Procissi D, Gadd S, Becher O J (2019) ACVR1 R206H cooperates with H3.1K27M in promoting diffuse intrinsic pontine glioma pathogenesis. Nat Commun 10: 1023.
10. Cordero F J, Huang Z, Grenier C, He X, Hu G, McLendon R E, Murphy S K, Hashizume R, Becher O J (2017) Histone H3.3K27M Represses p16 to Accelerate Gliomagenesis in a Murine Model of DIPG. Mol Cancer Res 15: 1243-1254.
11. Ahronian L G, Lewis B C (2014) In vivo delivery of RCAS virus to mice. Cold Spring Harb Protoc 2014: 1167-1169.
12. Hua Y, Sahashi K, Hung G, Rigo F, Passini M A, Bennett C F, Krainer A R (2010) Antisense correction of SMN2 splicing in the CNS rescues necrosis in a type III SMA mouse model. Genes Dev 24: 1634-1644.
13. Deland K, Starr B F, Mercer J S, Byemerwa J, Crabtree D M, Williams N T, Luo L, Ma Y, Chen M, Becher O J, Kirsch D G (2021) Tumor genotype dictates radiosensitization after Atm deletion in primary brainstem glioma models. J Clin Invest 131: e142158.
14. Grasso C S, Tang Y, Truffaux N, Berlow N E, Liu L, Debily M A, Quist M J, Davis L E, Huang E C, Woo P J, Ponnuswami A, Chen S, Johung T B, Sun W, Kogiso M, Du Y, Qi L, Huang Y, Hütt-Cabezas M, Warren K E, Le Dret L, Meltzer P S, Mao H, Quezado M, van Vuurden D G, Abraham J, Fouladi M, Svalina M N, Wang N, Hawkins C, Nazarian J, Alonso M M, Raabe E H, Hulleman E, Spellman P T, Li X N, Keller C, Pal R, Grill J, Monje M (2015) Functionally defined therapeutic targets in diffuse intrinsic pontine glioma. Nat Med 21: 555-559.
15. Hua Y, Sahashi K, Rigo F, Hung G, Horev G, Bennett C F, Krainer A R (2011) Peripheral SMN restoration is essential for long-term rescue of a severe spinal muscular atrophy mouse model. Nature 478: 123-126.
16. Scharner J, Ma W K. Zhang Q, Lin K T, Rigo F, Bennett C F. Krainer A R (2020) Hybridization-mediated off-target effects of splice-switching antisense oligonucleotides. Nucleic Acids Res 48: 802-816.
17. Rigo F, Chun S J, Norris D A, Hung G, Lee S, Matson J, Fey R A, Gaus H, Hua Y, Grundy J S, Krainer A R, Henry S P, Bennett C F (2014). Pharmacology of a central nervous system delivered 2′-O-methoxyethyl-modified survival of motor neuron splicing oligonucleotide in mice and nonhuman primates. J Pharmacol Exp Ther 350: 46-55.
18. Doktor T K, Hua Y, Andersen H S, Brsner S, Liu Y H, Wieckowska A, Dembic M, Bruun G H, Krainer A R, Andresen B S (2017) RNA-sequencing of a mouse-model of spinal muscular atrophy reveals tissue-wide changes in splicing of U12-dependent introns. Nucleic Acids Res 45: 395-416.
19. Lin K T, Krainer A R (2019) PSI-Sigma: a comprehensive splicing-detection method for short-read and long-read RNA-seq analysis. Bioinformatics 35: 5048-5054.
20. Yoshimi A, Lin K T, Wiseman D H, Rahman M A, Pastore A, Wang B, Lee S C, Micol J B, Zhang X J, de Botton S, Penard-Lacronique V, Stein E M, Cho H, Miles R E, Inoue D, Albrecht T R, Somervaille TCP, Batta K, Amaral F, Simeoni F, Wilks D P, Cargo C, Intlekofer A M, Levine R L, Dvinge H, Bradley R K, Wagner E J, Krainer A R, Abdel-Wahab O (2019) Coordinated alterations in RNA splicing and epigenetic regulation drive leukaemogenesis. Nature 574: 273-277.

### Example 5: Antisense Therapy in Mouse Models of Histone H3.3 K27M Diffuse Midline Glioma Inhibits Tumor Growth, Promotes Neural and Glial Differentiation, and Increases Survival

Diffuse midline gliomas (DMGs) are pediatric brain tumors with dismal prognosis. Those that occur in the pons are frequently caused by a dominant somatic mutation in a non-canonical histone, H3.3 K27M, which inhibits K27 trimethylation of all histone H3 proteins. Antisense oligonucleotides (ASOs) that target the mutant oncohistone mRNA and consequently reversed the epigenetic changes were developed. Using two different mouse models, ASOs administered to the cerebrospinal fluid after DMG tumor onset resulted in reduced tumor growth, neural-stem-cell differentiation, and increased survival. This data demonstrates the importance of H3.3 K27M for tumor maintenance, and provided a preclinical proof of principle for DMG antisense therapy.

Diffuse midline gliomas (DMGs) are pediatric high-grade brain tumors in the thalamus, midbrain, or pons; the latter are called diffuse intrinsic pontine gliomas (DIPG). The brain-stem location limits the clinical management of DIPG, resulting in exceedingly poor outcomes. A heterozygous point mutation in one of two non-canonical histone H3.3 genes is present in most DIPG tumors. This dominant mutation alters H3F3A, replacing lysine 27 with methionine (K27M), and results in global reduction of tri-methylation on K27 of all wild-type histone H3 proteins, which is thought to be a driving event in gliomagenesis. A lead antisense oligonucleotide (ASO) was developed that directed RNase-H-mediated knockdown of H3F3A mRNA. ASO treatment restored K27 trimethylation of histone H3 proteins and significantly reduced tumor growth, promoted neural-stem-cell differentiation, and increased survival in two different DIPG mouse models. This data demonstrated the involvement of the H3.3 K27M oncohistone in tumor maintenance, and the reversibility of the aberrant epigenetic changes, in addition to providing a preclinical proof-of-concept for DMG antisense therapy.

Pediatric high-grade gliomas (pHGGs) represent 10-15% of pediatric brain tumors, and have exceedingly poor outcomes (1,2). About half of pHGGs, termed diffuse midline gliomas (DMGs), exhibit a diffuse pattern in the midline, including the thalamus, midbrain, and pons; the latter constitutes an especially severe subgroup termed diffuse intrinsic pontine gliomas (DIPG). Most DIPG patients die within two years, with a mean survival of nine months. The brainstem location limits the clinical management of DIPG: surgical resection is impossible, and localized chemotherapy is ineffective and has severe side effects. Thus, new effective therapies are urgently needed.

A specific heterozygous point mutation that affects the non-canonical histone H3.3 is present in 70-80% of DIPG tumors. This dominant somatic mutation occurs in H3F3A-one of two genes encoding identical H3.3 proteins-replacing lysine 27 with methionine (K27M). H3.3 K27M is an oncogenic gain-of-function mutation that inhibits the EZH2 methyltransferase subunit of the Polycomb repressive complex (PRC2), leading to global reduction in di- and tri-methylation of all histone H3 proteins (3,4). This mutation thus activates many downstream genes, and appears to be a driving event in tumorigenesis (3,4). Moreover, genetic knockout or knockdown studies suggested that the mutant histone could be a therapeutic target (5,6). These observations led us to develop and test a pharmacological approach to directly target the mutant H3F3A mRNA. H3.3 K27M gliomas are more aggressive than H3.1 K27M (HIST1H3B K27M) gliomas, which are less prevalent and drive distinct oncogenic programs (7). Importantly, the K27M mutation correlates with poor patient outcomes, and can be diagnosed by MRI and stereotactic biopsy (8).

Targeted therapies for patients with the H3K27M mutation are currently in clinical trials, such as the HDAC inhibitor Panobinostat (LBH589) (9), but they target downstream genes that undergo epigenetic reprogramming. To achieve more direct gene-specific or allele-specific targeting for H3K27M gliomas, the potential of antisense oligonucleotides (ASOs) in H3K27M DIPG patient cells, using chemically modified “gapmer” ASOs that hybridize with complementary RNA sequences and mediate RNase-H cleavage of the target transcripts was explored. The chemical composition of the gapmer wings is the same as that of nusinersen (Spinraza®), a splice-modulating, uniformly modified ASO that promotes inclusion of exon 7 in SMN2 mRNA (10,11). Nusinersen was the first approved drug for spinal muscular atrophy, and the first disease-modifying therapy for neurodegeneration. In some embodiments it is described how lead ASOs identified in systematic screens specifically delayed the growth of H3K27M+ patient-derived cells grown as neurospheres. Furthermore, intracerebroventricular (ICV) administration of a lead ASO in two different DIPG mouse models significantly reduced tumor growth, promoted neural-stem-cell differentiation, and increased survival. These findings provide preclinical proof of principle for an antisense therapy for DMG.

### Example 6: CRISPR-Cas9 Depletion of H3.3-K27M Rescued H3-K27 Trimethylation and Delayed the Growth of Patient-Derived Neurospheres and Orthotopic Xenografts

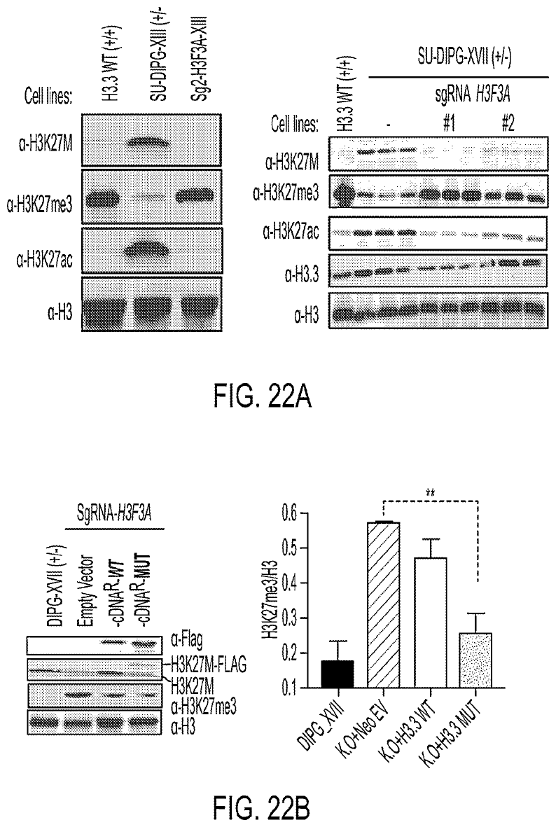

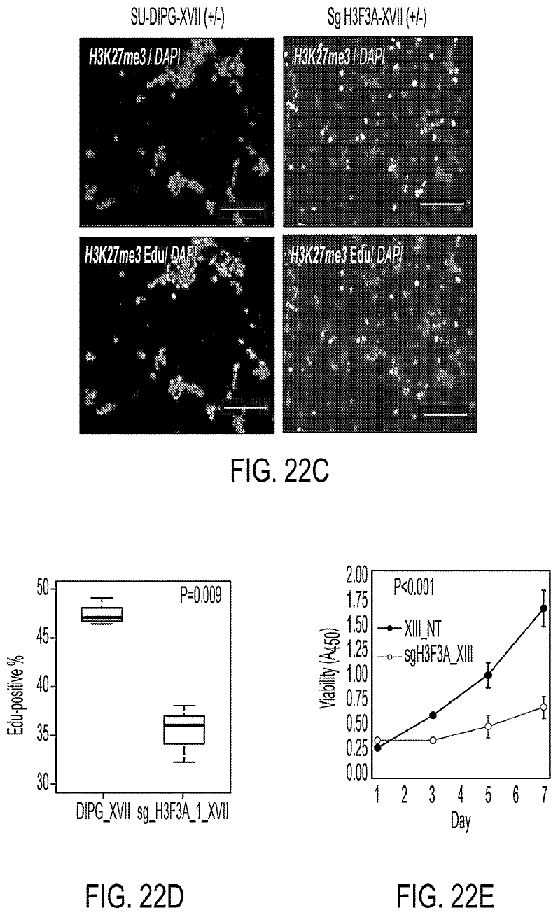

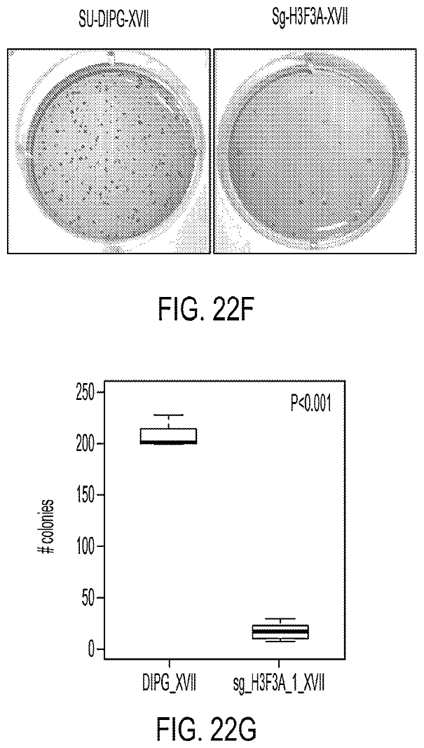

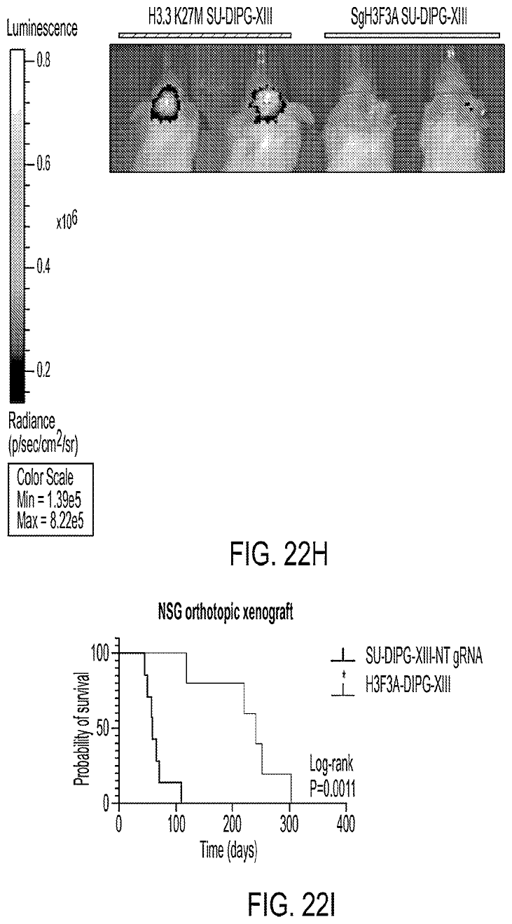

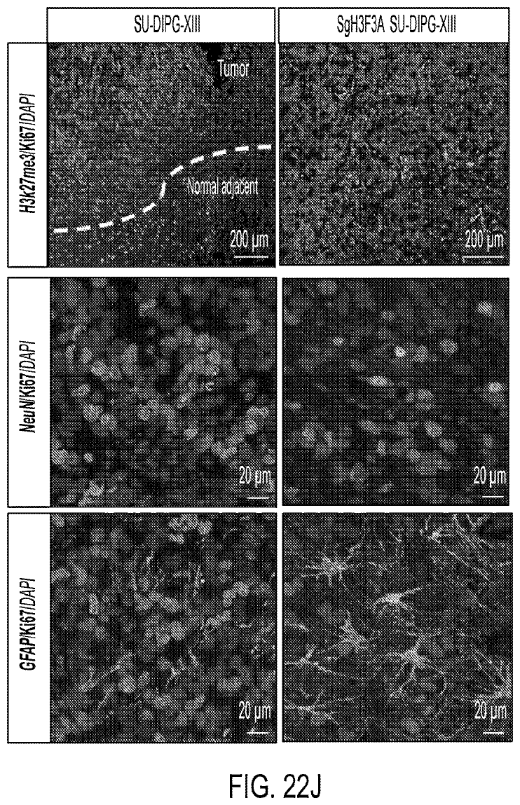

CRISPR-Cas9 with sgRNA targeting both mutant and wild-type alleles, but leaving H3F3B (which encodes the identical protein) unaffected in two DIPG-patient cell lines (SU-DIPG-XIII, XVII (9)), was used as a control for the biological consequence of complete genetic knockout of H3F3A in the models. The dominant-negative effect of the K27M mutation on the overall levels of H3K27me3 is well documented (1-4). In both patient-cell lines harboring the H3F3A heterozygous mutation, H3.3 K27M knockout restored the repressive H3K27me3 trimethylation mark and reduced the permissive H3K27ac acetylation mark, detected by immunoblotting (FIG. 22A) and immunofluorescence (IF) (FIG. 22C). Overexpression of gRNA-resistant H3F3A mutant cDNA decreased the restored H3K27me3 mark, consistent with an on-target effect (FIG. 22B). The knockout reduced cell proliferation, detected by Edu staining (FIGS. 22C-22E), and soft-agar colony formation (FIGS. 22F-22G). To determine whether H3.3 K27M is required for tumor maintenance in vivo, orthotopic transplantation of patient cells or H3F3A MUT knockout cells into immunocompromised NSG mice at postnatal day 3 was performed (9). The SU-DIPG-XIII line was used, because its slower proliferation rate extends the therapeutic window for treatment. The transplanted knockout cells showed significantly reduced growth, resulting in increased survival (FIGS. 22H-22I). The H3.3 K27M tumors histologically resembled patient tumors, with a global reduction of the H3K27me3 mark, compared to normal adjacent tissue. This effect in H3.3 K27M tumors was alleviated in the knockout tumors, and correlated with elevated expression of a mature-neuron marker (NeuN+) and an astrocyte marker (GFAP+), detected by IF (FIG. 22J), suggesting that the knockout cells are less proliferative and more committed to differentiated lineages in vivo. These results confirm and extend previous studies (5,6), and underscore the potential of mutant H3.3 K27M as a therapeutic target.

### Example 7: PS-MOE Gapmer ASO Screen to Reduce Mutant H3F3A mRNA and H3.3 K27M Protein

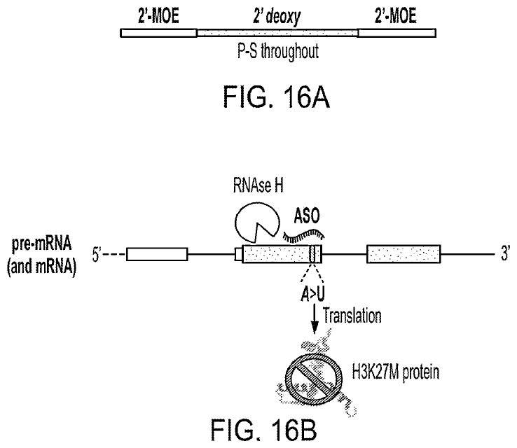

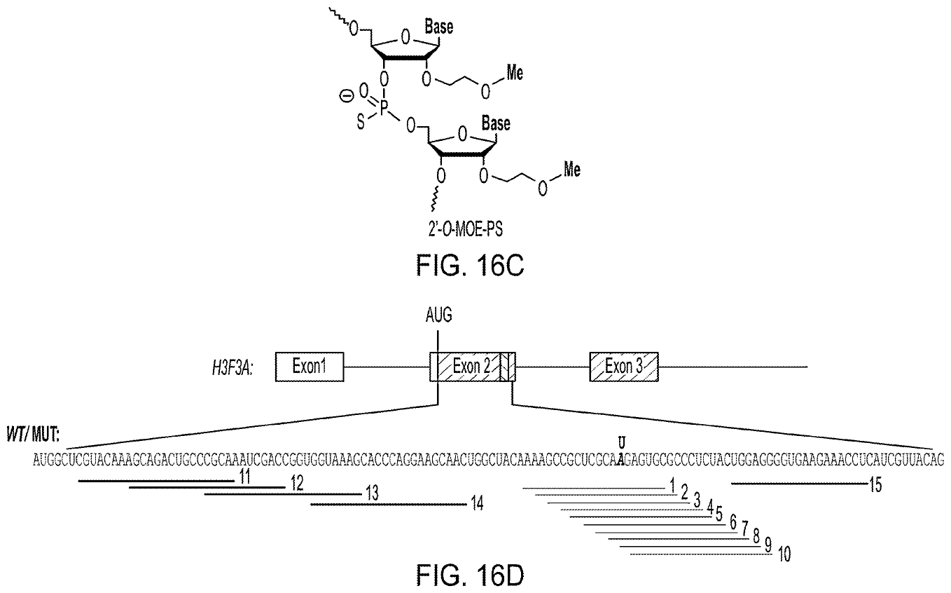

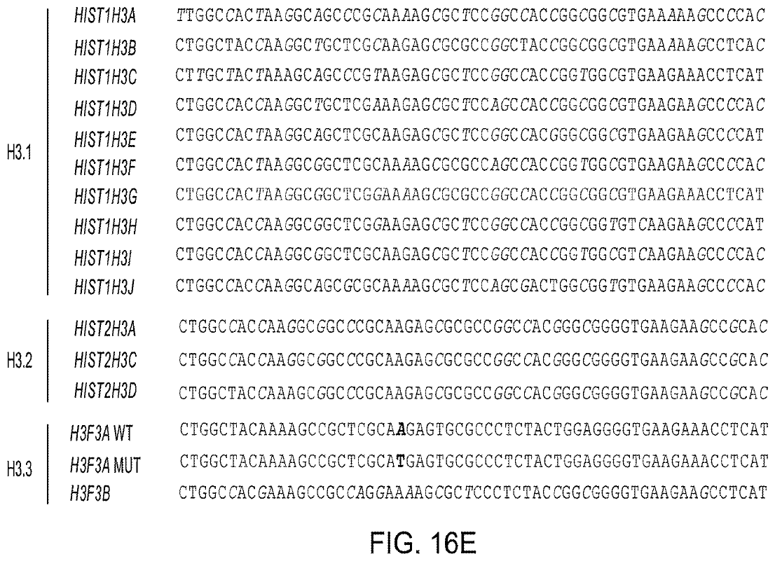

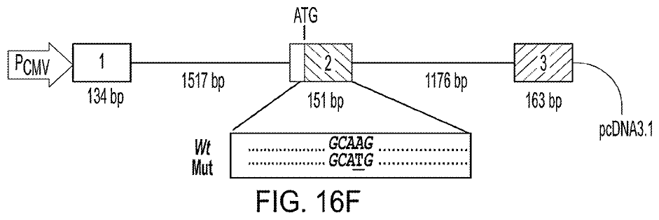

To pharmacologically target H3F3A mRNA, the potential of ASOs in DIPG patient cells was explored. Gapmers with 2′-O-methoxyethyl (MOE) wings and a phosphorothioate (PS) backbone (FIGS. 16A-16B) were designed and tested. These gapmer ASOs have a DNA-like central region that directed cleavage of the complementary mRNA (or pre-mRNA) target by endogenous RNase H, and chemically modified wings that promoted tighter RNA binding, enhanced stability, and improved cellular uptake (FIG. 16C) (12). To develop targeted allele-specific knockdowns, 10 overlapping 20-mer PS-MOE-ASOs (#1-10) that span the mutation site in the H3F3A exon 2 and flanking nucleotides, such that the mutation is across the ASO gap were designed. These sequences were fully complementary to the H3F3A mutant allele, and had one mismatch to the H3F3A WT allele. Second, to develop targeted gene-specific knockdown-considering that the wild-type H3.3 protein was still expressed from the H3F3B gene five additional overlapping 20-mer PS-MOE gapmer ASOs (#11-15) to target exonic regions upstream or downstream of the mutation site in exon 2 (FIG. 16D) were designed. These ASOs targeted only H3F3A but not H3F3B transcripts, or other gene transcripts that encoded H3.1 or H3.2 canonical histone proteins (FIG. 16E). In addition, a wild-type H3F3A minigene and a mutant H3F3A minigene with the A-to-T mutation were generated by cloning genomic fragments comprising exons 1 to 3, with intact introns 1 and 2 (FIG. 16F).

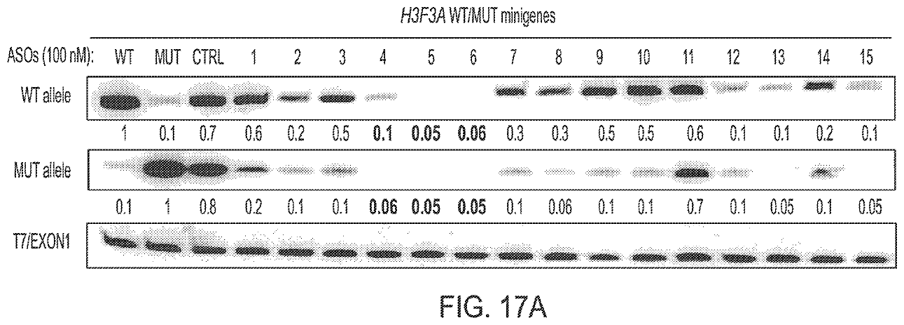

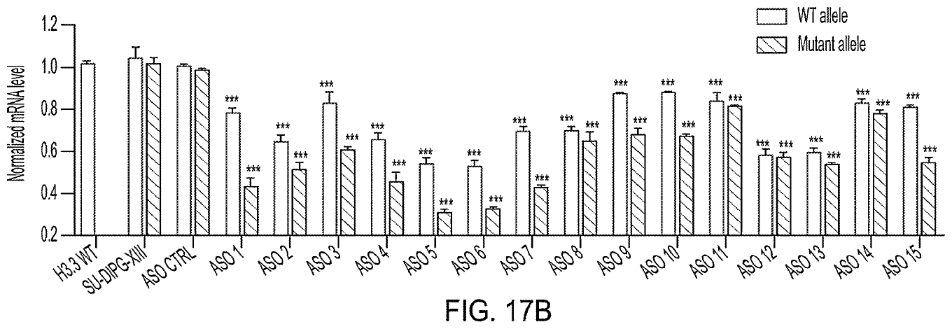

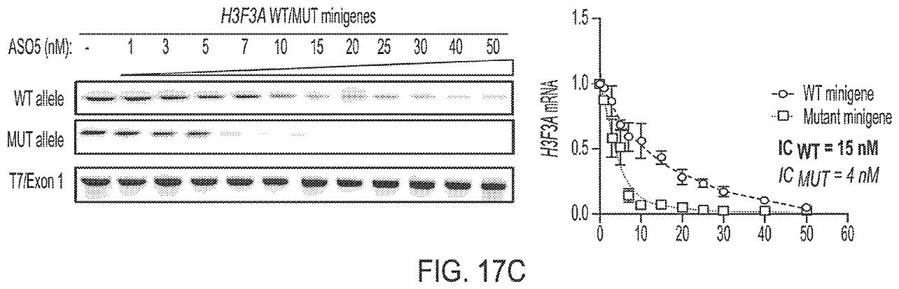

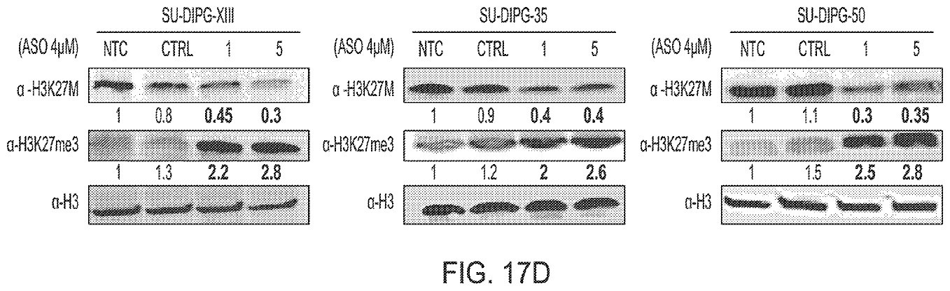

To identify whether some of these ASOs mediated RNase-H1 cleavage of mutant H3F3A mRNA, or both mutant and wild-type, individual ASOs (100 nM) with the H3F3A WT or H3F3A MUT minigene were transfected into HeLa cells. These ASOs were delivered by free uptake (4 μM) into patient-derived neurosphere cultures. The latter method relies on scavenger-receptor-mediated endocytosis, and requires much higher ASO concentrations (12). The initial minigene screen identified three consecutive ASOs (ASO4, 5, and 6) with allele-specific design, and two consecutive ASOs (ASO12 and 13) with gene-specific design, that achieved robust H3F3A knockdown (FIG. 17A). All the allele-specific ASOs achieved more robust knockdown of the mutant than the wild-type allele in patient-derived cells, whereas at this concentration, ASO4, 5, and 6 robustly knocked down both alleles in the minigene context. The most potent ASO (ASO5) was titrated in HeLa cells, and allele-specific knockdown between 3 to 40 nM in transfection experiments was observed. The wild-type allele had an IC₅₀ of 15 nM versus 4 nM for the mutant allele (FIG. 17C).

Similarly, ASOs delivered into neurosphere cultures by free uptake knocked down expression of the endogenous mutant allele by 50-70%, and expression of the WT allele by a lesser extent (30-40%) (FIG. 17B). Surprisingly, one gene-specific ASO, ASO15, also promoted allele-specific knockdown, suggesting steric blocking of a putative regulatory RNA-binding protein(s) that binds within its target region (FIG. 17B). Two allele-specific lead ASOs were selected for testing in two additional patient-derived cell lines, and developed primer pairs for H3F3B, and for total H3F3A, amplifying a region downstream of the mutation. The two lead ASOs, delivered by free uptake at 4 PM, behaved similarly across the three different patient cell lines: they selectively knocked down H3F3A, but not H3F3B, and depleted the mRNA from the mutant allele to a greater extent than the wild-type allele (FIG. 17D).

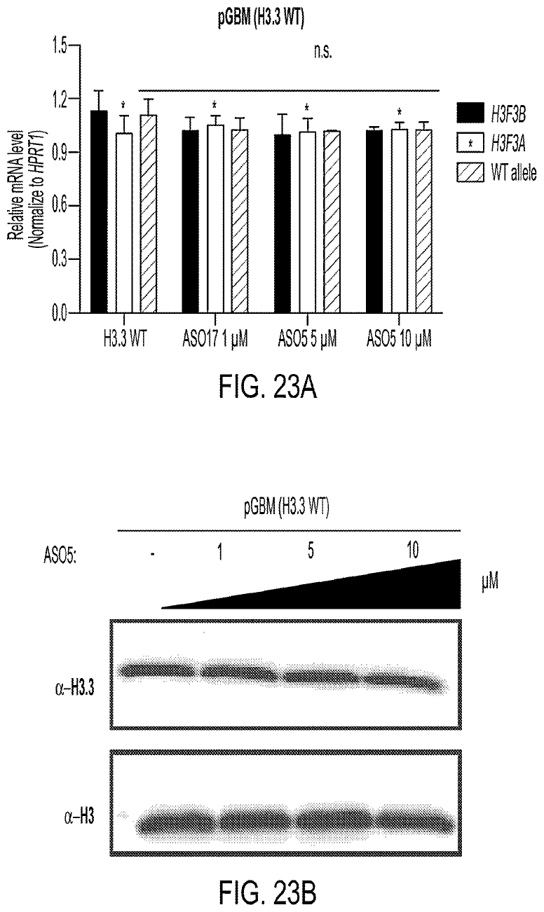

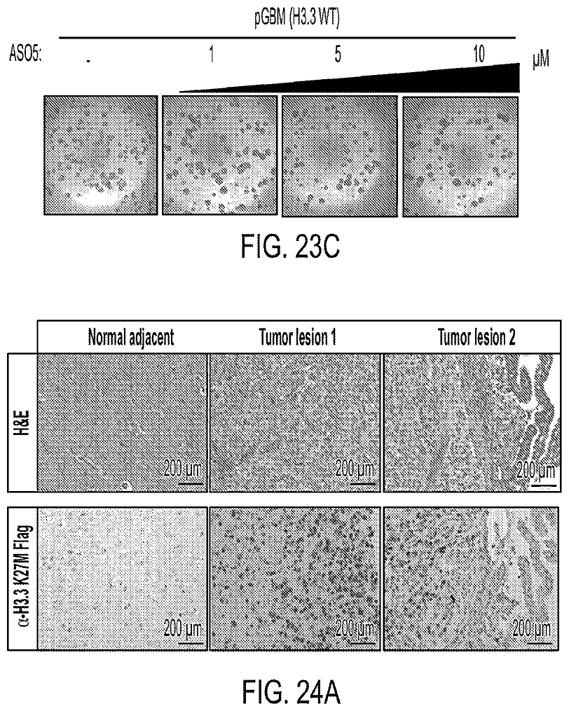

Finally, the level of histone H3.3 protein was measured by immunoblotting with an antibody specific for H3.3 with the K27M mutation, and downstream epigenetic changes were measured using antibodies to tri-methylated H3K27 and total H3 histone proteins as normalization controls. The two lead ASOs knocked down ˜60%-70% of the mutant protein, resulting in 2-3-fold H3K27me3 elevation across three DIPG lines (FIG. 17E). In contrast, higher doses up to 10 μM of the lead ASO (ASO5), delivered by free uptake into H3.3 WT glioma cells, did not significantly reduce H3F3A mRNA or H3.3 protein levels (FIGS. 23A-23B).

### Example 8: ASO-Mediated H3.3 K27M Depletion Delayed Neurosphere Growth and Changed Cell Morphology

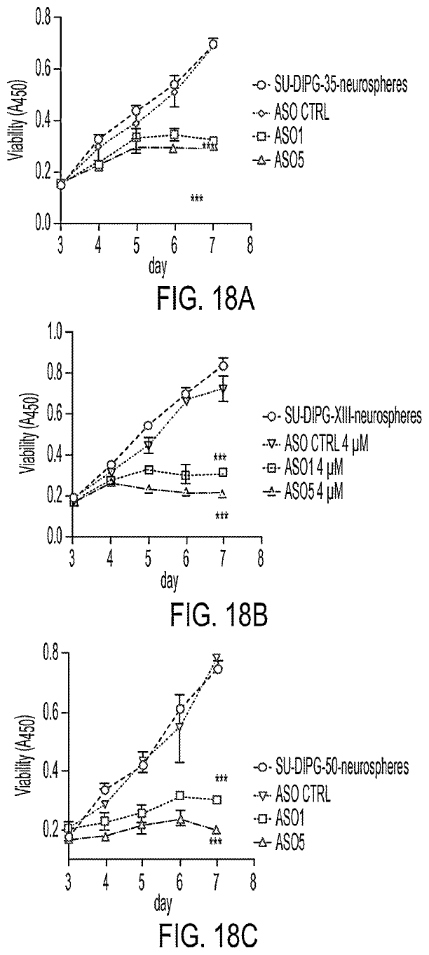

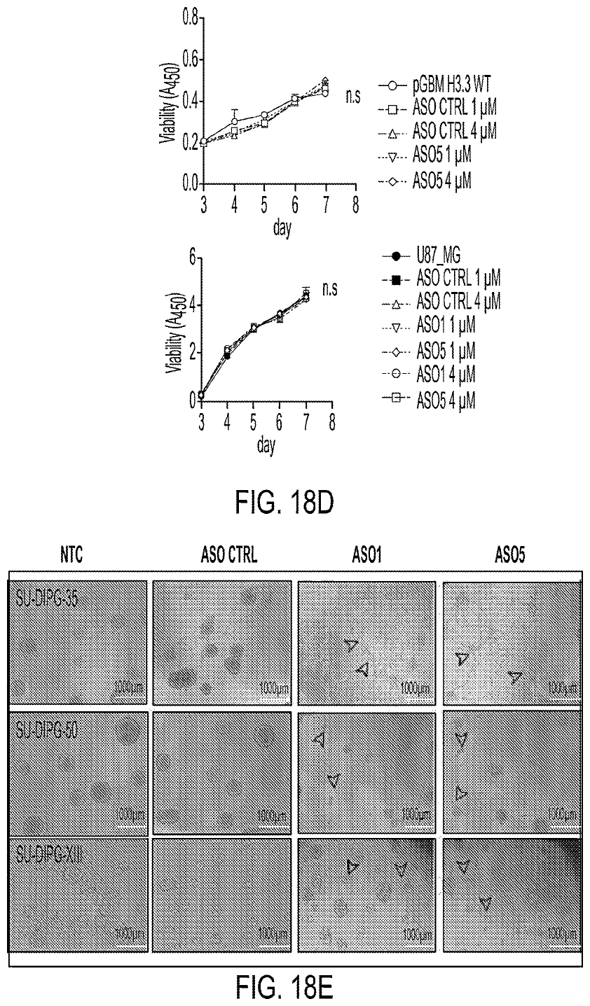

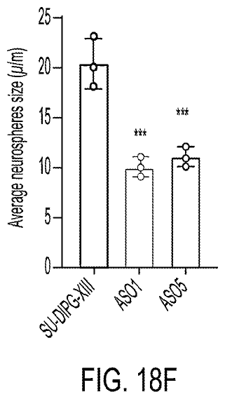

Using several H3.3 K27M DIPG-patient-derived cell lines grown as neurospheres, it was observed that the lead ASOs (ASO1 and 5) delayed tumor-cell growth, compared to scrambled control ASO. Slower proliferation was observed for SU-DIPG-XIII, SU-DIPG-35 and SU-DIPG-50 cells treated with 4 μM ASO1 or ASO5 (FIGS. 18A-18C). In contrast, neither low (1 μM) nor high (4 μM) doses had measurable effects on proliferation of two control H3.3 WT glioma lines grown as neurospheres (FIG. 18D). At the 5-day time point, the cellular morphology changed, with neurite-like processes consistently forming with all three DIPG patient-derived lines, but not with the control H3.3 WT glioma line (FIGS. 18E and 23C). This morphological change was consistent with the differentiation phenotype we observed in the CRISPR-knockout orthotopic xenografts (FIG. 22J). In addition, the DIPG lines treated with the lead ASOs formed smaller neurospheres (FIG. 18F). The reduction in H3.3 K27M mutant protein displayed phenotypic consequences in patient cells, including slower proliferation and a differentiated morphology.

### Example 9: ICV Injection of Lead ASO Promoted H3.3 K27M Depletion, Lower Tumor Grade, and Differentiation in an RCAS-Tva Mouse Model

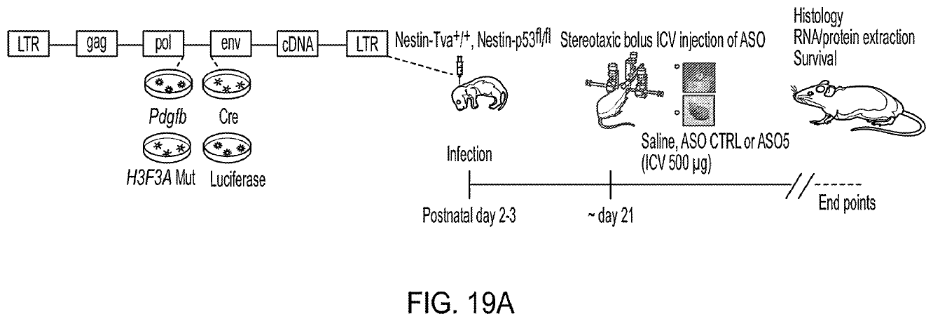

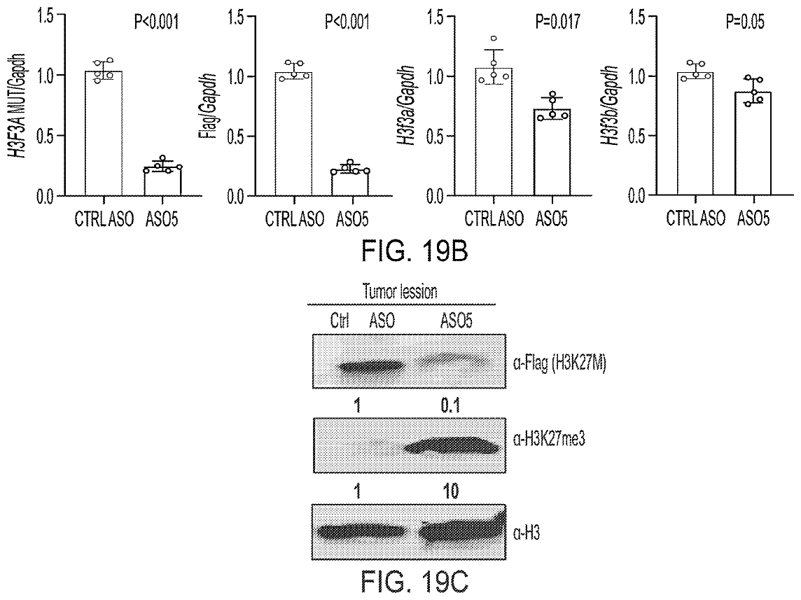

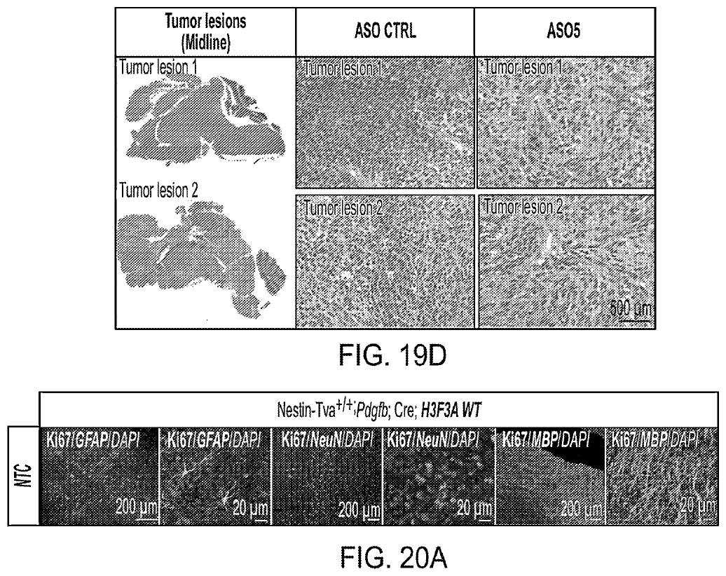

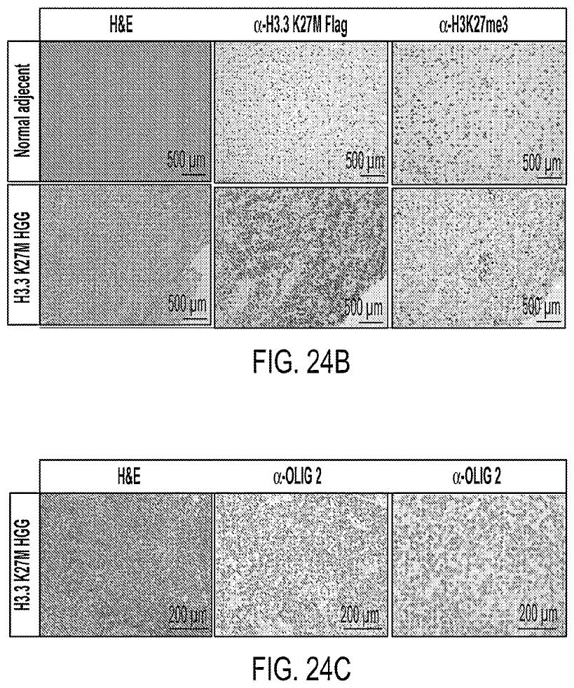

To assess lead ASOs in vivo, the RCAS-Tva system was employed to establish a mouse model of DIPG. RCAS, or replication-competent avian sarcoma-leukosis virus long terminal repeat (LTR) with splice acceptor, is a viral vector that only infects cells expressing the avian Tva receptor (13). This system was previously used to show that murine histone H3.3 K27M or H3.1 K27M accelerates gliomagenesis, consistent with results in another genetic mouse model (14,15,16). The system was adapted by introducing instead a human H3F3A K27M cDNA, whose transcripts can be targeted by the human-specific ASOs. Chicken DF1 cells producing four viruses encoding Cre recombinase, H3.3K27M, PDGFB, and luciferase, were delivered into the brainstem of neonate mice with Tva driven by the nestin promoter, and a p53-floxed allele (FIG. 19A). Mice developed high-grade gliomas within 3-6 weeks, localized in the midline region. FLAG-tagged H3.3 K27M was present in the glioma lesions, as seen by histological analysis (FIG. 24A). Approximately 90% of H3.3 K27M tumors showed global H3K27me3 reduction, compared to the normal adjacent tissue, a pattern that resembles patient-tumor histology (FIG. 24B). Tumor cells also showed robust expression of the oligodendroglial lineage marker OLIG2 (15) (FIG. 24C).

To determine the effectiveness of the lead ASO in vivo, stereotaxic ICV injections were performed to deliver it directly into the cerebrospinal fluid (CSF) (17,18). A single dose (500 μg) of lead ASO5 or CTRL ASO was injected in saline into a lateral ventricle, at the time of tumor onset, detected by bioluminescence imaging (FIG. 19A). RNA and protein was extracted from the tumors or normal adjacent tissue at preset timepoints. ASO5 significantly knocked down the human H3F3A mutant mRNA and FLAG-tag, and to a lesser extent the endogenous murine wild-type H3F3A, but not H3f3b (FIG. 19B). At the protein level, ASO5 significantly knocked down human FLAG-tagged H3.3 K27M, and increased H3K27me3 levels (FIG. 19C).

Mice treated with the control ASO developed highly proliferative and aggressive gliomas, with numerous mitotic figures, extensive vascular proliferation, occasional necrosis, and pseudo-palisades around necrotic areas. These tumors were non-encapsulated and poorly demarcated, with some tumor invasion at the tumor-brain interface. In contrast, mice treated with ASO5 showed an extended latency of tumor growth, and the tumors exhibited elongated morphology (FIG. 19D). In these tumor lesions, mitotic figures were rare, and necrosis was not prominent. Moreover, the cells in ASO5-treated tumor lesions morphologically resembled glia and mature neurons. It was determined that the lead ASO significantly knocked down mutant H3F3A in vivo, resulting in lower-grade tumor formation and a more differentiated appearance.

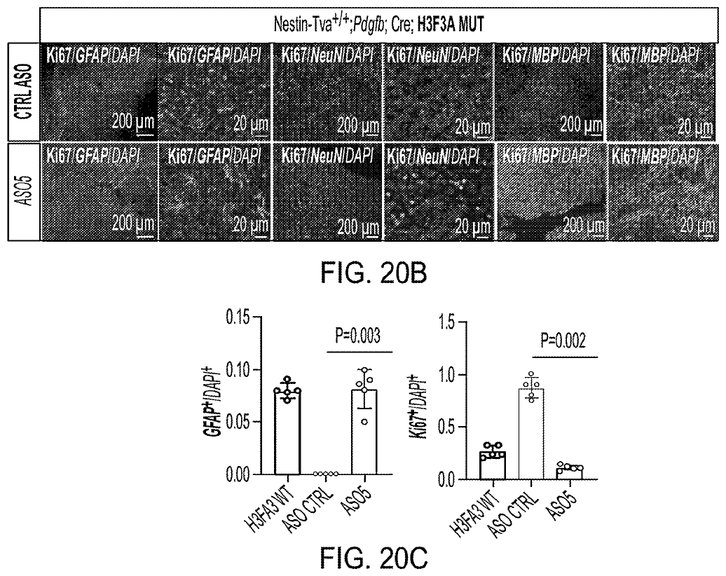

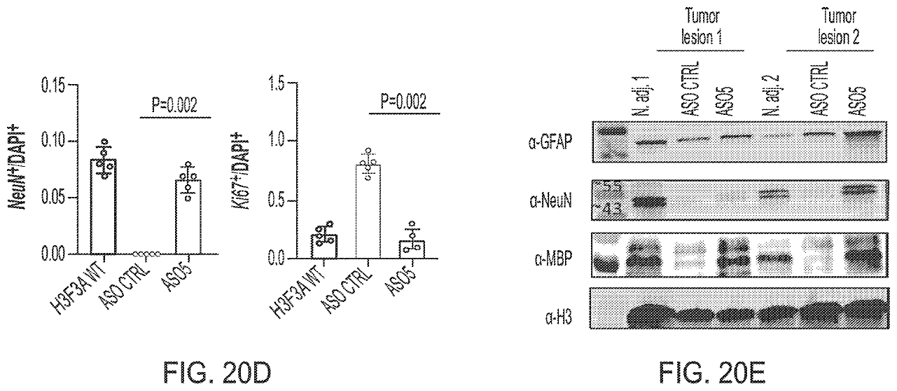

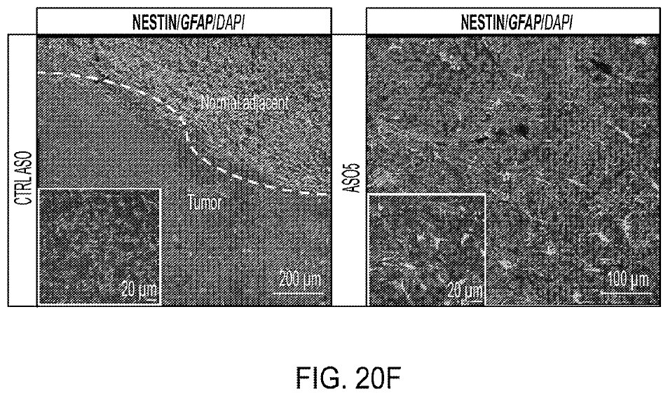

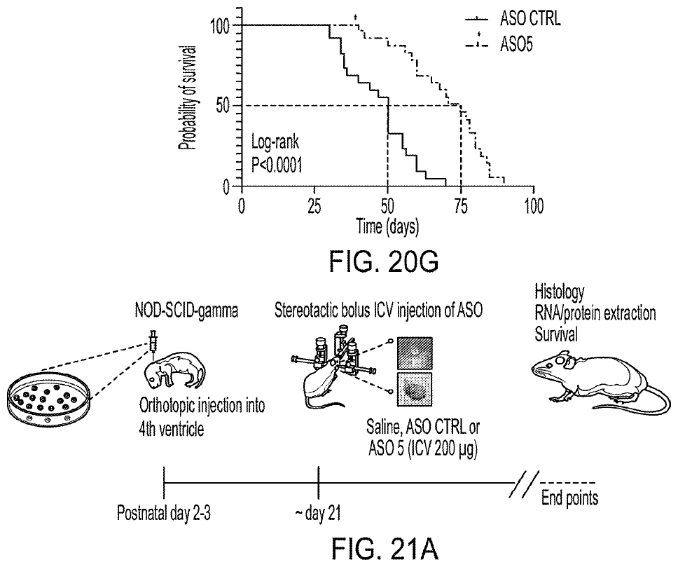

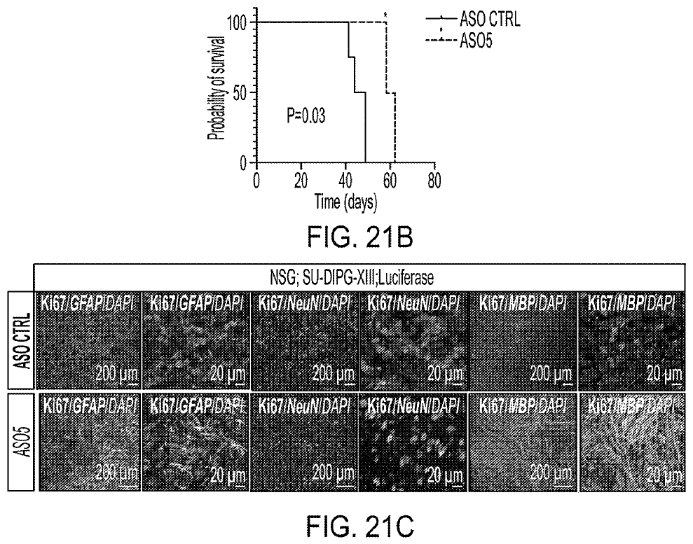

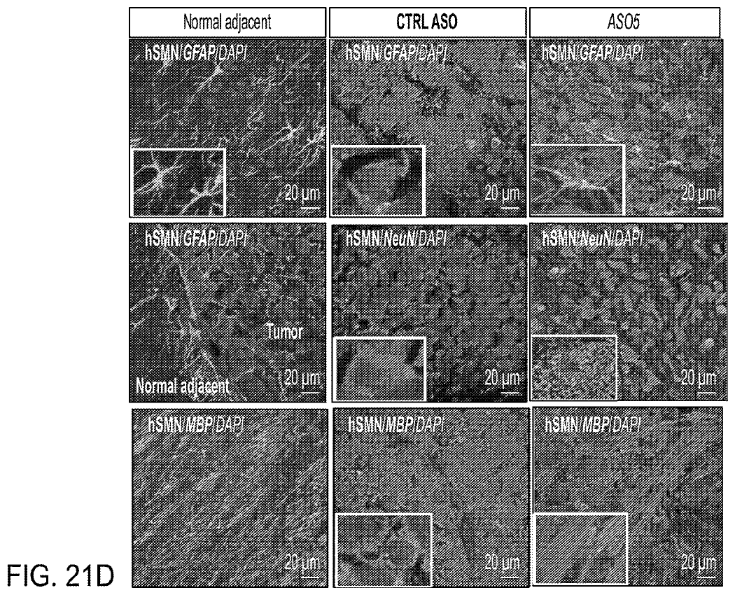

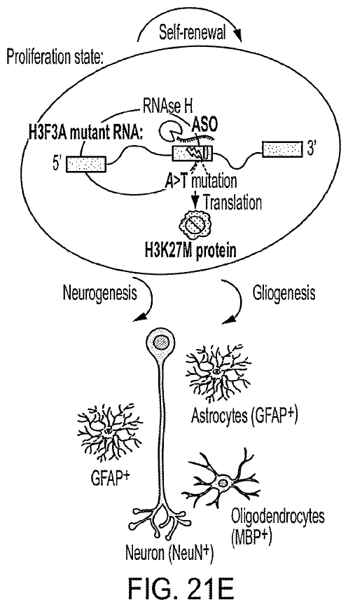

To further characterize the phenotypes observed by histology, IF staining was performed for known differentiation markers. Notably, in mice of the same genetic background but bearing H3.3 wild-type tumors, markers of mature astrocytes (GFAP, glial fibrillary acidic protein (19)), neurons (NeuN, neuronal nuclear protein (20)), and oligodendrocytes (MBP, myelin basic protein, (21)), were detected which suggested the occurrence of neurogenesis and gliogenesis in H3.3 WT gliomas (FIG. 20A). In contrast, there were few detectable GFAP⁺, NeuN⁺, and MBP⁺ cells within tumor lesions in the presence of H3.3 K27M. After a single ICV dose of ASO5, numerous GFAP⁺, NeuN⁺, and MBP⁺ cells were detected, which correlated with a reduction in proliferating cells marked by Ki67, a nuclear cell-proliferation-associated antigen expressed in all active stages of the cell cycle (FIGS. 20B-20D and 21A-21D). This data was confirmed by western blotting of total protein extracted from normal adjacent tissue and tumor lesions (FIG. 20E). These results suggested H3.3 K27M blocks astrocyte and neuron differentiation, and ASO-mediated H3.3 K27M depletion restores the differentiation programs.

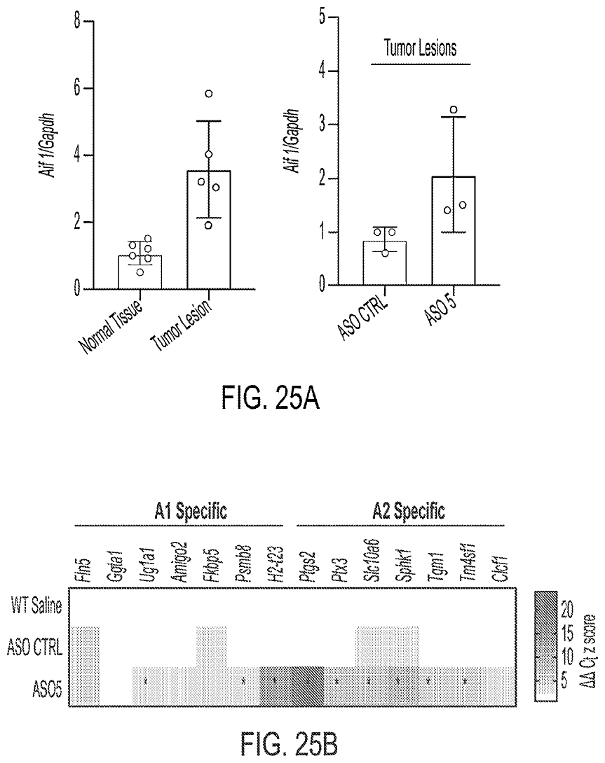

In addition, elevated Aif1 induced by H3F3A ASO was observed compared to scramble-control ASO in the mRNA from tumor tissue, but not in normal adjacent tissue (FIG. 25A). Further, the elevation of several murine genes related to cytokines and A2-specific reactive astrocytes (19) (FIG. 25B) was observed, suggesting that the ASO treatment triggers a tumor-intrinsic immune response.

### Example 10: ICV Injection of ASO5 Decreased the Nestin⁺ Cell Population and Extended the Latency of Tumor Growth

DMGs arise within defined spatial-temporal contexts and tend to occur during middle childhood. The cellular origin and the microenvironment are essential for tumor growth (22,23). Retrospective clonal analysis revealed that Nestin⁺ cells are enriched in the human midbrain, pons, and medulla throughout childhood, with peak density in the ventral pons; thus, the Nestin⁺ cell population corresponds strikingly with the spatial and temporal incidence of DIPG (23). In the mouse model, the Nestin⁺ cells were highly enriched at the location of tumor lesions, relative to the normal adjacent brain tissue. After ASO5 treatment, the Nestin⁺ cell population was drastically reduced, inversely correlating with the high number of GFAP⁺ cells (FIG. 20F). Moreover, ASO5-treated mice had significantly longer survival than control-ASO-treated mice (FIG. 20G). These results suggested that H3.3 K27M tumors originate from neural stem cells (Nestin⁺) in the context of p53 loss and PDGFR signaling.

### Example 11: ICV Injection of ASO5 Promoted Astrocyte, Neuron, and Oligodendrocyte Differentiation, and Decreased Tumor Proliferation in a DIPG Patient-Derived Xenograft Model

To confirm and extend the above observations, ASO5 was also tested in an orthotopic xenograft mouse model, as described (9), using one of the SU-DIPG patient lines shown in FIGS. 18A-18F. 10⁵ luciferase-expressing SU-DIPG-XIII cells were injected into the 4^{th} ventricle of postnatal-day-3 immunocompromised mice, and used bioluminescence imaging to follow tumor onset. Because these mice did not tolerate the high ASO concentration used in the RCAS-Tva model, a single ICV injection of 200 μg control ASO or ASO5 (FIG. 21A) was administered. The mice treated with ASO5 survived longer than the control-ASO-treated cohort (FIG. 21B). Similar to the RCAS-Tva mouse model, these ASO5-treated mice exhibited a differentiation phenotype in the tumor lesions (GFAP⁺, NeuN⁺, and MBP⁺), with fewer proliferating cells. Neurogenesis and gliogenesis were compromised in the control-ASO-treated cohort, and the majority of the tumor cells remained in a highly proliferative state (Ki67⁺ (FIG. 21C). The xenograft model were used to determine whether the differentiated cells seen after ASO5 treatment were of tumor origin, or were murine cells recruited to the lesions. To this end, IF staining was performed with a human-specific monoclonal antibody against SMN protein (17). No hSMN⁺ cells were detected in normal adjacent tissue, but a large fraction of the tumor cells were hSMN⁺. Moreover, when hSMN was co-stained with the above differentiation markers, human SMN in astrocytes, neurons, and oligodendrocytes (FIG. 21D) were observed. The treatment with ASO5 resulted in depletion of H3.3 K27M protein, leading to markedly restored neurogenesis and gliogenesis, longer latency of tumor growth, and significantly increased survival in this mouse model. (FIG. 21E).

### Example 12: Discussion

The recognition that H3K27M-mediated aberrant gene activation or de-repression is an oncogenic driver in DMG motivated the development of a direct strategy to deplete the mutant histone H3.3. Using genetic knockout with CRISPR-Cas9 and sgRNA targeting both H3F3A alleles, it was determined that H3K37M is required by tumors. In agreement with previous work (5,6), H3F3A-knockout DIPG cells remained viable, but became less proliferative and more differentiated, thus extending survival in an orthotopic-xenograft mouse model. The H3F3A K27M mutation is dominant-negative, whereas the H3F3A wild-type allele might be redundant in the tumors and normal cells, because H3F3A and its paralog, H3F3B, encode identical H3.3 histone proteins, and are ubiquitously and similarly expressed across different cell lineages, including in the central nervous system (CNS). Moreover, single knock-out H3f3a or H3f3b male and female mice are normal and fertile; only double knock-out mice show developmental retardation and embryonic lethality (24). Thus, targeting H3F3A would still allow H3F3B to express normal H3.3 protein to carry out its functions in various tissues. Therefore both allele-specific and gene-specific targeting of H3F3A was explored, using chemically modified gapmer ASOs. The systematic ASO screen with allele-specific design showed preferential targeting of the mutant allele. In contrast, the gene-specific design had similar effects on both alleles, except for one ASO that somehow preferentially knocked down the mutant allele. Further, two lead ASOs targeting the H3F3A mutant allele specifically delayed the growth of patient-derived cells grown as neurospheres, and promoted a differentiated morphology.

Depleting mutant H3K27M restored the balance of post-translational modifications at K27 on all histone H3 proteins, normalizing or detoxifying the expression of downstream genes (9, 25). The differentiated phenotype observed in H3K27M-depleted patient cells and mouse models treated with gapmer ASO suggested that some downstream genes were associated with neurogenesis and/or gliogenesis. To characterize the differentiated cellular phenotype, various differentiation markers for astrocytes (GFAP), neurons (NeuN), and oligodendrocytes (MBP) were investigated. Tumors from mice bearing H3K27M had deficient expression of all tested differentiation markers, and a higher proliferation rate (Ki67). In contrast, markedly elevated GFAP⁺, NeuN⁺, and MBP⁺ cells were identified in ASO-treated H3.3 K27M tumors. Notably, control mice with identical genetic background but with glioma xenografts expressing H3.3 WT also expressed these differentiation markers in the tumor lesions.

A previous study employed genetically engineered mice to show that H3.3 K27M enhances neural-stem-cell self-renewal and accelerates spontaneous brain-stem gliomas (16). The same group also reported a paired isogenic comparison using shRNA knockdown of H3.3 K27M to address the underlying mechanism of the mutation-specific effect on the transcriptome and epigenome. The authors used ChIP-seq and RNA-seq analyses to identify highly enriched genes associated with neurogenesis and nervous-system development upon K27M shRNA knockdown (6). Likewise, another study reported that H3K27M gliomas are derived from oligodendrocyte precursor cells (OPC); single-cell RNA-seq of primary patient cells showed that large undifferentiated OPC-like cells are over-represented in H3K27M-gliomas, and exhibit more significant proliferation and oncogenic properties than their more differentiated counterparts, in the presence of PDGFRA signaling (22). The ASO-mediated H3.3 K27M depletion rescued the impaired differentiation programs, resulting in slower glioma proliferation. Taken together, these studies suggest that the H3K27M oncohistone drives tumorigenesis by enhancing the self-renewal capacity of neural stem cells and blocking neural/glial differentiation.

Administration of a lead ASO to an immunocompetent DIPG mouse model generated by viral transduction significantly reduced tumor growth, promoted neural-stem-cell differentiation, and increased survival. The RCAS-Nestin Tva mouse model develops midline high-grade gliomas by viral infection of endogenous Nestin⁺ cells in a relevant brain-development window and environment (14, 15). This model was adopted by incorporating human mutant H3F3A cDNA-whose transcripts can be targeted by the human-specific ASOs-in addition to PDGFR cDNA and TP53 depletion. Nestin marks a neuroepithelial stem-cell population with self-renewal capacity and the potential to generate differentiated cells. In the mouse model, Nestin⁺ cells were highly expressed in the tumor lesions, reflecting the self-renewal capacity of neural stem cells, but were absent in the normal adjacent tissues. When mice were treated with ASO to deplete H3K27M, they showed a significant decrease in Nestin⁺ cells and increase in GFAP⁺ cells. These indicated that Nestin⁺ cells are overrepresented in tumor lesions that maintain a highly proliferative state in the presence of the H3.3 K27M mutation.

Several neural stem-cell markers, including Nestin, are expressed in tumor-forming cells in patients (23). Moreover, DIPG-like gliomas can develop from iPSC-derived iNSC, when overexpressing H3.3 K27M with TP53 depletion (26). These studies showed that H3K27M can block neural stem-cell differentiation and keep the Nestin⁺ tumors in the self-renewal state. ASO treatment significantly extended survival by converting highly proliferative neural stem cells into more differentiated cells. This differentiation process was identified using a different mouse model generated by orthotopic transplantation of DIPG patient cells. In this case, it was further demonstrated that the differentiated cells were of human origin, by co-staining the tumors with a human-specific SMN antibody and antibodies to various differentiation makers, which showed co-localization.

ASOs with appropriate chemical modifications have a long duration of action in the CNS. For example, nusinersen (Spinraza®), which targets SMN2 pre-mRNA, maintains its effect for 6 months after ICV infusion or injection in adult SMN2-transgenic mice (17,18). Similarly, the lead “gapmer” ASO promoted robust H3F3A knockdown for at least 90 days after a single 500-μg dose in the RCAS-Tva mouse model. ICV injections were performed to directly deliver the ASO into CSF, bypassing the blood-brain barrier (BBB). This route allows ASO penetration into the brain and other CNS tissues, but much less so in peripheral tissues (which express only wild-type H3.3 histone). Subsequently, ASO is cleared through the CSF flow tracts to the venous blood (27). Some ASO in the blood circulation may then return to the tumor through its vasculature and compromised BBB, potentially contributing to the overall knockdown effects in the tumors. Indeed, it was observed that robust vascular proliferation dissects the tumors into pseudo-lobules in the RCAS-Tva mouse model, suggesting angiogenesis in the tumor lesions.

Microglia, the most abundant innate immune cell in the CNS, can influence BBB function; when microglia are activated, they cause either BBB repair or disruption during inflammation, the latter increasing BBB permeability (28,29). However, DMGs are “cold” tumors, characterized by immunosuppression (30). Interestingly, ASO treatment triggered neuroinflammation in the RCAS-Tva mouse model, as seen by Aif1 elevation. Importantly, the lead ASO did not cause microglia activation in normal adjacent tissue, so the response in the tumor tissue is not directed to the ASO. Upregulation of genes related to cytokines and A2-specific reactive astrocytes further suggested that the ASO treatment triggers a tumor-intrinsic immune response.

In summary, this study is the first that utilized gapmer ASOs to directly target an oncohistone gene in vitro and in vivo. The lead ASO efficiently degraded H3F3A mutant mRNA, reducing H3.3 K27M protein in patient-derived cells and mouse models. The decrease in H3.3 K27M protein level resulted in markedly restored neurogenesis and gliogenesis, a longer latency of tumor growth, and significantly increased survival in the mouse models. The pharmacological intervention was less effective than complete genetic knockout, because ASO treatment reduced but did not eliminate expression of the mutant protein. Furthermore, unlike the pre-implantation genetic knockout, therapeutic ASO was administered after tumor onset—a more realistic scenario. It is possible that maximal clinical efficacy will likely require combination therapy. For example, DMG patients' symptoms transiently improve after radiotherapy, and deletion of Atm radiosensitizes p53-deficient brain-stem gliomas in an RCAS-Tva mouse model (31). Therefore, ASO treatment in combination with radiotherapy may result in enhanced effectiveness and increased survival. Another possibility is to combine ASO treatment with immunotherapy. For example, systemic administration of GD2-targeted CAR T cells cleared engrafted tumors in patient-derived H3-K27M⁺ DMG orthotopic xenograft models (32). A clinical trial using this approach is ongoing (NCT04196413) (33). Potentially, such immunotherapy in combination with antisense therapy could achieve greater efficacy to inhibit tumor growth and increase survival.

### Example 13: Materials and Methods

Study plan. The aim of this preclinical translational project was to develop lead ASOs as a targeted therapy for H3.3 K27M pediatric brain cancers. For in vitro experiments, qRT-PCR, radioactive RT-PCR, viability, EdU staining, and other experiments were performed in biological triplicates. For in vivo analyses, tumor growth was monitored via bioluminescence imaging. Mice were monitored daily and euthanized with CO₂ when they became symptomatic (including an enlarged head, ataxia, or weight loss up to 25%) or at 6 months post-injection if they remained asymptomatic. Mice were excluded when hydrocephalus was observed. For both models, mice were randomized to each group before treatment (CTRL ASO (n=5) or ASO5 (n=5) treatment for IHC/IF, RNA, and protein analysis; CTRL ASO (n=22) or ASO5 (n=21) treatment for the survival study in the RCAS-TVA model; CTRL ASO (n=5) or ASO5 (n=5) treatment for the survival study in the DIPG orthotopic xenograft mouse model. IF/IHC analyses were performed blinded.

RCAS-TVA mouse model. DF1 cells (AATC Catalog #CRL-12203) were cultured in DMEM supplemented with 10% FBS, 2 mM L-glutamine, 100 units/mL penicillin and 100 g/mL streptomycin, and incubated at 39° C. and 5% CO₂. 5 μg of each RCAS plasmid was transfected into DF1 cells using X-TremeGENE 9 (Roche) following the manufacturer's instructions. The Nestin-Tva; p53^{fl/fl} mouse strain was used. For the generation of midline gliomas, transfected cells were passaged at least three times prior to injection; 10⁵ virus-producing DF1 cells were injected intracranially into a depth of 1-3 mm below the lambda suture of neonatal N-tva; p53^{fl/fl} pups (postnatal days 3-5) in 1 μL, using a Hamilton syringe (7659-01) and a 30-gauge needle. Four virus-producing cells expressing RCAS-Pdgfb, RCAS-Cre, RCAS-H3F3A mutant, and Rcas-Luciferase, were injected together in equal amounts.

DIPG orthotopic xenograft mouse model. This procedure was carried out as described (9). Briefly, a single-cell suspension of luciferase-transduced SU-DIPG-XIII-luc neurospheres (pLenti PGK V5-LUC Neo (w623-2); Addgene, plasmid #21471) were prepared, and 105 cells (50,000 cells/L) were injected into the fourth ventricle/pons of immunocompromised NOD-SCID-gamma (NSG) (strain 005557; The Jackson Laboratory), cold-anesthetized, postnatal day 3 mouse pups by injection through a 30-gauge burr hole (stereotactic coordinates: 3 mm posterior to the lambda suture and 3 mm deep).

Antisense oligonucleotides. PS-MOE-ASOs were purchased from IDT (Coralville, Iowa). ASOs synthesized in large scale for animal work were purified by HPLC. We dissolved the ASOs in water and diluted them in saline before use. A list of oligonucleotide sequences is provided in Supplementary Table Si. The ASOs tested in mice were: ASO5 (MOE/PS-DNA/PS-MOE/PS:5-10-5; GGCGCACTCATGCGAGCGGC (SEQ ID NO: 98)) and Control ASO (MOE/PS-DNA/PS-MOE/PS CCTTCCCTGAAGGTTCCTCC (SEQ ID NO: 93).

TABLE 3

| SEQ ID NO | Name | Sequence |
|---|---|---|
| 94 | ASO1 | CACTCATGCGAGCGGCTTTT |
| 95 | ASO2 | GCACTCATGCGAGCGGCTTT |
| 96 | ASO3 | CGCACTCATGCGAGCGGCTT |
| 97 | ASO4 | GCGCACTCATGCGAGCGGCT |
| 98 | ASO5 | GGCGCACTCATGCGAGCGGC |
| 99 | ASO6 | GGGCGCACTCATGCGAGCGG |
| 100 | ASO7 | AGGGCGCACTCATGCGAGCG |
| 101 | ASO8 | GAGGGCGCACTCATGCGAGC |
| 102 | ASO9 | AGAGGGCGCACTCATGCGAG |
| 103 | ASO10 | TAGAGGGCGCACTCATGCGA |
| 104 | ASO11 | GGGCAGTCTGCTTTGTACGA |
| 105 | ASO12 | CGATTTGCGGGCAGTCTACT |
| 106 | ASO13 | TACCACCGGTCGATTTGCGG |
| 107 | ASO14 | TTGCTTCCTGGGTGCTTTAC |
| 108 | ASO15 | GAGGTTTCTTCACCCCTCCA |

Primary pediatric human glioma cell lines. SU-DIPGAXIII, SU-DIPG-35, and SU-DIPG-50 patient cells heterozygous for the H3F3A mutation (A>T) and derived from autopsy tissue were obtained, in accordance with informed-consent protocols and in compliance with Stanford University and Cold Spring Harbor Laboratory Institutional Review Board human-subject protocols. The cells were grown as tumor neurospheres in tumor stem media (TSM) consisting of DMEM/1F12 (Invitrogen), Neurobasal (-A) (Invitrogen), B27 (-A) (Invitrogen), human-bFGF (20 ng/mL; Protech), human-EGF (20 ng/mL; Peprotech), human PDGF-AB (20 ng/mL; Peprotech), and heparin (10 ng/mL; Stemcell). The point mutation in H3F3A was confirmed by Sanger sequencing using primers listed in Table 4.

TABLE 4
Primers for gDNA H3F3A minigene

| SEQ ID NO: | Sequence |
|---|---|
| 78 | Fwd 5′-GGATCCGGCGGCGTGTGTTGGGGGATAGCCT |
| 79 | Rev 5′-CTTGAATTCTCACTGCAAAGCACCGATAGCTGC |

Primers for H3F3B

| SEQ ID NO: | Sequence |
|---|---|
| 80 | Fwd 5′-GTGCTGGTTTTTCGCTCGTC |
| 81 | Rev 5′-CTTTCGTGGCCAGCTGTTTG |

Primers for HPRT1

| SEQ ID NO: | Sequence |
|---|---|
| 82 | Fwd 5′-TGACCAGTCAACAGGGGACA |
| 83 | Rev 5′-TGCCTGACCAAGGAAAGCAA |

Primers for H3F3A mutation

| SEQ ID NO: | Sequence |
|---|---|
| 84 | Fwd 5′-AAGCAGACTGCCCGCAAA |

Primers for gDNA H3F3A minigene

| | |
|---|---|
| 85 | Rev 5′-CGCTCTGGAAGCGCA |

Lentivirus preparation and Infection. Two 20-nucleotide gRNA pairs against human H3F3A were annealed and cloned in the pSpCas9 (BB) vector (pX459; Addgene plasmid #62988) expressing Cas9 lentiviral constructs; Cas9-resistance gRNA pairs were annealed and cloned in the lentiV-neo vector (Addgene plasmid #108101). Lentiviral particles were generated by co-transfection of lentiviral-expressing constructs with packaging plasmids (pspAX2, VSV-G) into HEK-293T cells, and then concentrated by polyethylene glycol (PEG-it) precipitation (SBI). For lentiviral infection, dissociated DIPG cells were seeded on a 1% Matrigel-coated plate (Corning), and incubated with gRNA-expressing lentivirus for 12 hours before replacing with fresh medium. Puromycin (0.5 μg/ml) was added at 48 hours post-infection to select infected cells. After 7 days, puromycin was removed and the cells were allowed to recover in regular growth medium. Bulk cells were used by immunostaining, western blotting, and functional assays.

Transfection and free uptake of ASOs. HeLa cells were grown to 70-80% confluence in 12-well plates, and transfected for 3 days with 2 μL of Lipofectamine 2000 transfection reagent (Invitrogen) and different amounts of ASOs, ranging from 30 nM to 150 nM, following the manufacturer's recommendations. For free uptake of ASOs, patient cells were dissociated into single cells using TrypLE Express (Invitrogen), and 15,000/well cells were seeded in a 96-well plate and incubated at 37° C. for 1 hour; 4-10 μM ASO was then added for 3 to 5 days, cell medium was replaced with fresh medium, and a second ASO dose was added on day 3.

Cell viability and proliferation assay. Primary patient cells were starved in TSM base with B27 for 3 days. Then, 3,000 cells/well were plated in a 96-well plate in TSM base with normal growth medium with EGF, FGF, and PDGF-AB, as described (34). Cell viability and growth were measured using a cell-counting kit (CCK-8; Sigma #96992) at set time points.

Bioluminescence imaging. D-Luciferin was reconstituted as per the manufacturer's protocol (Goldbio, LUCK-100) and administered intraperitoneally (10 μg/g bodyweight) into isoflurane anesthetized animals, 12 minutes prior to imaging. Animals were excluded if no tumors were present, and the remaining animals were randomized into control and treatment groups with equivalent distribution of sex and initial tumor sizes.

Intracerebroventricular injection of ASOs. The presence of tumors was confirmed through luminescence imaging, as described above. Mice bearing tumors were then randomized and treated with a single ICV injection of CTRL ASO or ASO5 (500 μg for the RCAS-TVA mouse model; 200 μg for the orthotopic xenograft model) using a Hamilton syringe with a 28-gauge burr hole needle in isoflurane-anesthetized animals (stereotactic coordinates: 1.0 mm posterior to the bregma, 0.2 mm lateral, and 3 mm in depth).

Immunofluorescence and immunohistochemistry. Tumor tissue was fixed in 4% paraformaldehyde, cut into 5-μm sections, and embedded in paraffin. IHC was performed using heat-induced antigen retrieval with sodium citrate buffer, followed by primary antibodies to GFAP (1:1000; Millipore Sigma, rabbit polyclonal, AB5804), NeuN (1:100; Sigma, rabbit, monoclonal, 13E6), MBP (1:5000; Abcam, rabbit monoclonal, EPR21188), Ki67 (1:50; BD biosciences, mouse monoclonal, B56), Nestin (1:100, R&D, mouse monoclonal MAB2736), hSMN-KH (1:50, MABE230, mouse), Olig2 (1:500, Millipore, rabbit polyclonal) or H3K27me3 (1:1000; CST rabbit polyclonal, C36B11). For IHC, the signal was visualized with HRP-labeled anti-rabbit polyclonal (1:200, Agilent, P0448) and DAB (Agilent, K346711). Slides were counterstained with hematoxylin (Sigma) and captured on a Zeiss Observer microscope. For IF, the signal was visualized with a fluoro-conjugated secondary antibody (Thermo Fisher) and captured on a Zeiss LSM780 confocal laser-scanning microscope.

EdU staining assays. Primary human glioma cells (SU-DIPG-XIII, 5′103 cells/well) were seeded onto 1% matrigel-coated 8-well chamber slides (Falcon) and treated with 4 μM ASO by free uptake for five days. On day 5, 10 μM EdU was added to the cells, and incubated at 37° C. for 2 hours. EdU incorporation was measured using a Click-it Plus EdU Alexa Fluor 594 Imaging Kit (Invitrogen) in accordance with the manufacturer's instructions. Images were captured on a Zeiss Observer microscope. All images within the same figure panel were taken with the same exposure setting, and identically processed using Image J software.

Soft-agar assay. Primary human glioma cells (SU-DIPG-XIII, 103 cells/well) were incubated in an upper layer of 0.3% agar (ThermoFisher Scientific) in TSM. The bottom layer consists of the same medium with supplements, but 0.6% solidified basal agar, in a 12-well plate. Plates were incubated at 37° C./5% C02 for at least 3 weeks, before staining with crystal violet. Visible colonies were then counted.

RNA and protein extraction. Cells or tissues were harvested at the end points and snap-frozen in liquid nitrogen. For RNA extraction, 1 mL of Trizol (Invitrogen, 15596-018) was added to homogenized brain tissue or cells, following the standard Trizol protocol with chloroform extraction, isopropanol precipitation, and 70% EtOH RNA-pellet wash. RNA was resuspended in 20-40 μL of nuclease-free water. For protein extraction, cells or tissues were harvested and lysed on ice using Triton Extraction Buffer (TEB: PBS containing 0.5% Triton X 100 (v/v), protease inhibitor cocktail (Roche)), followed by centrifugation at 6,500×g for 10 minutes at 4° C. to spin down the nuclei; the supernatant was removed and discarded; the pellet was resuspended in 0.2 N HCl to perform acid extraction overnight; the supernatant was collected after centrifugation at 6,500×g for 10 minutes at 4° C.; and the protein concentration was measured by Bradford assay (Bio-Rad).

Radioactive RT-PCR and RT-qPCR. Total RNA was extracted from cells or tissues as described above, and reverse-transcribed with ImProm-II reverse transcriptase (Promega) using oligo-dT primers. Total H3F3A cDNA was amplified with AmpliTaq™ DNA polymerase (Thermo Fisher) using Fwd 5′-GGACTTTAAAACAGATCTGCGCTT (SEQ ID NO: 171) and Rev 5′-GTCTTTTGGCATAATTGTTACACGT (SEQ ID NO: 172) primers that sit on exon 3 and exon 4 (downstream of the H3F3A mutation site in exon 2), respectively. The H3F3A WT allele was amplified using Fwd 5′-GCTACAAAAGCCGCTCTCAA (SEQ ID NO: 173); the H3F3A mutant allele was amplified using Fwd 5′-GCTACAAAAGCCGCTCGAAT (SEQ ID NO: 175); and the same Rev 5′-CCAGACGCTGGAAGGGAAGT (SEQ ID NO: 175) primer was used for both mutant and WT allele amplification. cDNA from minigenes was amplified using vector-specific (pcDNA3.1) primers, listed in Table 4. For radioactive PCR, 0.16 μL of fresh [α-32P]-dCTP was added to a 20-μL PCR reaction. Amplicons were separated by 5% native PAGE (Bio-Rad), followed by phosphorimage analysis on a Typhoon 9410 phosphorimager (GE Healthcare). Band intensities were quantified using Image J, and the values normalized for the G+C content according to the DNA sequence. For RT-qPCR, 2×SYBR green master mix (Applied Biosystems) was used, and the cDNA was analyzed on a QuantStudio™ 6 Flex Real-Time PCR system (ThermoFisher Scientific). Fold changes were calculated using the AACq method.

Western Blotting. One microgram of acid-extracted protein was run on a 8-20% precast protein gel (Bio-Rad), transferred onto a nitrocellulose membrane, and probed with rabbit polyclonal anti-H3K27M (1:1000; ABE419, Millipore), rabbit monoclonal anti-H3K27me3 (1:1000; C36B11, CST) or rabbit polyclonal H3 (1:1000, ab1791, Abeam) antibodies. The membranes were incubated with infrared-dye-conjugated secondary antibodies (1:10000; LI-COR Biosciences), and protein bands were visualized by quantitative fluorescence using Odyssey software (LI-COR Biosciences). Molecular weight markers confirmed the sizes of the bands. Band intensities were quantified using Image J and normalized to total H3 protein.

Statistical analyses were performed with advice from a biostatistician. For immunoblots, RT-qPCR, and IHC/IF experiments, the measurements for each experimental group/treatment were analyzed by ANOVA, followed by pairwise comparisons using two-sample t-tests. For viability assays, P-values were adjusted for multiple comparisons by controlling for family-wise error rate using the single-step method. For in vivo experiments, the Kaplan-Meier estimator was used to calculate survival differences between cohorts using a log-rank test, median-survival rate, and hazard ratio. For RT-qPCR experiments, the measurements for each experimental group/treatment were analyzed by Welch's two-sample t-test to compare the H3F3A MUT expression normalized to the GAPDH loading control between CTRL ASO and ASO5 treatments. For IF quantification, cells were counted in 5 randomly picked fields at 40× magnification. The cell counts were analyzed by ANOVA, followed by t-tests for the pairwise comparisons. For all tests, p values of less than 0.05 were considered significant. Adjusted p-value calculations were employed for multiple-hypothesis correction. Significance codes: 0.001 ‘***’; 0.01 ‘**’; >0.05 ‘n.s.’.

### REFERENCES FOR EXAMPLES 5-13

1. Phillips, R. E, Soshnev, A. A, Allis, C. D. Epigenomic reprogramming as a driver of malignant glioma. Cancer Cell 38: 647-660 (2020).
2. http://www.danafarberbostonchildrens.org/conditions/brain-tumor/diffuse-pontineglioma.aspx
3. Lewis, P. W, Müller, M. M, Koletsky, M. S, Cordero, F., Lin, S., Banaszynski, L. A, Garcia, B. A, Muir, T. W, Becher, O. J, Allis, C. D. Inhibition of PRC2 activity by a gain-of-function H3 mutation found in pediatric glioblastoma. Science 340: 857-861 (2013).
4. Herz, H. M, Morgan, M., Gao, X., Jackson, J., Rickels, R., Swanson, S. K, Florens, L., Washburn, M. P, Eissenberg, J. C, Shilatifard, A. Histone H3 lysine-to-methionine mutants as a paradigm to study chromatin signaling. Science 345: 1065-1070 (2014).
5. Harutyunyan, A. S, Krug, B., Chen, H., Papillon-Cavanagh, S., Zeinieh, M., De Jay, N., Deshmukh, S., Chen, C C L., Belle, J., Mikael, L. G., Marchione, D. M., Li, R., Nikbakht, H., Hu, B., Cagnone, G., Cheung, W. A., Mohammadnia, A., Bechet, D., Faury, D., McConechy, M. K., Pathania, M., Jain, S. U., Ellezam, B., Weil, A. G., Montpetit, A., Salomoni, P., Pastinen, T., Lu, C., Lewis, P. W., Garcia, B. A., Kleinman, C. L., Jabado, N., Majewski, J. H3K27M induces defective chromatin spread of PRC2-mediated repressive H3K27me2/me3 and is essential for glioma tumorigenesis. Nat Commun 10: 1262. (2019)
6. Silveira, A. B., Kasper, L. H., Fan, Y., Jin, H., Wu, G., Shaw, T. I., Zhu, X., Larson, J. D., Easton, J., Shao, Y., Yergeau, D. A., Rosencrance, C., Boggs, K., Rusch, M. C., Ding, L., Zhang, J., Finkelstein, D., Noyes, R. M., Russell, B. L., Xu, B., Broniscer, A., Wetmore, C., Pounds, S. B., Ellison, D. W., Zhang, J., Baker, S. J. H3.3 K27M depletion increases differentiation and extends latency of diffuse intrinsic pontine glioma growth in vivo. Acta Neuropathol 137: 637-655 (2019).
7. Castel, D., Philippe, C., Calmon, R., Dret, L. L., Truffaux, N., Boddaert, N., Pages, M., Taylor, K. R., Saulnier, P., Lacroix, L., Mackay, A., Jones, C., Sainte-Rose, C., Blauwblomme, T., Andreiuolo, F., Puget, S., Grill, J., Varlet, P., Debily, M. A. Histone H3F3A and HIST1H3B K27M mutations define two subgroups of diffuse intrinsic pontine gliomas with different prognosis and phenotypes. Acta Neuropathol. 130:815-827 (2015).
8. Fontebasso, A. M., Papillon-Cavanagh, S., Schwartzentruber, J., Nikbakht, H., Gerges, N., Fiset, P. O., Bechet, D., Faury, D., De Jay, N., Ramkissoon, L. A., Corcoran, A., Jones, D. T., Sturm, D., Johann, P., Tomita, T., Goldman, S., Nagib, M., Bendel, A., Goumnerova, L., Bowers, D. C., Leonard, J. R., Rubin, J. B., Alden, T., Browd, S., Geyer, J. R., Leary, S., Jallo, G., Cohen, K., Gupta, N., Prados, M. D., Carret, A. S., Ellezam, B., Crevier, L., Klekner, A., Bognar, L., Hauser, P., Garami, M., Myseros, J., Dong, Z., Siegel, P. M., Malkin, H., Ligon, A. H., Albrecht, S., Pfister, S. M., Ligon, K. L., Majewski, J., Jabado, N., Kieran, M. W. Recurrent somatic mutations in ACVR1 in pediatric midline high-grade astrocytoma. Nat Genet 46: 462-466 (2014).
9. Grasso, C. S., Tang, Y., Truffaux, N., Berlow, N. E., Liu, L., Debily, M. A., Quist, M. J., Davis, L. E., Huang, E. C., Woo, P. J., Ponnuswami, A., Chen, S., Johung, T. B., Sun, W., Kogiso, M., Du, Y., Qi, L., Huang, Y., Hutt-Cabezas, M., Warren, K. E., Dret, L. L., Meltzer, P. S., Mao, H., Quezado, M., van Vuurden, D. G., Abraham, J., Fouladi, M., Svalina, M. N., Wang, N., Hawkins, C., Nazarian, J., Alonso, M. M., Raabe, E. H., Hulleman, E., Spellman, P. T., Li, X. N., Keller, C., Pal, R., Grill, J., Monje, M. Functionally defined therapeutic targets in diffuse intrinsic pontine glioma. Nat Med 21: 555-559 (2015).
10. Hua, Y., Vickers, T. A., Okunola, H. L., Bennett, C. F., Krainer, A. R. Antisense masking of an hnRNP A1/A2 intronic splicing silencer corrects SMN2 splicing in transgenic mice. Am J Hum Genet 82: 834-848 (2008).
11. Bennett, C. F., Kraner, A. R., Cleveland, D. W. Antisense oligonucleotide therapies for neurodegenerative diseases. Annu Rev Neurosci 42: 385-406 (2019).
12. Roberts, T. C., Langer, R. & Wood, M. J. A. Advances in oligonucleotide drug delivery. Nat Rev Drug Discov 19, 673-694 (2020).
13. Ahronian, L. G. & Lewis, B. C. In vivo delivery of RCAS virus to mice. Cold Spring Harb Protoc 2014: 1167-1169 (2014).
14. Hoeman, C. M., Cordero, F. J., Hu, G., Misuraca, K., Romero, M. M., Cardona, H. J., Nazarian, J., Hashizume, R., McLendon, R., Yu, P., Procissi, D., Gadd, S., Becher, O. J. ACVR1 R206H cooperates with H3.1K27M in promoting diffuse intrinsic pontine glioma pathogenesis. Nat Commun 10: 1023 (2019).
15. Cordero, F. J., Huang, Z., Grenier, C., He, X., Hu, G., McLendon, R. E., Murphy, S. K., Hashizume, R., Becher, O. J. Histone H3.3K27M represses p16 to accelerate gliomagenesis in a murine model of DIPG. Mol Cancer Res 15: 1243-1254 (2017).
16. Larso, J. D., Kasper, L. H., Paugh, B. S., Jin, H. J., Wu, G., Kwon, C. H., Fan, Y. P., Shaw, T. I., Silveira, A., Qu, C. X., Xu, R., Zhu, X. Y., Zhang, J. Y., Russel, H. R., Peters, J., Finkelstein, D., Xu, B. S., Lin, T., Tinkle, C. L., Patay, Z., Onar-Thomas, A., Pounds, S. B., McKinnon, P. J., Ellison, D. W., Zhang, J. H., Baker, S. J. Histone H3.3 K27M Accelerates Spontaneous Brainstem Glioma and Drives Restricted Changes in Bivalent Gene Expression. Cancer Cell 35, 140-155 (2019).
17. Hua, Y., Sahashi, K., Hung, G., Rigo, F., Passini, M. A., Bennett, C. F., Krainer, A. R. Antisense correction of SMN2 splicing in the CNS rescues necrosis in a type III SMA mouse model. Genes Dev 24: 1634-1644 (2010).
18. Hua, Y., Sahashi, K., Rigo, F., Hung, G., Horev, G., Bennett, C. F., Krainer, A. R. Peripheral SMN restoration is essential for long-term rescue of a severe spinal muscular atrophy mouse model. Nature 478: 123-126 (2011).
19. Liddelow, S. A., Guttenplan, K. A., Clarke, L. E., Bennett, F. C., Bohlen, C. J., Schirmer, L., Bennett, M. L., Munch, A. E., Chung, W. S., Peterson, T. C., Wilton, D. K., Frouin, A., Napier, B. A., Panicker, N., Kumar, M., Buckwalter, M. S., Rowitch, D. H., Dawson, V. L., Dawson, T. M., Stevens, B., Barres, B. A. Neurotoxic reactive astrocytes are induced by activated microglia. Nature 541: 481-487 (2017).
20. Qian, H., Kang, X., Hu, J., Zhang, D., Liang, Z., Meng, F., Zhang, X., Xue, Y., Maimon, R., Dowdy, S. F., Devaraj, N. K., Zhou, Z., Mobley, W. C., Cleveland, D. W., Fu, X. D. Reversing a model of Parkinson's disease with in situ converted nigral neurons. Nature 582: 550-556 (2020).
21. Makarava, N., Chen-Yu, C., Molesworth, K., Baskakov, I. V. Posttranslational modifications define course of prion strain adaptation and disease phenotype. J Clin Invest 130:4382-4395 (2020).
22. Filbin, M. G., Tirosh, I., Hovestadt, V., Shaw, M. L., Escalante, L. E., Mathewson, N. D., Neftel, C., Frank, N., Pelton, K., Hebert, C. M., Haberler, C., Yizhak, K., Gojo, J., Egervari, K., Mount, C., Galen, P. V., Bonal, D. M., Nguyen, Q. D., Beck, A., Sinai, C., Czech, T., Dorfer, C., Goumnerova, L., Lavarino, C., Carcaboso, A. M., Mora, J., Mylvaganam, R., Luo, C. C., Peyrl, A., Popović, M., Azizi, A., Batchelor, T. T., Frosch, M. P., Martinez-Lage, M., Kieran, M. W., Bandopadhayay, P., Beroukhim, R., Fritsch, G., Getz, G., Rozenblatt-Rosen, O., Wucherpfennig, K. W., Louis, D. N., Monje, M., Slavc, I., Ligon, K. L., Golub, T. R., Regev, A., Bernstein, B. E., Suva, M. L. Development and oncogenic programs in H3K27M gliomas dissected by single-cell-RNA-seq. Science 360: 331-335 (2018).
23. Monje, M., Mitra, S., Freret, M. E., Raveh, T. B., Kim, J., Masek, M., Attema, J. L., Li, G., Haddix, T., Edwards, M. S. B., Fisher, P. G., Weissman, I. L., Rowitch, D. H., Vogel, H., Wong, A. J., Beachy, P. A. Hedgehog-responsive candidate cell of origin for diffuse intrinsic pontine glioma. PNAS 108(11) 4453-4458 (2011).
24. Jang, C. W., Shibata, Y., Starmer, J., Yee, D., Magnuson, T. Histone H3.3 maintains genome integrity during mammalian development. Gene & Dev 29:1377-139 (2015).
25. Brown, Z. Z., Müller, M. M., Jain, S. U., Allis, C. D., Lewis, P. W., Muir, T. W. Strategy for “detoxification” of a cancer-derived histone mutant based on mapping its interaction with the methyltransferase PRC2. J. Am. Chem. Soc 136, 39, 13498-13501 (2014).
26. Haag, D., Mack, N., Silva, P. B. G. D., Statz, B., Clark, J., Tanabe, K., Sharma, T., Jager, N., Jones, D. T. W., Kawauchi, D., Wernig, M., Pfister, S. M. H3.3-K27M drives neural stem cell-specific gliomagenesis in a human iPSC-derived model. Cancer Cell 39(3): 407-422 (2021).
27. Laterra, J., Keeo, R., Betz, L. A., Goldstein, G. W. Blood-Cerebrospinal Fluid Barrier. Basic Neurochemistry: Molecular, Cellular and Medical Aspects (Lippincott Williams & Wilkins; 6th edition 1998).
28. Iadecola, C. The neurovascular unit coming of age: a journey through neurovascular coupling in health and disease. Neuron 96, 17-42 (2017).
29. Haruwaka, K., Ikegami, A., Tachibana, Y., Ohno, N., Konishi, H., Hashimoto, A., Matsumoto, M., Kato, D., Ono, R., Kiyama, H., Moorhouse, A. J., Nabekura, J., Wake, H. Dual microglia effects on blood brain barrier permeability induced by systemic inflammation. Nat Commun 10, 5816 (2019).
30. Wang, S. S., Bandopadhayay, P., Jenkins, M. R. Towards immunotherapy for pediatric brain tumors. Trends Immunol 40, 748-761 (2019).
31. Deland, K., Starr, B. F., Mercer, J. S., Byemerwa, J., Crabtree, D. M., Williams, N. T., Luo, L., Ma, Y., Chen, M., Becher, O. J., Kirsch, D. G. Tumor genotype dictates radiosensitization after Atm deletion in primary brainstem glioma models. J Clin Invest 131: e142158 (2021).
32. Mount, C. W., Majzner, R. G., Sundaresh, S., Arnold, E. P., Kadapakkam, M., Haile, S., Labanieh, L., Hulleman, E., Woo, P. J., Rietberg, S. P., Vogel, H., Monje, M., Mackall, C. L. Potent antitumor efficacy of anti-GD2 carT cells in H3-K27M+diffuse midline gliomas. Nat. Med 24, 572-579 (2018).
33. Majzner, R. G., Ramakrishna, S., Yeom, K. W., Patel, S., Chinnasamy, H., Schultz, L. M., Richards, R. M., Jiang, L., Barsan, V., Mancusi, R., Geraghty, A. C., Good, Z., Mochizuki, A. Y., Gillespie, S. M., Toland, A. M. S., Mahdi, J., Reschke, A., Nie, E. H., Chau, I. J., Rotiroti, M. C., Mount, C. W., Baggott, C., Mavroukakis, S., Egeler, E., Moon, J., Erickson, C., Green, S., Kunicki, M., Fujimoto, M., Ehlinger, Z., Reynolds, W., Sreevidya Kurra, S., Katherine, E., Warren, K. E., Prabhu, S., Vogel, H., Rasmussen, L., Cornell, T. T., Partap, S., Fisher, P. G., Campen, C. J., Filbin, M., Grant, G., Sahaf, B., Davis, K. L., Feldman, S. A., Crystal, L., Mackall, C. L. & Monje, M. GD2-CAR T cell therapy for H3K27M-mutated diffuse midline gliomas. Nature 603, 934-941 (2022).
34. Nagaraja, S., Quezada, M A., Gillespie, S M., Arzt, M., Lennon, J. J., Woo, P. J., Hovestadt, V., Kambhampati, M., Filbin, M. G., Suva, M. L., Nazarian, J., Monje, M. Histone variant and cell context determine H3K27M reprogramming of the enhancer landscape and oncogenic state. Molecular Cell 76, 965-980 (2019).

### EQUIVALENTS AND SCOPE

Those skilled in the art will recognize or be able to ascertain using no more than routine experimentation many equivalents to the specific embodiments described herein. The scope of the present invention is not intended to be limited to the above Description, but rather is as set forth in the appended claims.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim.

For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All references cited herein, including patents, published patent applications, and non-patent publications, are incorporated by reference in their entirety.

### Other Embodiments

Embodiment 1. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof, and does not hybridize to a H3.3 histone B (H3F3B) allele, or a product thereof, (b) comprises one or more chemical modification(s), and (c) is from about 10 to about 40 nucleosides.

Embodiment 2. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof, more than it hybridizes to a corresponding wild-type H3F3A allele, or a product thereof, or to a H3.3 histone B (H3F3B) allele, or a product thereof, (b) comprises one or more chemical modification(s), and (c) is from about 10 to about 40 nucleosides.

Embodiment 3. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof, and does not hybridize to a H3.3 histone B (H3F3B) gene, or a product thereof, (b) comprises one or more chemical modification(s), and (c) is from about 10 to about 40 nucleosides.

Embodiment 4. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof, more than it hybridizes to a H3.3 histone B (H3F3B) gene, or a product thereof, (b) comprises one or more chemical modification(s), and (c) is from about 10 to about 40 nucleosides.

Embodiment 5. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof, and does not hybridize to a H3.3 histone B (H3F3B) allele, or a product thereof, and (b) is a gapmer ASO comprising a 3′-wing, a gap segment and a 5′-wing, wherein the gap segment comprises DNA and one or more chemical modification(s) in one more internucleoside linkage(s) of the nucleic acid sequence.

Embodiment 6. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof, more than it hybridizes to a corresponding wild-type H3F3A allele, or a product thereof, or to a H3.3 histone B (H3F3B) allele, or a product thereof, and (b) is a gapmer ASO comprising a 3′-wing, a gap segment and a 5′-wing, wherein the gap segment comprises DNA and one or more chemical modification(s) in one more internucleoside linkage(s) of the nucleic acid sequence.

Embodiment 7. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof, and does not hybridize to a H3.3 histone B (H3F3B) gene, or a product thereof, and (b) is a gapmer ASO comprising a 3′-wing, a gap segment and a 5′-wing, wherein the gap segment comprises DNA and one or more chemical modification(s) in one more internucleoside linkage(s) of the nucleic acid sequence.

Embodiment 8. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof, more than it hybridizes to a H3.3 histone B (H3F3B) gene, or a product thereof, and (b) is a gapmer ASO comprising a 3′-wing, a gap segment and a 5′-wing, wherein the gap segment comprises DNA and one or more chemical modification(s) in one more internucleoside linkage(s) of the nucleic acid sequence.

Embodiment 9. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof and does not hybridize to a H3.3 histone B (H3F3B) allele, or a product thereof, and (b) is a splice-modulating ASO.

Embodiment 10. An antisense oligonucleotide (ASO) comprising a nucleic acid sequence complementary to a region that comprises a mutation in a mutant H3.3 histone A (H3F3A) allele, or a product thereof, wherein the ASO (a) hybridizes to the mutant H3F3A allele, or a product thereof more than it hybridizes to than a corresponding wild-type H3F3A allele, or a product thereof, or to a H3.3 histone B (H3F3B) allele, or a product thereof, and (b) is a splice-modulating ASO.

Embodiment 11. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof and does not hybridize to a H3.3 histone B (H3F3B) gene, or a product thereof, and (b) is a splice-modulating ASO.

Embodiment 12. An antisense oligonucleotide (ASO) comprising a nucleic acid sequence complementary to a region that comprises a mutation in a mutant H3.3 histone A (H3F3A) gene, or a product thereof, wherein the ASO (a) hybridizes to the mutant H3F3A gene, or a product thereof, more than it hybridizes to a H3.3 histone B (H3F3B) gene, or a product thereof, and (b) is a splice-modulating ASO.

Embodiment 13. The ASO of any one of embodiments 1-12, wherein the ASO comprises one or more chemical modification(s) on one or more nucleoside(s) of the ASO.

Embodiment 14. The ASO of embodiment 1 or embodiment 13, wherein the one or more chemical modification(s) is 2′-O-methoxyethyl (MOE) modification, a locked nucleic acid (LNA) modification, S-constrained ethyl (cET) modification, a phosphorodiamidate (PDA) morpholino oligomer (PMO) modification, or a 5′-methylcytosine modification.

Embodiment 15. The ASO of any one of embodiments 1, 13 or 14, wherein the ASO comprises one or more chemical modification(s) in one or more internucleoside linkage(s) of the nucleic acid sequence.

Embodiment 16. The ASO of embodiment 15, wherein the one or more chemical modification(s) is a phosphorothioate (PS) modification.

Embodiment 17. The ASO of embodiment 16, wherein all of the internucleoside linkages comprise PS modifications.

Embodiment 18. The ASO of any one of embodiments 5-17, wherein the ASO is from about 10 to about 40 nucleosides.

Embodiment 19. The ASO of any one of embodiments 1 or 13-17, wherein the ASO is a gapmer ASO comprising a 3′-wing, a gap segment and a 5′-wing, and the one or more chemical modification(s) is on one or more nucleoside(s) of the 3′-wing; on one or more nucleoside(s) of the 5′-wing; or on one or more nucleoside(s) of the 3′-wing and one or more nucleoside(s) of the 5′-wing.

Embodiment 20. The ASO of embodiment 19, wherein the 3′-wing is from about 5 to about 10 nucleosides.

Embodiment 21. The ASO of any one of embodiments 19-21, wherein the 5′-wing is from about 5 to about 10 nucleosides.

Embodiment 22. The ASO of any one of embodiments 19-21, wherein the gap segment is from about 5 to about 20 nucleosides.

Embodiment 23. The ASO of any one of embodiments 1-22, wherein the ASO is from about 10 to about 30 nucleosides.

Embodiment 24. The ASO of any one of embodiments 1-22, wherein the ASO is about 20 nucleosides.

Embodiment 25. The ASO of any one of embodiments 1 or 13-24, wherein the ASO is a splice-modulating ASO.

Embodiment 26. The ASO of any one of embodiments 1-25, wherein the mutation in a mutant H3F3A allele is at position 2604 of the nucleic acid sequence of SEQ ID NO: 89.

Embodiment 27. The ASO of any one of embodiments 1-26, wherein the mutant H3F3A allele encodes a mutant histone 3.3 (H3.3) protein comprising a Lys to Met mutation.

Embodiment 28. The ASO of embodiment 27, wherein the Lys to Met mutation is at position 27 of the amino acid sequence of SEQ ID NO: 91.

Embodiment 29. The ASO of any one of embodiments 1-28, wherein the region is within exon 2.

Embodiment 30. The ASO of any one of embodiments 1-29, wherein the region is within the nucleic acid sequence of SEQ ID NO: 88.

Embodiment 31. The ASO of any one of embodiments 1-30, wherein the ASO comprises the nucleic acid sequence CACTCATGCGAGCGGCTTTT (SEQ ID NO: 1), GCGCACTCATGCGAGCGGCT (SEQ ID NO: 4), GGCGCACTCATGCGAGCGGC (SEQ ID NO: 5), GGGCGCACTCATGCGAGCGG (SEQ ID NO: 6), ACCCCTCCAGTAGAGGGCGC (SEQ ID NO: 58), CAGTAGAGGGCGCACTCATG (SEQ ID NO: 59), or AGTAGAGGGCGCACTCATGC (SEQ ID NO: 60).

Embodiment 32. The ASO of any one of embodiments 1-30, wherein the ASO consists of the nucleic acid sequence CACTCATGCGAGCGGCTTTT (SEQ ID NO: 1), GCGCACTCATGCGAGCGGCT (SEQ ID NO: 4), GGCGCACTCATGCGAGCGGC (SEQ ID NO: 5), GGGCGCACTCATGCGAGCGG (SEQ ID NO: 6), ACCCCTCCAGTAGAGGGCGC (SEQ ID NO: 58), CAGTAGAGGGCGCACTCATG (SEQ ID NO: 59), or AGTAGAGGGCGCACTCATGC (SEQ ID NO: 60).

Embodiment 33. The ASO of any one of embodiments 1-30, wherein the ASO comprises the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

Embodiment 34. The ASO of any one of embodiments 1-30, wherein the ASO consists of the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

Embodiment 35. The ASO of any one of embodiments 1-34, wherein the product thereof is pre-mRNA or mRNA.

Embodiment 36. The ASO of any one of embodiments 1-35, wherein the ASO reduces the expression of the mutant H3F3A allele and does not reduce the expression of the H3F3B allele.

Embodiment 37. The ASO of any one of embodiments 1-36, wherein the ASO is a single-stranded ASO.

Embodiment 38. The ASO of any one of embodiments 1-37, wherein the mutant H3F3A allele is a dominant mutation that encodes a point mutation in non-canonical H3.3 protein found in/characteristic of pediatric diffuse midline gliomas.

Embodiment 39. An antisense oligonucleotide (ASO) comprising a nucleic acid sequence complementary to a region that comprises a mutation in a mutant H3.3 histone A (H3F3A) allele, or a product thereof, wherein the ASO comprises the nucleic acid sequence of any one of SEQ ID NOs: 1-77.

Embodiment 40. An antisense oligonucleotide (ASO) comprising a nucleic acid sequence complementary to a region that comprises a mutation in a mutant H3.3 histone A (H3F3A) allele, or a product thereof, wherein the ASO comprises the nucleic acid sequence of any one of SEQ ID NOs: 94-170.

Embodiment 41. The ASO of any one of embodiments 9-41, wherein the ASO hybridizes under physiological conditions.

Embodiment 42. A method of reducing mutant-allele specific expression in a cell comprising contacting a cell with an effective amount of an antisense oligonucleotide (ASO) comprising a nucleic acid sequence complementary to a region that comprises a mutation in a mutant-allele, or a product thereof, wherein the ASO hybridizes to the mutant-allele, or to a product thereof, and does not hybridize to an allele that does not comprise the mutation, or to a product thereof, to reduce mutant-allele specific expression, or a product thereof, in the cell.

Embodiment 43. A method of reducing mutant-allele specific expression in a cell comprising contacting a cell with an effective amount of an antisense oligonucleotide (ASO) comprising a nucleic acid sequence complementary to a region that comprises a mutation in a mutant-allele, or a product thereof, wherein the ASO hybridizes to the mutant-allele, or to a product thereof, more than it hybridizes to an allele that does not comprise the mutation, or a product thereof, to reduce mutant-allele specific expression, or a product thereof, in the cell.

Embodiment 44. A method of reducing expression of a mutant H3.3 histone A (H3F3A) allele in a cell comprising contacting a cell with an effective amount of a splice-modulating antisense oligonucleotide (ASO) comprising a nucleic acid sequence complementary to a region that comprises a mutation in a mutant H3F3A allele, or a product thereof, to reduce expression of the mutant H3F3A allele, or a product thereof, in the cell.

Embodiment 45. A method of reducing expression of a H3.3 histone A (H3F3A) gene in a cell comprising contacting a cell with an effective amount of a splice-modulating antisense oligonucleotide (ASO) comprising a nucleic acid sequence complementary to a region of an H3F3A gene, or a product thereof, to reduce expression of the H3F3A gene, or a product thereof, in the cell.

Embodiment 46. A method of increasing cleavage and subsequent degradation of a mutant H3.3 histone A (H3F3A) allele in a cell comprising contacting a cell with an effective amount of a gapmer antisense oligonucleotide (ASO) comprising a nucleic acid sequence complementary to a region that comprises a mutation in a mutant H3F3A allele, or a product thereof, to increase RNase H-mediated cleavage and subsequent degradation of the mutant H3F3A allele, or a product thereof, in the cell.

Embodiment 47. A method of increasing cleavage and subsequent degradation of a mutant H3.3 histone A (H3F3A) gene in a cell comprising contacting a cell with an effective amount of a gapmer antisense oligonucleotide (ASO) comprising a nucleic acid sequence complementary to a region in an H3F3A gene, or a product thereof, to increase RNase H-mediated cleavage and subsequent degradation of the H3F3A gene, or a product thereof, in the cell.

Embodiment 48. A method of reducing expression of a mutant-allele with a gain-of-function mutation in a cell comprising contacting a cell with an effective amount of a splice-modulating antisense oligonucleotide (ASO) comprising a nucleic acid sequence complementary to a region of a mutant-allele with a gain-of-function mutation, or a product thereof, under conditions under which skipping of an exon occurs to reduce expression of the mutant-allele with a gain-of-function mutation, or a product thereof, in the cell.

Embodiment 49. The method of 48, wherein the exon comprises a mutation that produces the mutant-allele with a gain-of-function mutation.

Embodiment 50. The method of 48, wherein the exon does not comprise a mutation that produces the mutant-allele with a gain-of-function mutation.

Embodiment 51. A method of reducing expression of a mutant-allele with a gain-of-function mutation in a cell comprising contacting a cell with an effective amount of a gapmer antisense oligonucleotide (ASO) comprising a nucleic acid sequence complementary to a region of a mutant-allele with a gain-of-function mutation, or a product thereof, to reduce expression of the mutant-allele with a gain-of-function mutation, or a product thereof, via RNase H-mediated cleavage and subsequent degradation in the cell.

Embodiment 52. The method of any one of embodiments 42-51, wherein the cell is a brain cell.

Embodiment 53. The method of any one of embodiments 42-51, wherein the cell is a brain tumor cell.

Embodiment 54. The method of any one of embodiments 42-51, wherein the cell is a midline glioma cell.

Embodiment 55. The method of any one of embodiments 42-51, wherein the cell is a cell from the thalamus.

Embodiment 56. The method of any one of embodiments 42-51, wherein the cell is a cell from the midbrain.

Embodiment 57. The method of any one of embodiments 42-51, wherein the cell is a cell from the pons.

Embodiment 58. The method of any one of embodiments 42-57, wherein the cell is in an individual.

Embodiment 59. The method of embodiment 58, wherein the individual is a mammal.

Embodiment 60. The method of embodiment 58, wherein the individual is a human.

Embodiment 61. The method of any one of embodiments 42-60, wherein the ASO is an ASO of any one of embodiments 1-601-48.

Embodiment 62. A method of treating pediatric high-grade glioma (pHHG) comprising administering to an individual with pHHG an effective amount of an ASO of any one of embodiments A1-C30 1-48 to treat the individual with pHHG.

Embodiment 63. The method of embodiment 62, wherein the pHHG is a diffuse midline glioma (DMG).

Embodiment 64. The method of embodiment 62, wherein the pHHG is a diffuse intrinsic pontine glioma (DIPG).

Embodiment 65. The method of any one of embodiments 62-64, wherein the individual is a mammal.

Embodiment 66. The method of any one of embodiments 62-64, wherein the individual is a human.

Embodiment 67. The method of any one of embodiments 62-66, wherein administration of the ASO ameliorates or eliminates a characteristic and/or symptom associated with pHHG.

Embodiment 68. The method of any one of embodiments 62-67, wherein the ASO is administered via intrathecal administration or via an Ommaya reservoir.

Embodiment 69. The method of any one of embodiments 62-67, wherein the ASO is administered via systemic administration.

Embodiment 70. The method of any one of embodiments 62-69, wherein administration of the ASO reduces the expression of a mutant H3.3 protein relative to a reference.

Embodiment 71. The method of any one of embodiments 62-70, wherein the effective amount is about 1 mg to about 100 mg.

Embodiment 72. A method of treating a disease or condition comprising administering to an individual with a disease or condition associated with a gain-of-function mutation in a mutant histone 3.3 (H3.3) protein an effective amount of an ASO of any one of embodiments 1-41 to treat the individual with the disease or condition associated with a gain-of-function mutation in a mutant H3.3 protein, wherein the gain-of-function mutation prevents methylation of a position associated with a gain-of-function mutation in the mutant H3.3 protein.

Embodiment 73. The method of embodiment 72, wherein the gain-of-function mutation prevents di-methylation or tri-methylation of the position associated with a gain-of-function mutation in the mutant H3.3 protein.

Embodiment 74. The method of embodiment 72, wherein the position is position 27 in the mutant H3.3 protein.

Embodiment 75. The method of embodiment 72 or embodiment 74, wherein the individual is a mammal.

Embodiment 76. The method of embodiment 72 or embodiment 74, wherein the individual is a human.

Embodiment 77. The method of any one of embodiments 72-76, wherein administration of the ASO ameliorates or eliminates a characteristic and/or symptom associated with the disease or condition associated with a gain-of-function mutation in a mutant H3.3 protein.

Embodiment 78. The method of any one of embodiments 72-77, wherein the ASO is administered via intrathecal administration or via an Ommaya reservoir.

Embodiment 79. The method of any one of embodiments 72-77, wherein the ASO is administered via systemic administration.

Embodiment 80. The method of any one of embodiments 72-79, wherein the disease or condition associated with a gain-of-function mutation in a mutant H3.3 protein is a disease or condition associated with H3K36me3 or H3G34R/V mutation.

Embodiment 81. The method of any one of embodiments 72-79, wherein the disease or condition associated with a gain-of-function mutation in a mutant H3.3 protein is cancer.

Embodiment 82. The method of any one of embodiments 42-81, wherein the ASO reduces the expression of the mutant H3F3A allele and does not reduce the expression of the H3F3B allele.

Embodiment 83. The method of any one of embodiments 42-82, wherein expression of the mutant H3F3A allele is reduced to a greater extent than expression of the corresponding wild-type H3F3A allele.

Embodiment 84. The method of any one of embodiments 42-82, wherein the ASO reduces the expression of the mutant H3F3A gene and does not reduce the expression of the H3F3B gene.

Embodiment 85. The method of any one of embodiments 42-82, wherein expression of the mutant H3F3A gene is reduced to a greater extent than expression of the H3F3B gene is reduced.

What is claimed is:

1. An antisense oligonucleotide (ASO) of about 15 nucleosides to about 30 nucleosides, wherein the nucleic acid sequence of the ASO is identical to the sequence of any one of SEQ ID NOs.: 1-15, and comprises one or more nucleoside chemical modifications, or an ASO of about 15 nucleosides to about 30 nucleosides, wherein the nucleic acid sequence of the ASO is at least 70% identical to the nucleic acid sequence of any one of SEQ ID NOs.: 1-15, and comprises one or more nucleoside chemical modifications.

2. The ASO of claim 1, wherein the nucleic acid sequence of about 15 nucleosides to about 30 nucleosides is at least 80%, at least 95%, or at least 99% identical to the nucleic acid sequence of any one of SEQ ID NOs.: 1-15.

3. The ASO of claim 1, wherein the nucleic acid sequence of about 15 nucleosides to about 30 nucleosides is 100% identical to the nucleic acid sequence of any one of SEQ ID NOs.: 1-15.

4. An antisense oligonucleotide (ASO) comprising a nucleic acid sequence of about 15 to about 30 nucleosides that is at least 80% complementary to a region of the nucleic acid sequence ATGGCTCGTACAAAGCAGACTGCCCGCAAATCGACCGGTGGTAAAGCACCCAGG AAGCAACTGGCTACAAAAGCCGCTCGCATGAGTGCGCCCTCTACTGGAGGGGTG AAGAAACCTCATCGTTACAG (SEQ ID NO: 87), wherein the ASO comprises one or more nucleoside chemical modifications.

5. The ASO of claim 4, which comprises a nucleic acid sequence of about 15 nucleosides to about 30 nucleosides is at least 95%, or at least 99% complementary to a region of the nucleic acid sequence
(SEQ ID NO: 87)
ATGGCTCGTACAAAGCAGACTGCCCGCAAATCGACCGGTGGTAAAGCAC
CCAGGAAGCAACTGGCTACAAAAGCCGCTCGCATGAGTGCGCCCTCTAC
TGGAGGGGTGAAGAAACCTCATCGTTACAG.

6. The ASO of claim 4, which comprises a nucleic acid sequence of about 15 nucleosides to about 30 nucleosides 100% complementary to a region of the nucleic acid sequence
(SEQ ID NO: 87)
ATGGCTCGTACAAAGCAGACTGCCCGCAAATCGACCGGTGGTAAAGCAC
CCAGGAAGCAACTGGCTACAAAAGCCGCTCGCATGAGTGCGCCCTCTAC
TGGAGGGGTGAAGAAACCTCATCGTTACAG.

7. An antisense oligonucleotide (ASO) comprising a nucleic acid sequence of about 15 nucleosides to about 30 nucleosides that is at least 80% complementary to a region of the nucleic acid sequence ACTGGCTACAAAAGCCGCTCGCATGAGTGCGCCCTCTACTGGAGGGGTGAAGAA ACCTCATC (SEQ ID NO: 88), wherein the ASO comprises one or more nucleoside chemical modifications.

8. The ASO of claim 7, which comprises a nucleic acid sequence from about 15 to about 30 nucleosides is at least 95%, or at least 99% complementary to the nucleic acid sequence
(SEQ ID NO: 88)
ACTGGCTACAAAAGCCGCTCGCATGAGTGCGCCCTCTACTGGAGGGGTG
AAGAAACCTCATC.

9. The ASO of claim 7, which comprises a nucleic acid sequence from about 15 to about 30 nucleosides is 100% complementary to the nucleic acid sequence
(SEQ ID NO: 88)
ACTGGCTACAAAAGCCGCTCGCATGAGTGCGCCCTCTACTGGAGGGGTG
AAGAAACCTCATC.

10. An antisense oligonucleotide (ASO) of about 15 nucleosides to about 30 nucleosides, wherein the nucleic acid sequence of the ASO of about 15 nucleosides to about 30 nucleosides is complementary to a region of a mutant H3.3 histone A (H3F3A) allele that comprises a mutation in exon 2 and the ASO of about 15 nucleosides to about 30 nucleosides comprises one or more nucleoside chemical modifications.

11. An antisense oligonucleotide (ASO) of about 15 nucleosides to about 30 nucleosides, wherein the nucleic acid sequence of the ASO of about 15 nucleosides to about 30 nucleosides is complementary to a region of a mutant H3.3 histone A (H3F3A) allele that comprises a mutation in exon 2, wherein the ASO of about 15 nucleosides to about 30 nucleosides hybridizes to the mutant H3F3A allele, and does not hybridize to a H3.3 histone B (H3F3B) allele.

12. An antisense oligonucleotide (ASO) of about 15 nucleosides to about 30 nucleosides, wherein the nucleic acid sequence of the ASO of about 15 nucleosides to about 30 nucleosides is complementary to a region of a mutant H3.3 histone A (H3F3A) allele that comprises a mutation in exon 2, wherein the ASO of about 15 nucleosides to about 30 nucleosides hybridizes to the mutant H3F3A allele more than it hybridizes to a corresponding wild-type H3F3A allele, or to a H3.3 histone B (H3F3B) allele.

13. An antisense oligonucleotide (ASO) of about 15 nucleosides to about 30 nucleosides, wherein the nucleic acid sequence of the ASO of about 15 nucleosides to about 30 nucleosides is complementary to a region of a mutant H3.3 histone A (H3F3A) gene that comprises a mutation in exon 2, wherein the ASO of about 15 nucleosides to about 30 nucleosides hybridizes to the mutant H3F3A gene more than it hybridizes to a H3.3 histone B (H3F3B) gene.

14. The ASO of any one of claims 10-13, wherein the mutant H3F3A allele encodes a mutant histone 3.3 (H3.3) protein comprising a lysine (K) to methionine (M) mutation.

15. The ASO of any one of claims 10-14, wherein the ASO of about 15 nucleosides to about 30 nucleosides comprises one or more nucleoside chemical modifications.

16. The ASO of any one of claims 1-3 or 10, wherein the ASO of about 15 nucleosides to about 30 nucleosides is from about 18 nucleosides to about 22 nucleosides.

17. The ASO of any one of claims 1-16, wherein the ASO of about 15 nucleosides to about 30 nucleosides is a gapmer ASO of about 15 nucleosides to about 30 nucleosides comprising a 3′-wing, a gap segment and a 5′-wing, wherein the gap segment comprises DNA and one or more chemical modifications in one more internucleoside linkages of the nucleic acid sequence.

18. The ASO of any one of claims 1-16, wherein the ASO of about 15 nucleosides to about 30 nucleosides is a splice-modulating ASO.

19. The ASO of any one of claims 1-18, wherein the one or more nucleoside chemical modifications are a 2′-O-methoxyethyl (MOE) modification, a locked nucleic acid (LNA) modification, a S-constrained ethyl (cET) modification, a phosphorodiamidate (PDA) morpholino oligomer (PMO) modification, or a 5′-methylcytosine modification.

20. The ASO of any one of claims 1-19, wherein the ASO of about 15 nucleosides to about 30 nucleosides comprises one or more chemical modifications in one or more internucleoside linkages of the nucleic acid sequence.

21. The ASO of claim 20, wherein the one or more chemical modifications in one or more internucleoside linkages comprise a phosphorothioate (PS) modification.

22. The ASO of claim 21, wherein all of the internucleoside linkages comprise PS modifications.

23. The ASO of any one of claims 11-22, wherein the ASO of about 15 nucleosides to about 30 nucleosides is from about 15 nucleosides to about 25 nucleosides.

24. The ASO of any one of claims 1-23, wherein the ASO of about 15 nucleosides to about 30 nucleosides is a gapmer ASO of about 15 nucleosides to about 30 nucleosides comprising a 3′-wing, a gap segment and a 5′-wing, and the one or more nucleoside chemical modifications is on one or more nucleosides of the 3′-wing; on one or more nucleosides of the 5′-wing; or on one or more nucleosides of the 3′-wing and one or more nucleosides of the 5′-wing.

25. The ASO of claim 24, wherein the 3′-wing is from about 5 nucleosides to about 10 nucleosides.

26. The ASO of claim 24 or claim 25, wherein the 5′-wing is from about 5 nucleosides to about 10 nucleosides.

27. The ASO of any one of claims 24-26, wherein the gap segment is from about 5 nucleosides to about 20 nucleosides.

28. The ASO of any one of claims 1-27, wherein the ASO of about 15 nucleosides to about 30 nucleosides is from about 19 nucleosides to about 21 nucleosides.

29. The ASO of any one of claims 1-27, wherein the ASO of about 15 nucleosides to about 30 nucleosides is about 20 nucleosides.

30. The ASO of any one of claims 1-23, wherein the ASO of about 15 nucleosides to about 30 nucleosides is a splice-modulating ASO.

31. The ASO of any one of claims 1-30, wherein the mutation in a mutant H3F3A allele is at position 2604 of the nucleic acid sequence of SEQ ID NO: 89.

32. The ASO of any one of claims 1-31, wherein the nucleoside chemical modification is a 2′-MOE modification.

33. The ASO of any one of claims 14-32, wherein the K to M mutation is at position 27 of the amino acid sequence of SEQ ID NO: 91.

34. The ASO of any one of claims 10-33, wherein the region is within the nucleic acid sequence
(SEQ ID NO: 88)
ACTGGCTACAAAAGCCGCTCGCATGAGTGCGCCCTCTACTGGAGGGGTG
AAGAAACCTCATC.

35. The ASO of any one of claims 1-34, wherein the ASO comprises the nucleic acid sequence CACTCATGCGAGCGGCTTTT (SEQ ID NO: 1), GCGCACTCATGCGAGCGGCT (SEQ ID NO: 4), GGCGCACTCATGCGAGCGGC (SEQ ID NO: 5), GGGCGCACTCATGCGAGCGG (SEQ ID NO: 6), ACCCCTCCAGTAGAGGGCGC (SEQ ID NO: 58), CAGTAGAGGGCGCACTCATG (SEQ ID NO: 59), or AGTAGAGGGCGCACTCATGC (SEQ ID NO: 60).

36. The ASO of any one of claims 1-34, wherein the ASO consists of the nucleic acid sequence CACTCATGCGAGCGGCTTTT (SEQ ID NO: 1), GCGCACTCATGCGAGCGGCT (SEQ ID NO: 4), GGCGCACTCATGCGAGCGGC (SEQ ID NO: 5), GGGCGCACTCATGCGAGCGG (SEQ ID NO: 6), ACCCCTCCAGTAGAGGGCGC (SEQ ID NO: 58), CAGTAGAGGGCGCACTCATG (SEQ ID NO: 59), or AGTAGAGGGCGCACTCATGC (SEQ ID NO: 60).

37. The ASO of any one of claims 4-34, wherein the ASO comprises the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

38. The ASO of any one of claims 4-34, wherein the ASO consists of the nucleic acid sequence of CACTCATGCGAGCGGCTTTT with the first 5 nucleosides and last 5 nucleosides each comprising a 2′-MOE modification and all internucleoside linkages each comprising a PS modification (SEQ ID NO: 94), GCGCACTCATGCGAGCGGCT with the first 5 nucleosides and last 5 nucleosides each comprising a 2′-MOE modification and all internucleoside linkages each comprising a PS modification (SEQ ID NO: 97), GGCGCACTCATGCGAGCGGC with the first 5 nucleosides and last 5 nucleosides each comprising a 2′-MOE modification and all internucleoside linkages each comprising a PS modification (SEQ ID NO: 98), GGGCGCACTCATGCGAGCGG with the first 5 nucleosides and last 5 nucleosides each comprising a 2′-MOE modification and all internucleoside linkages each comprising a PS modification (SEQ ID NO: 99), ACCCCTCCAGTAGAGGGCGC with each nucleoside comprising a 2′-MOE modification and all internucleoside linkages each comprising a PS modification (SEQ ID NO: 151), CAGTAGAGGGCGCACTCATG with each nucleoside comprising a 2′-MOE modification and all internucleoside linkages each comprising a PS modification (SEQ ID NO: 152), or AGTAGAGGGCGCACTCATGC with each nucleoside comprising a 2′-MOE modification and all internucleoside linkages each comprising a PS modification (SEQ ID NO: 153).

39. The ASO of any one of claims 1-38, wherein the ASO reduces expression of the mutant H3F3A allele and does not reduce expression of the H3F3B allele.

40. The ASO of any one of claims 1-39, wherein the ASO is a single-stranded ASO.

41. The ASO of any one of claims 1-40, wherein the mutant H3F3A allele is a dominant mutation that encodes a point mutation in non-canonical H3.3 protein found in/characteristic of pediatric diffuse midline gliomas.

42. The ASO of any one of claims 11-41, wherein the ASO hybridizes under physiological conditions.
